SEQUENCE LISTING (159 sequences)

SEQ ID NO: 1 - DNA, 608 bp, Homo sapiens
  misc_feature: Incyte ID No LG223939.12000FEB18
gtgaaataatggaattagtcctggtgaaataccagggcaaaaactggaatggacatttccgcatacgtgatacactaccagaattctttcctgtgtgtttttcttctgactccacagaagtgacgacagtcgacctgtcagtccacgtcaggagaattggcagccggatggtgctgtctgtctttagtccctattggttaatcaacaagactacccgggttctccagtatcgttcagaagatattcatgtgaaacatccagctgatttcagggatattattttattttctttcaagaagaagaacatttttactaaaaataaggtacaattaaaaatttcaaccagtgcctggtccagtagtttctcattggatacagtgggaagttatgggtgtgtgaagtgtcctgccaacaatatggagtacctggttggtgttagcatcaaaatgagcagtttcaacctttcacgaatagttaccctgactcccttttgtaccattgcaaacaagtcatcattagaactagaagttggcgagattgcatctgatggctcaatgccaactaataaatggaactatattgcttcttcagagtgccttccattttgg

SEQ ID NO: 2 - DNA, 755 bp, Homo sapiens
  misc_feature: Incyte ID No LG397140.12000FEB18
tatggaaatgcactttatatttatttgcacacatcaagaatacttttggaaaccaggatttagaaaaataaaagatatttctttcctggggacaaagtgacctgcaaatgattggcttctgtacaatgttgcaatcccttaatgtcagactcttaggaaaatgggtgggacatctgtgtgtttgtgttggagtgaatgtgcccataaccctgtgctcccataagtctgtgtgcccgtgatcatatgcatatgggtgaatgtttgtgtgcccatgattttatgcatataggtgaatatttgtgtgcccatgagcatatgcatatgggtgaatgtttgtgtgcccatgagtatatgcatacgggtgaatgtttgtgtgcccatgagtatatgcatatgggtatatgtttgtgtgcccgtgattatatgcatatgggtgaatgtttgtgtgcccgtgattatatgcatgtgggtgaatgttcatgaacaaacaggtacactcggagtagaaaacaaatgaagcaaaagtttagcatcctctcatgtgtgtcaacattgtagttttacatgtactttcttctaatctattaaacataagtatggctccatttttttagtattaccagaagccctcccaatacggcaatattaccttctgtgtaatccacttgccgacagcatgttttctgttgaaatgggacatcaagaattttgaatccaaggccatatgctgcatgctcatgatttacaatggtttg

SEQ ID NO: 3 - DNA, 1078 bp, Homo sapiens
  misc_feature: Incyte ID No LG1094205.12000FEB18
ctcccaagatggcggctcctccgggcgagtacttcagcgttgggagccaggtgtcgtgccggacgtgccaggagcagcggctgcagggcgaggtggtagcctttgactaccaatccaaaatgctggctttaaaatgtccctcttccagtggaaagcccaaccatgcagacatcttgctcataaacttacagtatgtttcagaagtggaaataattaatgaccgaacagaaacccctcctcccctagcttcactcaatgttagtaagcttgccagcaaagcacggacagagaaggaggagaagctgagccaggcctatgcaatcagtgctggtgtctctctagagggccagcagctcttccagaccattcacaagaccattaaagactgtaaatggcaagaaaaaaacatcgtagtcatggaagaagttgttattacacccccatatcaagtggaaaactgtaaaggcaaagaggggagtgcactgagccatgtacgcaaaatagttgaaaaacattttagagacgtggaaagccaaaagatactgcaacgttcacaagcccagcaaccacagaaggaggctgccctgtcatcctgagtccgtacacatggaggactcttccagcatccagccaaggaggcgctggtggtggcttcagcggaggcaggccgggggagggaagggatcctatatgtcctgttggctcttaacaaagggtcagcatttccaactcttagacttgaacaaacaaaacacccaacaaaaaagaacaagagaataatttaaaagttgaatcattacttgactaacagacttcggtccaccatgtccttttcacagccctcttctggaacagtcaccttggtaattttattttggaaaattattttcccactctgccctttacttctgactttcctttcctagattgttcctgccattctgtgtttataagtggctacacttgccttctgaatgattgaaagaaacttttacatcttttcttccaaaataaaagtaacaagctgactgtgattcttaaattgagaccagagcagcaaacgtctcactttaaattt

SEQ ID NO: 4 - DNA, 533 bp, Homo sapiens
  misc_feature: Incyte ID No LG481361.52000FEB18
gttcttttccccttttctccagagataaatgtgttgatcttaaacatttgcttctgtgagaagctccagcacctgtgtgtcactgtcatcgagatcagtaggctgtttgatggtgatgaagatggtggcctccttactcagaatgcttttttctcatttcctagtgggattttagtgtgcactgatgtgatggcccggggaattgatattcctgaagtcaactgggttttgcagtatgaccctcccagcaatgcaaggtatggtacgaggctgccacccactctctgttgaggaatttagccaccacatgtgaaaattacaattactttcttcaaagtcagcatatattcctaatacatgcttccacgcataggaaaatgcctataaacccccaagtgttgatgattactcctgaggttattggtcattttcattttattctttgtatttttcattgtttcccagattttccactggggactatgtcccacctgtatgatcagagagaaagatacaagttatacttaagagta

SEQ ID NO: 5 - DNA, 2149 bp, Homo sapiens
  misc_feature: Incyte ID No LG981170.12000FEB18
aatttttgactttctcctttgtaattaatctctatctgggtttctctctttctctgtgccatttggtttccttaattagttccctgtgccagcccatagtcagagccataattggctctggggaagatccaagttattttctgagtaagatattaggcttccatatgatccagagatgcaaagaaatccctagagagtgtaggagttgtctaaatccatgtgtcagatgtagccaacgaattatgtcagaagcagagagaaaaggcctgaaaagcagctctctcccactcctcaggcccttgtctccaaccttacatgaggctttttgaacatctcctcctggcccagctggggtgagagcaagtcctcgaaggcactgcctttgagccttgctcagcccatctgaactatcccaactctagaattgactgctttcgaattgtgtgaccttgggaatgttatctggcttcaaccacaatgccctacccccagctcctctcccaaatgatcctagatacagggctgcttccccccgaccctaccccacctcgggacacaggctcatggcctcatggcacttcaccaccagaagtggtgctcagagttcctatttccacatctaaccccctaattcctgggaaagtctgaggcctggtccccccagtgctttccctggctggcctctccacattttcatctgatggtggagtgagatcaggaaaaataggacaggagctttgccttgggggagaagagagttaagtgtggaaaggggtgagttataggaggttaagcagtccaagatttctctctctgtgtaggaggccatttcctgatgtgaggggtctgaacccaattatgatgggacagggttgggcattgacttcccatctcttctctctgtttttctcccactatctgtagcccaaaactcttatggaggactttgatctttagtataggctattggtcagggccataggaactaaccccgatcctcactccaccaggatctaccacatcccctacacacaaacacatgctgtggggagggagttttcccctgggtcaagttgaggatccttagatcaccttgtgctcctgtggactggtgtgtgcgtgtgtgtgtgtgtgtgtgtgtgtgtgtgtgtgtgtgtgtgtatgtggggaaacttagctttcagagaatgtctatgggctctcattttctctctcacacaaaaatactcgggacttctccaagtccctgaggagcctgaccactgaagctgatcatgagatgactgtatgctgacacacccccttcaggggcctggccttgacttagggctgcactgtatcctcagcaacggccttgcaggagccccttttggactgctttccctattcagcccagagttggggtggtgggagaagaggggttggagtgaatccatctctattcaaattccagctgggattactctaggagtcttcctggcttgttttgggctcaaacttagctacattgtttattggctcccaaagtcgggattgaagagtgaaaagatgcaggcaatgaatccttctgcacactcctccccaacctttccagcgcttttctacttaggaggccagtggaagggaggagaggccatgccctagcccacaggggacaaggttcattgttcttccaggcttggttcactctgcttttgattcagaagctctttccctacccagcaagactacactttcttgccttctttctattttttctttttgtgcgtataaatggtatgttgtgatatattctcagtgcttgtgcccaccttggaactctgttcttgctcttcattccgcatgtgatactctggtccaagatcttggccaggtgccttctgctcaaatatcgtctcagaggtgcttcccttgaaaactcggtgctgtttccatagttactctatttgatcactctaagtttggttgtcttcatagcacttgtcaccctctggaactattctattcatttatttacttgtttaatgcttggctcttttcccctcctaacgtaaactccatgattgccaacacctgtttacttactacagttccccctccccccacattcctgacactagtaagaaccaataaacacttgttgacggaa

SEQ ID NO: 6 - DNA, 669 bp, Homo sapiens
  misc_feature: Incyte ID No LI197613.12000FEB01
tgcatcctctgggcacgctccacttgccgcttccacccggaaagccccccaggctgagtgcggcatgatctccatcaccgaatggcagaagattggtgtggggatcaccggtttcggcatcttcttcatcctctttggaacactcctgtactttgattccgtgctcctggcctttggaaacctgctgttcctgacgggcctgtccctcatcattggcctgaggaagaccttttggttcttcttccaacggcacaaactcaagggaaccagcttcctcctggggggtgtggttatcgtgctcctacgctggcccctcctcggcatgttcctggaaacctacggattcttcagcctctttaactgttccggagacttcaaggcactagctcgatggtctgaaaaacagagatgagctccttgaacttggatcattggttgagggggctagagggagaatgggaaccaccccctcagtcccctgcactgactcactccccgacatatccggacctccccaagtccagaaggaaggaatggagctgagcaactgacgtcaaatccccaagtcgactcaagaggctgccaggaagcagagatgcagaccccaaggagactgggctggggctggtatcacaccctcacacgcacaacttggggtctcgagtctag

SEQ ID NO: 7 - DNA, 463 bp, Homo sapiens
  misc_feature: Incyte ID No LI902682.12000FEB01
taagtaatgaggagccaaatgctaatcaccaaagcaatggggaaaatgtctccagggcatgtcagagaccttcacagcagcctctcccatcataggcctggagacctaggacagaaaaatggtttcctaggctgctgctattctgtgcagtgtcagaacatgttcgcctgtgtcccagtgacttcagcttcagctgtgggctgaaagaggccaaggcacagctcagacccttgcttctaagggtgtaaaccctaagccttgcagtttcccacatggtgtttggcctgttggtgcatagaagtcaaaaatcgaggtttgagaacctccacctagatttcggaggatgtatggaaatgcctggatatccaggcagaattttgctggagaggcagagccctaacagagaacctctgctaaggcagtgcagaagggaaatgttgggtcagaactcccaggcagagtc

SEQ ID NO: 8 - DNA, 2271 bp, Homo sapiens
  misc_feature: Incyte ID No LI212029.12000FEB01
ctaccctgctcctcagcctgtgagaacagatgtggccgtcctgcggtaccagcnaccccctgagtatggggtaacgagccgcccatgccaacttccgttcccatcaaccatgcagcagcacagccccatgtcctcccagacctcttccgccagcgggccactgcactctgtctccctgccgcttccactcccgatggccctgggtgctccacagcccccgcctgccgcctcccccagccagcagcttggtccagatgcctttgcgattgtggagcgagcccagcaaatggtggagatattaacagaggagaaccgggtgcttcaccaggaacttcagggttactacgacaatgccgacaagctccacaagtttgaaaaagaacttcagagaatttcggaagcctatgaaagtctggtcaagtctaccaccaagcgagaatcgctggacaaggccatgagaaacaaattggaaggcgagattagaagacttcatgatttcaacagagacctccgagatcgactagagactgctaacaggcaactatccagcagggaatacgaagggcatgaagacaaagctgcagaggggcattatgcttcccagaacaaagaattcttgaaggaaaaggagaaattagaaatggagttagcagcagtgcggactgcaagtgaggaccatcggagacacatcgagatcctggaccaggctttgagcaacgcccaggccagggtcatcaagctggaagaggagttacgagagaagcaagcatatgttgagaaagttgagaagctgcagcaggccctgacccagctgcagtctgcatgtgagaagcgagaacagatggagcggagactgcggacttggctggagagagagctggatgcactgagaacccagcagaaacatggaaatggccagccagccaacatgccggaatacaatgccccagccctcctggaacttgtgcgggagaaggaggagcggatcctggccctggaggccgacatgacaaagtgggagcagaagtacctggaggagagcaccatccgacactttgccatgaatgccgcagccactgcagcagctgagagggacaccacgatcatcaaccactcacggaatggcagctacggagagagctcgctggaggcccacatctggcnagaggaggaggaggtggtgcaggccaacagaaggtgtcaggacatggaatacactattaaaaatctccatgccaaaatcatagagaaagatgctatgattaaggtcctgcagcagcgatctcgtaaagatgccgggaagacagactcctccagcctacgtcctgcccgctccgttccatccatagcagcagctactgggacacactctcgccagacctctcttaccagcagccagctggctgaggaaaagaaggaagagaagacctggaaggggagcataggattgctgctggggaaggagcaccatgagcatgcctctgccccactgctgctacccccacccacctcagcactgtcctccatagcctccactacggcagccagcagtgcccacgccaagacaggcagcaaggacagcagcacacagactgacaagagtgccgagctcttctggcccagcatggcctcccttcccagccgcggccggctgagcacgacccctgctcacagccccgtcctgaaacacccagcggccaaagggaccgcagagaaactggagaactctcctggccatgggaagtcgcctgaccacagaggccgggtcagcagcttgctgcacaagcccgagttccctgatggagagatgatggaagtcctcatctaactgccatccctgtggaatttcagtacagaacactgacaaacaaggaaagcggcagagaaagaagaaagacctagaaggttgtagatgggaaatcaggaatgatttgaactgataaagatttcagactcataagaacacattttataaatgttaaacacaaaaactacatgactgaagatagaagagaatgcgatggattttattacacatggtggaagagagaagaggcgtgtaggtttgcaaacaaagttaagaaataggaaactgaatttttcattgtacagaaaatgtatctcttggggagggcctgtgtacccccattctctgattataaacagataaacccaaatgtggatcatccttggaatacccccattcgccttgcctcttagcatgtttgatttaagagaagcctccaggaaatcttgagngcctgtgttggc

SEQ ID NO: 9 - DNA, 1240 bp, Homo sapiens
  misc_feature: Incyte ID No LI249170.12000FEB01
tccgcctgtgctggacactccctttctcctgcagccatggatgccgctctgctcctgaacgtggaaggggtcaagaaaaccattctgcacgggggcacgggcgagctcccaaacttcatcaccggatcccgagtgatctttcatttccgcaccatgaaatgtgatgaggagcggacagtcattgacgacagtcggcaggtgggccagcccatgcacatcatcatcggaaacatgttcaagctcgaggtctgggagatcctgcttacctccatgcgggtgcacgaggtggccgagttctggtgccacaccatccacacgggggtctaccccatcctgtcccggagcctgaggcagatggcccagggcaaggaccccacagagtggcacgtgcacacgtgcgggctggccaacatgttcgcctaccacacgctgggctacgaggacctggacgagctgcagaaggagcctcagcctctggtctttgtgatcgagctgctgcaggttgatgccccgagtgattaccagagggagacctggaacctgagcaatcatgagaagatgaaggcggtgcccgtcctccacggagagggaaatcggctcttcaagctgggccgctacgaggaggcctcttccaagtaccaggaggccatcatctgcctaaggaacctgcagaccaaggagaagccgtgggaggtgcagtggctgaagctggagaagatgatcaatactctgatcctcaactactgccagtgcctgctgaagaaggaggagtactatgaggtgctggagcacaccagtgatattctccggcaccacccaggcatcgtgaaggcctactacgtgcgtgcccgggctcacgcagaggtgtggaatgaggccgaggccaaggcggacctccagaaagtgctggagctggagccgtccatgcagaaggcggtgcgcagggagctgaggctgctggagaaccgcatggcggagaagcaggaggaggagcggctgcgctgccggaacatgctgagccagggtgccacgcagcctcccgcagagccacccacagagccacccgcacagtcatccacagagccacctgcagagccacccacagcaccatctgcagagctgtccgcagggccccctgcagagccagccacagagccacccccgtccccagggcactcgctgcagcactgagccccctgaggcccacagccacccaggcagggcagcaagtggcctggtc

SEQ ID NO: 10 - DNA, 1564 bp, Homo sapiens
  misc_feature: Incyte ID No LI813218.12000FEB01
aggcaagggacatggactcttctcataggatgcacagtcaagtcacgtgacaggaatacataatgttaatacatttgaggaataatcctaacacatcaagttaaagtaagctgcttttgatcccccttttgaaaaggatgccagtttagttgttacataccaagtgccagaggctgagtttttttttttcctggtgaagatattgcattcgttgaaaacaaaaccacactgagttaccatttcatgttcactaggatgactgcagtcaaaaagatggaaaattgcaagtgctgttgaggatgtgaancaattggaannttcatgcactactggtnggaaagtgaaatggtgcagtcactttgggaaagtttggcagttcttcaaatggttaaacgtagagctactatatggcctagcaattccccttacaggcatatgcccaagataattgaaaatatatgttcagggctaccctctttgggtcccctccctttgtatgggagctctgttttcactctattaagtcttgcttctgcactcttctggtccatgtttgttaaggctcgagctgagcttttgctcgccgtccaccactgctgtttgccaccgtcgcagacccgctgctgactcccatccctctgaatctggcagggtgtccgctgtgctcctgatccagtgaggcgcccattgccgctcctgatcaggctaaaggcttgccattgttcctgcacagctaggtgcctgggttcgtcctaattgagctgaacactagtcactgggttccatggttctcttctgtgacccacagcttctaatagagctgtaacactcaccgcatggcccaaggttccattccttggaatccataaggccaagaaccccaggtcagagaacacgaggcttgccaccatcttggangcnacccaccaccatcttggaagtngctcgccaccatcttgggagctctgtgagcaaggacccctggtaacactttggtgaccacgaagtgancctccaaggtgaattgaaactgtaaaactacaaatggttcatcaaatggagccccagatgcagtccatgactaagatccaccgtagacccccggaccggtctcccagcccatgctctggtgttaatgacatcgaaggcacccctcccaaggaaatctcagctgcacaacccctcctatgccccagttcagcaggaagcagttagagcagtcatcggccaacctccccaatagcacttgggttttcctgttgagagtggggactgagaggactagctggatttcctaggccgactaagaatccctaagcctagctgggaaggtaactacatccatctttaaacatggggcttgcaacttagctcacacccaaccaatcagagagctcactaaaatgctaattaggcaaaaacaggaggtaaagaaatagccaatcatctattgcctgagagcacagcgggagggacaaggatcaggatataaatccaggcattccagccagcaatggcaaccccctttgggtcgcctccttgtatgggagctctataagtactc

SEQ ID NO: 11 - DNA, 284 bp, Homo sapiens
  misc_feature: Incyte ID No LI902522.32000FEB01
gtggtctgagtttgtggctgcatttttatctctggtggctctgctacggcggcgcagaaatgaggcagaagcggaaagggtgaaaaccaagacagctgccaaatatggcctttctgcccagccccgcctggtggatatcattgctgccgtccctcctcagtatcgcaaggtcttgatgcccaagttaaaggcgaaacccatcagaactgctagtgggattgctgtcgtggctgtgatgtgcaaaccccacagatgtccacacatcagttttacaggaaatatat

SEQ ID NO: 12 - DNA, 1209 bp, Homo sapiens
  misc_feature: Incyte ID No LI474304.12000FEB01
attttaggtactttgtttatggatctgttgaatctgaggagaagtgattgctttgagttagctagttttaaattcatgacactttcccaacataattaggtatttgaacctaagtgtgtgctagtgtattgtatatttttcattaattactttattgaaagttgctttgggaatcaattcacttcaatatattttactgttctcagtgaagaatgtgactaataagtatttggactttcaaaaagtgatacagtctttgtgatagttttttacctttacattaatgagttggaaaataactttgttcaaaggaatagaaatgcatattttgcttaaatattaacaatatgtttttaattcagtgtgtctcagtaaactatgttttggattgctgtgtttttacctcatgtttattttttctgtctacctggtaatttgagaaccctcgcttgcaacatatcaccatattattcgcctgtttgatcaacctggagaccctttaaagagatcatccttcatcatttatgatataatgaatgaattaatgggaaagagattttctccaaaggacccggatgatgataagttttttcagtcagccatgagcatatgctcatctctcagagatctagaacttgcctaccaagtacatggccttttaaaaaccggagacaactggaaattcattggacctgatcaacatcgtaatctctattattccaagttcttcgatttgatttgtctaatggaacaaattgatgttaccttgaagtggtatgaggacctgataccttcagcctactttccccactcccaaacaatgatacatcttctccaagcattggatgtggccaatcggctagaagtgattcctaaaatttggaaaggtcagttttactttttatgtgtccgggtgcatctcacctcaatatcctctactttgataatgaatgtgctaacattactgttagggatgaaaacttccattttgactgaaccaaatgtgttagttgagatgtacttcaaggtcatgtgctcatgttgcatgttggctatcaggagaaaattttgttgaaaatgacttaagtcagtcccttcgtgacataggaatgagctgagttttgtgcacctgttgcaggatatgacccaaagaaaataaatgctaatggcgtggcaaaccctaaatctgaataaaggtctttgacccgg

SEQ ID NO: 13 - DNA, 1358 bp, Homo sapiens
  misc_feature: Incyte ID No LI027320.12000FEB01
actccgacggcggctcggacgccgacttcggaggtgggtccggggagcccgactcggaccgcggaggtgagcgggagctgaggctgaggagaggngagcttggggggcgcctgctgccaagggcagcggaggaggaaatggcaggtcctaatcaactctgcattcgccgctggactaccaagcatgtagctgtgtggctgaaggatgaaggcttttttgaatatgtggacattttatgcaataagcaccgacttgatggaatcacattgctaacattgactgaatatgatctccggtctcctcctctggaaatcaaagtcttaggggacattaaaaggttaatgctctcagtccgaaaattgcagaaaatacatattgatgttttagaagagatgggctacaacagtgacagtcccatgggttccatgacccctttcatcagtgctcttcagagtacagactggctctgtaatggggagctttcccatgactgtgacggacccataactgacttgaattctgatcagtaccagtacatgaatggtaaaaacaaacattctgttcgaagattggacccagaatactggaagactatactgagttgtatatatgtttttatagtatttggatttacatctttcattatggttatagtccatgagcgagtgcctgacatgcagacctatccaccactcccagatatattcttagacagcgttcctagaatcccatgggcctttgccatgacggaagtatgtggcatgattctgtgctatatttggctcctggttcttcttcttcacaagcacagatatatggcagtgtatgggagaaattacatcgagcctttgccatttggagtggctttggtatgaccctgactggcgttcacacatgtggagattacatgtttagtggccacacagtcgtcctaactatgctgaatttctttgtcaccgaatatacaccaagaagctggaatttcttgcacactttatcctgggttctcaacctctttggaatcttcttcatcttggctgcccatgaacattattctattgatgtgtttattgctttttatataacaacaagactctttttgtactaccatactctggccaataccagagcatatcagcagagtaggagagcaaggatttggtttcccatgttctctttttttgaatgcaatgttaatggcacagtacctaatgaatattgttggccattttcaaaaccagcaataatgaaaagactaattggatgaatactatctttctaatgaatttgtgattaaatatatcatagttgttgaaaatgagtaactttgcgttctccccctaggttcntcttaaagc

SEQ ID NO: 14 - DNA, 1035 bp, Homo sapiens
  misc_feature: Incyte ID No LI228319.12000FEB01
caaagctggtttcaagccactttcctagtctttgttgaatggtaaatgttccttcaagagttgcaggaagagcatgccttgcttgcgccatggggtggggctgcaccatcttgcttgctgcgtgtgcttgtattgttcatcatacacaattcatggtttcttttcaggaatttttgaagtcgcttgtccattctatgaaggtcagtttggttaaaattaaagcattttaactgagtacttacaaactgtagtagacccagataggctgaaagcaaaagtaggatggagggcttggcttcggtgaggaccaagcctcttctgtcagccactcctgtgcgactgtcaggcccagggatccactcagggaaggtacctgtgtcaacctcaatattaactgttatcaaagcttcctgtctcttttattttagccagaagcaatacttttaatggattccttccacgtccaaatggatcatgtgtacccctcctgggcaaccaccggaagactctgggtattgtgtgcgttgtccgctcagtgacgaggatcttcataatttccttcctgcaaaaaggacattagttttcagtgtttccaacaattatccagcaataccatctaaacaccatttctgtaatctaggagcaagctgaagaggaaaagaagcccaaggatagcacaaccccttttgagtcacggttatcccaatccagaaaattttcctggacagaatacctggaagctactcaaaccaatgcagttcctgccaaagtttttaaaatgaggttgcctcatggttttctgccaaatatgaaacttgaagttgtggataaacggaaccccaggttaattcgtgttgctacgattgtagatgttgatgaccaaagagtaaaggttcattttgatggttgggaccataagtatgactactgggtggaggcagacagccctgatatccacccgatcggattgtgtgatgtcacagggcatccactggaagtgccaaagcgaacgaaatgacctgaagatccttccag

SEQ ID NO: 15 - DNA, 912 bp, Homo sapiens
  misc_feature: Incyte ID No LG197267.22000MAY19
cgtgctgcagccgccgctgctgcngctcctgctggcgctgctgctggcggcgctgccgtgcggtgccgaagaggcctcgccgctgcgccccgcgcaggtcacgttgtcgccgccgccggccgtgacgaacgggagccagccgggcgcgccacacaacagcacgcacacgcgtccgccgggggcgtcgggctcggcgctgacgcgctccttctacgtgatcctgggcttctgcggcctgaccgcgctctacttcctgatccgggcgtttaggttgaagaagcctcagcggaggcgatacggcctcctcgccaacactgaggaccccacggagatggcctcgctggacagcgacgaggagacggtctttgagtcccggaatctgagatgatgctgagccagggaggcggcccttccagcagccatgagggaaggacaggagatggggcccaccccagtgcccagcaaccccctgctccaccgctcattcccctgctggccccggggctggtctcacccagtgcccagcaaccccctgctccaccgctcattcccctgctggccccggggctggtctcacccagtgccaacccgagagctccttttggaacctgcacagcccgccgacctgttgccacctgcacccaccgctggaccatgcagcctcgcctcctggatgctgtcccagcctggccgagggtcccaggtgaagactggagggaccccaacagccaccgcccaggacgctgaggctcccttgcctgactgtgacttgtgcctctctcctgcccccgtggggacatggcagcccagagccaaggctgggtgggcaggtgacccaaggaacctttctgggaacaccttctcgccgggctgggaacaataaatgcagccatgtctctgcagctggtgctg

SEQ ID NO: 16 - DNA, 1615 bp, Homo sapiens
  misc_feature: Incyte ID No LG403332.12000MAY19
cgggctgttcctgtaaggcggggagacaatgagtaaactctccttccgagcgcgggcgctggacgccgccaagccgctgcctatctaccgcggcaaggncatgcctgatctcaacgactgcgtctccatcaaccgggccgtgccccagatgcccaccgggatggagaaggaggaggaatcggaacatcatttacagcgagcaatttcagcacagcaagtgtttagagaaaaaaaagagagtatggtcattcctgttcctgaggcagagagcaacgtcaactattacaatcgcttgtacaaaggagagtttaaacagccaaaacagttcattcatattcagcgcatttggggacactaccaaccggagaccacgttaaagtttttgcttgtttgttttgttcatttgtttttggaccacagtattagctttaatttaggctgcagatctgctcaaggttcagttctcaggaagattttttgcttctcctttctacctaagggcaagttgagaaatacaaaattctttgctttccctttctgcatggctaatttatttctttaatcttacactgagggcatagccctttggtaatgtatctttattcagtagcattctatactaatattaacacacaagcataaagcttttttctttcttatggtatatatagtaatgttgtgtctttaattgatggcagtttagatttgatagaattcaataatagtcatattctctagtatgaatattaggttctttgtaatttgttcccgctttctcctttaacgtcatttatttttgctcttcccttcactctgtgtaccatagggactacgaaaattaccctcattcaccttctttcatatttaacatttccttggatgtgctattgaagtcttctttcccttcctatagctcattaaagacttaactcccttatgaaagccaccttgacttttctcactctacctgtcttctttgcttatcgtagtatcttttacatacttctgttagaggactcaccatactttattgcaattatttgtaaccattctttctctccttttagacngtgagctccttgagggcaggaactatatattttattttcttttgtgttttcagagtaaagtgcttggtttatagatgctcaataaatgatgtgtttgtcgactgaattgttttaaaatggctttaaaaagtcttttggtggtacagccattttccacagatttcttttctctgaaatagtctagagaaacacacatgcgaaaattatttatgtatatttttttctcctgtgttctttgactttgagtcctattagctttttgaatttgcctttaccttctatcagtcaggaaggagataactattcataatagaaattttaaggatctctcttaagccaaagtatcaattattttgtattttgataatattgcagttaggcaggaagaccatttgtaaaaaccattctaggaaaccaagaactaaaaatcgggtgtaactcatgtgggtcaggcccatcgaatacaataaacattattcttgtgttggtttttttcatttcattttattctattattgtaaaaaacaataaaaataatagaaatt

SEQ ID NO: 17 - DNA, 880 bp, Homo sapiens
  misc_feature: Incyte ID No LG983076.32000MAY19
aatggaacttaatgtctattcctcttttctcttttctgtgttaccaaagggatctagagccttatgggtactacttagaaaatgagcactcatacatatacaatatctggaaataccttgaaagtaatcacgaatactccttctgtttcacttccatactgctgaattgcctcttcatcttctgtcaccataacaattggcatcctgtcctggcagtgatgatagtaccaaacagctgcgttgtatatgctcctggtctgccacttctccatggactctcctctttcccgtggcagatagcagcattgctggaattcattagcaaagagaatgcaatcatgacgcgcatccttcagcaggtttcgcagtttgttatactgtctgtgacacggaggagaaaaataactctaacaaagcaggtactcatccactgctatctcttgctaaggatcaaaaaaggatggggtattttagagccaaagattgattactttaaacctgaagaacatgtatttatattggtctcagtgtgctcagtcctatcgggctgctattgtttctcgattttgcctgctagaaggtgcttgggcagggcctagttaattagtttgcagatcaattaaactccttccctctcctttccccttcagagacttccagccattttattatgtaattataccaccttgccctgcctagccccttcttcaaagcctgagtgcacggaggcaagcagagagtttagcactttatttttggacctcactttgttctataaatgtcattaagcattaatacatttgactttttgctggcagccctagacaagaacatgtaaggtaaataagtttggggatttatctgtgaatttaatttatgc

SEQ ID NO: 18 - DNA, 1049 bp, Homo sapiens
  misc_feature: Incyte ID No LG216612.32000MAY19
cgggtaccgggcctcacggatccgcgccgcgccccccacctgtgtgctgcgcgcggggtgggctgcgctcccctgggcggcgccgggcgcccggggctggtggcgagatgggccgctactctggcaagacgtgccggctgctcttcatgctggtgctcaccgtcgccttcttcgtggcggagctggtctccggctacctgggcaactccatcgcgctgctctccgactccttcaacatgctctccgacctgatctcgctgtgcgtgggcctgagcgccggctacatcgcccggcgccccacccggggcttcagcgccacctacggctacgcccgcgccgaggtggtgggcgcgctgagcaacgcggtcttcctcaccgcgctctgcttcaccatcttcgtggaggccgtgctgcgcctggcccggcccgagcgcatcgatgaccccgagctggtgctcatcgtcggcgtcctggggctgttggtcaacgtggtggggctgctcatcttccaggactgcgccgcctggttcgcgtgctgcctccggggacgcagtcgccgcctgcagcagcggcagcagctgtcggagggctgtgtccccggcgctttcggggggcctcagggcgcggaggacccgcggcgcgcggcggacccgacagccccaggctcggactcggccgtaaccctccgggggacctcggtggaaaggaagcgggagaagggggcgaccgtgttcgcaaacgtagcaggtgattccttcaacacccagaatgagccagaagacatgatgaaaaaagagaaaaagtctgaagctctgaatatcagaggtgtacttttgcatgtgatgggagatgccctggggtccgtggttgtggtcatcatcattttgtcatctgccttcccgcttatcaaggagaccgctgccattctgctacagatggtcccaaaaggagtcaacatggaagagctgatgagtaaactctctgctgtgcctggaattagcagtgtacatgaagtgcacatctgggaacttgtaagtggaaagattatt

SEQ ID NO: 19 - DNA, 958 bp, Homo sapiens
  misc_feature: Incyte ID No LG322465.12000MAY19
acttccagaatgtcacactgcttttgcacactctcaaataggcatctgcttgtgtgggagctgtgattccctctcccctccccactgtcctctagtattgtttaaaaatgacggaagaaacaatagctagctccacagaagtcatcctcacaagccagatgtcaagtgagggctggcaacgttggggcaaaattaaaagtgatggagggggaaacagtggtgtgccccttgggtggatagaaggtttcattaatgtctgtgatggaatcatgaaccacaacagtgaggcatttccgcctctggcctttttttggtttttgttttttttagaatcaggatctcaaaaaaaataaaaataaagaaaacaaattaaagttcatcagtcatgaagcctccattccaggccctagctccaggatgacaattctagacacatcctgggccagaagggaatctgctgccctgaagggaaggtcccagtcctagcagcatacatcacctgataactgaagagcccttgggccctgaataaccagcagagagccaggcgcggtggatcacacctgtaatcccagcactttgggaggccaaggcaggtagatcacttgagtccaggagttcaggaccagcctagccaatatggtgaaactccctctctactaaaaatacaaaaattagccaggcgtggtggcacacacctgttaatcccagttacttgggaggctgaggcatgagaatcacttgaacctgggaggcggaggcggcagtaagccaagatcacgccccttcactccagcctgggcaacaaagtgagactctgtctcaaaaaaataaatttaaaaattaaaaaataatcagcagtgacacccaggtacccaagtactacattggagagcttggtgattataaaagaccttatcttttataatgccagaagcaagtcaatcttatttcaaac

SEQ ID NO: 20 - DNA, 830 bp, Homo sapiens
  misc_feature: Incyte ID No LG093477.12000MAY19
ggagcctggtttgttagatgttactttagaaataataacagcactttattgctgttattattgagtgcttactatatactaggtgcattattcatcataatcattaacaatgaattttttttttgagatggagtctctctctgttgcccgggctgagtgcagtgccgtgatcttgactcactgcaacctccacctcttgggttcaagcaattctcctgcctcagcctcctgagtagctaggactacaggtgcacaccaccacgcctggctaagttttgtatttttagtagagatggggggatttcaccatgttggccagactggtcttgaactcctgacctcaagtgatccacctgcctcggcttcccaaagtgctgggattataggagtgagccaccccacccagccaacaacgaatattaatgcagtacttatgatgtaccaaactcataagcacacttacctcaccaggtcttagaagaacactccctctggagccatcacgtacgcgtgaggacattgaggccctgagcttagataacttgttcaaaatggccctgctgataagctacatggacgaagtcagcggttttgggcatgtctttccaccagcttctggaagaacagcacacaggccgttcccctctcccccattttaaagtggcaggagaaggtggaatgaagaaaataaacagggctgttcctggctctgtgtggtcagagggtgatactggttcagggtccagcttatcagtgctgcactgatattcacagagcattcggctccggaggctttgcactgtttgtaatgcattgtcaaaacttggtgt

SEQ ID NO: 21 - DNA, 2591 bp, Homo sapiens
  misc_feature: Incyte ID No LG222880.12000MAY19
cgtcgcccaaggtcgcgggccgcttggggagtcagcagcgcgccaggccccttcgggccccacacgcattaggtgccttctagaagggtacggagtgaacgcgggcggcggcgggaccgaggcagcgcccagtttgtaaccgccgcgccgcccgtgcccgcgcgcgccacaccccagcgcgcttccggccgggccacgtgaccgcgcgtgcacgtgttccggcctctccgcttcgccgctccgaacctcctcctggtcgtcccggcattcgtccacgcggagccggcttgggcggggcccgggaggcggcggccggagaagccgcggagacgcgagcgccgagcgtcgcgagggagcaggcccgggcaggcaagcggcggcctccgccatgaaccccaggggcctgttccaggacttcaaccccagtaagtttctcatctacacctgcctgctgctcttctcggtgctgctgcccctccgcctggacggcatcatccaatggagctactgggccgtctttgcccccatatggctgtggaagcttctagtcgtcgcaggcgcctccgtgggcgcgggcgtttgggcccgcaaccctcgctaccgcaccgagggagaggcctgtgtggagttcaaagccttgctgatcgctgtgggcatccacctgctgctgctcatgttcgaagtcctggtctgcgacagggtggagaggggcacccacttctggctgctggtcttcatgcctctcttcttcgtgtcccccgtgtccgtggctgcctgcgtctggggctttcgacacgataggtcgctggagctggagatcctgtgctcggtcaacatcctgcagttcatcttcatcgccctaaagctggacaggattattcactggccgtggctggtggtgtttgtgcccctgtggatcctcatgtcgttcctttgcctggtcgtcctctattacatcgtctggtccctcctgttcctgcggtccctggatgtggttgccgagcagcggagaacacacgtgaccatggctatcagttggataacgattgtcgtgcctctgctcacttttgaggtcctgctggttcacagattggatggccacaatacattctcctacgtctccatatttgtccccctttggctttccttactaactttaatggccacaacatttaggcgaaaggggggcaatcattggtggtttggcattcgcagagacttctgtcagtttctgcttgaaattttcccatttttaagagaatatgggaacatttcatatgatctccatcacgaagatagtgaagatgctgaagaaacatcagttccagaagctccgaaaattgctccaatatttggaaagaaggccagagtagttataacccagagccctgggaaatacgttcccccccctcccaagttaaatattgatatgccagattaaactcctagagaggacccaggcacacacagactccacttggccttcgcctcttgttcattcatcccaaacctggaaatggaaacaggcttcaaacactcgtctcacgccgtgtttgagatcaccgcctcatcagtatgcatcatagatggaggtggtttcagtatgtgggtgtgtgtgatgtgtacctgggtaagagacttgctttccaggttcgcactttcaggtgtagctgggggcagtaagtcgaattgttttagtaggtcctcaaaaggaataaccacacagctgtttgtttaaatgctactgtacctatcaaaactattgtttaaaaagtatttttatacactgctaatctaaaattgtatttcagattgtgcctgtcataacaatagcaaatgtaaaaagttctctttcccaccacttgtttataaacctcatagttgatatttttagtgttcctactgttaaaatactctctccttgggctttgctgatactggtctttaatattctgataggtgaatttttctaatggaatgaacccatgcatatatagtatttatatgaatattttagcagtgtaatatgttgaattctagttctctgcattaccattattacgttaaagtattttttaaagcttaggtgtgaagatatgtgtctattgcagatgtccttgaaaactgcataaaacagtatgtgcctggtgtggatcttaccaaagtactaggcatgaatgtagggactgcaaatcccatgggtcttaatatttaggtgttagtaaccaaggtctctggtagtacccgttagtagaggaagaggccactgcccttgggaacttgtgacaggctctagtgtggtaccaggccataaagtgacactgttatttagcaacttgaatttttccacacaggtagtaactgtgtggaaataagcaacaagtggtttgtccatttctaagaatcttaaactattagttggctgtagtgtgaagcattacttgtcattggaaagatggagagagtggccttaaccggaagtggtcagtagaagcaggtgtcattttaagggccaaactttaatctgtcagcaatagggaaacaactgttcaaattatctttgtagataagaacagtgtttcttttttctt

SEQ ID NO: 22 - DNA, 2482 bp, Homo sapiens
  misc_feature: Incyte ID No LG898320.32000MAY19
ccctctctttcgctgtttgagagtctctcggctcaaggaccgggaggtaagaggtttgggactgccccggcaactccagggtgtctggtccacgacctatcctaggcgccatgggtgtgataggtatacagctggttgttaccatggtgatggccagtgtcatgcagaagattatacctcactattctcttgctcgatggctactctgtaatggcagtttgaggtggtatcaacatcctacagaagaagaattaagaattcttgcagggaaacaacaaaaagggaaaaccaaaaaagataggaaatataatggtcacattgaaagtaagccattaaccattccaaaggatattgaccttcatctagaaacaaagtcagttacagaagtggatactttagcattgcattactttccagaataccagtggctggtggatttcacagtggctgctacagttgtgtatctagtaactgaagtctactacaattttatgaagcctacacaggaaatgaatatcagcttagtctggtgcctacttgttttgtcttttgcaatcaaagttctattttcattaactacacactattttaaagtagaagatggtggtgaaagatctgtttgtgtcacctttggattttttttctttgtcaaagcaatggcagtgttgattgtaacagaaaattatctggaatttggacttgaaacagggtttacaaatttttcagacagtgcgatgcagtttcttgaaaagcaaggtttagaatctcagagtcctgtttcaaaacttactttcaaatttttcctggctattttctgttcattcattggggcttttttgacatttcctggattacgactggctcaaatgcatctggatgccctgaatttggcaacagaaaaaattacacaaactttacttcatatcaacttcttggcacctttatttatggttttgctctgggtaaaaccaatcaccaaagactacattatgaacccaccactgggcaaagaaagtatccctttaatgacagaagccacattcgatactctgcgactctggttaataatcctgctgtgtgctttgcggttggccatgatgcgtagtcacctgcaagcttatttaaatttagcccaaaaatgtgtggatcagatgaagaaagaagcggggcgaataagcacggttgagctacagaaaatggtaatcattcctggggtatttatccagaatctatctctaccttaccagtggataatagtctactgtccaattctgtttactctgaattaccatcagctgaagggaaaatgaaggtaactgttacacaaataacagtggcactgagcagcttaaaaaatatttttactcctcttctttttcgaggacttctgtcttttctgacctggtggattgctgcttgcctcttttctacaagcctttttgggcttttctatcaccagtatctgactgtggcatgaatctcagttaacaaaaaagcatatccaaatcaccctttaaattaaaatatctgtgcccttaaagggctgatgaaaaccagaagaaagcaaatacaatgggaaaaaaaaaacatatcagaatgtcttgtattaaatgtttcctctgtattctcagggtgaattaatgtagtaatatttaaaattacaaaatagattgttaactgttacactgtggcattggaattttaactctttgtatttactggtatgagagggctatctacaagggtaatatttctgattaccctggtttacagaaacctccagcagtctttgaaacatctcacatgactctagttattgattgcttttaatggttttatggtactgttgatagtcatagtggctgcctatagaacaatcttcaaactgagccatgctttaggggagggaaaggggctaaagtctcttctgttggtaatttattagttactcttgaaacaataaaatccaacagaaaggaagagatagctactgtatattacagtaaagaaagctgcatagttattttaaatttaatggagatgaatatggttaaaatatataactactgctgcttgagaatagcaagagtattgttttaaaacatattccacccaacttgagagttcttttaaaatgattggccatatgaacatttgtaatcttgccattaggtttggacctgccatattttgttttattctgtgatcctaactagttccttttaataggctaaaatatttatcaatactgatcagactttaaagaaattactttgtaaacctgctgactacctgtatgtattgtatatatattatatattaaatatataatatattgagattataaaagatgaaaatattgaatccttataatattttaagttgcagaatgtatgttaaaaagtgacttgaatgagatgtatttgtatctagaaattttatttctttttggaatgagattaanatacattttgaaagttca

SEQ ID NO: 23 - DNA, 1334 bp, Homo sapiens
  misc_feature: Incyte ID No LG1327047.12000MAY19
aggcaagggacatggactcttctcataggatgcacagtcaagtcacgtgacaggaatacataatgttaatacatttgaggaataatcctaacacatcaagttaaagtaagctgcttttgatcccccttttgaaaaggatgccagtttagttgttacataccaagtgccagaggctgagtttttttttttcctggtgaagatattgcattcgttgaaaacaaaaccacactgagttaccatttcatgttcactaggatgactgcagtcaaaaagatggaaaattgcaagtgctgttgaggatgtgaancaattggaannttcatgcactactggtnggaaagtgaaatggtgcagtcactttgggaaagtttggcagttcttcaaatggttaaacgtagagctactatatggcctagcaattccccttacaggcatatgcccaagataattgaaaatatatgttcagggctaccctctttgggtcccctccctttgtatgggagctctgttttcactctattaagtcttgcttctgcactcttctggtccatgtttgttaaggctcgagctgagcttttgctcgccgtccaccactgctgtttgccaccgtcacagacccgctgctgactcccatccctctgaatctggcagggtgtccgctgtgctcctgatccagtgaggcgcccattgccgctcctgatcaggctaaaggcttgccattgttcctgcacggctaagtgcctgggttcgtcctaattgagctgaacactagtcactgggttccacggttctcttccgtgacccatgacttctaatagagctgtaacactcaccgcatggcccaagattccattccttggaatccgtgaggccaagaaccccaggtcagagaacacgaggcttgccaccgtcttggaagcagcctgccactatcgggagctctgggagtaaggacccccagtaacatttggtgaccacgaagggatctccaaagcaatgggaaacgttccccccaaggcagaaacgcccctgagatgtattctggagaattgggaccaactggactctcacatgctaagaaataaacgacttatattcttctgcagtaccacctggccacgatatcctcttcaagggggagaaacctggcctcctgagggaagtataaattataacactagcttacagctacacctcttttgtagaaaagaaggcataaggagtgaagtgccatatgtacaaactttcttttcattaagagacaactcgcaattatgtaaaaagtgtgatttatgtcctacaggaagccctagagtctacctccctaccctggt

SEQ ID NO: 24 - DNA, 937 bp, Homo sapiens
  misc_feature: Incyte ID No LG235157.212000MAY19
ttccacgcccgagggcatcgcgctggcctacggcagcctcctgctcatggcgctgctgcccatcttcttcggcgccctgcgctccgtacgctgcgcccgcggcaagaatgcttcagacatgcctgaaacaatcaccagccgggatgccgcccgcttccccatcatcgccagctgcacactcttggggctctacctctttttcaaaatattctcccaggagtacatcaacctcctgctgtccatgtatttcttcgtgctgggaatcctggccctgtcccacaccatcagccccttcatgaataagttttttccagccagctttccaaatcgacagtaccagctgctcttcacacagggttctggggaaaacaaggaagagatcatcaattatgaatttgacaccaaggacctggtgtgcctgggcctgagcagcatcgttggcgtctggtacctgctgaggaagcactggattgccaacaacctttttggcctggccttctcccttaatggagtagagctcctgcacctcaacaatgtcagcactggctgcatcctgctgggcggactcttcatctacgatgtcttctgggtatttggcaccaatgtgatggtgacagtggccaagtccttcgaggcaccaataaaattggtgtttccccaggatctgctggagaaaggcctcgaagcaaacaactttgccatgctgggacttggagatgtcgtcattccagggatcttcattgccttgctgctgcgctttgacatcagcttgaagaagaatacccacacctacttctacaccagctttgcagcctacatcttcggcctgggccttaccatcttcatcatgcacatcttcaagcatgctcagctacgagtcctcggcggaaatcctgcctcataccccgaggctcacccacttccacacaacatacga

SEQ ID NO: 25 - DNA, 2811 bp, Homo sapiens
  misc_feature: Incyte ID No LG085713.12000MAY19
ctttgaccaagtcatttgacctctctgggcccatctttattggaaaatgtcaagtagatggtctctaataaatttttgtccagttctaacattatgtgattctaaatgtatttatactgaccttcttaacttgaactctttgttttttcagcatttagtatgagaacctatgtttgccatatttgtagtattgcttttacatctttagatatgttccggtcccacatgcaaggaagtgaacatcaaattaaagaatccattgttatcaatctagtgaagaattcaaggaagacacaagactcttaccaaaatgagtgtgcagattacatcaatgtgcagaaagccagaggactagaggccaagacttgtttcagaaagatggaagagagttctttggaaacccgtagatacagagaagtggtcgattccagacccagacatagaatgtttgaacaaagactcccatttgagactttccggacatacgcagcaccatacaatatttcacaagcaatggaaaagcagttacctcattcaaagaagacatatgactctttccaagatgaacttgaagattacatcaaagtacagaaagccagaggactagatccaaagacttgtttcagaaagatgagagagaactctgtggatactcatgggtacagagaaatggttgattctggacccagatcaagaatgtgtgagcaaagattttcacatgaggcttcccagacctaccaacgaccataccatatttcaccagtggaaagccagttacctcagtggctaccaacccattcaaagaggacatatgattctttccaagatgaacttgaagattacataaaagtgcagaaagccagaggactagagccaaaaacttgtttcagaaagataggagatagctctgtagaaacacacaggaacagagaaatggttgatgtcagacccagacatagaatgttggagcaaaagctcccatgtgagactttccagacctattcaggaccatatagtatttcacaagtagtggaaaaccagttacctcattgcttaccagctcatgatagcaaacagagactagattctattagctactgtcaactcaccagagactgtttcccagaaaaaccagtacccttgagccttaatcagcaagaaaataactctggctcatacagtgtagaatctgaagtttacaagcacctctcttcagaaaacaatactgctgaccatcaagcaggtcataaacggaaacatcagaagagaaaacgacacctagaagaaggcaaagaaaggccagagaaagagcagtccaagcataaaaggaaaaagagttatgaagatacagatttagacaaagacaagagcatcagacaaaggaaaagagaggaggatagagtcaaggtcagttcaggaaagcttaagcatcgaaaaaagaaaaaaagccatgatgtaccctccgagaaagaagaacgtaagcacaggaaagagaaaaagaaatctgttgaagaaaggacagaagaggaaatgctttgggatgagtctattcttggattttgaatgtttagttttgtttacccaaggttgaattgaaaaaaaaaaacagtcaatatggatttagaaaaaggaacacctgatgaagaaaaggagaggtagatacagtcagtgtcacttcaggacacttaggttttttttgtataaaaatttaaattgaattaaaagaaggaaaaaaaaagcccaaacttaacctctgagaaaagaacataagaactcaaggagaacataagagaaaaggaaacctgttacagaaaagacaagaatctgtgttttggaatgagtctattcttgggtattgaacttttagttttgtttgcccaaggattaattgaggaaatcagctaagaaaatggactttagacaaaagcaagaggatcagatgaagaaaaggagaggtagatacagtcagtgtcacttcaggaaagctatttaaaaaaacttgaaatttaattgaaagaagaaacaacaacaaaaaagcctaaacctagcctctgaacaacactaacatgagaacacaagaacttaagagaaaaagaaacctactcaagaaaagacagaagagacagtgatttgggatgagtctactctaggattttcaactttttagttttgttccttcaaagttgaaggaaaaaaagtttggttttataaaattcatgttattgtaatttttctaggtggatggctactttaatctctaaaaaagccaagtgaagtaaaagtattcagtatgccttttcctcaagttactttccttcattttcttaaaaaagaaaaaaaattattaaatgtttctcacatatctcacatataatgtaatttccctaaatgaagttgtctctacttctgctcatcaaattgctgtgatagtgaattatttattcatgggagataatttattttaaaggacagaattaccaagcgttacaaaatcagttctttccttggttttgtgttagtgttggtggtattttattgttgtttttctgtgtttatgtgtctcagctttctccaaggaatatgtatgaaataacttaaactgattttttctttgttaaatctaatttgcagtgtatttttgcattttctagttctgaaagtggaaaatgaaacagtctataataaacttagatgatatatagttttaaaacggtctcaaaaagtactgatataaggtcagtctatattctggaaatgtttatattaaagtgttttaatttctaaaaaanaaaaaaaaanagaaananaaaa

SEQ ID NO: 26 - DNA, 2963 bp, Homo sapiens
  misc_feature: Incyte ID No LG482421.12000MAY19
gaggtgccttgaaattgtaaatgtattttttttcttttattttaagctggtaaatacagggtaaaatctctttagatctcaatcatgcctattagacacacaattttatttaaactatttaacctgtcagatttaagaaaataatttggaaaactcacttttgtaacaaattgcaggtacaaaaattactcatttggaatagtcagaatacagatgctactcacctgtgagcacagatatattcatatttctatgtcagaccaaaatcctgcttaaacacacaaacattcctaacaatgtcataatgtttactctttctcctggcttaataaactatatttctgatattattggaagattattgttagaattgaaggagataatacaggttgagcagcccttatggaaaatgcttgggaccaaccaatagtgtttcagattttgatttttttcaggttatggaatatttgcatacacataaggagataccttggtgttgggacccaactctaaacacaaaattcatttatgtttcctatacacctttacacagagcctaaaggtaatttatacaatattttaaataagtttgtgcatgaaaaaagtttgtgtacattgaaccatcagaaagcaaaggtgtcactatctcagcgacccaagtggtggtgtcagcactcaaaaagttttggattttggggtatttcagattttagatttttgtatgaggaatgttcaacctgtatttgaacaagcattaccaaatatcattgaatattaatatcttttgcgtaaaaactgctattatcagcatcatagtttctctaaaaagaaaacttggggatcatagccgatagagagacttgctaaaatataaatcagcctctgcaaaactgtttacatatttattggtttacatattttattggtttatttctatcccctgttcactttttctcttccacttccaattatgaagagaaaatatttgttcagggttgtccccccgccccccgtcactgcataatttctcctcttacaagctgcttttggctttcattaataacagcttccttttagaaggtctgataaggatatttaaggaagaagagaatgactctgttattaaaggtggcatggagactgtggagggaatattttttaaagcactactcatatcctttaaactaaattttgccaaagcccgagacaacattaaggagaaattgtaccttaagttagtaattccaaatctatctgagttgtatacccatcaaagacaatacagttattaacatagatgaaggtatgctataggcatcattcattatctctatattgaataggtgaaagataactgtagtcaggtgaaaggcattcattatttttaagctgaaaaggggatccttgaaaacactgaaaacctctacaacaatcttcaggaagcctgctatcttgggattcactaataataggccaagaacaaaggcaagcatccattcctcactccaccacttttctatttcagtgggtgtcgttgctacgatgaagactttggaaatttcctttctcttttaggacagggtcaggatttaggactcatagcctgaaagctcattacatactccttgtaaccatcagtccaaggttcagttcactaaagtgcatgttctaaaacaagagctatcctcattccaaattttaaaatatgtactctggtcggttgcagtggctcacgcctgtnatcccagcactttggcaggccgagatgggcggatcttttgaggtcaggagtttgagaccagcctggccaacatggtgaaaccccgtctctactaaaaatacaaaaattagccaggcatggtggcatttgcctgtaatcccagctactcgggaggctgangcaggagaatcacttgaacctgggaggcagangttgcagtgagctgagattacaccactgcactccagcctnggtgacagagtgagatccatctcaaaaactgaanataaaaataaaaatatgtattctcctaactgaaatatttacttaatctggaaaacaatgtaactatttttaaagtggttacatctattcttgctgaagaacaataaacagaattttttgacgaagcataaccaaatttcagaacagtctaatcaatgccaagtatccaaggcaaactctaatacccatccattgtgcaaaaccacaagcacgcaagtattaaataagagcaagctgtcctgagcccatacctaatgaatttgtgtcttaaatattgtacattgtgtttgaggcttgtcaaaactgggattatggcaagaaaggttgcctaactcatacctttctgcctcaaattccaggtgctaaaggctaatggcattttaaacatcttacatttttaaaaatttatattgcctctgccaaacaggcctaatagttaaaagcaagttgagacaaaccaggcagattcagtgtgtggaacaggaaggatgtgctttaaaaaaaggtggaatccctcaaaaaattctatagggagacagcagccttaatctacataattcttcatctcgccaattcagccgcagcctttaaagagttagtgttaatggctttctggtttgaaaacaaaaatgcatctatgtggttgaaagtttgggaggagattcaccaatatctgaggagaagatggagtgaagggaattcttactttttgctttatacctttctataatatttagatttttttttactgtaagtatggatcaaattgcaaaataaagaaaaatgccaaccttagaaaagacaataaatgcacaaaagatataaacaggaacagcaaatatttatattttttccattttgctctttttaaatctatgtttagaactttatatcttgggacttatgtatatatataccttttaaataaaataaattttctaaataaaaagttgaaaaa

SEQ ID NO: 27 - DNA, 643 bp, Homo sapiens
  misc_feature: Incyte ID No LG330944.42000MAY19
tagcaggaattgaacaacattaccaagctcaatgaacacttcagcaaatttggaactattgttaatatccaggttgcttttaagggtgacccagaagcagccctaatccaatatcttaccaatgaggaggccaggaaagccatttctagcacagaagcagttctaaacaaccgattcattcgagtcttgtggcatagggaaaataatgagcaaccgacactacagtcctcagcacagctgctcctgcaacaacagcaaacacttagtcacctctcacagcagcaccatcacctgccacagcatctacatcagcagcaggtgctagtggcccagtctgctccttcaacagtgcacggaggtatccagaagatgatgagcaaaccacagacatcaggtgcatatgttcttaacaaagttcctgttaaacatcgtcttggacatgcaggtggtaaccagagtgatgcatcacatttgttgaatcagtctggtggtgctggagaagattgccagatattttcaactccaggccatccaaaaatgatttacagctcctcaaacttaaagacaccttcaaagctctgttcagggtctaaatctcatgatgttcaagaagtgcttaaaaaaaaaaaacaaagcggccgcc

SEQ ID NO: 28 - DNA, 1966 bp, Homo sapiens
  misc_feature: Incyte ID No LI223060.12000MAY01
gggacaaatgactttttcttgttgaggcaaatagcgaanactctgcttaatttccacggaatttgtgccagttttcaaagactacaatttgaagaggcattatatgcaaaaacgtgctgacaaatttggtgtgtatgtgttgtaaggacaaaatagcagaactgaaaaaaagtctgtcttttcaacaaaaaagattttttagaaaagttacaactcggactcaatcgtaaggtaaaacctagttatgtggtagcaaatttaatagcaaaaaaatcaaaaccatttactgatggtgagtttattaagcaatgtctggaagatgtggtagatattatttgccttgagaaactgatatttctaaaatcagtttgtctcaccagactatagccaggagaattggagaaattgggaaatctattgaaagacgtttggagagtaaaactgctaatttaaaattttatgctttggtgatggatgaaggcactgacactacagatacggcacaacttgctatttttattagaggtattgatgatgaatataatgtcactgaccgaaatcgaaatgtcaccatgctattaaaagacacaactaaatcaagagatttatatgaagcagtgaaaaatatgttaaagcaattttctttgtcctttgtaaacatatgtgatatagctacagatgctgccctggcgatggtaggtaaaaagagagggacttgtacaattaatagatgatgcagttgccacacaaaactcacatttgatgaagtattattgcataatacatcaagaaaatccatgtgcacaagctttaaaagtagataacgtcatgcaaattgtcatcaaggctgtaaatttcacaagggcccaggaattgaatcatcgctagtttcaggaattccttaaaagtatggatgctgactctagcaagtcattcactttttagaagtaagatggttaagtcaaggcaaaatgtaaagattttatgctttgtgatgtgaaatcaagctgtttatggtatcaaacaaaattggtgccagaacttgacaatgaaaactgggcttacagatttagcatttttagtgggtttgacggcccatttaagtgagttaaacctgtgtcttcaagttgaaaacccactttaccaatacaatgtttcaaaccataacagcattccatatgaaactgaaattatgggaaggctcaaattaaggcaaacaattttatgcatttcgacatgttggctaaacatggtcctgtgaacagccaaaaatacgcagccttgcttttcaatttgatacaggaatttgaaaacaggtatttcaagatttctgaaaaaatcatcaatattttggtatatttgcaactccattttcagtcgacatatatatgttacctggcaattttgaaatggaatgcctagagctgcaatctgatgttcaacttatagaaaaatttgattttgcctctttactggacttttgtaagacctgtcttcccaatgacaaatatcccttgcttcacaatcacaccttgttcatgtcattgcttttggggagcacttacatttgtgagctactattttcaaggatgaagaacacgaagagtaaaattagaaccaaaatatctgatgagcaccttgagaactcactgagaattgcaactacttccatcaagccagatactgatggattacgtttctcaaaaacaatgtcaagtaccccactagttttatgttgttctcttttcttttataataaaaaatatcaaaaaagtagtgaagtttagctagatatgtacattttctgtatcagtgattgcaaacttggaacctgttcaatgattttgaaagaccctctgaatggggcagtgcataattaggattattatgcaagggacatttttgcttatctgtggtggtagatatataatgaaaactatacacagaccatttgtgtgtgtgtgtg

SEQ ID NO: 29 - DNA, 897 bp, Homo sapiens
  misc_feature: Incyte ID No LI213087.12000MAY01
gtcgaggcgcagcgctgccatggctgggggccgtggggcccccgggcgcggccgggacgagcctccggagagctacccgcaacgacaggaccacgagctacaggccctggaggccatctacggcgcggacttccaagacctgcggccggacgcttgcggaccggtcaaagagccccctgaaatcaatttagttttgtaccctcaaggcctaactggtgaagaagtatatgtaaaagtggatttgagggttaaatgcccacctacctatccagatgtagttcctgaaatagagttaaaaaatgccaaaggtctatcaaatgaaagtgtcaatttgttaaaatctcgcctagaagaactggccaagaaacactgtggggaggtgatgatctttgaactggcttaccacgtgcagtcatttctcagcgagcataacaagccccctcccaagtcttttcatgaagaaatgctggaaaggcgggctcaggaggagcagcagaggctgttggaggccaagcggaaagaagagcaggagcaacgtgaaatcctgcatgagattcagagaaggaaagaagagataaaagaagagaaaaanaggaaaganatggctaagcaggaacgtttggaaattgctagtttgtcaaaccaagatcatacctctaagaaggacccaggaggacacagaacggctgccattctacatggaggctctcctgactttgtaggaaatggtaaacatcgggcaaactcctcaggaaggtctaggcgagaacgtcagtattctgtatgtaatagtgaagattctcctggctcttgtgaaattctgtatttcaatatggggagtcctgatcagctcatggcgcccaaagggaaatgtattggcagtgatgaacaacttgg

SEQ ID NO: 30 - DNA, 2792 bp, Homo sapiens
  misc_feature: Incyte ID No LI405330.12000MAY01
ctgtccttaagatggtcaccatattcttattcaggctgttgtcattttccccagagatggtttgtttaacgaatgataggctctgtgcctggggatgttagcagactctggggtttgtacagtgatgccttctccctggcccagagctgaatattcatctagaattaaagttggatttgatataacaaatttctttctatacaggttttacataagagagacagtaataatgtcaaggatagcctgtgtgggcagaaattggtaatccggcttttggattgtcagctgtagagccaactctgattatctagccattgatcatacaaattgatagaaacattagtcagtaattttagcttcttgccaaattgttcacaacatctaaatgtaatgtgatgtgatgaagataagtagtacaaagagaccaaaataatttgggagaattaggaatgatgacaattttttttaacaactttacctctaatagggttacttggatgagccaactccgcttccttcccatggatatggaaagggactctgtgtattattcaggtttattggcacgaagatacttgttttaagttccttgagaacccatgatggacagttgacagaatgcttaaacctgtcaaaagatgagtgattttgtgtgggaaaagccttcccaggcgtctgtaccgaaaggatgcagcaaacaaggggctaatccatgagcagtgttctgtaggctctgtgacatctttggtttataggattttggagccttttattgtccgggaactatttgaggggtttcattataggccttggttctctccaggggccagatgagtttattgtggaatctttgaaaggacaaggcctctgtgaatgaatcagtcccagggaagcatttggtggtggcggcagtggaggattgcccggtgaacctataaatcagcagtctcttgggcagaggagcaagcccctcgaacatgatttcaaacaagcaggtcctcttctctcatctcacgtccttagtctctgttaatgaacatactggatgtggagtttaataaattacctactatcatctggccacttagattattatcacaccactgtggactgttcctggggggagaagaacagaccgatttgaaagattcaagggagaaagattaaggatcaggattgcatgaaagaagaaaatccttcaatatttaaaatgtttcttacaatacccacggagcacttttatggttccagccgagcgttcctgaaatgaactgaccattaacagcgcctctttgataggttaccctgatgctgctaaagtaaagccttaagtgtgtttttgggacaacgtgctgcttattccacctcagccacatatgtgtttgtgtttaggatattgtaaatctttgctaagtagtgttttccttggtgaatgaagtcattgttgtcttcaagtgtaccatctgcctagcaaaaaattgctacaaactttctcttatggcaatagtccttgggtacttctaatatttttaggcaagagacaattttctgtactaggaatcttccactgccaggaaaacacagtgccagtaagggttctacatacacactgaccatctgcttaatagacatgtatttcctttgagtaggacattagcttttgattataaagctcaactagtataagcaaaaatataacatctagaagcacagttttagccaggatgtttaaaaattacagttttgtgagacttaagggtctttttaacctaggtaagtttatatgacctaacttaattgtagccctattcttggtaccttcccttttggaaagtagaggttgcggtaaacaagcgctagggtattataagagacttttcctgaggcacctgtttggaatctggttttctcagcggcagcttgacatgtgcacccttttgtattaaacactgcaagggtgattgcaggggagcaggaaagccatcctaaactcactactgagtacgattcagtatgttcctgtggatgtctgctgtgactaatataaatttcttgcagaatcagctacacttaattatgttgctgatagacaagcatccacgcttcagctgggcactaagtgttttcattgtaggatcagcagcaggttaaagactgaacggttagtgaagactaaatgtcttaagagggtgcgatgtctaggttgggcttgtgacgttcttagtggcctagcgcttctggatggcaccttgagaagtgaacttctagagaatctacatttaaaaggcgaagcgtttagaaagcagagctagtctattctagttagctgatgcgaactaaaattctgtagtttcttaagatggagccactgacgagatgtcacagtatagagcctgcagtctcaactcattgtgatcctaatggtctgggtgattggatggtttgagttgttagggattttgaagtttttcattttaattgcatatcctgggttggatgttagaactaaaggaaaccccaggaatatttacctgggtgttacatttaatatttaatgtaactggtcctagcaacatttaagggggatttctgaagcccaactccgggaggctgtgggctgcacattttgcactgtttttatatacttgtattcatatcctcttatcacctcagactcagacacaaggccttttacatggaaattttacaaattacttccatttatgtaaaataacgtcctgtgaccaagttgtttaaatggaaaataaagtgctttctttaaggaaaaaaaa

SEQ ID NO: 31 - DNA, 9549 bp, Homo sapiens
  misc_feature: Incyte ID No LI350243.22000MAY01
tgggcatgtactgaccaatgtggcaggtctgagaacatagctgaagctgaaaataggaaagctgggggcaaggaagagccttgaatcttgaggtgggacgttgactctaagatgttcttgaaccagtgggagcctccggagggaaaggagtggatgcaaacccggttgagacatacgacagtggggatgaatgggacattggagtagggaatctcatcattgacctggtacgccgatctggaaaaggaccagcagaaactggaaatgtcaggctcaaaggaggtggggataccggctcccaatgctgtggccacactaccagacaacatcaagtttgtgaccccagtgccaggtcctcaagggaaggaaggcaaatcaaaatccaaaaggagtaagagtggcaaagacactagcaaatccactccagggacttccctgttcactccaagtgagggggcagctagcaagaaagaggtgcaggggcgctcaggagatggtgccaatgctggaggcctggttgctgctattgctcccaagggctcagaagaaggcggctaaggcatcccgcaagtgtagccgggttcccaaaaggagaaggagaacagctcatctaagagcaagaaggagagaagcgaagtgagctggtgtgacttgttccagacaaatggattcctggggtcctccatgccatgttcccattgtggaaggactggtgtgtgtgtcagtatgatggaagtgcaagggttgatacaaggagctgtggcagccacttgggagtatagctattgagcctggggcagctgctcaatcctttgggaactatcacggatcctagagtgaaggcgggaatgagtgtcgccttgctaacagaaagtcanatgtctgaataggatggagtccccttgtttccacacccagcagtgctgccaatacaccttttggtgcccagtggtcaacaatgacatctcatctccttgttgtagcagatcatggttcgtgaccccatcagagtggggtgctcaccactatgtgatgtggctctggccacagagcctgagtgcttgggcccctgtgaacctggaactagcgtcaaccttgaaggcatcgtgtggcaggaaacagaagatgggatgttggtggtaaatgtaacgtggagggaacaagacaataatgtaggtacacgtcctgtgacgtgcacacgacatgatgntgggcacccccgcaggttctgtgactccccgaccagtgacctggagaatgcgcaatggccggggtagaggcaaacgcatgcgtcccaacagtgatacacctgtcaatgagacagccacagcctcgtgacagcaaaggcaccgagtgacagcagctattcactgggctaggagccaatagcaaaggccgtcggggcagccagaattcttcagagcaccgcccacctagccagcagcacttctgaggatgtacaaggccagcccctcctcagctaataagcggatttacaaacccctttcagacatggagctgaattctagctcagaggactccaaagggaccaagcgtgtccgtactaattccatgggctcagccactggcccccttcctgggacaaaggtagaacccactcttctggacagaaactgcccctccccccgtcctaattgaactgtccccacccaaactgcaacaagaagtacaagcacatcaatggacttaagtaccaccaagctcatgcccatacagatgatgacagcaagccgggaagcgggatggggacagtgagtacggagaggaacctattctccatgcagatcttgggagctgcaacggtgcatctgtctctacaaaaaggttccttgtcccctgcccgctcagcataccccctaagttcgacttgtagagcccccatagcccttcgtccttcaagcaaatttcagcacaaaaggcctctgtaagaaaaagttgagtggggaaggggacacagaccttgggggccttatcccaatgatggctctgatgatggaccctcagtgatggatgaaacaagcaatgatgcctttgattctttagacaggaagtgtatggaaaaagacacaatgtacaaacacctctagtttaaaaccgtgaaaagattccattccaagagcctaaagtcagcccagtcccattgcccctgccatccccccacagcaaatctacaccttccagacagccaccttcactagcagcgagcccaggctcttcctcaggcttgaccgccacagtggcacaagccatgccctacagtccccaactcaagcccattcagcccatagcccactgttatgggagaacctttcacagtcaaccctgccttgactccagccaaggacaagaaaaagaaagacaaataatagaaggaatcttcaaaggaacttgtaagtcctctgacccctgggacacccttgtcgagcagatggtaagtcattatagcccattcagggaatcttcaggaaatgggatgaaaatggaggggctcctaaatggctcatcagacccccaccaaagccgactggctagcatcaaggctgaagccgacaagatctacagtttcacggacaatgcccccagcccttccattggaggcagtagccgccttgaaaacactacccctactcagcccctgactcccttacatgtggtgacccagaatggagctgaagccagctcagtcaaaaccaacagccctgcatactctgacatctctgatgctggggaggatggggagggcaaggtagacagtgtcaaatcaaaggacgccgaacagttggttaaagaaggggctaagaaaactctttttccccctcagcctcagagcaaagactcaccatattaccaaggctttgagagttactattctccaagttatgcacagtccagccctggggctctgaaccccagcagccaggcaggagtggagagccaggccctgaagacaaaaagggatgaggaacctgagagcatagaagggaaagtgaagaacgatatctgtgaagaaaagaagcccgagctgagcagttccagtcagcagccctcggtcatccagcagcgtcccaatatgtacatgcagtccctgtactacaaccagtatgcctatgtacccccctatggctacagcgaccagagttaccacacccaccttctgagcactaacacggcttaccggcagcagtacgaagaacagcagaaacgccagagcttagagcagcagcagcggggagtggacaagaaggcagagatgggcctgaaggagcgggaggcagcactcaaggaagagtggaagcaaaagccgtcaattccaccaactctcaccaaggcccccagcctgacagacctggtgaaatcaggacctggcaaggccaaggagccaggggctgacccagccaaatcagtcatcattcccaagttagatgactcttcaaaactcccgggccaggcccctgaaggccttaaagtgaagctgagtgatgccagccacctaagcaaggaggcctctgaggccaagacaggtgctgagtgtggtcgacaggcagagatggatccaatactctggtaccgacaggaggcagagccccggatgtggacatatgtttatcctgccaagtactcagacatcaagtcagaggatgagcggtggaaggaggagcgggaccgcaaattgaaggaggaaaggagtcggagtaaggactctgtccccaaggaagatgggaaggaaagcacaagtagtgactgcaagctgcccacgtcagaggagtctcgccttgggagcaaggagccccggccaagtgtccatgtgcctgtgtcctccccacttacccagcaccagtcctacatcccctacatgcacggctattcctacagtcagtcctacgaccccaaccaccccagctaccggagcatgcctgctgtgatgatgcagaactacccaggttcctacctgccttccagctactctttttccccatatggcagcaaggtctcaggtggtgaagatgctgacaaggcacgagccagccccagtgtgacttgtaaatccagctcagagtccaaagccctggacatcttgcagcagcatgccagtcactacaagagcaagtctcccacgataagtgataaaacttctcaggagagagatcgaggaggctgtggggtggttgggggtggtggcagctgtagcagcgtcgggggagcaagtgggggtgaacggagtgttgaccggccccgcacctctccttcccagcgcctgatgtccacacaccaccaccaccaccacttggggtactcattgctcccagcacagtacaacttaccctatgcagcagggctttcttctacagccattgttgccagccaacaaggctcaactccctcactctacccaccccccaggaggtgagaatgacaccaagtgcccggataaagtcagcttcacgggcccggactggcttacccaaggaggtgctgaaggtgccgtttagacatcagttaaatggtgttgatcatcctgtttgccgtttccaccatgactgaaggcagacccttggctatctcacctccaccagacctccggactacctgaccctacctcttcctcaggagctggagagctggtacttagcaaaaatatttattctctcagccacagttatgactattgtggcctctgtggagatgaaggcacgggaagcaaccaggggaacatggcctcagcccagagaagccactgctctgttccccaagcccttggtctgctgctggagcagtaccagcccccccgcccaccagggagggacccccacccccaagacactgggtaaggtctgaagacagcacagcagccatacccctcaccatcattaccaccatcaccagattctgcatctccctagtgctttgcaccctgggaattggcagcatgtggaggaactagaatctcaggaaagaaattgggggttgttttctacataattgtgaaaacaaggtcttcaaatgtggagacttctccccatttacatgagcacatataaacgctcacaacctagcctggaaagggaagaccaaggcatctgccccaacatggccttgagctgcctgtgaggcagggggcaggggttccaacaccagcacagggctccccagggacactgggagcaagctggtgctggagcatgaatgacgtctgtgaagtagaacctgcgtccccactaagtcctgctggcttcttattccccacactccttgcccttttcccttccctcctaaccccttggtgcctttgcccagggggatccccacactgggtcttgcctcttcttttccactgctgggctcttaagcctcagaccagataaactagtattccccccagcttggggagaccttggagtctgccaggtcaccttagggcaaggcccagaaggcagcccctgggagcacccagcagttcttggagatgtcctgtcatctagccatctgatatcttcctcatttgaggccacagatatatacagcccaattcctctgtctacaagtacatgattttatatagctcagtctataacctccatgtgggccaatataagctgtgtttcttgggtaacacatatccttgtttgaggggcccactggccatgggaggttatttgttccttagaccctggaataacacatccaagccattacttattagagtctcagaatgtactcagtggagctgtgctttgaggcagccaacatttctctgctctccttagaaatgcagtctcccaatggaagctttatactctttgtactgggaaagtgaggatgatttggtagctttattggggtcatgtcttccccaaggtgtggggagcttagcttacttggcttttgaggtatcatccctctgttctcccctcctatctttccatgaccctctggattgagagagagagataaagactgacagacaccagtgtaggctggaaaagggagtgtgtgaccagagtgccaaaagtgactaggagcaggaacttggctccgacttcagtttggaaaactgggaaatacggggcacagctaagcacaatgcccagtagtagttgatttccaaggaccctggaaccctacacttgagaggcttagggtcaccatctgctcaagaggatcccctctgatctacaggccttttccctaggtttctgcctcctcgtttttgttcaagttgggttctgagtcctccccaaaaaccattgttttagacctcttggcaggggccccaaaacagcctccctcatacccatcattccgtctgccttctgctgccctcatgggcagtgctctgagcagtgacctccctttcctccgtggaagtagctagtgcagacaccgtcatcccaccccacctgagtcaccccaaccaagaggggtgacttgaatttcagcctgattatgccctcctggggctcctgtgaggtggagccaaggttcccttcttcttgttccctgtattgtttttaaatattgttgtgtgttttgtatctgtggcactggcctgcagcatactctgtatatattgtaaaaggaaaccgttaggagtaattttcttttgcattggggcaggcatggccctgcattcctgccctttccactacattctgtaacacagaggacgaacttctgtattaactggggcagccttgggttctccagaagaagaacaggtttttcttttccttggtgaatttttcttcttaaacattttggctctttgatcctcatatccaagttctcccctgaagagtaggagctgctcagaagagcaggttgaaagccaccatgggcagatcctgatgcctggccgggcctagtcttccctctgaaataacatgaagcagcagctgtggagattcttgacaagtgctgagtgaaagatgttgcttgccacacctgctacatggtggatggagataacatcatggtgtggagcttaccatgtggcatccatgacctagtcagaggggatgagatgctcagcaggggtcactcacatgcgctatcccaccccacaaaatcataatgtgcatggataaaatttgctaccaagggcacaagaccaccagaccaagcctgtttatgagccacaccactgcccaggccctcacagaccattgctcacggggcttcccatagaggagaagctaaagagggagggggcctcatccccagatagatcaggcaaggcttgggagagctgctctttaggattccacatcaactacttcctcattttaaggtatggcagttcccttcatccccttttcctgccttgtacatgtacatgtatgaaatttccttactcttaccgaactctctccacacatcacaaggtcaaagaaccacacgcttaggaagggtaagaggggcaccctatggaaatgaaatgggtgatttcttgagtctcttttttccacgtttaaggggccatggcaggacttagagttgcgagttaagactgcagagggctagagaattatttcatacaggctttgaggccacccatgtcacttatccccgtataccctctcaccatccccttgtctactctgatgcccccaaagatgcaactgggccagttaagttgggcccccataattctgggggcctttgttgtttgttttaattacttgggcatccccaggaagctttccaagtgatcttcctaccatgggcccccctcctgggatcaagcccctccccaggccctgtccccagcccctcctgccccagcccacccgcttgccgcttggtgctcagcccgcagcattgggagcaggtcggctccacactggaggcccgggctggaggggcagtgttgctgttcatagattttgttccattggcgttgctctgttgaatttaatttcaagtcttcctgattcttcccttctgtaaaagtgtacattaccaagttccttgtttttttatatatatatntgggtgnntatatatacaancngtactctttttgcctttgtacattcaggcaagaagagaaaataaatctttttaagagacaatcacaaatctgtgagggctgctggttatttctcctggagtttgctgctgagctgcctcttccttcctcccaattttcctgttctccctcagctctcctgatcttcctggccctgctccatatgcatcctcagcttcactttcccgtggctgatggcaagctgtggaatccagtgtccagactacctgccttgtaacccttttctgcccagcattgttttctggcttggccactggcttagcccaggagctttactctgtgcccctggcctcccctctcttcacctttagatttccattcaccgaagtggctttggacccctgggtactctgggacctgtttcctggaggccctggcttgggacactcacctgagaaaactatgcagctgggagctctctgcctaagagtttgcactatttaaacctgcctgggagttaggacggatggttttaggaatgaccggaaaaactacccctaaaactcccccgacattccagcctctagaatgctctgatccagagctcagtggatgattcccagctggtggactcctgtggctaccccatcaagacaaagggctaggggtttatgggtcaagagtatttgatcagaattttaaagggtggtatactctgaaacacagcccaacctaaaccattgtttggccgctttctcttttcctctaccttcctcatccccacttttttccctttctctctacttcctcttcttaattggctttggaattgaaatatatttttaaattatttgttgtatttattgaataaagtttttaatgtccctgttcttaaaaaaaaaaaaaaaaaaaaaaaagtttgggacactggactcccgtgagctggaaggaacagatttaatatctaggggctgggtatccccacattaactcatttggggggtccagggaccctgggcaatataagtattctagctcagtgtcgtggagatcatctacccagtgctggggcttctgtggacagtgcgaggacccacgtgaccctggaggagctggtccaggtggactgaactcccggcatctttacaagagcagagcgatgatcagcattcctgccgctgcttgctggggctcagccatgggctgcacaggagccagtgtggcttcgtggcccatgtgggaacgcacctgtctgttggatgatgctgggactccaaaggatttcacatactgcatctccttcaacaaggatctgctgacctgctgggatccagcaggagaataagcatggccccttgtcgaacttgggggtgctgaatagcttggcgaatgtcctctcacagcacctcaaaccaaaaagacaccctgatgcagcgcttgcggcattgggcttcagaatggtgccacacacacccagcccttctggggatcactgaccaacaggacacggccaccatctgtgcaagtatgccaaaaca

SEQ ID NO: 32 - DNA, 1818 bp, Homo sapiens
  misc_feature: Incyte ID No LI445188.12000MAY01
gtcagctattttctacatgcttcatttgcagtgtaatattggattgtatgagactttgggttttgtgttaatacctacagaaaatgttgatattttctcttagcaggctgtcaaccaggttaggttcaggtcataagtttctacccacattctttgaactgtagttgtcattttagtttatttttcaaaaacttttgcagtacctttttggtctgtcttgtgtgtgccttgcagtgaacagtctggatttggacagtggtctgtctgttagttcagtttctcaagcctttgtcacactaataggattggatttatgtatgtccagcttgggaattattacaggaattaaaaacaactttttagagtgctttcctgagctctctttctatttgttcccccttctactttttgcttccctgtggctgctgtttctatcctccagccagagagctagtgtttattttctccattgtgttacacacttgtgcagctgcaaccaccatatccagggcccaatggtaggaggtagagaagaaaagcaaaagggattggcctcatcctcttacaacgatagttccattgaatagagagaaaggttttcctgcctcagagtgttggctgcactaggcttttgttactgtagtctggccctgttaccatgggattgcttgcatgtggggatacaggagaattcagaaaagaaaaaaagatttgctatttctacattctccctgagcattaagacttcccttgcccattcctcaattcaaagctaaggcttcttctggagctgcctctgtgggcggttcgggagataccaaaggagaaaaagtaccactgttgatatggtggtatttcaaattctggtctaccctatttcacatgccttgtttacttttcagagctgacagattgctgctccatgcattctgtccagtttcctaagagagacagcttggagtatgcttaatccatcttacctgggactgaaacagctgcttattttgccgttaaaaattacatgcagtttactgcgtggctccgggtttgtttgtctgtttttcctcctttaataggtttattcagaaaacatgtccactgcaattagggaggtaggagtttggagacagaccagaacacttctactgaagaattacttaattaaatgcagaaccaaaaagagtagtgttcaggaaattctttttccactattttttttattttggttaatattaattagcatgatgcatccaaataagaaatatgaagaagtgcctaatatagaactcaatcctatggacaagtttactctttctaatctaattcttggatatactccagtgactaatattacaagcagcatcatgcagaaagtgtctactgatcatctacctgatgtcataattactgaagaatatacaaatgaaaaagaaatgttaacatccagtctctctaagccgagcaactttgtaggtgtggttttcaaagactccatgtcctatgaacttcgtttttttcctgatatgattccagtatcttctatttatatggattcaagagctggctgttcaaaatcatgtgaggctgctcagtactggtcctcaggtttcacagttttacaagcatccatagatgctgccattatacagttgaagaccaatgtttctctttggaaggagctggagtcaactaaagctgttattatgggagaaactgctgttgtagaaatagatacctttccccgaggagtaattttaatatacctagttatagcattttcaccttttggatactttttggcaattcatatcgtagcag

SEQ ID NO: 33 - DNA, 2198 bp, Homo sapiens
  misc_feature: Incyte ID No LI244378.12000MAY01
gagattttggaatctggtgatactgttgtttattacactagcctattacattttctttctttataataactgtttatgtgagtttcattgaaaatcgtggctctattgctttattcatggtttccacaactctttttgtaacaccttaattttctctcttaggtattttcctgttaaataagatatcaaaagcatcaatcacaagacagagaaaacgtattaggaaaaacttgtactagtatattctaaactgaaatggaaaaactcattattcatattttaataaataaaactcccaaatgatcttgctaaattatcccactatttagaagtagaaaataagagaagagtgctttcaaggaaatttgtcatattctcccttctatctagaagttgacaatgaattgcaataaaactaagacataactgaatgctgaaaaataccatggattaatgttatggcttctttatttttgtatatccattcccacaaaattgaatttaagattattaatgaaaattctatattaagtaataatgcttgtattagtaatgggcaaagaagtctttctttgttacaagtaaccgttagtaaaagatgtactttgacatttctgaaggttaaaccttatatgtaaatattgtaatttaaatatgattggacagctgttaagttttcggggagggaagcagatgccagcgttctctgctgaaaagaacaagatggcgcttgacggaccacagcagatggagctggaggaggaaaaggcaggcagtggactttgccagtattatcttgtcccaggattggagaactccagggtccactggcatattaattgggtggggggaaagccacaaggaacagttctgaactccaggtgcatttgcagctgattttgaatgataagacccaaaacctctggaggcggcaggtgcacgggaatgaactaaatgtttaaagtgtgccttattacaggaggagctacagctgctcccaggaattaggactccctgtgtacagggaagtagtccaggccatgggtaagaagaaagtgttggtcaaggtgcatctcaaggacaagtttgtcatagactgtggacaaaaatatcagcatcagtgatgtgacacaccagttccttggtggttctaagaaatggaggtatactttgtgcacaaggatccttaccccacaaggtggactccttggtgtcactggtgtattgtgtagagaaagtgtcccagtatctcagtcttatagaaatgattgggtagacctggataaggcagaatcacagaagcatcaacagaagtgatctgacctgcctgctaagcatcccgagctctttcaaagcactggggcattgcacagccccaagtggaatgcgtgcttaggaagagactgttggccctgggctctggctcattattaggggctggggtcccttaatccaggtctctggcttttgaatgggtccagaaattcattggggaatttctgtgatggccagagaaaatgctcatctgtcatcttcatggatgaaattgactcttttggctcctcagagctggagggggatgctggagggaacagtgaagtgcagcagatgatgctttcgagggtggctttgaggccaccaagagtatcacgcacggtttatcacgggtgcgaatacgagatgagatacctcgtgactccggctgtgtttgtcgcccaggggcaccattgacagaaaaattgaattcccaacctgcaatgagtaggcctggctggtcactttgaagattcattctacagaaatgaaccctgacccagggggatcaacctgagaaaaattgctgagcttatgccaggagcatcaggctgaagtgaagggcatgtgcaccagaaaccggcatgtatgccctgagtagcgagtccatgtcactcagaaggactttgaggtagtccaaagtcctatctcatgccaaaggacagtgaggaaaaacatgtccatcaagaaactatggaattgagggcacatcctttgtgtgtatcacatacaataaaactcagtgggacaagcaaaaaaaaaaaaaaaaaaagttttggggagggatgatctgctagtgtataatcaataacttttaacatttagcttgtatttagtaacatttcattgtgttttgaaaaaaagtaaaaaatcttttgaagcac

SEQ ID NO: 34 - DNA, 431 bp, Homo sapiens
  misc_feature: Incyte ID No LI236574.152000MAY01
tataaatggcatttcttaatcttactgtatttccatggaataaaagtaattcttatgcacactgaagttaaaaaaaaatagcatttaaaatttctgctcaggaaagtagtataatttttaacataagtgagtttctctttgtgttataatgtaatgaattcttatacgcatatggagaggaaaatgacatttttctatttatggttttagttcagcctttaagataccttgatgaagacctggactattgaatggagcagaaattcacctctctcactgactattacagttgcatttttatggagttcttcttctcctaggattcctaagactgctgctgaatttataaaaattaagtttgtgaatgtgactacttagtggtgtatatgagactttcaagggaattaaataaataactcagaatgttattg

SEQ ID NO: 35 - DNA, 3730 bp, Homo sapiens
  misc_feature: Incyte ID No LI010100.202000MAY01
ctgaggttattttggaggggaaggggggcttctagatgtaccttttatgctttattaaaaagtatattacctattaaagtttgtttaaatctatttgagttaaaaatttgagatgtaccgtgttttctaaacactgtctccagtggcttcaaaaacacaggtactgttggaaggcattcacttttgagtggcctttgacctcagttttaatctgattaattcagtcattttataatcttgctcttggtgagtttaaactagatttggctaaaatagaggtgggagtgttggcccttcaaagacagtttgatgtgttcaaaattgtagtgcatttaaaagggttagacttctgtgatatttgtccaacgtcaatcagtactcattgcatttttttacttaggctcttgagtgtgagtggactactggtatcatatgcaacttctgaactaagttttaagagaatatgaatcatcagttgaataaaaacctgaacagtggtgaatgcaactttgagacatatggaaaggctagagttggcatccaggggacacaaatagtttgaagactctggtagtagaatgatgcaagtttccttaaaattaggcaatatataatattgatactacgaaattcagtacttttccggtaggagggggtcaggtctcattgcctttgtccgtaccccaaaaatatgaaaaggtggtctacaagtttggttgtggcattgtcacagcagttggccccatgttacatttcctcttgtggcacctctgagaggtgtgtcttcattacctcttgacaagagtatattttgattgactgctctatgtgcttgttgatcttttatagcatttaaatcaaacggtatcgagatggattgcgttatgaaaccataatggtcccagatgacgctagtgatgggacagtacgacttcgtggactaccatttggttgcagcaaagaggaaatagttcagttctttcaaggtacctctagtattagtcaaagtttagtagtattgtaattttatatttgtattgttttactgtttttaatttgtaaaacccatttgattttggaacttttaagtttaactatgatagtcttggttaatgtattaaaataatatgctccagttaatattcaatacagaatgtgtagaatatgtaaaacctaattaatgtgcagtaacatatttgagaattgtgatgaaatgaaccgtgaattagtatatggagcatatatttgattttgtagcccttttttcatgtaatataggtgggcttttagagtgtgtaattacacagtgttgtttacactaggtcgcaaagaaatttatttaatccagtaatatttgaaaaaatctttcatttgtattatggggtgatgggaaactaagcttttttgttttgttttgttttgttttgtttagacttgttttaaatatttgattcagatgggaatgtttcagcctttaacactgttccccttgatggggttatttgtccttgggttaaagggttggaaatcgtgccaaatgggataacattgacgatggactaccaggggagaagcacaggggaggccttcgtgcagtttgcttcaaaggagatagctagaaaatgctctggggaagatataaggaaagaatagggcacaggtggcgatggagagtttgggatggtgttaaatttttatttttgggggttgtgggtcactattgcttaaatgggggggtagccataacatttttctggggtagtttaaaagaattgataattatctaaatttaacttggaaactacagatttgggtggggaattaatgctaatagttaaatgtcaaaattagggattgtttcccattttctcctgtagggatgttgttagataaaatgcttttagtataaatcaccctatttcccttttaaaacaccctattatgtacttaaaaggagacaaagctaaaaatatacttttatgaattgtttttccgtgactaaattcctttggtaaaacttaaatagtcggaattaagtttgtaggtttaaactgttgacttacggtgtattttaggcaatttaaatttggggttatgtatctagatgatagaaaaacttactgcattttgcccatgtatctaatttacactaatacatttatgctggaaaccctgcaccttaaaaacatttttaaaataggtatattggagaccttcagaagtagcaggaggtcaaaccaaaggattttatgatccacccaagaagattgccggggacagcgacccggggaccattatgatagacccattaggaggaaaaaggggggttattatgggagctgggccgtggaagtatgtatgacagaatgcgacgaaggaggtgattggatatgatggtggttatggaggttttgatgactatggtgggtataataattaacgggtatagtggaattaatggttttgatgacagaatgagagatggaagaggtatgggaggacatggctatggtggagctggtgatgcaagttccaggtttccctggtggtcatttcgtacatattagagggttgccttttcgtgcaactgaaaatgacattgctaatttcttctcaccactaaatccaatacgaagttcatattgatattgggaagctgatggccagagccacaggagaagcagatgttagagtttgtgacacatgaagatgcagtagctgccatgtctaaagataaaaataacatgcgtaagtggtgtctttggcacacaatcttatttcctaaacgtgaatttataaaataagaggctctaagatctgtaggtaacgcttggcagttgttggggatttaaaaccatttgacctctataatggctttctgtgggctttatatatcgcttgtactagtaattaataagcatactttgtttaatattaccttaattgatcttttttacaaagcttgtattgatgaattttatccatcattcctttctcccctgttactctttctttttttcttaaagaacatcgatatattgaactcttcttgaattctactcctggaggcggctctggcatgggaggttctggaatgggaggcctacggaagagatggaatgcggcatgtaaagtttttaaaatatgtcagggttagctgcttatcgatgagtctcaattttttttcttttttctttttaaagataatcagggaggctatggatcagttggaagaatgggaatggggaacaattacagtggaggatatggtactcctgatggtttgggtggttatggtaagtatctctagttcagtttgtgttagtccgcatatgtagtgcaaactttaaagtgcaggtattacttttattattttatgcagatatctcctgctgagtgattcttaatatctttttcttaaggccgtggtggtggaggcagtggaggttactatgggcaaggcggcatgagtggaggtggatggccgtgggatgtactgaaaaccaaaaacaccaacatacaagtcttgacaacagcatctggtctactagactttcttacagatttaatttcttttgtattttaagaactttataatgactgaaggaatgtgttttcaaaatattatttggtaaagcaacagattgtgatgggaaaatgttttctgtaggtttatttgttgcatactttgacttaaaaataaatttttatattcaaaccacaaaaaaaa

SEQ ID NO: 36 - DNA, 790 bp, Homo sapiens
  misc_feature: Incyte ID No LI037940.62000MAY01
ggaagagtgtagaacagagtttttctttctgagtgttctttttaacttcagtgttagtgctagtagtaaccttatatgtcatctctccagaggtcttcatcaaggattcacatggcagtcctgatctgaagggcatatttaaaatgtacttgtccagatttcactgctaaagattatgattcagtaggtctaggatgggcttcagccgtttgggttgattgtgatagcatacatgtctttagttgaggactgctggtattacctgcaattttgagatagagaaaagtgcaggaaaatcttcctccaggatcactaaagctgagccctagcttgttacctgtggaggcattgagacatttgttcacagaaagtacagtttggatttttggttttttttagaagaaagctcattatttagaaattttgcaatgttaacttttctactaataacacaatcatcttcacttatatttttaggctcatgctgaagattcggttatggaccatcatttccggaagccagcaaatgatataacgtctcagctggagatcaattttggagacccttggccgcccaggacgtggcggcaggggaggacgaggtggacgtgggcgtggtgggcgcccaaaccgtggcagcaggaccgacaagtcaagtgcttctgctcctgatgtggatgacccagaggcattcccagctctggcttaactggatgccataagacaaccctggttcctttgtgaacccttctgttcaaagcttttgcatgcttaaggattccaaacgact

SEQ ID NO: 37 - DNA, 8059 bp, Homo sapiens
  misc_feature: Incyte ID No LI228550.32000MAY01
ggcagaggaatctgttcctcaaggcattcacggacttcctggccttcatggtcctctttaactacatcatccctgtgtccatgtacgtcacggtcgagatgcagaagttcctcggctcttacttcatcacctgggacgaagacatgtttgacgaggagactggcgaggggcctctggtgaacacgtcggacctcaatgaagagctgggacaggtggagtacatcttcacagacaagaccggcaccctcacggaaaacaacatggagttcaaggagtgctgcatcgaaggccatgtctacgtgccccacgtcatctgcaacgggcaggtcctcccagagtcgtcaggaatcgacatgattgactcgtcccccagcgtcaacgggagggagcgcgaggagctgtttttccgggccctctgtctctgccacaccgtccaggtgaaagacgatgacagcgttagnacggccccaggaaatcgccggacggggggaaatcctgtgtgtacatctcatcctcgcccgacgaggtggcgctggtcgaaggtgtccagagacttggctttacctacctaaggctgaaggacaattacatggagatattaaacagggagaaccacatcgaaaggtttgaattgctggaaattttgagttttgactcagtcagaaggagaatgagtgtaattgtaaaatctgctacaggagaaatttatctgttttgcaaaggagcagattcttcgatattcccccgagtgatagaaggcaaagttgaccagatccgagccagagtggagcgtaacgcagtggaggggctccgaactttgtgtgttgcttataaaaggctgatccaagaagaatatgaaggcatttgtaagctgctgcaggctgccaaagtggcccttcaagatcgagagaaaaagttagcagaagccctatgagcaaatagagaaagatcttactctgcttggtgctacagctgttgaggaccggctgcaggagaaagctgcagacaccatcgaggccctgcagaaggccgggatcaaaatctggggtttcaccgggagacaagatggagacgcccgcggccacatgctacgcctgcaaggctcttccgcaggaacacgcagctgctggagctgaccaccaagaggatcgaggagcagagcctgcacgacgtcctgttcgagctgagcaagacggtcctgcgccacagcgggagccttgaccagagacaacctgtccggactttcagcagatatgcaggactacggtttaattatcgacggagctgcactgtctctgataatgaagcctcgagaagacgggagttccggcaactacagggagctcttcctggaaatctgccggagctgcagcgcggtgctctgctgccgcatggcgcccttgcagaaggctcagattgttaaattaatcaaattttcaaaagagcacccaatcacgttagcaattggcgatggtgcaaatgatgtcagcatgattctggaagcgcacgtgggcataggtgtcatcggcaaggaaggccgccaggctgccaggaacagcgactatgcaatcccaaagtttaagcatttgaagaagatgctgcttgttcacgggcatttgttattacattaggatcgtgctgaggctcgtgcagtacttgcttctataagaacgtctgcttcatcttccctcagtttttataccagttcttctgtgggttttcacaacagactttgtacgacaccgcgtatctgaccctctacaacatcagcttcacctccctccccatcctcctgtacagcctcatggagcagcatgttggcattgacgtgctcaagagagacccgaccctgtacagggacgtcgccaagaatgccctgctgcgctggcgcgtgttcatctactggacgctcctgggactgtttgacgcactggtgttcttctttggtgcttatttccgtgtttgaaaatacaactgtgacaaggcaacggcgcagcatatttggcaaacgtggacgtttggaacgctggtattcaccgtgatggtgttcacagttacactaaagcttgcattggacacacactactggacttggatcaaccattttgtcatctgggggtcgctgctgttctacgttgtcttttcacttctctggggaggagtgatctggccgttcctcaactaccagaggatgtactacgtgttcatccagatgctgtccagcgggcccgcctggctggccatcgtgctgctggtgaccatcagcctccttcccgacgtcctcaagaaagtcctgtgccggcagctgtggccaacagcaacagagagagtccagactaagagccagtgcctttctgtcgagcagtcaaccatctttatgctttctcagacttccagcagcctgagtttctgatggaacaagagcccaggctaccagagcacctgtccctcggccgcctggtacagctcccactctcagcaggtgacactcgcggcctggaaggagaaggtgtccacggagcccccacccatcctcggcggttcccatcaccactgcagttccatcccaagtcacagctgccctaggtcccgtgtgggaatgctcgtgtgatggatggtcctaagcctgtggagactgtgcacgtgcctcttcctggcccccagcaggcaaggaggggggtcacaggccttgccctcgagcatggcaccctggccgcctggacccagcactgtggttgttgagccacaccagtggcctctgggcattcggctcaacgcaggagggacattctgctggcccaccctgcgcgctgtcatgcagaggccattcccccaggcctgtgtcttcacccacctgccgtcattggcctttgctgtcactgggagagaagagccgtccagggacccatggtggcccacatgtggatgccacatgctgctgtttcctgcttgcccggccaccacccatgccctccatagggtgaggtggagccatggtggtgcgtcctttactcaacaaccctccaatccggatgctgtgggaagggccgggtcactcggataccatcatccctgcggatgcaccgccgtaccctgctcatctgggagtggtttccctgcggttacgtccaagcccgcctgccctgtgtgttggggctggctgagtttcggtctccccatcaccggccgcctcgtggagaaggcagtgccacgtgggaggacaaggccacgccggcagcttccagccctgccgcagaagtgccaggatgtccatcagccactcgccagggcacggagccgtcagtccactgttacgggagaatgttgatttcgcgggtgcgagggccgggagacagatacttggctgtgatgagcagacatcctctgtccccgtggaggggtcaacaccaaggtggtgttcgtgcaccagaacctgtctcgggctgacgggggtggcacacaggacacgggtggatcccaacaggcagcaccgcacctccgcccgcctcccgcactgcagctccgcccgccgggctctgcgtctccacgtcccctcgtcccatccccacgtcccctcatcccgtcacctcgtccccacatccccttgccccgtcacctcgtcctcatgtccccttgtcctgtcacctcgtccccacgtcccctcgtctcatccccacgtcctctcgtccccttgtcccgtccccacataccctcgtccccatgtccccacgcaggggctctccttcgtcttaggatctgttcagcgctgctctgggtgggttagcaaccccagggctgctgtgataggaagtccctgttgttctccgtactggcatttctatttctagaaataatatttgacatagccttaatggtccttaaagaagacatttcagtgtgagattcagacttcagacgctgaaactgctgccttttaggaaagcaccaccaacgctggaggaggagccggccctcacgcccgccccgcgccacgctgtggaacggggctccggcaagtgaaacccagagggtgtttccgaagtgtcttcaccattagttatttttggaagctcagatttcaccaatttgattgtataatcttttacctataaaatatttatttgaagtagagggtaaatcagcggtaagaacagtgaacacagtggttgggataaaataaggtgacaaacatcacccccaaagatgagggtagcgagcaactggcttgagcagacagaacgtgggaagactccatctctgtcccgaggggggcaggcggagggggtccccaggggccaccctgcccttgaggtccttgtgttggccgcccttggtttggcagccctgtgcccaacgcttgcccccgggcaaacaagtggtgtgtgcgtttttacagcccctttttaggaacccaaatatgggcataaatgtaacacctatagcgggggcagattctctgtatgtccagttaacaaattatgggtaaggtatttgttgagaaatcttaaaatggccttggcacggaagtatttccatagctgttaatatctcttttatccatttattgaacatactggtctaaattttaacaaataggtttttaaacgctttcatttttaagtttatgaaatttggggcactttaacatttaaaattctggtgagagttttgactgaatgttccaatctctgagtgtatacgcaaatttctacagattagattttatctctctacacacccctcttcttttctgggtatttctggtggcagtgattagttgaacagcacatttaaggcacgataattgctaacaccttttcctttaacaatttggtggcaatttcatctgctttcctaattgttttcattgttaattgccatccttcagccttaaaaatagaagattctcacgtgaaggtttagtaagttggggtcccaggctttggccggttgttggagattgtcccccatggttactttgtgaacaccaggttttaagctgtgaaagtcactaaaacttttacacactcccaaaaggtctttttaacaaattgctgtgggaaattattacnaatgaatgtgcctgatgatttggaacatagaccaaggcggcactgagataaaaaacagaacaggatgcagatagatccctcaggtgaactggacgttgccagggtcttccatgccaatgtttccacctcgcagtagttagtatttacttgccattaaactaactttgaagcaagtaatgtgcacactttgagcactttgttgagttttgaaaaatcttatttgttgctgcaccagggttaataaataaccaattttgtaattccagcatgttggtccagagacaccggtcactgattcacaccccagtccctgccacagaccggccacagaccgtctcagacacgcacagtgggcctgctgccaatgattcacaccccagttccctgccacagaccgtctcagacacggcacagttgggcctgtgggtagtgatacacccacgagtccctgccacagaccgtactcagacacgcaacagtgggcactgcttgcattcgcgttgttaccctgggcttttggctccacgcgtcactccatagccactgtccaccatgggggactatgcaccacaggaatcactcactatattgtactattgtacccaccacaaagcgtgcaagctctcgtgcacactatgccatgcacacaaacggtggtacaccaagttgttgaagctcctacacgctatacacacgacacacgtgtacattgcaccaagagcagtgtgtgacccctacagacattgcagaacgatgcagcggtgttttacacattaccccacagaccacgccccgtgtgacgatgctcccctacacaattacattattgcaccattattcatggaaccagcggtaaggttacctaacacacgggacgctggtgctacagtgcagtgtaccacggtaaaagcacactgtacaacgctctctacagggcttgcgtcctcacacactgtgttatgccacagcagaagagcacgtatgagcttctactggcacacatgcacacacatcacgcacatgtacatttcactacacactgtgcagcctcgcgtgcaacacgggtgcactattttccagtgtgtgtacaacccaaacaaattgatgttcccaagaacgtgtgtaaacctacacggttgcacactatctgtacacattgtgagctcccacacgttacacacaggatggcacatgggacacaccccaacacacgacacacggactccctaacagcaccactagacacaccacgatgttacaccaccaaacgagctcccagacatgtaaaccacacgtctcccaacacccgtgagcttcccacacatgtacacatgcacatgtacgcaccaccaacactatggcggaagctcctgcaggcgtgaatacacacatgcacacacatatacacacacggtgccacaaacaagtgcacactgtcctggtgtccctggcaactgcatcctggcctccttgctgaggggcccctggtgagaggcctctggatgggcatgggaagatgggctccctggccccccagcccatgcctccctgggatgaagagtccccctcctggcagaatgtctgggctttgcagaagcaggccccgggagtgaagtcgcagcttcacttacaccaggctgctctgtgagcaaggcttggtgccctggacaaggcccttccccctttagggaggtccagccttcgcaagctgaaacctcccctcggctcagccctatacccgggcggccacagaaggactggccacacccacgccgcacctcaatccgtgcacgcgtcggagcccccgccagccttctgccacgagccagctgggaagggccgcggctgcctaaagccccagtcaaccccagcctgtgtctgagccagacacaggcgaaccagccaggccacaccgttctcgagggaggaggcccagattcggccagcgtctccaacaggggtgaccatcccgctcggcttgctgagccgtttaaaccaaatgtttagaccaggctgtggggactcccctgagttgagccttggccaaggggtccggtgctgtcgccgggaaacctcccagcccttgttcttccaaacccactcagctcaatgtgttttgcactgaactagtactggataatacaaccactcttaatttaaatgttagtattaatttaatttgacaaactcagtgtctaacagctttgatatgcaggtccttgcattccctacatttcctttaggaagttacccatttgtaacttttaaaaacaggaaaaatatcaagttggccaaaagccaatctttttgtttttttaagctaaagggtggggtgaactgggaatgaaaacctttcctgatgtggtggtctataagcagccttggatgggatatggttagaagtggtcatggaaagtgcgtggattctacttttggcagaaaaatctaaagatcaatttatatagcttagattttttactttatcaagggtatacagaaattttaatatgcatatattggtgtctgacttaaaattataatggtctgggtcaccatttaaaatggtcggttcattatgtaattgtaataaaagaaggtcttcaaaaatgtatttaagcatgaatgggtatccatagttgtcatcatcataaatactggagtttatttttaaaattattaaacatagtaggtgcattaatcataaatcagtctcccacacagtaacatttaactgataattcattaatcagctttgagaaaattaacattgttcaattaagaccaatctaacatttcagtaaagtttattttgtatgcttctgtttttaactttttatttctgtagataagactgactggataatattataattggactttttcttcttagattatctaagcaggagacctgaatctgcttgcaataaagaataaaagtctgcttcagtttctttataaagaaactcacacaaaaaaaaaaaaaaaga

SEQ ID NO: 38 - DNA, 1423 bp, Homo sapiens
  misc_feature: Incyte ID No LI027320.12000MAY01
gcggccgggacgatgcctgcgcgcagtcgcaccgccccgcctccactccggctccccgccccgggctccgcccccgccgcttgaggcgcttcactccggcgaggcggggagggccccggactccgacggcggctcggacgccgacttcggaggtgggtccggggagcccgactcggaccgcggaggtgagcgggagctgaggctgaggagaggggagcttggggggcgcctgttgccaagggcagcggaggaggaaatggcaggtcctaatcaactctgcattcgccgctggactaccaagcatgtagctgtgtggctgaaggatgaaggcttttttgaatatgtggacattttatgcaataagcaccgacttgatggaatcacattgctaacattgactgaatatgatctccggtctcctcctctggaaatcaaagtcttaggggacattaaaaggttaatgctctcagtccgaaaattgcagaaaatacatattgatgttttggaagagatgggctacaacagtgacagtaccatgggttccatgacccctttcatcagtgctcttcagagtacagactggctctgtaatggggagctttccccatgactgtgacggacccatagactgacttgaattctgatcagtaccagtacatgaatggtaaaaacaaacattctgttcgaagattggacccagaatactggaagactatactgagttgtatataatgttttataatatttgggatttacatctttcattatggttatagtccatgagcgagtgcttgacatgcagacctatccacgactcccagatatattcttagacagcgttcctagaatcccatgggccctttgccatgaccggaagtatgtggcatgattctgtgctatatttggctcctggttcttcttcttcacaagcacagatatatgggcagtgtatgggagaaattacatcgagcctttgccatttggagtggctttggtatgaccctgactggcgttcacacatgtggagattacatgtttagtggccacacagtcgtcctaactatgctgaatttctttgtcaccgaatatacaccaagaagctggaatttcttgcacactttatcctggggttctcaacctctttggaatcttctttatcttggctgccccatgaacattattctattgatgtgtttattgctttttatataacaacaagactctttttgtactaccatactctggccaataccagaagcatatcagcagagtaggagagcaaggatttggtttcccatgttctctttttttgaatgcaatgttaatggcacagtacctaatgaatattgttggccattttcaaaaccagcaataatgaaaagactaattggatgaatactatctttctaatgaatttgtgatt

SEQ ID NO: 39 - DNA, 1594 bp, Homo sapiens
  misc_feature: Incyte ID No LI321475.12000MAY01
ctcactcatttctaaggaggactttaagcaaatgagtccagggatcatccagcagctcctcagctgctcctgccacttacccaaggaccaacaagcaaagctgccacctaccactctggagaaatacggctacagcacggtggctgtcacccttctcacactgggctccatgctggggacagcgctggtccttttccatagctgtgaggagaactacaggcttatcttacagctgtttgtgggcttggccgtcgggacactgtctggggacgctctgccnccaccttatccctcaggttcttgtgtttacataagcaggaagccccagaatttgggcatttccatgaaagcaaaggtcaatatttggaaactgatgggattaattggaggcatccatggatttgttcttgatagaacaaatgttttattcttcttgtatcaccaaatgacaagcagggcctgtcattggttaatgggcacgtgggtcattcccaccatcttgcactcaactctgaattaagtgaccaggcaggcagaggcaaatctgcttcaactatccagttgaaaagcccagaagattcacaggcagctgaaatgcctataggccagtatgacagcctccagacagaaaatgtaaagccattagctttgttagcaatcatgattctggttggggacagcctgtcataattttgcagatggcctagccataggagcagccttctcatcatgcatccgagtccaggagtgaccactacgattgctatcttgtgtcatggaaatcccacatgaaatgggagactttgccgtgctcttaagctctggactttctatgaagactgccatcctgatgaattttataagctccctaactgccttcatgggattatacattggcctttccgtgtcagctgatccatgtgttcaagactggatccttcacagtcactgctgggatgttctaatatttatcccttggttgaaatgcttcctgaaatgactcattgttcaaacacaacgaccctggcatgatgtttctgcctgcaaaaactttggattgatcctaggttggctttctctcctgctcttggctatatatgagcaaaaatattaaaatataagtgaggatcttcaacatctttcaaaaatgcatttatatagtcttactttgtttctttcattgcactctataatgatttttaaattaagaattttttatcttaggcaaagtgtgtctctttcaattcattaacttattaattttataatgccggtttattttttggaaacatataaatatcagactgtccttaattgaaattttgtctttggtttccaacaccatgatgaagctcttgcttttttaaaaagtagttagtaaattctgcatgaagttttagtaaactttaaaaaatagattttttccctaaggaaagaatggtcttggtagaaatttacaagtgggacaggatgcctgtcgggggtaaaatcaactgcaacctttttgatggttaatttttttccctgtgcaattataaaactataagcaagtcaagtgccaagccaatgttataaagactagttttaaaaaaaa

SEQ ID NO: 40 - DNA, 2138 bp, Homo sapiens
  misc_feature: Incyte ID No LI899552.52000MAY01
gtgaatatcaagtctccccaaccccacctggaggggtttcatccagcaagagcttgccttctgctcagcacactcctgggagtaacctatgagtgtcacagctgcaactccgaggtgccaaccaagcccagggctctggcttcttctggggtcaggccgctcttcagggttcttcgcactgcagtggtggtgcaggtcacacccaaagggatgagaagaaaaggaagtatgagcagcaaggcacagggcagtcaagactccagtccccccttctccccagggacttccagagaagccagagggttcacactctgtaccttcctgctcttgctgaagtgccctgcagtctgtctgccaggacaaaagtttggggctacacaggctggcctaacaatgccacaggtgctgatttggagggccgagaggagtgatgcgggcacttataaagaggagagaaaggaggagaggctcaccctcacgaggcttctgagaagggggtgaactgcagaagtgcagagggcaggagagacctcagcatctacccagtatgaggagtgtatcagagctgggaaggtgattccagagcaggggaaaatgcaagctccactaatacaaatgaggtgaggcaaccagtgcacagcgaggggttccacaagacccaacaacctcacaaatccaacagacgaccaacaatagctggggacatgctcaagacccacagagcaagtgcatgaagccagggcaaggggcagcagtgaggacaaatctctccatgagtactgtgggtcttagcctcaactcactaaaaggcctctagagacaataattaaaataaccagtggctcctcgaggaatggaggctaatgagcaaactgccaaacttattctggctggaattggtggctgctttctatggaacacacaagtctcaagaatggccaatgaccactctcactggggggttgcagctggcaactggagggatgctcatatttaactgacttagaattggcttgtgtctagcttggcatgatggtattcccattgtttagtatttcaatttcaacatcaaaataaaggctggataagggtgaatgaggaggtagctgcctcacccaagacaagtcttcatttgaaagggtggctaattacattttccttaatttacaaattgtatttgacccactgtgtatttctttaaagttcgcacagagtttggggtattcattttcagaagagaaacgttattggatctgccttcaccctaaatccctaaatcagccagagttcctggaaatgaccagggcaggctgttgatcaaagttcagagaaagtaagctcaccttaatgtctccccagttcctttgccaactcccctcatctactccccagaggccagggcccacctcatgcatctgcaggccacaccccatggtctatgccttcccatcatcccctaaatataagctcactcagcctgtggacaaaagatctgaaagacctatgtccatggcagaatcaatggggactaatacctattatgaaaataaagggctccttctcttccaggaatcagcacttggaacaggggtcaacataatatccatcaattggtcaaccaacaaaatgatttattgagaacccattgtgtgctcagcatttaactcaaagaatcaaaacacagacatcaagtatgggtttttagagagacatgggatatgtggtgaggggttctcgtcaaatcgtgttactgcaactactgtatttccaagccttacctttgggccctgaagcaatctatctgtagctgttcttgcactagctcctctttcccccacccctgatcccttggcctccagactgaatgtttgggctcaacaagacaaaatgtttggctcctaagtctcagctggagacagattacattcaactgagcaacagaatttgttctatggggcagcaataaaaatactagtttgtaataaccttttatgttctttaaagactccttagagtgtttgttaaagaaagataattttaacaacagtaaataagataaaatatctctcttgacaagaaagacctcttgctgcaacttcttaaccaagatacaaaatacttccttaaaaagtccatgc

SEQ ID NO: 41 - DNA, 1345 bp, Homo sapiens
  misc_feature: Incyte ID No LI1071848.12000MAY01
ggcgggaaggcgccggccgctaagaagccgaaagatgtccaggtcgggcgcggcggctgagaaggcggactccagacagcgaccccagatgaaggtaaatgaatataaagaaaatcaaaacatcgcttatgtgtctctgagaccagcacagactacagttttaataaaaacagctaaggtctatcttgcccccttttcactcagtaattaccagctagaccagcttatgtgccccaaatccctatcagaaaagaattctaacaatgaagtggcgtgtaagaagactaaaataaagaaaacttgcagaaggattatacctccaaagatgaaaaacacatcttccaaggcagaatccacgctgcaaaattcatcctcagctgttcatactgaaagtaacaagctacaacccaagagaacggcagatgcgatgaatctcagtgttgatgtggaaagtagtcaggatggagacagtgatgaagataccacaccatccctggatttttcggggattgtcaccctacggaaggaagagactgaagaacatatcagaaaacgcagacttttttgcttctcttcagttgtctgagtctgctgcaagactccgtgaaatgatagagaagagacagcctcctaaatccaaaagaaagaagcctaagagagaaaatgggattggatgtagaaggtcaatgcgattactaaaagttgatccttcgggagtttcattaccagcagctccaacaccgccgacattagtagcagatgaaactcctttgttacctcctgggcctttagaaatgacttctgaaaatcaagaagacaacaatgaacgatttaaaggatttctgcacacatgggcaggaatgagcaagccaagtagtaagaacactgagaagggattatctagcattaaaagctaccaaagccaatttaaatggcatggtcattagtgaagataccgtttacaaagtttaccacaggcccaatattctctatggctctccatccatcagaaactagaactttggtagcagttgggcccaaatttgggcaagttggactttgtgatttggtaagttattaaatttcttgaatatattatagtttgactaaagcaaataggctggaagagaataggctagagccatgtgtttataaatgttgcgtgagacttacaattttgggctttatgatgctttatgattccaaattttagaaatctggaagaatttaaatttgctttatagaactttaatatttttagcttgaatatcattaaccatctggtcataaattaactgccagaaaactttgttacactttgtgtgatcttttcacatatacattt

SEQ ID NO: 42 - DNA, 4707 bp, Homo sapiens
  misc_feature: Incyte ID No LI1072337.22000MAY01
ttttctatatgatcagaagagcagctactgtgattcctgggctaatgaagcctagaccccagctggattggaagcaaaggcctttccttcactgggaaaggtgctgctgctacccatctcattattctaagagtgattgagaatagaggggctgaagggaaacgaaagtaggagaatgttggccagagctagtagagaaagaacgtaatattgagaaagcagacatcagtgctaggtgaggtgctttctttagagagctgtgtgtatagactggggaggagtctgtctggggacaggaatgctgattccttttttttccctgggcagaatcctaatgtacctggctagctggtggtgagtaggggctttggggccaacttggtgggctccccaaggaaacccctttgaaaccaatggatgcattcacgggctcgggtctcaagaggaagttcgatgatgtggatgtgggctcatcagtttccaactcagatgatgagatctccagcagtgatagtgctgacagctgcgacagcctcaatcctcctaccactgccagcttcacacccacatccatcctgaagcggcagaagcagctgcggaggaagaatgtacgctttgaccaggtgactgtatactactttgcccggcgccaaggttttaccagtgtgcccagccagggtggtagctctctgggcatggcccagcggcataactctgtacggagctatacactctgtgagtttgcccaggaacaggaggtgaaccatcgagagattctgcgtgagcacctgaaggaagagagactccatgccaagaaaatgaagctgaccaagaatgggacagtggagtcggtggaggctgatggcctgacgctggatgatgtgtcagatgaagatattgatgtggaaaatgtggaggtggatgattacttcttcctgcagcctctgcccaccaaacggcgacgggccctgctgagggcttctggggtccaccngtattgatgctgaagagaagcaagaacttcgagccatccgcctgtcacgggaagaatgtggttgtgactgccgactgtattgtgacccagaagcgtgtgcctgcagccaggctggggattaaatgccaggtcagtggatcgcatgtcctttccatgtgggctgcttcccgggatggctgtgggaacatggcaggacgcattgaatttaatccaatccgggtccggactcattacctccacaccattatgaagctggagctggagagcaagcggcaggtgagccgcccagcagccccagatgaggagccctccccgactgccagttgcagcctgacaggagcacagggctctgagacccaggacttccaggagttcattgctgagaatgagacagcagtgatgcacctgcagagtgcagaggaactggagcggctcaaggcagaagaagattccagcggctctagtgccagcctggactcgagcatcgagagcctgggtgtgtgcatcctagaggagcctctggctgtccccgaagagctgtgcccaggccttacagcccccattctcatccaggctcagctgcccccaggctcctctgtcctgtgttttaccgagaactcagaccacccaactgcctcaacggtgaacagcccatcctacttgaacagtgggcccctggtctattatcaagtggagcagaggccagtcttgggagtgaaaggagagccttggtacggaagaaggctcagcctctttcccaaaggagaaggatctgaatgtcttctctctccctgttacctcactcgtgggcttgtagctccacagacccagctgccctctgtaaatcagaggtggggaaaacacccaccctagaagctctattgcccgaagattgtaagccctgaggagcctgaaaatgaagacttccacccttcctggtccccctcaagcctccccttccgcacggacaatgaagagggctgtgggatggtgagggaggtcccacgcagaatgaggatcggcccccctgaagattcttccattagaactccctctggcagtgtggacaggcgctagaggtcctgcctcttacccattctctatttattcccttattttatctaacaccatttcaaaacaaaactgtagaagcagctgcttgctcccccagtgattattttattggggtctttggggaatgggtgggaaaggattgtttgagtattttttaaaagggaaacagtaccaaggagaccagccctaccttgatctggggatagtcttggggcaaagaaagctgcgtagtgcgtgaacattaagctttctgggccactttgaacaaagaactaggatctcacaggaaaagctgggtaactcaagcagctattctttctgtagggacccagaacacgagaatttgaagagcatggcaaagccctttctctcccaagccccaggcagagtacaagctcatttttctcggtgggttattctgatatcccattttggtgtgtcataatacttcaaactggaaagtcacctggtcgagtctagggaggagggagtgggaagggtctctgcttctgtacaaaatctccaggatttacaaaaatgtaacctgccttccttcaatcccatatttgggtaaataaagttaatatttgacatgtttggtgttctctgacctgttccccacactggggatatcctggtctgtaacttgaattgtttcttccacatgttcttaagtactagagttaaacttgtctatgtgtcttttttctctccatcccttttttccttcggagaaagagctggattgggctggggatatggtataacgagcctgagcctttctgtacgtgtccgaccattagcactgtcaggcagctttcggctggggctcacccatcccataggaggcctatgtggagcattaactgtggccaaaggaggcagcgggaacaggctgcttgctatctggaaagatgacatatttgtgccatgcttgttttttcacccaggtttttccctctttgtgagatctgagctttagtggaatgtagaatgtggcaatcttaaggtcaccaatcagtttttgttcttttttcctggatctaccctcatggtcgttttcaacatagcgggttagagttcttatactgttccagttcaattccaagaattgcaaatgtttattggacccctccatcttgatgatatataaaaattggaagccaaaccagttcttaaatgctacatgcaactcccagattccaaaggattcaagttctaattataacctttgaagcgtatagaatggaactcattcagtttgattcagttcttacctctgtcaggagggaatacagttcctgttcatcctgtgttctaagatctccattgaacagcactgatttcctttcattaggcaaagtgatctttgtctctgttctaaaatgggagggcagtaggggagcaaaacctattcttctcaagcatccctttacaccatactttacttctgggtatgggtttattaaccacacacacacacaaaagtgaaaaactgtgttgggggggagacactactcctactgtatcttcgctccataaagccttatctggatgggatatataagggcctgggtgtcttaccagaaacccatcaccgtaatgtcgccagccagccattgtcacccgctgtaaaggtcttcgtaaactccntggaagcncctgagaaagggaatttgtaaatttaatacacataacattttttagttcattctgtggtttcctatttgttccagtttttgcttggttttagtttggaggggaacttaagtacacagatccttaatctctccccatcccctagcctcacaaaacacagttgagagtctttttaagtacctgagctctcgaaggttacccagaactggaactagactcccctaccttagactgggtaccctcaaaacacaggactgaagcttaattggggaatttggctttatgggagaaaaggaatctttttcacaagtttgtgtcggaaaggggagggtgaggtttgcccactgtctctgcaggaaaggctccggcttaaatctagggaagtaggattccagctgcacgatgagggaacacattagcttttgggatcaaaccaggaatatgaatctgttaattatttaaggctcattgccaacccacaagatatgtttctgaaaacctgtagtttcttaatttaagtccatccccttcattaacgctacagttgtgactcacactgatcccaaaacttttaagtgctaaatattaacatttagcattaactgtcttgtcaagcgaaaggcccttctctacaaccctagtcccatcctcaccttctggtgcctaccctgagttggacagaattcctagcctcatgggttgcctagggaaagctaggcctctgatcataagaagcaaatagctttcagtcccagtctaggcctagataaatgactcttattaccacaacccttattattttccaatttcctttctcacatactgtacacaggtagtattttcaatgtgaatccaaagcttgtctggttctctgaaaataatttttttcccctttaggttctttacattgtgataatgctgtatttaaagagaatatttaaatgtaatattaaagaaatattcaaaagaaaaaaaaaaaa

SEQ ID NO: 43 - DNA, 1069 bp, Homo sapiens
  misc_feature: Incyte ID No LI251489.52000MAY01
gaattcttggaaacttagtcagctgttacttaagagttacgtattatcctatcattatgtattaatattaattttttcatacacatgtccatgcagaacatattaggtatcttggtgtagaaaacactaatgttgaaatgaactaaattccaatcttttagtattctcccagtttagtatcataaacttttgtatcttttccatgacctgcctttcattagaggccttcgaggaagaggcccacctccttcatgggcctctgagcctgaacgcccatccattcttagtgcatcagaactgaaggagcttgataaatttgataacctagatgctgaagctgatgaaggttgggcaaggtgctaagatggaagtagattatacagagcaactgaatttcagtgatgatgatgaacaaggaagttacaggccttaaagagaataaccagtgaggatcaaggttcaaaagcctctgaaaacaacggaaaacaaaaaagaaacagatgaagtttccaacactaaatacatcttcccaaatacctgcccaaaccatcagtagcaaaagttccctatgggaaaaggaccttcatttaatcaggaacgtggaacatcttcacatctgccaccacctaccaaagttgcttgcacagcagcatccacctccagatcgacaggcagtacctgggaagaccaggcccctttccctccaagcagcaaagtagctgatgaagatgaaatattggaagcaaagacgaagacgacaatcagaaatttctgcagcagtagaatcgtgctcgtaaacggcgtgaagaggaagagcgaagaatggaagaacaaagcgaaggcagcttgtgcggagaaactgaaacgattggctgacgaagcttggcatcctggaaaaacaaccatctccagaggaaatcagggaaagggagcgtagtataaaagaacgggagcgtgtagaaagaacttgaaaaagaacaagaacaggagcgagagaaggagagggaaaaagacagagagtagacagcaggaaaaggagaaagagctggagaaggagcaggaaaaacaa

SEQ ID NO: 44 - DNA, 4375 bp, Homo sapiens
  misc_feature: Incyte ID No LI902018.1072000MAY01
cttccagttgtcttgaacagcctggcagtgtcagggccgagtgggggtttctccccactgtccggcaagcgtcggtcagccgaacactgtgtgcctggtggtgtgtgttcacatgtgtgcgtgcgtgtgcccggcagagcagcaaacagctgcgcccgagagctgtggcgctttccctncctatggaagttccttttatgcgttggaggaaaaacatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaagatcaaagaaggaaagaagaaggggaagaaaagaaggggaagaagatcaaaacccaccatgccccagggctcagcagggagctgctggatgagaaagggcctgaagtcttgcaggactcactggatagatgttattcaactccttcaggttgtcttgaactgactgactcatgccagccctacagaagtgccttttacatattggagcaacagcgtgttggcttggctgttgacatggatgaaattgaaaagtaccaagaagtggaagaagaccaagacccatcatgccccaggctcagcggggagctgttggatgagaaagagcctgaagtcttgcaggagtcactggatagatgctattcaactccttcaggttgtcttgaactgactgactcatgccagccctacagaagtgccttttacatattggagcaacagcgtgttggcttggctgttgacatggatgaaattgaaaagtaccaagaagtggaagaagaccaagacccatcatgccccaggctcagcagggagctgctggatgagaaagagcctgaagtcttgcaggactcactgggtagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaagcatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttatgtattggagcaacagcgtgttggcttggctgttgacatggatgaaattgaaaagtacaaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccgtatggaagttccttttatgcattggaggaaaaacatgttggcttttctcttgacgtgggagaaattgaaaagaaggggaaggggaagataagaaggggaagaagatcaaagaagaaaagaagaaggggaagaaaagaaggggaagaagatcaaaacccaccatgccccaggctcaacagcgtgctgatggaagtggaagagcctgaagtcttacaggactcactggatagatgttattcgactccatcaatgtactgtgaactacgtgactcattccagcactacagaagtgtgttttactcatttgaggaacagcacatcagctttgcccttgacatggacaataggttctttactttgacggtgacaagtctctatctggtcttccagatgggagtcatattcccacaataagcagcccttactaagccgagaggtgtcattcctgcaggcaggacctataggcgcctgaagatttgaatgaaactatagttccatttggaagcccagacataggatgggtcagtgggcatggctctattcctattctcagagcatgccagtggcaacctgtgctcagtctgaagacaatggacccacgttaggtgtgacacgttcacataactgtgcagcacatgccgggagtgatcagtcggacattttaatttgaaccacgtatctctgggtagctacaaaattcctcagggatttcattttgcaggcatgtctctgagcttctatacctgctcaaggtcagtgtcatctttgtgtttagctcatccaaaggtgttaccctggtttcaatgaacctaacctcattctttgtgtcttcagtgttggcttgttttagctgatccatctgtaacacaggagggatccttggctgaggattgtatttcagaaccaccaactgctcttgacaattgttaacccgctaggctcctttggttagagaagccacagtccttcagcctccaattggtgtcagtacttaggaagaccacagctagatggacaaacagcattgggaggccttagccctgctcctctcaattccatcctgtagagaacaggagtcaggagccgctggcaggagacagcatgtcacccaggactctgccggtgcagaatatgagcaatgccatgttcttgcagaaaacgcttaacctgagtttcataggaggtaatcaccagacaactgcagaatgtagaacactgggcaggacaactgacctgtctccttcacatagtccatatcaccacaaatcacacaacaaaaaggagaagagatattttcggttgaaaaaaagtaaaaagataatgtagctgcatttctttagttattttgaaccccaaatatttcctcatctttttgttgttgtcatggatggtggtgacatggacttgtttatagaggacaggtcagctgtctggctcaatgatctacattctgaagttgtctgaaaatgtcttcatgattaaattcagcctaaacgttttgccgggaacactgcagagacaatgctgtgagtttccaacctcagcccatctgcgggcagagaaggtctagtttgtccatcaccattatgatatcaggactggttacttggttaaggaggggtctaggagatctgtcccttttagagacaccttacttataatgaagtacttgggaaagcagttttcaagagtataaatatcctgtattctaatgatcatcctctaaacattttatcatttattaatcctccctgcctgtgtctattattatattcatatctctacactgcaaattttgggtctcaatttttactgtgcctttgtttttactagtgtctgctgttgcaaaaagaagaaaacattctctgcctgagttttaatttttgtccaaagttaattttaatctatacaattaaaaccttttgcctatcactctggacttttggattgtttttcacattcagtgttataatatntgattatgctgattggttttggtgggtactgatgcgaattaataaaaacatttcatttcaa

SEQ ID NO: 45 - DNA, 3220 bp, Homo sapiens
  misc_feature: Incyte ID No LI220495.12000MAY01
acaggctcggggccagccgggcgcgcatccccgggcgccctgcgcggtggagagcttggcgggctgcgggtgccgcaggacaggagtggacaaagcaagatggcagggatcttagcctggttctggatttagaggttttggctcccgcacaatgtcacctgggcggacctgaagaacacggaggaggccaccttcccgcaggctgaggacctctatctcgcttttcccctggccttctgtatcttcatggtgcggctcatcttcgagagatttgtagccaaaccgtgcgccatagccctcaacattcaggccaatggaccacaaattgctccgcccaatgccattctggaaaaggtcttcactgcaattacaaagcatcctgatgaaaagagattggaaggcctctccaagcaactggactgggatgttcgaagcattcagcgctggtttcgacaaagacgcaatcaggagaagccaagcacgctgacgaggttctgtgagagcatgtggagattttcattttacctttatgtatttacctacggagtcagattcctgaaaaagaccccctggttgtggaatacgaggcattgctggtacaactacccctatcagccactcacaactgaccttcactactattacatcctggagctgtcgttttatgggtcttggatgttgttctcagttcactggatatcanaaagaatcaggacttgtggcgattaatgttccctgcaccaccttgtatctattttcttgattaccttttcatatgtcaacaatatggcccgagtaggaacgctggtccttggtcttcatgattcagctgatgctcttctggaggctgccaaaatggcaaattatgccaagtttcagaaaatgtgtgatctcctgtttgttatgtttgccgtggtttttatcaccacacgactgggtatatttcctctctgggtgttaaataccacattatttgaaagctgggagatcgttggaccttacccttcctggtgggtttttaacctactgctattgctagtacaagggttgaactgcttctggtcttacttgattgtgaaaatagcttgcaaagctgtttcaagaggcaaggctgggaagtggaaccctttacatgtgtccaaggatgatcgaagtgatatttgagtctagctcagatgaggaggagtcagaacctccgggaaagaatccccacactgcgacaaccaccaatgggaccagtggtaccaacgggtatctcctgactggctcctgctccatggatgattaattacttcaaaacttacaagtcccaaggcaaagtgaactatttgttcctggaagtatttaataagttgcaaatgccagttcctttcataatatctcagcaccagaaacaaaaattaagattatcaaacgcattttgaatacgtgcactgccatgtgtcctgtctgtgcactgaagacgaattaccattctctctttgtaggcatgctgtatgtaatttgacacaagggaacagtatttgcatttgtactggtcttagaatattatttattttttttgtatttgtaaatctgtggacaaaagagggtttcctcactccttttactcactggggctcatgacagtgaaggagatgctccatctggcttctccccctttctcttgctgtagtccaatgtgctatgagcatcagcttactttgtcacttagagcaagcaaaacccagtgcaagagtctcgttcagctcttaaatagggtttgctttcttttagttacagtgcccattttgaaattgcctatacagtcttagtgaccatttaaaccggacgaactaggtgtttaattttcactcttcatgttcaattagcagttcaaattaaagaagatggttattggagaacttttttgaatggttttgtattaaattgctttgaaatagatttcatttcttgtgcacacagcccaagatttcttcaatgggtgtgagctagttgagggttaaccttgtaggttgcagagtgtatttgtttgtttgtttgtttttctcngtgatgaggtcagtgctctgattttgaaggaggatattcactgaagctcatagttataaacaaggaaatcactgttaagaatgggaatttgtcctgtgttctgggaataacataaagagagcaactgatttcagcccaggttttgccactaccctataattagtgcagtcttatgttataaaagaaagaagttaactatatttggggacaaaaaaatatttcaagagttgataaagattacctgtgcagtgcagagcactttaatgcaaccagctttcaagaaaaagccctatctagtacttgatgttgatgtttttattttgctgagcaaaataaagccaatgggagaaagactattttaccctttgcttttctccttaaacgtaatccagatgactttcctgttactaaacactgagcagcattacactacaatgcttctttggtttccaggaatttttttcaaatggggctgtttctggaaaaatgaaaaattctattggacaatggcaatatcaacaatgaggaaaattactgaagaataagtttccataagtctcctacatagcagtgttatttatgtacagataagaaaaccatatgtcagccaaagcattttatctcttcttctaacttttagtcacgaggcaaaaggggttataaaacctttcattaattcaggaaggccatcatttagaaggaaccccaaaaaaaattgccatattataaatgtgtgaatcagggctgtgaaggacaaaacagggcagcacacatgcgtaacaagcgttgcagaaacaccagagagtgcgtatcctgctcagaaccattcacatttaattcaattcttggaaaaaattaaagctttttgcccacaatttgcaatctgtgggttaatagttaaaagaatgttcccaaccaaaaaattcttaccgtaatattatatcttggccctacttatttacaaaataatatgtttctgttatggtccttagtaataattgaagaggcttagaaatacatctgcttgtttattgagaaaacgatgcgaataattctgcttttagagccttgttattttatttcacaaaacaggcatatgtctaggagtgtaatatgtggatggttgagtttgtaagaacaatcataaaaggacttgttagtctccaga

SEQ ID NO: 46 - DNA, 2961 bp, Homo sapiens
  misc_feature: Incyte ID No LI399478.12000MAY01
tgatcagttattgtgaccttgactcactcatttctgatgaactttctggggcccgatcaagcatctgccatcactggttgttgacagggggctgatcagtctattatggcctcagctaaagtgactcagctttcctctatggttcttgtatccttcagtaggctagcttgagcttgttttcatggccatgacgtggttccaagagagataagaagcacacaagacctttgtaaggatcagtcccaccgctggccacaatggttcatgcttataacttcacctctttaggaggctgacgtgtgagggtcacttgaaaccaggaatttcagaccagctgggcaacaaagggagacctcatctctgatatttccagtgtcagagggacacagccaacgtggggtcccttctaggctgacagccgctctccagccactgccgcgagcccgtctgctcccgccctgcccgtgcactctccgcagccgccctccgccaagccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctgacatgaatggctattgtttgcatggacagtgcatctatctggtggacatgagtcaaaactactgcaggtgtgaagtgggttatactggtgtccgatgtgaacacttctttttaaccgtccaccaacctttaagcaaagagtatgtggctttgaccgtgattcttattattttgtttcttatcacagtcgtcggttccacatattatttctgcagatggtacagaaatcgaaaaagtaaagaaccaaagaaggaatatgagagagttacctcaggggatccagagttgccgcaagtctgaatggcgccatcaaacttatgggcagggataacagtgtgcctggttaatattaatattccattttattaataatatttatgttgggtcaagtgttaggtcaataacactgtattttaatgtacttgaaaaatgtttttatttttgttttatttttgacagactatttgctaatgtataatgtgcagaaaatatttaatatcaaaagaaaattgatatttttatacaagtaatttcctgagctaaatgcttcattgaaagcttcaaagtttatatgcctggtgcacagtgcttagaagtaagcaattcccaggtcatagctcaagaattgttagcaaatgacagatttctgtaagcctatatatatagtcaaatcgatttagtaagtatgttttttatgttcctcaaatcagtgataattggtttgactgtaccatggtttgatatgtagttggcaccatggtatcatatattaaaacaataatgcaattagaatttgggagaagcaaatataggtcctgtgttaaacactacacatttgaaacaagctaaccctggggagtctatggtctcttcactcaggtctcagctataattctgttatatgaggggcagtggacagttccctatgccaactcacgactcctacaggtactagtcactcatctaccagattctgcctatgtaaaatgaattgaaaaacaattttctgtaatcttttatttaagtagtgggcatttcatagcttcacaatgttccttttttgtatattacaacatttatgtgaggtaattattgctcaacagacaattagaaaaaagtccacacttgaagcctaaatttgtgctttttaagaatatttttagactatttctttttataggggctttgctgaattctaacattaaatcacagcccaaaatttgatggactaattattattttaaaatatatgaagacaataattctacatgttgtcttaagatggaaatacagttatttcatcttttattcaaggaagttttaactttaatacagctcagtaaatggcttcttctagaatgtaaagttatgtatttaaagttgtatcttgacacaggaaatgggaaaaaacttaaaaattaatatggtgtatttttccaaatgaaaaatctcaattgaaagcttttaaaatgtagaaacttaaacacaccttcctgtggaggctgagatgaaaactagggctcattttcctgacatttgtttattttttggaagagacaaagatttcttctgcactctgagcccataggtctcagagagttaataggagtatttttgggctattgcataaggagccactgctgccaccacttttggattttatgggaggctccttcatcgaatgctaaacctttgagtagagtctccctggatcacataccaggtcagggaggatctgttcttcctctacgtttatcctggcatgtgctagggtaaacgaaggcataataagccatggctgacctctggagcaccaggtgccaggacttgtctccatgtgtatccatgcattatataccctggtgcaatcacacgactgtcatctaaagtcctggccctggcccttactattaggaaaataaacagacaaaaacaagtaaatatatatggtcatatacatattgtatatatattcatatacaaacatgtatgtatacatgaccttaatggatcatagaattgcagtcatttggtgctctgctaaccatttatataaaacttaaaaacaagagaaaagaaaaatcaattagatctaaacagttatttctgtttcctatttaatacagctgaagtcaaaatatgtaagaacacattttaaatactctacttacagttggccctctgtggttagttccacatctgtggattcaaccaaccaaggacggaaaatgct

SEQ ID NO: 47 - DNA, 2099 bp, Homo sapiens
  misc_feature: Incyte ID No LI229648.22000MAY01
ttggtttccttaattagttccctgtgccagcccatagtcagagccattaattggctctggggaagatccaagttattttctgagtaagatattaggcttccatatgatccagagatgcaaagaaatccctagagagtgtaggagttgtctaaatccatgtgtcagatgtagccaacgaattatgtcagaagcagagagaaaaggcctgaaaagcagctctctcccactcctcaggcccttgtctccaaccttacatgaggctttttgaacatctcctcctggcccagctggggtgagagcaagtcctcgaaggcactgcctttgagccttgctcagcccattctgaactatcccaactctagaattgactgctttcgaattgtgtgaccttgggaatgttatctggcttcaaccacaatgccctacccccagctcctctcccaaatgatcctagatacagggctgcttccccccgaccctaccccacctcgggacacaggctcatggcctcatggcacttcaccaccagaagtggtgctcagagttcctatttccacatctaaccccctaattcctgggaaagtctgaggcctggtccccccagtgctttccctggctggcctctccacattttcatctgatggtggagtgagatcaggaaaaataggacaggagctttgccttgggggagaagagagttaagtgtggaaaggggtgagttataggaggttaagcagtccaagattttctctccctgtgtaggaggccatttcctgatgtgaggggtctgaaacccaattatgatgggacagggttgggcattgacttcccatctcttctctctgtttttctcccactatctgtagcccaaaactcttatggaggactttgatctttagtataggctattggtcagggccataggaactaaccccgatcctcactccaccaggatctaccacatcccctacacacaaacacatgctgtggggagggagtttttccctggggtcaaattgagggatcccttaggatcaacttgtgctcctgtggactggtgtgtgcgtgtgtgtgtgtgtgtgtgtgtgtgtgtgtgtgtgtgtgtgtatgtggggaaacttagctttcagagaatgtctatgggctctcattttctctctcacacaaaaatactcgggacttctccaagtccctgaggagcctgaccactgaagctgatcatgagatgactgtatgctgacacacccccttcaggggcctggccttgacttagggctgcactgtatcctcagcaacggccttgcaggagccccttttggactgctttccctattcagcccagagttggggtggtgggagaagaggggttggagtgaatccatctctattcaaattccagctgggattactctaggagtcttcctggcttgtatggggctcaaacttagctacattgtttattggctcccaaagtcgggattgaagagtgaaaagatgcaggcaatgaatccttctgcacactcctccccaacctttccagcgcttttctacttaggaggccagtggaagggaggagaggccatgccctagcccaccaggggacaaggttcattgttccttccagggcttggttcactctgcttttgattcagaagctctttccctacccagcaagactacactttcttgccttctttctattttttctttttgtgcgtataaatggtatgttgtgatatattctcagtgcttgtgcccaccttggaactctgttcttgctcttcattccgcatgtgatactctggtccaagatcttggccaggtgccttctgctcaaatatcgtctcagaggtgcttcccttgaaaactcggtgctgtttccatagttactctatttgatcactctaagtttggttgtcttcatagcacttgtcaccctctggaactattctattcatttatttacttgtttaatgcttggctcttttcccctcctaacgtaaactccatgattgccaacacctgtttacttactacagttccccctccccccacattcctgacactagtaagaaccaataaaacacttgttgacggaa

SEQ ID NO: 48 - DNA, 1704 bp, Homo sapiens
  misc_feature: Incyte ID No LI025643.22000MAY01
ggccagacatggcggaagaggaggtggccaagttggagaagcacttgatgcttctgcggcaggagtatgtcaagctgcagaagaagctggcggagacagagaagcgctgcgctctcttggctgcgcaggcaaacaaggagagcagcagcgagtccttcatcagccgtctgctggccatcgtggcagacctctacgagcaggagcagtacagcgatctgaagataaaggttggggacaggcacatcagtgctcacaagtttgtcctggcagcccgcagtgacagctgggagtctggctcaagcttgtgcctgtcccacctaaaagacgttggacgctgtcagatgtggcaactcctgaggtgacgatgacaaagtgttcgctggaatctatacagatgagctggagttcagagaggatgatgtgttcctgactgaactgatgaaactagcaaatcggtttcagctacagctcctcagggagagcatgtgagaagggtgttcatgtcctctagttgaatgtccaggaacgtggattccgcgttctaccagaacgggcagcaggagcttgaatgccaacgcacacttgatgaactactggtggcagacaattattggcaacgtccattggcgacagaccttgagcgaaggaggatttcaacgcagcattgagccgctccaagtggttatacacaaatgatcaaatccaagaccagagtacccgcgtacattaaagccatcaaaggtggcgagagaagacgtggtcttcctgtaatctgattgaaatggattcccagctcccctggaaagctgaatgaagcggatcataacggagatctggcacttagaatcttagcccctctcacgacgactggagaagtaaattgccaaccacgctggttagtcaccaagctgatgtggacatggtggaccaagagtggctggagccttgttacaccaaggggattccaaagaaggagatctcttgtgctgccactttcctacattaacgaatggcggccttgtgtcaacgctgctacactgggtgccccaggagacaccacttgcacctttgtgggcctttgtaccagttccacaagaaacactcagctagatgtgatgcctgagatggccgcagattgcagaggcccttctgcaggctgggtgccaacccccaacatgcaggacagcaaggggaggactcctgttacatgtgtgccatctatggccgcgggatatgatatatgtgtctcagtcagctgctccagtcgcaaacaactagatttagaactcaatagaccacgagggcaagcacaggctactgtggcatggctagtgcagcatatcacaagtgtcttctgaaccagtctgggaacccccttcgaagatgtccccggtggtaaactgggacttcactttgagtgagaacacggtttgcgagccagaactccatccatgcgcgggcagtccactacatgactgcaccttgacacggcgacaggtaaagcacgtgcctcccgaaggggcgacgctggggtcctgcaggcgccaggaaatggcatgtaaatgccttcaccccaaatttagaaactgaacggcatactatgaggataattatttggtacttaattttatatacagtaaaagcctgggttgtcatttttaaaaatcctgtgcttcaaacaattgagcagacatcaagtccctg

SEQ ID NO: 49 - DNA, 2276 bp, Homo sapiens
  misc_feature: Incyte ID No LI233942.12000MAY01
agagaataacaagtctgggactgacgtaggaaaaacagtagtctgtttaagttgagaggaggaggatgtggacgagagaagggaggagagggaagaactgaaatgtctcctcacccagggtggttattgggcccaacttgggatggggttgagtacgaaggttaaaaaacatatactttggtattatattggtcattgtaatagcagttgtaaaatatggggggaaaagggatcatttgctttcctttcatggtgttaatgtgtgcctgaatctatagccgctaaaatgtaaggctgtccacctgacttatttaaatattatcagagaatagaaagtaatcactttttaagtgtacctatgtgctgccccttaaaacattcatggcatctggcaaatagacccttgtaggctggcctggcatattcagatgttgccgcatcatcacagagaaggtccaatgaagatgtaaccattgctgctcatgtgtcttctctaagcaacaaggggagcgggatgtacgagagaagttgctaatgatactgtacaaaggatggattaaataatgaagtcaatttcattgatagaaaatgcaaattttaagattctcagtgtatgaggtctcagcctcgggagcatttattttttactttcctgctaaaaatccactggggaggtataaaattcactccaaagaagtttgcctgggccaaaagcctccaggttccttctgagaacaaaatactaatgatttttttttttttttggctgcttgggagagactttcagcattctttcacgatatcccagatatgtcccttcagcacacacactcaactttctcatctggttcgtttcatcctcccgaaagaacggtgctctcccactcttactctatttcaaagagctactctgtctcaaccacagcagccctgccaagaaaaggcaagatgaaaggctagagcatgtgctaccttgagatttcaagaagtgtcctacctcagctgaagatggagaaacggggagtggagaggggatcagcccagggtaccatgtgatgactaatatgccactcattaagggcttttacctcggctgtccacatccagtcatgcatttggggccaggcatgcctgcgaaatgattgacattcctgtaggtgccccatagaggtgtggcattgagaaattatgtctggaaaatacagccagtgtagtgaccagctgagttaccatatagcctgatgattccccttctgtaacagaaggtgacaacaactttgtgaactttctatatctttatctgatgccccaagtggcatcttctttatgtacatgccttagttgagaatatgagactgctctaatctttgtgaatgccttcacacttcagctgaggatgtcaccttctctgtcactccccatcctgcaacctctggcctttgctctgtctaccccagcaattttcatagtgttatccaggtactgtgctaaaaggtcagctcttctgtaaaatgccatgcttcgcagcttcaaaaagcaaaattctgtatttcatggagggagggaagggtcattctcttctaagaaaaatagactggagcaggcgaaatgcatgcatgcatgtgtttagagttgcttctcagagagacataagagatgatagcaactattacaatatgactgaatgactgagtgaatgagttgaaggcctagctcgggctgataaagcttttgcctntagtagaaggtttatgtcaacaccttcaggagggaagcccttatttctgggttgaactccccttcccatgatattatgctagccattgccatcacttagttcctggcgaaggcagaaaaagaaaccaaaaaatcagcattgtcattgacaaaactggatgctgaagataaagtaaaaattgtcttttcccttgtccccacatttttggggattaaanccccctaangggnnaaaaaanntttggacataggaaatacagtactatttctgaagttgatagccgatcttttaagaagaccctgtctttagaatgaaactagcaggcaaacacctaaataatcgaaaattaccaaagttcaaccacgaaacaagaaaacctaaagatttttacttcctattcttattcctttccttattactgtgacaccccttgcattgctcagttgcttgtgtgtcaaaataacttgtggacgtctggaaattatttgaaaatgtattgtgaaaaatgtaataaaatgatgatatttttatacaaaaa

SEQ ID NO: 50 - DNA, 4111 bp, Homo sapiens
  misc_feature: Incyte ID No LI089158.12000MAY01
cactccaggagttccccatggagttttcaacaagcagagaataaaaataaatgtttgtgattgacaacttttttccaaatgtttgaaacttcacatagaaaactgtaaagagatggccctccctcactgtatattaacattttaagtaaaaccctttgtcccagtgttatacaagaaagttcaacaacaaaaggaaggaaaaaacaaagtgagaataagcttcttggataccatccctagatgatgaagtgcagaaaactggaaaaacctgacatcctgcatggagacccaggaaataacatggcatggcaaatcccagggaagaaaactcatgtgtaaatcaaacagagctcaaggaaaagactgaaatttcttctcttcatgtttcaaaaagaattccagaggtcaaaggaacaggtgcaggtgcaaatgtatataaaaataatgagcagcgctcatgacaaatcaggatagctacattcagtcatctttcatgtatgtctaaactcaggccaaggggcaataagtgtccactgtgctttaaaaggtctagaaatagaaaaatcaacccccaaatcttttctaattccacatggccatttgtcacgagatgactaacaatgtctgtggggtcaggtttaatacatttgcactgagctcgtaccaggcagtccataaaactgttgtcttaaaatttctccagtccccacatgtggctgttttagaggcatttccaaacttaaaacccagcttctatagctctccatacctcttttacctgcttagaaatgaatcccaccagacctccacgaatccaactttgttaaatcatcccctattgctgatcaaactcactgctccagcattcaaaaaacagaaggcataaaggtgaatacaaacataagaaaggagtttacatcgtggtcttgcagagctttccatagtagtctcataaagtcctggtgaatcccccagcccgcatatcttgtgcagagccaacttgagtgttatcaatcgcgtgtcaaacagtgataggagcttggtgtatgggccgccgtcgctctgggaggatggtggtgtaggaagtgaacttgactctcttcctctttgggcccgaagaattcataggttcatttttaatttctttctcatagacatagctcagagggtctgagtagttgactatgaaaagtcttctgggaactgccattcagaaggaagttcctctcctcgaactgcaggcccctgtctatcatggttgtgcactcctctgatgggagtgtaatgtcaacagggttctccaaaaagttccacttcattcccaagccagacccagtcgtgggaatgggggatgttgccctgctcactcacagcaaaccttttgtgtctgtatttccaggcaaacgccacgcagttgatcaagaagaccagaatggccagacagaagacgcagagcaaggcatacatgccaatctccaagtccgttagcccccttgaggtcactgtgaggtcactaggattattgggggtccggtgacttcccttgagtggggaagcttgtaaaggcatctggaccaccacttttgagtaacttattcttcccttccatgggagactggggggttgtgctttcgttggtactttcctctttggcaacaggtgtgccacggtggaaccattcctggactgctctctcctggtttccttcgcgctctatggaattactgaggtggtctttatattcccgatttatgccctcaatatcattgctgcctccttggtgctcatcactacttggttcgaatttgaccttgacatttcctttacccacggcaagaacactcttcctcttggtcttctggacaaggttcacttatcatcatttctaacttaatcaaaggcccttgtccttcaccctctgcaaccacaattggccattgtggactcaaggtttgcctggacagacaccaccatttcatccaatgatgagacagtaacagaataatccttaggatcgtaaatgtctaaaggtgtcaccgaaccatcactgaacaaaatccaagaacttactattgcttcctgctgtggggactgaagaacatccagggcagcagctgtggagacgatggcccttttgtctgctcggtgtggctgcagggagagagacatgccagctacgagctgcactcccagctccacgatggtgactcggtcatccaggacaatcaccgtcttctcagccaggatggagtcagacaacggcgagaggacctgcaccgtggttattcccggctcccgaccagccagggtcctgccgtcctgtaactgagcgattttcggctcctccaccttcatgaactcggtcacaaagttccagtgatgtcaaactgccagtcggggcccagcattngtaggtcagctagccctaagnttccagngnttgaaaaccttccgggggcccaagcaaaancttgggtgaggacacgcactgtggcgtgctggtactgcagggatgcagcctcagtcccttcttctcctcatcgtcctcgttccatttcgctttcccgggtaggccttctgttggcagcaaccgggatcctccagcccttgatctggctcagctcggtgtctgagatctcaatctgcagggggagcctgggtgcccagacagtgacctcgaactgggaggtgaagtgctggtgggtgaagttcacaatcgtgtccactttgctcttgcatttccttcccagttcacagaaaatggaatcacagttgttggaaaccttaatgacatcttcatcgcgcagacttgcattgccacagactcatgacacatcgaccacagaaccatcctcgctggacccccacgactttgacaggaactgaaacaggcctttccagtgagaatggcggtgttcaaaacctcggtgtccatggcaagagggacgatgcccacgaaggttgtctggctgacgaagatctcggagacgaccagctcactcatggagtcctcaatcgggtactccacctgccaggtaatctgctgggccccagccaggtcactgctattatcaattccaaagtccacttgcaagatctcatagaaggatccatttaccctgctctgcgtgtccgggcgatggcccatgcaggtgagggtggccgacgtctgagtgctgccattatcaatttcctcctggactgcccattggtcctcactgctgactctcactgccgttatcttcacacctgctgccgccttaattctaagagtgaactggtctgccacagagctactggtcagagagaccaaaaaggtggccgtgtccccttcccggactagattcagaggtaccgagatgaccacattctcgtccaagctcaccagggaccacttcagatcatccttggttggttagaccaccacactcccaatcctctctcgggctggaaacgcttgctgggggctctccaggcccgagtggatattgttctcccacttgccttcctcctccagaggacactggccagccttgtcagctgggtagagcgtgaagaagagctccattcgtggtgcccccgaacagggctgggatttccttctttggtttccagttccagccttaagttgaaccattcggccaccagttccagttcaagccaacacacagcccctgggccccttgcagccgacagctggctgccacttccctggcctcagggaaagcaaacatcttgacacagggtagatcttccgtaaggtcactgtcatcccagcccatgccagtgacataaaacaaggtctgcactttgggtctgttggagtagatggagctgtcaaggatgtgggatttcaatttccagttgaagggaaatttgtccatgtttccaaaggctgtagatgtcaacaggagctcctgggggattatcttctccactgaaaatgggccatagctggcattgataatagggggtgtcctggctcggtagatgaagaatggctccacccgggcctgcagactggagttccttgtgaggtcttggttggcttctttaaggaaaaaggactcctctgcattggagatgtgcaagttcgtggggaggtaagcag

SEQ ID NO: 51 - DNA, 2353 bp, Homo sapiens
  misc_feature: Incyte ID No LI101046.12000MAY01
gcagccaggaagcacccagaactcttctgccacagttgtagaattgtaggacccagtgggcaccacctttgtctcctgggcaagtccctttaccttccaaggcctcctttctcccaccctgctccacttttctcagcctcctgaatgaggatggaggcttaatagacatgagaggattttgttttcagttcaatgacatagagttaagtgagctgtcagggaagacgagaggcatagctgctgtgagctcctgagaccctgaaaatgctggtttgcttcacaggtgccatctagaacttaacaggcactttcggaaaaagttggctgaggaggaacctgctggggtattagaagctgccaccagcgggttggtgattgatgttctttggggccacggtgaggcatctttttcattaaagtggggctttgtgtcctacaagtgctttgagttcagagaaaagatgaaccaaaaatgaagaaaggaagtggctcttgcttccccagcaaagagttacttgccttctgaaacacaggccttgttgccacataggaccagcccctccagagtctcactcctcaagttctgtgagacgctgcagaggacctggccatggccttgatcttatctagaccttgggaacacccctcaaccatcctcacctcacttcagcagaatggctgtctaggactttaaaaatgaatgttcacctttatcgtgccttacatcttggaaaaagatgtttcataagtttactcttctcagggtgaaataggcctttttatttgtccttcgcctgtctttttcagatttcaagggagattccttaattcttacattccaggagtcagtgagacaatctatgcttcccctccctgaccctttttgtgtttctcggtgttgatatttcacatgcttggtccttctctttgaactttttagtgcttctgggtcatcatcctgatctctctgtggcaatgccttcctccagctcccacctctaaatcatcttggcttcccactgcctggccttttctaaaagaaccccaatcttcattgcctttaaaacagccagatttgagtcaatttcatattggtggccagactgtttaaacacaaattctctagctatttacagggctcagagctgaggaggaagaaactcaagtccccaaataatgactaatcacaattcattctcaccagcaagagccatgaaattctaaggagctggaatttgggatggcttcaagtggccagtacaattgaagcgtctggcagacaggcaaagaagcctaccttctcccctccccacggtggcatcaatggaatcactttcttgggtccctcacttaatatgaatcatattcacctggggcaaccaacgcctgacagagttgtagtgcagaaggcacctctttgaagtgaaaaccaggagatcttttatcacttccccagccctgcaggctcagtcacatgtcaaatagataatgaataaatgcatcgccaatgcactctattcatttatttttgacaagtgagttggtgacagtgcccccttgcatgctaataaaatatacagcagccacacttgtaaagtctttgaaatacgaggcttcatgcaagtactccactattaaatctttgcaggaaaatggggtaaccagactgctgggactcttgcaaaatgattacgtttgaagattagtgccattctcctctacacaggctatggtttcagacagcaccatggctttgataaagtggctcccagctcataggaacttgataattaatgtcctctgtgttgcatagagatcaggcatcaaaaactcggtggtgctgagacgccaacagaacaggattcttctggtgtctggaccactgagggctggttgtcaaggagttatcttctcttggtcgcacctagtccacgtgagggagaggaaggcttcacattgtctttacttgggcccttgagattctaggctttagacaatctgaagctgttagacctatcagccaggcttctctctgaccaaggacaacccaaggggctccttagaccagcctcacctgtctggatcaccattctcctccagcttcaaaagtccatccttcctgcaaatctcatcaagactcgcggctcttaaagataaccctctccttcctgcagcacttctatgcttgaaggggaaggggctggcacacatcttctctgtgtggcctcctcctcctggccacacggcctttttcctggcccctttccttacactgttgacatgacaagggatctgaaaagggaataagtccaggttaaagcctcaaatggcacagatcaaaacccactctttc

SEQ ID NO: 52 - DNA, 3301 bp, Homo sapiens
  misc_feature: Incyte ID No LI368676.22000MAY01
ccagggcggacgcacgtgcgccgggcttgcggcgccctggggcccaccgattccccaaccgtcgttttccccaccgaggccgaagcgtcccggagtcatggcccggcctaaactgcggggtctctatcgcactgcctaggggttctgctgcggggttgcggggcgcctgccgcgcggggcagaagcttttgagattgctctgccacgagaaagcaacattacagttctcataaagctggggaccccagactctagctggcaaaaccctgttaaccgggtcatttctaaaagacatataaccatgttgtccatcaagtctgcgagaaagaatagtctttacctttagctgccagaagtccttgagagtcactttgtcatagagatcccagaaaaaatattgaactgtatgtcaggcccatgtcccttttgggggaggttcagcttcagccctcgacatcgttgttgcctaccctcaacagaactttcatctgggatgtcaaagctcataagagcatcggtttagagctgcagttttccatccctcgcatgaggcagatcggtccggtgaagagctgcccagacggagtcactaactccatcagcggccgaatcgctgctaccgtggtcaggatcggaaccttctgcagcaatggcactgtgtcccggatccaagatgcaagaaggagtgaaaatggccttacacctcccatgggttccaccccagaaaatgtctccggcttcagcattgaaacccgctcatctataaaacgtctgtgcatcatcgagtctgtgtttgagggtgaaggctcagcaaccctgaatgtctgccaactacccacgaaggctcccctgaggatgagctcatgacgtggaagtttgccgttcctgcacacctgcgggccagcgtctccttcgtcaacttcaccctctccaacttagagaggaaggaggagcgggttgaatactacatcccagggctccaccacccaaccccgaggtgttcaagctggaggacaagcagcctgggaacatggcggggaatttcaaccttctctctggcaaagctgtgaccaagatgccccaaagtccagggatcctgccggctgcagttccaagttttggtccaacatccacaaaatgcacagcaataaaatctacgtggttgacttgagtcatgagcgagccatgtcactcagcatcgagccatggcccgtcaaacagagccgcaagtttgtccctggctgtttcgtgtgtctagaatctcggacctgcagtagcaacctcaccctgacatctggctccaaacacaaaatctccttcctttgtgatgatctgacacgcctgtggatgaatgtggaaaaaaccataagctgcacagaccaccggtactgccaaaggaaatcctagctcactccagggtgcccgagtgacatcctccacctgcctgtggagctgcatgacttctccctggaagctgctggatgcccagggacaggcttcagcctggtggctggtgccagcccagaagctgcagcagcatacacacgagaagccctgcaacaccagcttcagctacctcgtggccagtgccatacccagccaggacctgtacttcgcctgcttctgcccgggaggctctatcaaagcagatccaggtgaagcaggaacatctcgggtgacccctcgcaccctttgcccccagcttccatacaagaggccttccaggcagggttgtgacgggcgtcctttataccttatttcaaaggaggaaggccgttttcacggtgacccctgacacaaaaagcaagggtctacctgaggacccccaactgggaccggggcctgccatccctcacctctgtgtcctggaacatcagcgtgcccagagaccaggtggcctgcctgactttctttaaggagcggagcggcgtggtctgccagacagggcgcgcattcatgatcatccaggagcagcggacccgggctgaggagatcttcagcctggacgaggatgtgctccccaagccaagcttccaccatcacagcttctgggtcaacatctctaactgcagccccacgagcggcaagcagctagacctgctcttctcggtgacacttaccccaaggactgtggacttgactgtcatcctcatcgcagcggtgggaggtggagtcttactgctgtctgccctcgggctcattcatttgctgtgtgaaaaagaagaaaaagaagacaaacaagggccccgctgtgggtatctacaatggcaacatcaatactgagatgccgaggcagccaaaaaagtttcagaaagggcgaaaggacaatgactcccatgtgtatgcagtcatcgaggacaccacatggtataatgggcatctgctacaggattccatgcggctccttcctgcagaccagaggtggacacctacacggccgttccagggcaaccatgggggtctgtcctcccgtccccacccaccatatgctccagggccccaacatgcaaaggttggccacatgaggagccacctcctcgctcccctcctgagtctgagagtgaaccgtacaccttctcccatcccaacaatggggatgtaagcaagcaaggacacagacattcccttactgaacactcaggagcccatggagccagcagaataacttgatccattccagacgctttgctggagtttcataaagcagggcgctgagacacccgtccgtgttcctaaccagaaatcccaaagaagaggaattatacagaaggaacagcaggaggttttcctgtgacaccgccaacttcacattgctcagtggactcattctaagggcaagacattgaaaatgatgaattccaatctggatacagtcatgacagctcatgtgctcctcaaactntaggcatgtgcaggtttagccaagccatgtaaatgagaggagagaggcctgagtcacctagcatagggttgcagcaagccctggattcagagtgttaaacagaggcttgccctcttcaggacaacagttccaagggcaaggagcctacctgaggtccctagtctcactggggtccccaggatgaaaacgacaatgtgcctttttattattatttatttggtggtcctgtgttatttggaggagatctgagtgtataaccacccagctccggtcaccggacttagtaataactcatactaactggtttggacgcatgggttgtgacattcatactgaccgctagataaacgtgtgcctgtcccccaggtggtgggaataatttacaatctgtccacccag

SEQ ID NO: 53 - DNA, 1834 bp, Homo sapiens
  misc_feature: Incyte ID No LI238713.12000MAY01
agaaaccattatatccccttggtaggcataaaaggggctgctttgcccaaactgcctatgaatttgcttcctaaagcatggggtgtgcctcaggaccttattaaaaagtacataaaacttgaagaggatggtggttgtgttattggaggtgacagaagtttgcaagataaatacttacttaggcttgttgctgctatggaagaagtctttatggacaaacatggtatccatcctagtttggttgctgatgtccatcagtatttctacagaaggactggagtgataggagttcagcctgaggaagtcacagcagctgctaaaaaaagcagtaatggataatcgccttcacaaatgtttgctctgtggtgccctttctgaacttcatgttcctccagagtggttggctcctggagggaaattgtataacctggcaaaaagtactcatggacagctgaggactgacaaaaattacagctttcccttgaacaatttggtttgctcatatgattcagtgaaagatgttctggtaccagactatggaatgagtaaactaacagcttgtaattggtgccatggcacatctgtgcgaaaggtcagaggagatgggtctattgtgtatttggatggagacagaactaattctaggtccactggtggcaaatgtggttgtggattcaaacacttttgggatggtaaggagtatgacaatctaccagaagctttccctattacttttagaatggggtggaagagtggtctagagttaacagttatattggttcctagtatgtaagtgattcatctttgaatagtaatgtttacgatgttgcatatgaaacttgttacctagcatctttgccaggtgaatttgggagtgaaatcctagttcagaaagttgtcctacactatattgcatcagtctgcctcattgtatcccgaatgattatactcctgtaaatatagatggtgctcacgcccaaagagttggagatgttcaaggacaagaatcagagtctcagctcccaactaaaattattcttactggacagaaaacaaaaacttgtgcacaaggaggagttaaacatgagtaaaactgaaagaactattcaacagaatattacggaaccaggcttctgtaatgcagaaacggaaaacagagaagttaaaacaagaacaaaaagggcaacccaggactgtttctcccagtaccattcgtgatggtccatcctctgcacctgctacacctaccaaggctccctattcaccgacaacttctaaggagaagaagatccgaatcacaactaatgatggacgacagtccatggttacccttaagtcttcaacaaacttttttgaacttcaggaaagtatagccagagaattcaacattcctccatatttacagtgtattcgatacgggtttcctcctaaagagttaatgccaccacaggcaggaatggaaaaggaaccagttcctttacagcatggcgacagaattacaatagaaattctaaaaagtaaagctgaaggtggtcagtctgctgcagcacactcagcccacactgtgaaacaagaagatattgctgttactggtaaactgtcatctaaggaacttcaggagcaagctgaaaaagaaatgtactccttgtgccttttagcaacattaatgggagaagatgtatggtcttatgcaaagggacttcctcacatgtttcagcagggtggtgtattctacagtattatgaagaaaaccatgggtatggctgatggcaagcattgtacttttccacatctgcctggcaaaacctttgtctataatgcttctgaagataga

SEQ ID NO: 54 - DNA, 755 bp, Homo sapiens
  misc_feature: Incyte ID No LI720928.12000MAY01
caaaccattgtaaatcatgagcatgcagcatatggccttggattcaaaattcttgatgtcccatttcaacagaaaacatgctgtcggcaagtggattacacagaaggtaatattgccgtattgggagggcttctggtaatactaaaaaaatggagccatacttatgtttaatagattagaagaaagtacatgtaaaactacaatgttgacacacatgagaggatgctaaacttttgcttcatttgttttctactccgagtgtacctgtttgttcatgaacattcacccacatgcatataatcacgggcacacaaacattcacccatatgcatataatcacgggcacacaaacatatacccatatgcatatactcatgggcacacaaacattcacccgtatgcatatactcatgggcacacaaacattcacccatatgcatatgctcatgggcacacaaatattcacctatatgcataaaatcatgggcacacaaacattcacccatatgcatatgatcacgggcacacagacttatgggagcacagggttatgggcacattcactccaacacaaacacacagatgtcccacccattttcctaagagtctgacattaagggattgcaacattgtacagaagccaatcatttgcaggtcactttgtccccaggaaagaaatatcttttatttttctaaatcctggtttccaaaagtattcttgatgtgtgcaaataaatataaagtgcatttccata

SEQ ID NO: 55 - DNA, 2522 bp, Homo sapiens
  misc_feature: Incyte ID No LI221874.12000MAY01
cggcggagatgcctcgttggggctgggaaactcctgcaaaactcgagaccaggaagccagcccgcaccccaacccccaccaaagccacctactcttcttctgtgggaggccagtccacatccgctctcacccgatgagagatattcagctggatccaaagtgactgatgaagggaaggaaatcatgtcaagcgaagcctttgaaaaagctgccctgagacggtgtcccgccgaaagaatgttggctcaattaagaaacatcagggagataaattcaacccagtgtgtctaaaaatgactaccaaaacggagtttgttttgtgcggttgggtaccatgtcgctgtctacggagggggaagaggagactgtcactactcttgattattctcattgcagctttagaacaagttccggaaagagatttttacttttgaaaaaactcttggaggaactctatttagatgctaatcagattgaagagcttccaaagcaactttttaactgtcagtctttacacaaactgagtttgccagacaatgatttaacaacgttaccagcatccattgcaaaccttattaatctcaggggaactggatgtcagcaagaatggaatacaggagtttccagaaaactatcaaaaattgtaaagtgtttgacaattgtggaggccagtgtaaaccctatttccaagctccctgatggattttctcagctgttaaacctaacccagttgtatctgaatgatgcttttcttgagttcttgccagcaaattttggcagattaactaaactccaaatattagagcttagagaaaaccagttaaaaatgttgcctaaaactatgaatagactgacccagctggaaagattggatttgggaagtaacgaattcacaggaagcgccctgaaagtacgttgagcaactaactcgattgactgagataattggagagtatgctaatagactgacttttattccagggtttattggtagtttgaaacagctcacatatttggatgtttctaaaaataatattgaaatggttgaagaaggaatttcgacatgtgaaaaccttcaagacctcctagtatcaagcaattcacttcagcagcttcgctgagactattggttcgttgaagaatataacaacgcttaaaatagatgaaaaccagttaatgtatctgccagactctataggagggttaatatcagtagaagaactggattgtagtttcaatgaagttgaagctttgccttcatctattgggcagcttactaacttaagaacttttgctgctgatcataattacttacagcagttgcccccagagattggaagctggaaaaaaaaaaactgggctgtttctccattccaatgaacttgagacacttccagaggaaatgggtgaaaagccaaaattaaaagtcattaatttaagtgataatagattaaagaatttaccccttagctttacaaagctacagccattgacagctaagtggctctcagaaaatcaatcccaacccctgatacctcttccaaaaggaactgattcagagaccccgaaaatggggcttaccaaatacaagttccctcaacagccaaggactgaggatgttaatttttaatatcagataatgaaagttttaacccttcattgtgggaggaacagaggaaacagcgggctcaagttgcatttgaatgtgatgaagacaaagatgaaagggaggcacctcccagggagggaaatttaaaaagatatccaacaccatacccagatgagcttaagaattatggtcaaaactgttcaaaccattgtacatagattaaaagatgaagagaccaatgaagactcaggaagagatttgaaaccacatgaagatcaacaagatataaataaagatgtgggtgtgaagacctcagaaagtactactacagtaaaaagcaaagttgatgaaagagaaaaatatatgataggaaactctgtacagaagatcagtgaacctgaagctgagattagtcctgggagtttaccagtgactgcaaatatgaaagcctctgagaacttgaagcatattgttaaccaatgatgatgtttttgaggaatctgaagaactttcttctgatgaagagatgacaatggcggagatgcgaccaccattaattgaaacctctattaaccagccaaaagtcgtagcacttagtaataacaaaaaagatgatacaaaggaaacagattctttatcagatgaagttacacacaatagcaatcagaataacagcaattgttcttctccatctcggatgtctgattcagtttctcttaaatactgatagtagtcaagacacctcactctgctctccagtgaaacaaaactcgtattgatgttaattccaaaatccggcaaggagatgaaaattttaacagccttttacaaaatggagatattttaaacagttcaacagaggaaaagttcaaagctcatgataaaaaagattttaacttacctgaatatgatt

SEQ ID NO: 56 - DNA, 2634 bp, Homo sapiens
  misc_feature: Incyte ID No LI1143545.32000MAY01
agcggagtgccaggctactcctcccgcagtgtgggtggttccgaggctggctaccctgcggcggcgcggctcgcagtccttctcagcatggaccgcacttgtgaggagaggcccgctgaggatgggagctgcgaggaggtacccagactccatggaagccccaacccggatccgggacactccggaagacatcgtgctggaagctccggctagtgggctggcgttccatccggcccgtggaccttactggctgcaggggacgtggacggggacgtgttcgtcttttcctactcttgccaagagggagaaaccaaggagctctggtcatcaggtcaccatctcaaggccttccgagctgtgggctttctctgaagatgggcagaaactcattactgtctccaaggacaaagccatccatgttctagatgtgggagcagggccaactggaaagacgtgttttccaaggctcatggtgcccccatcaatagtcttctgctggtggatgagaatgttctggccactggggatgaccccaggtggtatccggtctctgggaccagcggaaggagggccccttaatggatatgaggcaacatgaagagtacatcgcagacatggctctggatccagccaaaaagctgctgctgacagccagcgggggaagggctgcacttgggcatcttcaacattaaagaggcgtcggtttgagctgctctcagaacctcagtctgggtgacctgacctctgtcactctcatgaaatggggggaagaaggtaagcctgtggctccagtgaaggtaccatctacctcttcaattggaatggctttggggccacaagtgaccgctttgcccttgagagctgaatctatccgactgcatggttccagtcaccgagagtctgctgtgtactggctccactgatggagtcatcagggctcgtgaacatcctaccgaaccagagtggtgggcagtgtgggccagcacacctggggagcctgtggaggagctggccctctcccactgtggccgcttcctggccagtagtggccatgaccagcgcctcaagttttgggacatggcccagctgcgagcctgtggtggtggatgactaccgtcggcgcaaaaaaaagggaggaccactgcgggctctgagcagcaagacttgggggccccgattacttctttcgcaagacttgagggacgagggagaagactccatggctcagcgaagaaaaggaggagactggggattaccggggctctgagggaatgaattgaatcttgagacgggtcctcaccagccccagccagaccacaggaggttgggccccagactcactgagtgcctgcagcagccgtacagacacagcatccttggccacctcatgcccatcccggccatctagggtcagcacaacccagatgaggccgctgaagggcaccgggatggccaggaatcaccaccgtggtaccagaagcggtgccagccagcaggtcctatgcccaaacacttggtgaggaaaccccagggttggcccagtttcattccgttggcacagcaatctgcagggtagccctgagccctcttggaaccctaacgttgtctccttggccacaagccacactgggctggccgtctggctgtggggaccgcaaggaagggacccaccaaagctgctcggcagaagtctgctgcattcaggtctggcttgaggccactccacagccacctgctctccacagaggttgtgactgccctgtaaaggaaaaaatgcaaagacaagggcaggtctaaaanccctgggcccaagcctccagggtggtgagccctttggaaagctacacagtcctgttaaatttgtagcctgtgccatttcctggtagtgtgatccatgggtaaagaaaagacaatgagagccttcagcctacattatgttgcaaaggtgagtagtaacagcaacgtgcgtacgcgttaacagagtcttgcctgtcggagatagtagctggtttattgccaggctcttgtgctgcctcagtaagccatccataatggcctaatcctttactggggaaaacttgcgtgtaagtccagtcagtgtgctatacgtacacgtagcacccacttagtgttaccttaacttgtcataaacagtctgaggaaacagattcctatagtagtaaaaaggctcagtttggattccccttgagcaaagtcaatttctctaacagtttctctcattagcttgagggtccctgtgccctattttgcggtgtgggggtgggggaaggttgagaattaagtccaggaggtaatgctcgggaagtgtcacaaatttaggtaagcggtggtggtagcaccattggaagttttaaaaatctgaggtgcaaatagtaaaaggttgcaaattctggcatgtttgactctaagatcttgtaactttaacagctatgctcagtataaagtgccgggcagaacgcgtttggccccaaccagcacccccgccccagcctaccttccaccagggcctttttggccatggcagcggcccggtgcgagctgaatttgagcagagcgatctgcccgggcgccggtccggggctgggcagcagccgcgcctcctgcaagccggga

SEQ ID NO: 57 - DNA, 2196 bp, Homo sapiens
  misc_feature: Incyte ID No LI1143605.12000MAY01
gcataatgatcaaatcaaggtaatgttaaatgcttgaggcgatgtatatctccttatttttctaactggacttaaattctaaagagactatatataagttaaaaatgaagaataaataagagctcaattaaacgggaattttaaaagttccaataaatttgggataaaatgtaatgtaaggtgtttcatatggagagccatccctgccttttgatcactgtttccagaaaagcacagacttgtgcacctccctctgtgctttcttatgttttgacagttgaacataccaaaacaggtgatgagcaaacttgcaagcagtttttactgatagtttttacatagcataatgaagtataatgttatcaaccaagaactgtaaaaaaaaaacactgtgaggcttatgcctcttccagacttgcttttttgaacttttccatacattttttgttttagacctattccatctgggctagcagttgtaaaaacaaagcagagtgcaatgaactccatccgtctgtttctgtggtacagattttagccttcatcctaataagaaacatccctggatatgcccgttcagtttacagttcattttttgcttggtttggaaaaatttcattagagctatttatttgccagtatcacatatggctggcagcggacacaaggggtatcttggtactgatacctgggaaaccctatgctcaacatcattgtcagcactttcatatttgtttgtgtggccacatgaaatttctcagatcactaatgatcttgcacagattattattcgctaaagataactcatctctcttgaaaaggttggcatgtatagctgcattttttttttgtggactcctcatcttatcatccattcaagataaatcaaaacattaggttccaaaaattctaaaaaacctaaactttttccaggctacctttggtgtggctccaaaaaaagaaaagcatctatctggagatataaatgtgtatgtaaatataaacgtttgtggcaagaggacagttctgtgacatctgttgaacatatgtggttgtaaatatgtgtaaatgtacatatcccaatatgaaatactaagacaaacaaacaaacaaaaaaccagaatgcattgtataggattgcatgtgaagtcttttctactgaatctatatttccatttgtaagtgattttaagttaacatatgaaggcagggaaatgattacctttccagtaaaaagtataggtaattttaattaacttagtgacaccaccaagtgtttttgatataactaaaatttgtggtaataaggcttgtcttgcacctgtattcattgtgggacttcctctttcattggaaaatttttttactcaagaatgacggcagtattgttttcttatatgtgcaatgaagtggaatgataaacagtatgcctttaatttatatgtgttcttgttctgatgttgtttcctgaaatgatttttcttcctaactgtggttttcgggtatgcaagcctttaatcttttgtaccactttgtctcacagaatagttctgaggctccatgacagggttttgtcattgttgatgtttattgttgcttcgttttataaaaaagccaaaattttttttccaatccaaacgttcacctgtttcctttcctcaagctataccagttgtaataccagttaccctgtgggatccatttaatatgttatccccactaaataaatttccatatattatttcccaatatttgggaaagcctctttatagcccatttggggtatttcctattacccacctcctattttaaatatttatcagtctaaacttgtgcagtgtagtaaacatgcaagttgttacgattgagctgtattacccctaagtagaatttctaagtaaacctggtgaatttggaccatagatgtttttgctttttgtttgatttttttttacaagctaactgttagaggtatacatttatttatctgttgtacagatatgattatgattttaatgtttgaaagattgcacttgtttgcttttactatatgtggggtaaaatatattttctgttcacagtatatgaaaatatggagtaatttaaacagtaaataaacattctgtggatgcttatttttgtattggcaaagtatcaattaaactatatgtgttctttttcaaaaaaaaaaaaaaa

SEQ ID NO: 58 - DNA, 2250 bp, Homo sapiens
  misc_feature: Incyte ID No LI474069.72000MAY01
cccgcccgccggagactcccgcggcgggacccgtcaaagccccggggatgccatggatcgtcctcgttggcggcttctggacactgagccactagagggcatcggtcccggtaaggccggacggcaggctccgggataccgccacgctctctatggcgcccagaaaggggcggggtggcgaccgcagtcgctttggctgaggctctgcngggaagggaaggggggcttttacgtcatcaatctgcgccaactgactcccaagtccctggcttggagtgggaagtgaggtgtgggaagtaggtcgctttctgatgaattcagtggcagtgaattgagaccggagggaatctggcccctagaggctggtacttgggcccgaaacccccatctccggcggagagaccgtccgaggtaattgtctgccacgagtggacattctgaaaacaggagattttagttcctaaaaaatgggaagaacctacattgtagaagagactgttggccagtatctttcaaacataaatctccaaggaaagggcttttgtctctggccttttaataggcacagtgttccgtcacaaaaggattatgtgatttcttgccactagaacggccacccaaagagggagcaaagtgagaacctcaaacatcccaaaagctaagtttggataacttggatgaagaatgggccacagaacatgcctgccaggtatccagaatgctaccagggggacttttagttcttgggagtatttattattactactttagaactggcaaatgattttcaaaatgccctgcgtagactaatgtttgctgtggaaaagtctataaatagaaagagattgtggaatttcacagaggaggaagtctcagaacgagtgacacttcacatttgtgcttctacaaaaaaaatattttgtcgaacttatgatatccatgatccaaagagttcagcaagaccagcaggattggaagtatcaaagtggattatcatcctcatggctttctttagagtgtacagttcacattaatattcacatcccactttctgctacttctgtcagctatactctggagaaaaatacaaagaatggacttacacgctgggccaaggaaatagaaaatggtgtttcatttcgcattaatggacaagttaaagatgaaagattgtgacctattagaaggacagaaaaaaatcttctagaggaaatactcaagcaactagtcattgttttgcatgtcnacgagtgctaacgcagttgcgtcctgaattcagaccacatgatccacagccacagtccacgatatcgtagcggttctgtaaaccttaacgggtgctgtgaacatcgcagagctttaatatccacagcagtaaacccaaagcttaaagatgctgtgcaggcacgtaaacgagggatatacttgaacacangcttgctgatcgttgtgaaactgctatttgacggatctgcttttgcaatgaaattcccagaaaaaaaaagttatgagtataaaattctgaaaaagagttccaccgtcctccctttatccgagtctttgttccccttcctggatccacctgtaatgttgtgtgattataaatttgacgatgagtcagctgaagaaatcagggaccattttatggagatgttggatcacacaattcaaatagaagatttgggaaattgcagaggaaacaaacacagcttgtattgagtcctcctatgaatagtcaagcttcattggacaacacagatgatgaacaacccaaaacaaccaattaaaactacaatgttattgaaaattcagcaaaacataggtgtgattgcagcatttacagttgcagtccttgcgtgcgggtatctcctttcattacttcagtgattagggtgaggcacaaagagtttcctgtgatcatccagagaacattgacagacaattatgaataataaagatgttaacaatccatctgtatttaaaacacctagccgccagatctgctgccatgatgcctatttggtgtgtttctgattaaaatgaaatcaccagctgccttgtttagcctgctttacattgtaggtggcccgcatttccagaaataacgttatgcatctaagatggaagctgcatgtaaccaatcattattatctatttttaaaagcttccaaatgatgggatatgatcatagattttagtcttactaatctgaatcacatattaatccaggacatttaaaactttaacaga

SEQ ID NO: 59 - DNA, 2963 bp, Homo sapiens
  misc_feature: Incyte ID No LI245193.32000MAY01
gtgaggctggcggtggcgacaaccgaggaggaggggcgggacgccgtggagcacggcgaccggctgagcgtcatggagggctcaggggagcagccgggcccacaaccacagcatcccggagaccaccgcatccgcgacggcgacttcgtggtgctgaaacgtgaagatgtgtttaaagcagtacaagtccagcggagaaaaaaagtaactttcgaaaaacagtggttctacctggataacgtcattggccatagttatggaactgcatttgaagtgaccagtggaggaagtctacagcccaagaagaagagggaagagcctactgcagagactaaagaagcgggcactgataatcgaaatatagttgatgatgggaaatctcagaaacttactcaagatgacataaaagctttgaaggacaagggcattaaaggagaggaaatagttcagcagttaattgaaaatagtacaacattccgagacaagacagaatttgcccaagataaatatattaaaaagaagaaaaaaaaatatgaagccatcattactgttgtgaagccatccacccgtattctttcaattatgtattatgcaagagaacctggaaaaattaaccacatgaggatacgatacactagcccagatgttgacgttgggaaatatccgtgctggcaacaaaatgattgtgatggaaacgtgtgcaggcttggtgctgggtgcaatgatggaacgaatgggaggttttggctccattattcagctataccctggaggaggacctgttcgggcagcaacagcatgttttggatttcccaaatcttttctcagtggtctttatgaattcccctctcaacaaagtggacagtcttctacatggaacattttctgccaagatgttatcttcagagcccaaagacagtgctttggttgaagaaagtaatggcacactggaggaaaaacaggcttctgaacaagagaatgaagacagcatggcagaggccccagagagcaaccacccagaagaccaggaaacaatggaaacaatttctcaagatccagaacataaggggcctaaagagagaggaagcaaaaaagattatattcaggaaaaacagaggagacaagaagagcagaggaaaagacatttagaggctgccgctctgctgagtgaaagaaacgcagatgggtgcgttgatggaagaaggcaggagactcctcagtgcatgtgggctctgcttcagacagattggcattctctttctccttgtctgctttgactctagtgggacccgagatgaggattttgtcttgggggtttttttaaagaaaggaggctgaggctggcaggggtggggaattgatgatgctggtttcagggtttcagaagaaattgaatcttctccatcactgcatcaagatgggccctgtggtcccactggatgcaggggatggggcactcaaatgctgcgatacaggctataacagatatccacaaagcagcttatgcagtgttggatttgtttccaggtgattttttttaaaaaatcaagaatgtcattaaatgcgacttagagttgccaagtaccacaagagcttctaacctagttcgcgtttcctagaaagaaatagctcatgattgtattctgttttggatttctagaatgttatatttaaatctcttctctttgtgagtcatgcaaattaaatacatgatcattttaaatgcattttaaaacctgtgctccactttgagggtgtagagaaataatttgtctggcattttccttacagtttaattgtagctagtcgtttccaccccactcccctgctgctgtctttgctggactttgtggccccttcaaggccgtttgtggtctactgtcagtacaaagaggtaatactggaatggaatgggcaggaattcttatgtctgaatgttgattcttaaactgcatcccaggaagtagttcttaacagaattctgttgttgtctgcagcctctgttggaatgctacacaaaactgcgggagaggggaggggtcatcaacctcaggctgtctgaaacctggctcagaaattatcaggttttgccagatcgaagtcatcctaaactgctgatgagtggaggtgggggttatcttctctccggcttcaccgttgccatggacaaccttaaagcagacaccagcctcaaatctaatgcaagcactttagaatcacacgagactgaggagcctgcagctaaaaaacgaaaatgcccagagtctgactcttaacccttttgagaattgctctgaattttgttctcaggcattatacagttagtaattaaactttaaatgccattactacttgttttttcatatcccaagaataagaacatgtctatgtacccgtttctaggaaggaggagtatatgccttttgtctatttctgacacaagattgctttgggccggtcaaaacctgcaaccatggggtatgtagtatgaccaactgcatttaacaaaactcctctaagtacttctaaatattctgtgcaaatatcttgagaacttcaacatcctaaaaatgtgtttctacaagactttatattttaagctaagtggaataatacaacgtaactaaaacatggcgggtaccgaaaggagggcaattcatggaagaaatttcctctgttcattccgttctcttcaacctaagcaatatggatgtgtgagtgtgtgatttattatggtaccaaaaatgtccaccttctttcctgcagcggaataagcttttttgaaatgctgaggttttattttaatagttgacaacctgggaaagttctgagtatttattttactgctctttttttgtgcatagaaaggtttagtagggtactttttttgcattaatttgccaatactcttattcatatgttctaaaaaaagggacctggaatttttgtgtactattcaaaaa

SEQ ID NO: 60 - DNA, 2569 bp, Homo sapiens
  misc_feature: Incyte ID No LI403872.12000MAY01
ccaggaggaggcagcggaggaagcagagcgcgggatgggggcccagcggcatctgtgatcccgcgcacctccgccccacgggcgcgcgcacaaacacggacacacacatacacacactcgcgcacacactcgcacaaacacacactcgtacacgcccgcggccgctcgctacgccggcttgctctgggcacgcaagcggaatgcagcagcgcctggagagcgtgtctcggaccgccgcctgaatgtacctcgctcccgggagccggacggcccagtagggcgcactggaggacgctccgctgcgggacgcttggacagttttttgacggtgcagtcttgctatatggtgtgagaaatggctgtaggaaacaacactcaacgaagttattccatcatcccgtgttttatatttgttgagcttgtcatcatggctgggacagtgctgcttgcctactacttcgaatgcactgacacttttcaggtgcatatccaaggattcttctgtcaggacggagactttaatgaagcccttacccagggacagaggaagaaagcttcatcaccccttctggtgcctctattgtgtgctggctgccaccccaactgctattatttttattggtgagatatccatgtatttcataaaatcaacaagagaatccctgattgctcaggagaaaacattcttggacggggaatggctggttccttgaacccccttacttcgaaggatcataagattcacaggggtgttcgcatttggacttttttgctactgacatttttgtaaacgccggacaagtggtcactgggcacttaacgccatactgccctgaacgtgtgtgcaagcccaactgacaccagtgcaagactgccaagcgcacccaccaggttagaaacaatgggaacattttgtactggggacctgggaagtgaagtagaaaaggctcggagatcctttccctccaaacacgctgctctgagcatttactccgcctttatatgccacgtatctatattacaagcaccaatcaagacgaagagcagtcgactggccaagccggtggctgtgcctcggaactctctgcgccagccttcctgacaggcctccaccgggtctctgagtatccggaaccactgctcggacgtgattgctggtttcatcctgggcactgcagtggcccttgtattctagggaatgtgtgtaggtacataactttaaaggaaacgcaaggatctccagtccggaccacagcactgaggatccccacgtcggagtacccctaatgccatcgtcccaatggagtagcaaagtccactctggataacctataagtgcaccagagatcacttcttgggtcccaatggcccggaaggtaccctgagagccggcgcttgaatgtgtcaaccaaacgctgttttttgaaaaactagtcgtttccccaagttctatatgcacttccgaaacaaccaccacggttgcgtccaatgtcccaaccgtgggggagccaaataattaacggttaattctaaggtatagagaagctataatagttttggaacacactttttgtaatgagaggagaatgagatgtatagctttccccggaattttttttttttttttggtcagctttaatatatttatgccagaattttaaaacccaacacaaattttcttgttcaagcgtgcattgaagaaccacatttattcaatggttgacgttgttttgtgatatttgtacacaaattttcttttctcagttttataaacacagaagtaaatataacaattcactttaaacttttattaccacagttgctgcctcctccagaatttttgaattttaataaaaggcaaacttttgagctgcaggaagggacaatgtctggttaataactaaatctcagagtcaattgtagaacagacaacaattgtcttcaagaaagaatgttggcactcctgatctccttaaacaaattgttacgttcatatagtttacacatgtgatatattaaccaacatgtcaccatagttataccaaccaattgtcagagaattctggatttggagggtatatggggttatatgattctttcttagataatggcctctactaaactaactcaagatctttctggaatgtcttctggcaggcaggtgccactgtcagcttttctccaaaaagcaggccaacatcagcctcccctgtcaactcaacagttttgtatctcatattatatggacctttatatgaaagatgaatattttaccagtttggcacagtattattttacagaaaaggaatcagagaatcctacaacatagggccccagaacaacagtttcactttgtggctttataattattctaggaattttaactgcatctcatttttctagcatggtgagaactaatatgtaactccttttgattgaaggagctcttttgtccgtacctatcaagaatgttttcttgacacttccatgttgggctcttctcagctttttttgtacatattttttttttctaaagagaagaacaaagttttcaccaaattgtccgacg

SEQ ID NO: 61 - DNA, 2545 bp, Homo sapiens
  misc_feature: Incyte ID No LI1086294.12000MAY01
gagcccgaggcggcagcgctcgcggccatcgtgggcccgagctgtgcgcctcggctcggagcccggaccgggcgggggcgccgacgtgcctcagcctgcttcgcgagggaggcgggagtgagcagaaaggcttagggcccagtgggcggggaaggggggccgggcctggatccggcggggaggccgagccggaggccggaccgtggctcgcgctgtccccgggacgcggatcgaacgttggcggcgcggatcgcacgtcggcggccggtggaacgcgggagctgggcggcgcgcggactggggccatggcgtctgtgcaggcgtcccgccgccagtggtgctacctgtgcgacctgcccaagatgccgtgggccatggtgtgggacttcagcgaggccgtgtgtcgcggctgcgtgaacttcgagggcgcggaccgcatcgaactgctcatcgaatgccgcccgccagctcaagcgcagccacgggctccccgagggccgctcgcccggggcccccggcccttaagcacccggccaccaaggacctggcggcggcagccgcacaggggccccagctgccgcccccgcaggcccagccccagccgtcagggaccggcggcggcgtgtcgggccaggaccgctatgacagggccacatcatcaggccgccgtccccctgccctcgcccgccactggagtacaactctggggtccccgcctgggccaatgggctgggccgtgaggaggccgtggctgagggggcgcgaagggccttggcttggctccatgcctggtttgatgccccctgggttgctggcagcttcagtgtctggcctgggaagccgaggcctgacgctggcacccggcttgagtcctgcccgtcccctcttcggctccgatttcgagaaagagaagcagcagaggaatgcggactgtctggcagaactgaagcgaggccatgcgaggccgggcagaggaatggcacgggcgccccaaagcagtgcgggaacagctactggcgctgtccgcctgcgccccgttcaatgtgcgcttcaagaaggatcacgggctggtggggcgagtgttcgccttcgatgctaactgcccgtcctccaggatacgagttcgagcctgaagctctttcaccgaatacccctgtggttccggcaatgtgtacgccggcgtcctggcagtggctcgccagatgttccacgatgctctgcgggagccgggcaaggcactggcttcttcgggcttcaagtacctcgaatatgaacgccggcatggatcaggagaatggcggcagctgggcgagctgcttaccgacggcgtccgaagcttccgcgagccagctcccgcggaggccctgcccccagcagtacccagagcggggccctggggctctctgtggcccacccccgcgagccccatcccggaacctggcgcccacgccgcgccgtcgcaaggcatcccccgagccggagggcgaggcggctgggaagatgaccaccgaggagcagcagcaacggcacttgggtggcacccggcggccctgtactccgcgtgagacccctcggtgtgccctcgcccattgccgccctgaagaatgtggccgaagccctgggccactcgacccaaggaccctgggcggaggcggggggcctgtgcgtgcaggtgggcgccagccctaacagcctaccaccacgagcccagaccgccaaccccagcatcgcctttgtggccgcgcaacaggcgaagcagcaagtcagtcccacaagcgggggccgaagctgtcaacgggggtggcagcaggcactggggcgacccctggggcccaccctgtgcttgtaccctgtgcagggagcggctagaagacaccccacttcgtcccagtgcccctcggtgcccggacacaagttctgctttccctgctcccgggagttcatcaaggcgcagggcccggccggggaaggtgtactgcccgagcggagacaagtgcccgctggtcggctcctccgtgccctgggccttcatgcagggcgagatcgccaccatccttgctggagacatcaaagttaagaaagaacgggacccctaggctaccactgcctccaggctactgccgntctgcccctttgccacccaccggctgccgcggataaattattcccctccccgacccagcccagtgccacctccatctgggtggaatgaagggagtatgtacacagatagaggtggcactggggtaagatgcattgtgggtgggggggagaaagcttgtggctcctgtccgagggggggtgtttgggggtcccttggccccctccagtttccctctagtcctccttggcttggctttgcttgctgcttgtagttagggggagaaggtgcccccctacctccttgagaatgggacctacctgaaaactctgctctttataaactgagtgcaaaagcattgtaattccacacccccgccacccccctgcgtcgccttcttgctccttcaataaaccgaaagatgggttttttttttcaaaaaaa

SEQ ID NO: 62 - DNA, 2114 bp, Homo sapiens
  misc_feature: Incyte ID No LI337514.32000MAY01
agcggctggagctgggccttggccgcgagaggccggcgaaagctatcttcctgcaccggcggccgggtgagggcggtgggagggagcggtgcctgcgctgtggccatgtctgtgtacgcaagggggccggggccccgcgaagctgtgccctcaggccggccccggccggacacccttacccctccatgggtccggcagagggcagtgaccggcaccttctgtgcgtcctggactcctctgagaaacaggagagctcaaaggatggctactgacatgcagaggaagagaagcagcgaatgccttgatggcacattgactccttctgatggacagagtatggagagaactgagagccccacaccaggaatggcccagggaatggagccaggagcgggccaggagggtgccatgttcgtccatgcccgttcctacgaggacctgactgagtcagaggatggggcagcttctggggacagccacaaggagggtaccaggggtcccccgccgctgcctacagacatgcgccagatcagccaggactttagcgagctaagcacccagctgacgggtgtggcccgggacctgcaggaggagatgctgccaggaagctctgaggattggctggaacccccaggggcagttgggcgaccagccacagagccccccagggagggcacaaccgagggggatgaggaggatgccacggaggcatggcgcctgcaccagaagcatgtctttgtgctgagtgaggcagggaagcctgtgtactcccgctatgggtctgaggaggcactttccagcactatgggtgttatggtggcccctggtgtccttcctggaggcagacaagaacgccatccgctccatccatgcagatggctacaaggtagtattcgtgcgccggagcccgctggtgctagtggcggtggctcgtacgcggcagtcggcacaagagctggcgcaggagctgctctacatctactaccagatcctaagccttcttaccggtgcgcagctgcagccacatcttccagcagaagcagaactatgatttgcggcgcctactctcgggctcagagcgcatcaccggacaacctgctgcagctcatggcacgagaccccagcttcctgatgggggcggcacggtgcctgcccctggcggcggccgtgcgcgacactgtgagcgccagcctgcagcaggcgcgtgcgcgcagcctggtcttctccatcctgctgggcccgcaaccagctcgtggcactcgtgcgccgaaaggaccaatttctgcaccccatcgacctgcacctgctcttcaacctcattagttcctcctcgtcctttcgcgagggcgaggcctgggacgcccgtggtgcctgcccaaattcaacgcagccggcttcttccacgcacacatctcttacctagatgcctagacactgacctactgcctgctgcttgtctccactgaccgtgaggacttctttgcagtctctgactagcgccgccgcttccaggaagcgccttcgcaaagcgcggagcccacctggccctgcgagaggcactgcgcacaccctactacagcgttgcccaagtgggcatcccctgaccctgcgtacacttcctctaataagtcacaagagctctgggactcttccaccagcccttgagattgaggccccatacaccagtgaagaggagcaggagcggctgctggggcctctaccagtacttgcacagtcgtgcccacaatgcctctcggcccactcaagaccatttactacacgggccccaacgagaacctcctggcctgggtgacaggcgcctttgagctctacatgtgttacgagccccctggggaccaaggcgtcagccgtcagtgccatccataagctgatgcgctggatccgcaaagaggaagaccgcctcttcattctcacgcccctcacctattgatgggaatgtgtgcgggctcagccttcctggacacactaggtgtgggaagccataggagcctcaacgatgggggctggcctctcttgcccgagccagcgggcagggactgtgggttggtagaatgcattaaagtgctttggggaagacaaaaaaaaaaaaaaaa

SEQ ID NO: 63 - DNA, 2496 bp, Homo sapiens
  misc_feature: Incyte ID No LI230711.12000MAY01
ggcagcatgagccgatcacccctcaatcccagccaactccgatcagtgggcgaccaggatgccctggcccccttgcctccacctgctccccagaatccctccacccactcttggagaccctttgtgtggatctctgccttggggcctcagctgtcttctggctctgcagcatgtcttggtcatggcttctctgctctgtgtctcccacctgctcctgctttgcagtctctccccaggaggactctcttactccccttctcagctcctggcctccagcttcttttcatgtggtatgtctaccatcctgcaaacttggatgggcagcaggctgcctcttgtccaggctccatccttagagttccttatccctgctctggtgctgaccagccagaagctaccccgggccatccagacacctggaaactcctccctcatgctgcacctttgtaggggacctagctgccatggcctggggcactggaacacttctctccaggaggtgtccggggcagtgcgtagtatctgggctgctgcagggcatgatggggctgctggggagtcccggccacgtgttcccccactgtgggcccctggtgctggctcccagcctggttgtggcagggctctctgcccacagggaggtagcccagttctgcttcacacactgggggttggccttgctggttatcctgctcatggtggtctgttctcagcacctgggctcctgccagtttcatgtgtgcccctggaggcgagcttcaacgtcatcaactcacactcctctgcctgtcttccggctccctttcggtatgtgtgggtctgggcagggcagtagaggtcagaagggctggcctggagtgctcactccatcccctaccttttggcttctgtctacccctgcaaggctggctcagaaggttttgggggaggagttcttttctcagtctcgcccctcaggtgctgatcccagtggcctgtgtgtggattgtttctgcctttgtgggattcagtgttatcccccaggaactgtctgcccccaccaaggcaccatggatattggctgcctcaccacaggtgagtggaaattggcctttgctgacgcccagagctcttggctgcaggcattctccatggccttggcaagcctgccaccagtgtccctggggctgcttatgccctgtgtggccggctgctggcatttggcctccccccaaccgtccaacatgcctgccagtcggagggctgagccctggaggggctgggacagtgttgctgggccgggctgctgggaaggccccatgggcactgcatccagctttccccaacgtgggcaaagtgggtctatatccaggctggatctcagcaagtgggctcactatagtggggctactctggcgtggggctatggactctcaccccaggtttggctcagctcctcaccaccatcccactgcctgttgattggtggggtgcatgggggtagaacccaggcatgtggtctttgtctcgctggattctcccagcttctacctggcatgacatagactccttgggccgaaatatcttcattgcgggcttctccatcttcatggccttgctgctgccaagatggtttcagggaaagccccagtcctgttcagcacaggctggagccccttggatgtattactggcactcactgctgacacagcccatcttcctggctggactctcaggcttcctactagagaacacgattcctggcacacagcttgagcgaggcctaggtcaagggctaccatctcctttcactgcccaagaggctcgaatgcctcagaagcccagggagaaggctgctcaagtgtacagacttcctttccccatccaaaacctctgtccctgcatcccccagcctctccactgcctctgcccactgcctgaagaccctggggatgaggaaggaggctcctctgagccagaagagatggcagacttggctgcctggctcagagggagccatgccctgaatctagcagagaagggtttaggtccctagaaatgtaccagtaacgccttacttctgccctggttaattttagcccttatactctctatctgctggagagtcagctcccttaactgttctttcttgtaggcagtaggatatgtgtgtgtgtattacatgggactgtctagaggttcctatttcccaatagggtgggttgcctttccttgtcttaattaggcctaactgttccagagcagaggccatgatttagtggaccatgaatgattgagattttgcgctgtgtactatcaatgccacttgaagccaagcattcactttaatacttactgagcatctcccattgtgcaaggtcctggaactacacgggataagacagggtccatgccgtctcgaaggcatttacggtttaaaaagacctttgtaattactaacgaaaatgcaaagcagaaagcagtctgtaataaagattaaaataatgccgtgggagc

SEQ ID NO: 64 - DNA, 1377 bp, Homo sapiens
  misc_feature: Incyte ID No LI040338.22000MAY01
gtcgcggcggcggggaaggaggggtgcttcaggtgcctgcgacctccgcgcctcccggtcggaagtgcccgaggtggccgcgatggagctgggggagccgggcgctcggtagcgctggcgggcaaggcatggcgccatgaccctgattgaaggggatgggtgatgaggtgaccgtccttttactcggtgacttgcctgccttactggtgctggcccttgcctgaggtctcaacgcacaccgctgagaggcggggacccacttgccccagcctgtcagggaccccaactgccatcccagcccagcgcagccatgggcaggttaccgacagcatgagaggggaggccccaggggcagagacccccagcctgagacacagaggtcaagctgcacagccagagcccatgcacggggttcacagcaacaccgccagccccgggactccccgcaggagccccatcgtgctacggctgaaattcctcaatgattcagagcaggtggccagggcctggccccactgacaccattggctccttgaaaaggacccagtttcccggccgggaacagcaggtgcgagctcatctaccaagggcaagctgcctaggcggacgaccaccagaccctgggcagccttcacctccctaccaagtgcgttctccactgccacgtgtccacgagagtcggtcccccaaatcccccctgcacgccgggggtcccgagcccggccccctccgggcgtgaaatcggcagcctgttgttgccccctgtgctcctgctgttgctgctgctctggtactgccagatccagtaccggccctttcttttcccctgaccgccactctgggcctggncggcttcaccctgctcctcagtctcctggcctttgccatgtaccgcccgtagtgcctccgcgggcgcttggcagcgtcgccggcccctccggacccttgctccccgcgccgcggcgggagctgctgcctgcccaggcccgccttttcggcctggcctcttcccgccgccctggagcccagccctgcgccgccagaggactcccgggactggcggaggccccgccctgcgaccgccggggctcggggccacctcccgggggctgctgaccctcagccccgcactgggagtgggctcctcggggtcgggcatctgctgtcgctcgccttcggccccgggcagagccgggccctcccgggggcccgtcttagtgttctgccggaggaccccacccgcctccaatccctgacagctccttggggtgagttggggacgccaggtcggtgggaggctggtgaaggggagcggggaggggcaaaggagttccccggaacccgggcagattaaaggaactgtgaagttttcaaaaa

SEQ ID NO: 65 - DNA, 1445 bp, Homo sapiens
  misc_feature: Incyte ID No LI399174.22000MAY01
gaaaccaaggacaatgtgtcctttaaaagtgtaaggttagaaggtgccctgactccagccgacctggaaaaggacgtgtcctgcatatctggaggtggcccacagggtccgggaaagatcggctgccttgcctgcatcccagggcgcttcccggcctccattaagaggtgaccattgcatccacgtgatttcttttgctccactatttccaggcacagtgcaactgactccctcttgaagctagaatgccctgcacacccgggatggacttctctcccctttcaaagatcgagtgaagttatgcaaatgtgaaaggaaggaattgcatttccagtggtaacctttgaaggggaatgacattgtcacaggacgtcaggagccacgtggggggagcccggtgtcctctgagtctcaacgccaccttgcattcccgcttggtgacggtcgatgggctgtgtccagcggtgaacgctttacacaaggactctacggtggggtaggcaagcgccacttcaatgtgaagacaaatgaactggcgtctcttgagatatgcccctgcctccaggacctctcaggccccgggccctggttactgccgtggaagctgcctgttcagacgtcatctctatcaccccttgaagggagacagagaggtgtttgcgagttggatgttttatcttgtctttcctgattctttgttagaacacccccagggcttggtttttgttctcaacctgtgattagaaataagatcctaaagtgtcagatctggaaggcccatgagagctgaggagatcaaatgcccggagctcgggagatcagatgacctggggtgtctggagccagtggtggcctcatctgcagcatcttcctctgtcctgcccaccacagagtccctgctggccccggggggcccctcttccgccacagggcccctgagcattgctgcgtttgtttctccagctcctccttcctgcagaagcagttcccagaggggcaatttctccagcctgcctggttggttgtgttgattggttatagttgctctttcaggtgaccgcagaccctctgaaggctgtggccgtgggtgtggctctatggccacctctctctcttctcctctcccaggtcgcgtctccttcttcacctcggggtccgagaacctacaccgcgtggagaaggtccactggggcacccgttacgccatcaccatcgccttcagctgcaaccccgaccatggcatcgaggacccagcgttcccgtagccagcagccgggccaaggtaacttcaggagggcccgtgtggaggcagccgcgccagtcgtgcacccacccccacgtccacccgctcctgttgcacagagtgccttaggaattctctgattttgatttgctgacgtgttaatcttttcatctgtgagtaaatgaagggaaccagcaaaaaa

SEQ ID NO: 66 - DNA, 1578 bp, Homo sapiens
  misc_feature: Incyte ID No LI197275.52000MAY01
cagtgctcttcagcatctggagtctttgcagaaacagtatctttttgtgtccaataagacaggaaccctacatgaagcctgtgaacagctcctaaaagaacagtcggaacttgttgatctggctgaaaacattcaacaaaagctttcctattttaacgaattggaaactattaacacaaaattgaattcccctacattgtcggtgaatagtgacggatttatacctatgctggccaagttagatgattgtataacatatatctcatctcatcctaattttaaagattatcccatatatttgctgaagtttaaacagtgtctttctaaagctttgcacctcatgaagacatatactgtgaacacactacagaccctcacaagtcagttactgaaaagggatccttcatctgtacctaatgcagacaatgccttcacattattttatgtgaaatttcgagctgctgcccccaaagtcagaactcttattgaacaaatagaactgcggtctgaaaaaatacctgaataccaacaactgctaaatgatatccaccagtgttaccttgatcagcgggagctccttttgggccctagtattgcttgcactgttgcagagttaaccagccaaaataatagagatcactgtgccttggttcgtagtggctgtgccttcatggttcatgtctgccaggatgaacaccaactttacaatgaatttttcacaaaaccaacatcaaaattagatgagcttttggagaaactgtgtgtgtcattgtatgatgtcttcaggccattgatcattcatgttattcacttagagactctgtcggaactttgtgggattcttaaaaatgaggtgctgaagatcatgtgcagaacaatgctgagcaactgggggcatccgcagctggagtcaagcagatgttagaagatgtacaggagcggctcgtctaccgaacccacatctatattcagacggacatcacgggctataaaccagctcctggagatctggcatatcccgataagttagtcatgatggagcaaattgcacagagtttgaaagatgaacagaagaaggtaccttcagaagcttcattttcagatgttcacttagaagaaggagagtctaacagtctgacaaaatctggttcaacagaatccctcaatcctagaccacagaccacaatttctccagcagatcttcatggaatggtggtatcctacggttcgaagaactcttgtctgtctctccaaattatacagatgcatagatagggcagtgttccaaggattatcacaggaagcattgtctgcctgcattcagtcccttacttggagcgtcagagtctatcagcaaaaacaagactcagattgatggacaacttttcttaattaagcaccttttgatacttcgtgaacaaattgctccatttcacactgaattcaccattaaggaaatttccctggacctcaagaaaactagagatgcagcatttaaaatcctgaaccctatgactgtcccaagattttttaggctgaatagcaacaatgccctgatagagttcttgttggagggta

SEQ ID NO: 67 - DNA, 1738 bp, Homo sapiens
  misc_feature: Incyte ID No LI336872.12000MAY01
aaaatagtcatccttaactggtttggaccatctttcattagtaaaatcaaaagcatatgtgcacagtaaaattcctagctgcagacgcaggctcacctcccacagccaaccccttagccccttgaactgctcgcatgaggacctcagacacacttctctaaacaccccttttcatatgaaaatactggaggcagagtgtccttccatttgtgggtttctatttttatttttgagacaggatctcagtctgtcacccagcctgtgatcgcaccactccactccagcctgggcaacagagtgagacttagtctcaaaaaaaagggaaggttgaatttttacttcatattcacccccaattaacttcagttggattaaagagctaaaactagaagcaaagtttttcttctaaaacattggaagacaatattgcattttttataatcaaagggtctttctgaaagtgacacacaagtcccccatctgtaagggaaaagtttgagatttgagtatgcaaattggtattttccataagacaaaagaaagcataaaggaaagcaatgagctgtataaaatattcacaaaatatctatccgaagatgtatatcagatatattgagagaccctaacaagatcagtaagacagaaagaaactcacttaaaaaaagtcagtgcgcttttctacattttttcccaaaataaaaggattggcaaagcacataagcagaccattcatcaaagaagtaaagatgactgattaataagaaaaagatggtaacatcactaataatcagtaaggccaggcacagttcatgcctgactttgggatcccttgaacccaggagttcgagaccatcctgggcaaaatggcgaaaccccatctctacaaaaaatacaaaaattagctgggcgtgatggcacgtgcctgtggtcccagccgcttggggggctgaggtgagaggatcacttgagcccaggagttcaaggctgcagtgagccgtgtttgcgccactgccctctagcctgagtgacaaagtgagacactattaaaaaaaaaatcagtaaaatgtaaatccttgcattcctgatgttgatacgcatggcttcttttttaaaaacgttcagtcttactagaggttctcaaataattttttcgagagcgcacagctcccgggttcattcatattccacattttggtgttcaaattcaatgaagtctgctttttggtcttcctttgttctgctaattgttggggtcacactttaacttaatcgctttttgtcggtatctcggtggaaagctccacgagagtaattgaattctgagaaaccctctttgtgcaataaataaaaagacacctctccataggccatgagtaatatgttccctcttcatagacacctggcacatatatctgggggcccacacggatatgttaataataggctcataaatattgcaactttcttctttgccctctgtgtaagaataaagagtttctggaatattttcttggagaaatctctctttggggaattaattatagacatataggtgtgttaacattcccaggcgcaatattctagagatcgtccctggtatcccctcttaatacatggaaatttagagtccaaatcccacatatgggccagaaaacaataacactttgggataggaattcaggttattttacaatcaattaagagttattttataaacccgggaaaaaaaaaaaaaagg

SEQ ID NO: 68 - DNA, 640 bp, Homo sapiens
  misc_feature: Incyte ID No LI1092901.12000MAY01
tttaggaaatagtataggagatacatgatcaagaatttatataagatggggttaatcaagaaatgagagtaaaataagaataataatggtaataattttgaacactgacaaaaggacattgatgacacagatctacttttttcaggtcttcaaaatgtttcttctatgtcattaggatgaacacacatttcacatgttatctttcaaaaggccttttgttttcaagctggcattggaatctcagccaacatctttcttcttctctggcacatttttacattctttaaggatcacaagcctaaaaaaccatgacctgatcatctgtcacttggtttctgtctacatagtgatgctagtcatggcagcagagttattgtctctagacgtgtttgcgtcacagaattttcagaataacttcagatgtaaggctgtgttctacatatacaaggtaatgaggggcctcttgatctgcaccacctctctcctgagcatgcttcagatcatcaccatcagccccagcacctcactgcatagtcgtgaaatcggcagaattcccgataagctctccaaccacgtattttctgcgtttttgatccagacccagatggtactgctctggcagactcttctggtactcttc

SEQ ID NO: 69 - DNA, 1950 bp, Homo sapiens
  misc_feature: Incyte ID No LI022387.52000MAY01
tgctttggggtcaggtgtggagatcagggtcggggaggtcatgaggtaggtaggggtcaggatgtggacagtcatgggctggtgggtgtggcagccgtgaggggtgggtggtggcctgggaggcagggcctgggcagggcttctgcacttctgctgcttacccagccccctctgggttcgaggactctccttgacaagggctgagctctcgggcactgcgttggactcagctgcctactgggaggctggggagaatggacctgtgagaccttgtttgtcctcttccctgcccagctcttgtagaccccaggcctggctgtccgcagggccaggagctgcagccatggcagccaccagcctcccacatgagccccagctcccagccccacacgcccactcctttgcccatgtggtgtcagctctggcctccgcttccacacatggaccctcagaccttgcacatctgttgtttcccttgcagggcagtgatgatgacatgaaatgaggtggggtcactcctgtttagggatgagactggggcccagggaggggcaggtgtgaagttgctgagggcagagggagacccagggctacctgctcctaatgaccagcctcacgttctccactgctcactccctgccgttcaccagctgcatgaccttgagcagtgcactcaactgtaaacagaggtgaaaacactcctttaccaactgttttaggggccactcttgtttgaggccttgaacctcctttcagttccgtggtggtcctgagcctctgaatctgggtatggcccctgctgggtcctggtctcttaggcagcacgatggcctccctgctcaaggccctgcacccaggggcagattgtgggaggcccactggccatggctgggccctatcgcagagagcctgtcagcacggccatttccacacccaggctggctgggaagcaagctacgaggtgctcaaaggccctctatgtctgcaggtagccccgtcccacacaggcgaggatcgttgggcctccttagtagccaaaggaactgaggtccagagcagggaatgtgttgcccagtgtcagcatggtctgctctggggtcaggcgtcttgccctggtcctaagccaccccaagccaaggcatgtccctgggcccatttcctctctcgaggtcttgggttccctatttctgatgtgtgtgaaaggaaggcctgtcctctggggtgaccatgaggctcagtgggaggagctgccaggcttgagctgggacttggcacctggccagccttttttctatttcattgtaccttgatgggctgtcagttctgaagagaaaaaatacactaggaatccattcttcctccaccattctccctggcagagggaagtgactacacccctatggtgtggacatactgacgctcctccatgaagcagcaaagttctgttgtttcaggagactgtgggccttgaggatgtggtagtgacctcaccagaggaggtggcaattcctaagtgctgcctggaggcctctacagagacataatgttggagaacttctgccacatggcagtgctgggtaagacccctctgtccccaccagtgcccttaaggttatgggtgttggtaggtctgtggcctccatttccatgacgatggtgttaaggactcatgtttgtgtccccccccaaagtttatatgttgaaaccctgacccccaggagggtggtattaggaggtggggcctttgggaggtgactaggcttaggtgagatcgtgagggtggggctcgccgatgagatcgagtccttataagaaaaggaaggaactagagcgagatcactttgtgctgtgtgagcatacaagaaaactgccatcttcaagctgggtgcagtgctcacgcctgtaatcccagcactggaaggccaaggcattaggattgcatgatccagt

SEQ ID NO: 70 - DNA, 597 bp, Homo sapiens
  misc_feature: Incyte ID No LI1188334.12000MAY01
gggatttgtcttcttgcactttattttttctgaaatgtacattgctagtatttgtgtaagccaagtggttaatcttttcataaaaatgcagtcttaagaaatcttaacaaaatttggattttaaaaaagttgatattagacccagtgaaagaactactttttttactgttatgttttgtttttcatgattttgtatattcttcaggacaagcagtgggaagagctctgttatcaatgcaatgttgtgggataaagttctccctagtgggattggccatataaccaattgcttcctaagtgttgaaggaactgatggagataaagcctatcttatgacagaaggatcagatgaaaaaaagagtgtgaagacagttaatcaactggcccatgcccttcacatggacaaagatttgaaagctggctgtcttgtacgtgtgttttggccagatcatcttcagtggctattcgatcaagttccggattcttatatgttgcttcaacaaaatgtgatccttcagtaacaaactccagtaactggtcaaagattgcagtaatcgccttcttagccagcacaaagtgcttcagtggagaaacagg

SEQ ID NO: 71 - DNA, 963 bp, Homo sapiens
  misc_feature: Incyte ID No LI1188664.12000MAY01
gctttcgatggcctccctaggatggtcatccagaggataagccaacgcacatcatcttcggttctgacagtgaatgtgaaacagaggagacatcgactcaggagcagagccatccaggagaggaatgggtgaaagtaagaagttcaggatgtctctgtggacattcatctacttcagcagatccccattacgaacaactggcaaactcaacataaaaccctcctgagagccccaatgagacccagggcttgccaggtcacctcatattgagactacaggggtcttggagatttttatatatgaacctatattctccacaacatctgcagcatgacatcaatattagtgaaacgctacaaaaactgttcttgcatgtaataactacctccccaaatccaggcttcactggagagcctctggattagtgatcccagaggccagttagatttcactgagtcaggaatttccacctgacctgccatttactcattcctggtagatgagtctgggttactggctttcaatgtgagggagaagattacgttatgaagggaaatactgcttgtttgccctctgcctattaataaatgttgtgtttccttcacaggagtcctatgggtaaaacatcagggaagctgtttgatagcagtgatgatgacgaatctgattctgaagatgacagtaataggttcaaaattaaacctcagtttgagggcagagctggacagaagctcatggatttacagtcgcactttggcaccgatgacagattccgcatggactctcgatttctagaaactgacagtgaagaggaacaggaagaggtaaatgaaaagaaaactgctgaggaagaagagcttgctgaagaaaaaaagaaagccctgaatgttgtacaaagtgttttgcaaatcaacttaagcaattctacaaacagaggatcagtagctgctaagaaatttaagg

SEQ ID NO: 72 - DNA, 1365 bp, Homo sapiens
  misc_feature: Incyte ID No LI247388.12000MAY01
tttgccaaaagttggggtcatccaaagaaatatctgtcctgtttccagaagcaacaaagcttgccttagcaaaggtggaaccttttccttcagattcaatggtgatggttgtagttcgtcttaacaagatctggtcaatgtcctcttcacaaaacttggagccttcatcatcttcctccatgatggctgcatatgctccttttcttaaaacatcttcaatctccttcttagagaactgttggattccagtaatgttgccatcccgaccactcatggattgaagcacagccttatccaaccccaacttgaggctggccttatcaaacatctctctctcgtaggaattacgagtgatgaggcggtacaccttcacagctttgctctgcccaattcgatgacatcgtgcctgggcctgcaggtcattttgtggattccagtctgaatcaaagatgatgcaggtatcagcagctgtaagattaataccaagtccaccagcccgggtacaccagtaagaagacaaagcggtctgagtcaggcttgctgaagcggtcaatggcagcctgtcgaaggttgcctctaactcgcccatcaatacgttcatataagtacctcctctggattaaataatcctctaggatgtctaggcagcgtaccatctgagagaagatcagaactttatggccaccagctttaagctttggaggcaacttgtcaataagaaccagtttgccggctgaacgaaccatggcctgcaggtgaaaggtcatgaggtataatatggcaagcttcacggaattctgttaggattttttcttcagcaccagcttttggaaagagctcactctagctgctatgataaacaagaacgaccagtgaggaagttttgaaactctcctctgcaagagaatggtggccagccaggcgtggtggttgtcgaatctgtggcacctgtgggaagtgggctcagccacagggactgccatttggatcccccagcattggcaccatacctaccctgggccctaaggtggccatgcttctctggatttgcttccctagcagtgagctctagtgcttgcacacatccctgcccacagcatggcctgggagcagctgagactggagggccagctcatcccgcgtctgattcctgtgaagtgttatcagtcgcctgttatggagtgctggacccatgagtggcagccttccctggcagctgggctgacctgtctagcttttccattgctcgctggttttgttcactgtagggcgtgagggggtgagtagctgctggcctccaagtcacatagctactcatgttgtacgctgttcacagggacctctaaggatgtacatcatcacaca

SEQ ID NO: 73 - DNA, 1728 bp, Homo sapiens
  misc_feature: Incyte ID No LI816339.42000MAY01
gggctaggtgtgggaaacagggaagagaactagaactgggaagtaaagcattgaagatgtggactggaattgctgcaatctcatggattcaaatttgaatgggatgagggagttgcttcttatggatgagcacagaaaagtggttttttgagatagaaattactcctggtgaagatgctgtgatcattgttgagatgacaacaaagaatttagaatattccataaacttaggtgataccgcagctactggctttgagaggactgactccaattctgaaagaagttctgtggataaaatgctgtcaaacagcatcacatgctacagagaaatctttctagaaaggaagagtcagttgatgcagcaaactttattgttctctaagaaattgccacagcttatcgtaatcttcaaccaccaccaccctgatgagtcagtacccatcgttaacgttggcgtaaaaccctccaccagcaaaaagattgttacgcattttttaacgcagtgatgtatttatcaattattcattatgctatttgtaccctttcttaatagactatggtatagtgtaaccataatttatgccctgggaaaccaacaaattcatgagattcgctttattgtagtgctctggaattgagcctgcccgggtttcttgaggtatacctgtggtatttagtatgttccttgtatataacaccccattttatagggtaggaaactaaggcttagagtttgagcaattttcccatagataacaccaataaattgagggctgcagagaccacttaagtggttctcttaacagtagtctagacatggacgtaatgagatttggcttaggagtgacaatggtaaggaagttaaaggattcaggaaccaaataccagttattgagaagaaagaatggcctcaaaaacatcacttctcaaggaagccttgataccctcacccttactacactgtatcctccatttgaatgttgtcaagagctcttctcacaattgtaaataataattgtgtaattttgttttttttttcctactttaggttgctccttgagggcaggacagttcttatggctttatccgttgcgtaaggcacagagaggtctgcgaggtcaaaactattgtcatgatagcaagatgcctgtctttttcatttattctcacggtgtgcagaggcctacatgatggtgtgatgacatcctcattgatgttaatgaatggtgctatacttgaattttctaaggtgtcattaggtaggtttaggtattgcatacttgtttttagaaattatttttttcctttttcccatttttagaaatcaactcattttttaggttatgccttcagtaatctttgcaacctcaatattgtgtaatggttactttaaaatactgtagttttctttgtgcctaagtataaacataagtacattttgttgttttgtattgcaatagtggtttaaatattttccctaaatatccaaatttaaaaagtctaaaactttagaatttaataaatttattctaaaatatttatatattttttcatttaagaatggatcggcttaaaaacactgcttctcatttacttactggctataccatctgtatactaataaaaaacaagatttgatttggaaggactgactcaaactaacgaaaatatgaaacctgtctttctgctttggagatatttacctaatcatgttctgcagtagaataa

SEQ ID NO: 74 - DNA, 747 bp, Homo sapiens
  misc_feature: Incyte ID No LI1188967.12000MAY01
atgagcttacagaaagcctggcttacatttactctgtttggatttcttcctcatcaagagactgctgcagtgcctgtcatgtgacagcggcatggacatatgccccaggctttcctgctggggtccatccatgagcctgcaggtgccctcatggagccccagccctgccctggtaagcttggctgagagcttcctggaggaggagcttcggctcaaatgctgtgagctggagccaagcttgcaggttttcgggagccttgtgggcgatcaatctacaatcccggtggcagttatgctatggcgagccgacatctgcaagctactgtgactcgtgctactgccaagggcccccagaggaagtactcttccttgggcatgaacccttggacctttttggcatggcccaagatctgtggtgcccttttggggcaagtaagcatgggtccggggacgtggttgggcattttgtggggccctgtgcgtgaccccttcgcccaagagcatcctaaacgacccagtgcctgggactggagtgcccacagtcagaaggacccaagacaaagcatgggacatgaaattcaagagtgaacttcttcatgcggaggctgcagctggatcagaggacacaatcccactgtgacagaagtgcaagtcagaagacctggcttttcatcccagctttgaaacttggaactttttgattgacaaattaataaacctctttatgccccaggctcctcctctgtt

SEQ ID NO: 75 - DNA, 1817 bp, Homo sapiens
  misc_feature: Incyte ID No LI236230.32000MAY01
tcgaagttcatcatccataatgtatatagtgttagcaagcagggagttgttattcttgatgacaagtcaaaagaacttcctcattgggtgctgtcagctatgaagtgtttggcaaattggcccaactgttctgatttgaagcagcctatgtacttgggatttgaaaaagatgtctttaaaaccatagctgattactatggtcacttgaaagagcctctacttacatttcatctttttgatgcttttgtcagtgtactgggtttgttacagaaggagaaagtggcagttgaagcatttcagatttgctgccttctcctacctcctgaaaataggagaaagttacagctattgatgaggatgatggcaaggatttgcttaaacaaagagatgccacccctgtgtgatggctttggtacccgaacactgatggttcagacattttcccgttgcatcttgtgttccaaggatgaagtggacttggatgagttattagctgctagattggtaacgtttctgatggacaattaccaggaaattctgaaagtccctttggccttgcagacctctatagaggagcgtgtggctcatctacgaagagtccagataaaatacccaggagctgatatggatatcactttatctgctccatcattttgccgtcaaattagtccagaggaatttgaatatcaaagatcatatgggctctcagggaacctctgggcagccttgttggaggaagtcataacagatgccaaactctccaacaaagagaaaaagaagaaactgaagcagtttcagaaatcctatcctgaagtctatcaagaacgatttcctacaccagaaagtgcagcactttctgtttcctgaaaaacccaaaccgaaaccacagctgctaatgtgggcactaaagaagcctttccaaccatttccaaagaactagaagttttcgaatgtaataatacttccacagcaacaggtgctagagaccactgtttgttgttttgagtgaatggtggttagggggaaagactttggtggtggaagaaagaaaagcataaaacaaagactactgaaatattagataaagattgccttagtttttaaaaatgtttggccattagtatttttataaaactcaatgctagttttaaagtgtataaattggttaaaatttatgagtcaaatatatagtgataatgttaacatgtttgtaattgctacagaatttaagggtatttttatctctgtgctctctttttcatggtgtttattaaataattgtgtatatacatccctagctactgatatctttattatagccttaagacttaattttaagtcttaaaaatagccgtgtatacttgaataagaaagacactgggtactgttactgtgatgctattgacttagtagccaattatcatttctcctgtataaattccagtttttattgctgcacataaattttttaatgtcttatattgtgatagctatgtcttttattgcagatttattggatgttatgacagattttactaaagctagtgatttttatgaacatatatattagatgcatgatttacctataagtggagtagattttcatctgccctgcaatggtataatttcagtcttagctaaaaatggaaagttgaactgggataaattctttggggtacccttaggacctctgattctaagtcaaatgcaaatggggttaaataaaatgaggactacttcctttataaatatattttcatccttttggaaagtaagtgaaatgtaaaataaacttatttttttttaaaaatgccaaa

SEQ ID NO: 76 - DNA, 2683 bp, Homo sapiens
  misc_feature: Incyte ID No LI246728.32000MAY01
ttaagaagacagtttagcacagagatggcaccagcctggccttccctactgtcccaggggagcagagctagggtcctcttttaccctccctaaggtcacccccttcccatctacacgtccctccccaagttcaagcacatagtcaatcatgaccactcaggagctcagtgagctggggccatgtccctgtggcctccagcccatccgcaggagggtcccttgcagtgacagcacaaagagtccccctacagtgaagcccctgtagggccagcatggaccttgagagccaggagggaatgggcctggtgccactctgttcccactcccacctaagattcctacccctgtgttctagcaggtgactcttgatctggccagcccacaacaaatacttgaactaggcagcatggggtggttggaggcctgcagaggcccactgggcccacggaactgagacatagggctttctcatgaggcctgatcaagctcccgcaggtatgacctggcctcctcgcctcgggctgccttaaagcaccgctaacccacgccaaggggctaggcagggcttcgcggggcctggcccaaggtctccagcagagcaataacatcttcccaggttgggttgccacagacaccctgccctggagcccccagcccatcccatctggtgctaccctgtctgcctggcagcctagcctcacccctcctgtccggtcatctgggggtccttggtaaggatgcagccacatggggccggtttatgcacctgggcgagaacctgactggggcaggggatgctcaggggcctgcaggtctcccacagggctgcttttctgcccgcctcaccctgtgggtttgtatttcttgccgggtcaaaataggcagtgactcgtgccttccctcctcagggccggcggcagggtgtggacccagctggccgctggaccgtccccttcctcattctccaagctcatcaacaggtctcagaggaacatttccataaaaggtgtggcctggcatgcagtgctgtctgcacgctgtcgcttcccccacagcaacctgccggtcagccttaactggtgtggaggtcgaggtggggagggggaggcgggcttccctccctggtgtctggcctctttttgctcagccctcagcacagccttcagcgtggagagcgagaccctgcgtgtcactggggcttggctgaggtctcccctgcaaggattaacaagtgacttcaaggaacccagaccccagtctggggcggttctaccttcccggctgggcccagcccccacaccacagtaccctggccacgctccatggcacaaaagcctcttgaggccacagctcagctatgaagctgatgtggcttatgtgcagtctcctcagggaagggggcagtgatgccatgcatgagatccgctgtgccataagaaagttgcttcctttagaatttctggtccaatgcagggaggtgaggcccagcctcatgcataacacatgcccatccaaggaaaagctgtggctaatgcttccaaaacaagtgaaaatcttgcaccaggccccaggggccatatctcattccggccccagcttactgagggaggggacagactgctggaggacaatttctcctagttaccactaaggggttacatggctactcagaatgaaagataaattgccaggcctgttgcaatcgtcttttttttgagatgggagtcttgctctgtcgcccaggctggagtgcagaggtgcgatctcagctcactgcaacctccacctcccgggtcaagcaattctcctgcctcagcttcccgagtagctggaattacaggtgcccaccaccatgcctggctaatttttgtatttttagtagagacggggtttcaccatgttggccaggctggtcttgaacacctgacctcaaatgatccgcccacctcggcctcccaaagtgttgggattacaggcgtgagccacctcaccaggccccgttgcaatcttactccacttcgtttttcacaggcagtcccgggacatgcccattattagggcctatttgataggatgatactgaggctcagagtcgggggggtcacaaggccaaagccacaccgctagaagggctcacggatcccatgccagctcccgtcctcttgggtggcatcaagcttgtgtgatatgaggtcttatggtctgctcatgacagccttaaaagcaataaacagataccaacttccagcaactttcccaactcagcttgcaaaggcttgcgggggggtccctgccctcgagcccataaggtgagcagcttagcccagtgccgggcccantgtggagaaggcctgaattgccctgtgaagctgggggttgccaggcagggaatattcatggaagcccctgaccatgtactcacactgctcttcctcagttcaggggttcttggatttgctcaaagttcaacccaaaatatcccttttacgattctcaggggtcccaacccttccagatccaatccccttaactttacataagagttcccacaaggctgtgacttcaaactgtgttgtaaactgcaacctgcagaatcaaatttagggttatgttttcagttgttatcagtcttacggctatcaagatattaaagagattcttttctggttatcaaagaaaaaaa

SEQ ID NO: 77 - DNA, 3328 bp, Homo sapiens
  misc_feature: Incyte ID No LI1190057.12000MAY01
ctgcggtggcggcagcggtgggagatgccggggcggccggcggcaggtccatgtggcgctcttaggtgggatgccagcgtcctgaaggcggaggccctggccctccgtcccctgcggcctgggcatggcattctcccagtcccacgtgatggccgctcggcggcaccagcacagccggctcatcatcgaggtggacgagtacagctccaaccccacccaggccttcaccttctacaacatcaaccagggccgcttccagccaccgcatgtgcagatggtggacccggtgcctcacgatgcccccaaacctccaggctacacccgcttcgtctgcgtctctgatacccactcgaggacggaccccatccagatgccgtacggcgacgtgctgatccacgctggggacttcactgagctggggctcccgagcgaggtgaagaagttcaacgagtggctgggcagcctgccctacgagtacaagatcgtgatcgcaggcaaccacgagctgacctttgaccaggagttcatggccgacctcatcaagcaggacttttactacttcccatctgtgtcgaagctgaagccggagaactatgagaatgtgcagtcgctgctgaccaactgcatctaccttcaggactcggaggtcaccgtgcggggcttccggatctatggctccccatggcagccctggttctacggctggggcttcaacctcccgcgaggccaagccctgctggagaaatggaacctcattcccgaaggcgtagacatcctgataacccatggaccaccactgggcttcctggactgggtccccaagaagatgcagcgggtgggctgtgtggagctgctcaacacggtgcagaggcgcgtccagccgcggttacatgtctttggccacatccacgaagggtatggtgtcatggcagatgggacgaccacctatgtgaatgcgtccgtatgcactgtgaactaccagcccgtgaacccgcccatagtcatcgacctccccacaccccggaactcctgactgctccccactgcccctgaccatgcacgcccgtgtcagctccacaggcctgggcccggccaactgttcccttccatgctgagttgcctgggacgacccatctggctgcggggactgggcctagcaggcaggtcagggccttggaacgactctttagccttctgtcacctggagttgggaccctgcgcatccccatcaggggttctgtgctcatttacgtgtttctgcgtgtacatcctgcgtgtacctcgtttaaaggacctactaagctcatggccctgtcatgtttgggaaatgactaagatcttcattcttctcccttggacgccctccctggttagggaagctgctgcatcttgaatgggtttcggaagttacacaaaatctccctctaaccacgggtctgtgtggcggcatgcccctgggattggggtgggtgggaggatttcgtgagctgcaacacagacagtccctttcgtgcccctcccaagtgagggaggagacaggcccaaaggcggaggccctccgagggagcatttagggacatctcaggaattcagaccgcacctggccaggcccacagctgttttgcctggcattgttcccccctttttcttcccataggcacttttgtttacttgaacaatgacctgagtgtcctaggcattcactcggagcctgggatgatgggggcatggttctacagaatgagccactggcattcgtgcccaggaccagtgagagcgttcccagtcatggggggatgacccttggtccccacggggaacacactgctctgtagaatgcggtacgcctggatggaagggtctgcaggcttaggctgcagcaggcctgccatggcgtgtgaacccatgtgccagtgcacgcaccgacacacatgcactgcacgcagccggtcttaattgaaccaagtgggtcctgtgtttctcttttctgccccgtagtgaggttctgttccttaggtgcgggcttacccttgcaccggttagggattcgtgtcttggcatgcgctgcagcctgagtgaagcccatggaccatgccggcccctgtgaatttggagggtggccacagggacccgagcgggcccccaggctcccagcagccccacctttggacagccttctttttccttctttgaattaggactggagggggtggggccagggcggtggtgggaaccggtaggcccttagatgagggggcagccagtcaggcatgaatatggggcaagttcagggctcagattctggtcggttatttaatattttggttcatcatggactttttctgcatttattttgatttttaacgttgcattaaagtagtaattatttgtcctgaattggtgacttttttttgcacccctttccgttgtacatctgagagaagggtgtcactccctcacccaggtcccagccctgggaaccagcctaccgtgagcccttttgcagatatagactcatttcatcctcagatggtccttcaaggtaggtactttagtcccattttagagatgagacgattgaggccagaggggtgtggtaacttgcctgggggctcacagcacaaaaggagccgaggcaggatctgacccttgttctctggcgctgactgccctcactttgccatgacccgaagttatgtccctacaaagcaatgcatggtccaaggctctttttattgtatttttatttttaagggtcctgttcaaaactggtgtgagctctgaggagtccttgaaccctgggtgcagcatccctagcatcctgggagtccttttctgcccacactgagctgggctcctcgaggggtggggctgctgtccctggaagcctggcagcagcactgtatcgggttggctgaagctgagcgccgtggggtgcagggctccaggaatcccgtttggctgaaggggttccctgtagcccgggatgtttatgaggtcctctctgatgccccaggcgcaggacatgtgtgcgggtggagaaaagcaggccctttcagtgccagctccactcaatttctatgtggaccaagaacgataaacttaaaaaatttttttttcctaaggtatcttcagaatatggtgtatttttatgtggaaaagaaaagttatgaaggcagctgttactttaagagaaaattcattagtagtaaaacgaggtatgaagatgacggcgtgcttctcaatcattttggcataacttgattgtggctgtaattcacacccttttttggtcaaccatgtcagacaataaactctttgtaaaaag

SEQ ID NO: 78 - DNA, 2792 bp, Homo sapiens
  misc_feature: Incyte ID No LI221836.32000MAY01
catgaagccacgcatgaaaatttttctgcgtcaaaagaaggtgactacccagatgattgccaagagcctggccaatgtagaatatgatacatataaacctaccttcacaaataagcaggtgagaatcacctttgggttctcttgcaagagtagtaaccagtttggaataatgatgtatcataacaaccgactcataaaatcttttgagaaggtgtgggtgccgggtgaagccaactcgtgtgagaaagtgatggcgagtaattggagtcattgagtgcaatttcctaaaacctgcctacaacaaacaagactttgagtataccaagggagtaccggctaacaataaatgcccttgcccagaagctcaatgcttactggaagaaaaaaacatctcaagataattttgagacctcaactgtagccaggccaataccgaaggttcctgaccagacatgggttcagtgtgatgagtgtcttaaatggagaaagcttcctgggaagattgatccatccatgttacctgcaagatggttttgttattataattcccatccaaagtaccaggagatgctctgttccagaggaaccagaaatcactgatgaagacctgtgcttgagcaaagctaagaaacaagaaccaactgttgaggagaagaagaagatgcctatggaaaatgagaaccaccaggtattcagtaatccaccaaagatccttactgttcaagaaatggctggattgaataacaagacaacttggatatgagggaattcatagccctagtgtgcttccttctggtggagaagaaagcagatcaccatctcttcaacttaagcctctggattccagtgttttacagttttccagtaagtacaaatggatcctaggtgaagaaccggtggagaaacgaagaaggctccagaaatgagatgacaacaccttctctagattattccatgcctgctccttacaggagggtagaagcacctgttgcctacccagaaggggagaacagccatgataaatcgagttctgagagaagtacaccaccataccttttcccagaatacccagaagcaagcaagaatacaggtcagaatagggaggtttcaattctgtatccaggggccaaagaccaacgccaggggtccctgcttcctgaagaattagaagatcagatgccaagattggtggcagaagaatctaacagaggtagcacaaccataaacaaagaagaagtcaacaagggaccttttgtagctgttgtgggtgttgccaaaggtgttagagattcaggagctcctattcagcttgatcccttgtaacagagaggagcttgctgagagacgaaaagcagttgaatcctggaacccagtgccttattctgtggcctctgctgcaatccctgctgcagccattggggagaaagcaagaggctatgaggagagcgaaggtcataatacaccaaagttgaagaaccagagagagctggaagaattgaagagaaccacagaaaaattggaacgtgttttggctgaaaggaatttgttccagcaaaaggtggaggagctggaacaggagaggaatcactggcagtctgaattcaagaaagtccaacatgaattggtgatctacagtacccaggaggcggaaggcttgtactggagcaagaaacacagtggattatcgccaagctgaattcccagatatctgaaagactgagctggaaagaagccaacagaggaaaacgcacgagttaaaagagaaactgaaggaaacagagacacaccctggaaatgctgccacgaaggctcacggtctccctacccggacccccagagggagatgacctagaaagggctttggcacaagcttacgcgcgctacgtataccacgtcagcatatctcccttacttctgtcctccctcacttggagcttcgtgagatcgggtatgactcagaaacaagtggatgggatccctgtacacggtgctgggaggcaaaatccccatactggattgaggcaccagactgtatacccttctcttctcttataattctgtctgttctcttttctctccctccctcangtntntntntntntntntntntctntntntntctntntctnancctcacctatatgccttatatagagaatctctgtgtaaatccgtggctcataatcagtctcctttttatcagtttttgggtgtggagaaagaggccagttttaaataggctttcaagaggtctaggggtcagaaaagcaatagtcacttaagctaggtgacctgaagagctttaattcttcatgacctgtatatgtgcgtctattgtatatctttcttctgaaatggtgttgtatatcattgtagtgttagatcaatcaggcagatagttggtgtccggtataccaggtattattggggggtaagcttaacaagtacaactcatgtttgcaagccttcgaagtatgtaacaatcgtcggtggaaatataagaccatatatcacattatacacaaaagtgtgtgatatgtacaagtgagtggtacagatatgacatgggaagatctgggggaggaagttcaaggaaggcaccacaccagaaatggggacctaaattaaggcttaaagaatgaggcatggccacactgtcagtgagtgttctttaggtggtaggactatggttgatattttttcttcttcctatcttcctgcctttttcagattttcaatattaaacttgttattcttacattggaaaaaaataaattgtttttaaaaagatc

SEQ ID NO: 79 - DNA, 918 bp, Homo sapiens
  misc_feature: Incyte ID No LI334047.32000MAY01
tctttatgtatttttaaaaaaactaatttaagttaaatggcagtaaaccatgataatttagaaaacatggtgaaactgagagtgaagagaaatgttctttacgtaaaccacactgtaaaccataaaacttatttttgtcaactgcttttcatgacagtggctgggaatttgacatttctactacagtaggcaaaaatacatgtaaaatacactctcccacataccacatcacatctctatttgtatttccaaactaaaagcattacagcccaaaaaagtgctgcgtgcactggtctagttacaccaagaagtgtatgttcacaccagcaacaccttaagctggtatctgtgtggcaaggccactgctggtggcccctgangccaagccctcttcaatggatccagtttggctcaccaanagaaatccactgctctttaacccaaaaggtggtaagcttttcaatggcctccagttaccacaagataaaacaaacaaaattctcaaacagattatgaaacccaactgaagtatccacatgacagcgttcaatactacctcgccacttcaaaatgtgtcagcagcaattagctatgaaaatgagtgttgtgaaaattcgcaatagtttgaaactgaatattttaaaactagatatctttaatatgacagtaaacaatgtgacagaaacatggctgtttctctcatcttgtctgagatatacattccatacaggtgactgggtcccataagttttctaagaaaaacttttcctttccacttaatgatcctaagaagtcagtgcaatctggttcttctccctgcagaagggttatttagtttgggatgtaacattttacaacaataatacaaacgtcctgggagaataccacaataaatgtctcaaaaaccactcagttaat

SEQ ID NO: 80 - protein, 202 aa, Homo sapiens
  misc_feature: Incyte ID No LG223939.1.orf32000FEB18
EIMELVLVKYQGKNWNGHFRIRDTLPEFFPVCFSSDSTEVTTVDLSVHVRRIGSRMVLSVFSPYWLINKTTRVLQYRSEDIHVKHPADFRDIILFSFKKKNIFTKNKVQLKISTSAWSSSFSLDTVGSYGCVKCPANNMEYLVGVSIKMSSFNLSRIVTLTPFCTIANKSSLELEVGEIASDGSMPTNKWNYIASSECLPFW

SEQ ID NO: 81 - protein, 91 aa, Homo sapiens
  misc_feature: Incyte ID No LG397140.1.orf12000FEB18
PCAPISLCARDHMHMGECLCAHDFMHIGEYLCAHEHMHMGECLCAHEYMHTGECLCAHEYMHMGICLCARDYMHMGECLCARDYMHVGECS

SEQ ID NO: 82 - protein, 197 aa, Homo sapiens
  misc_feature: Incyte ID No LG1094205.1.orf32000FEB18
PKMAAPPGEYFSVGSQVSCRTCQEQRLQGEVVAFDYQSKMLALKCPSSSGKPNHADILLINLQYVSEVEIINDRTETPPPLASLNVSKLASKARTEKEEKLSQAYAISAGVSLEGQQLFQTIHKTIKDCKWQEKNIVVMEEVVITPPYQVENCKGKEGSALSHVRKIVEKHFRDVESQKILQRSQAQQPQKEAALSS

SEQ ID NO: 83 - protein, 98 aa, Homo sapiens [sequence omitted]

SEQ ID NO: 84 - protein, 121 aa, Homo sapiens
  misc_feature: Incyte ID No LG981170.1.orf12000FEB18
DDCMLTHPLQGPGLDLGLHCILSNGLAGAPFGLLSLFSPELGWWEKRGWSESISIQIPAGITLGVFLACFGLKLSYIVYWLPKSGLKSEKMQAMNPSAHSSPTFPALFYLGGQWKGGEAMP

SEQ ID NO: 85 - protein, 136 aa, Homo sapiens
  misc_feature: Incyte ID No LI197613.1.orf22000FEB01
ASSGHAPLAASTRKAPQAECGMISITEWQKIGVGITGFGIFFILFGTLLYFDSVLLAFGNLLFLTGLSLIIGLRKTFWFFFQRHKLKGTSFLLGGVVIVLLRWPLLGMFLETYGFFSLFNCSGDFKALARWSEKQR

SEQ ID NO: 86 - protein, 71 aa, Homo sapiens [sequence omitted]

SEQ ID NO: 87 - protein, 608 aa, Homo sapiens
  misc_feature: Incyte ID No LI212029.1.orf22000FEB01
YPAPQPVRTDVAVLRYQXPPEYGVTSRPCQLPFPSTMQQHSPMSSQTSSASGPLHSVSLPLPLPMALGAPQPPPAASPSQQLGPDAFAIVERAQQMVEILTEENRVLHQELQGYYDNADKLHKFEKELQRISEAYESLVKSTTKRESLDKAMRNKLEGEIRRLHDFNRDLRDRLETANRQLSSREYEGHEDKAAEGHYASQNKEFLKEKEKLEMELAAVRTASEDHRRHIEILDQALSNAQARVIKLEEELREKQAYVEKVEKLQQALTQLQSACEKREQMERRLRTWLERELDALRTQQKHGNGQPANMPEYNAPALLELVREKEERILALEADMTKWEQKYLEESTIRHFAMNAAATAAAERDTTIINHSRNGSYGESSLEAHIWXEEEEVVQANRRCQDMEYTIKNLHAKIIEKDAMIKVLQQRSRKDAGKTDSSSLRPARSVPSIAAATGTHSRQTSLTSSQLAEEKKEEKTWKGSIGLLLGKEHHEHASAPLLLPPPTSALSSIASTTAASSAHAKTGSKDSSTQTDKSAELFWPSMASLPSRGRLSTTPAHSPVLKHPAAKGTAEKLENSPGHGKSPDHRGRVSSLLHKPEFPDGEMMEVLI

SEQ ID NO: 88 - protein, 396 aa, Homo sapiens
  misc_feature: Incyte ID No LI249170.1.orf12000FEB01
SACAGHSLSPAAMDAALLLNVEGVKKTILHGGTGELPNFITGSRVIFHFRTMKCDEERTVIDDSRQVGQPMHIIIGNMFKLEVWEILLTSMRVHEVAEFWCHTIHTGVYPILSRSLRQMAQGKDPTEWHVHTCGLANMFAYHTLGYEDLDELQKEPQPLVFVIELLQVDAPSDYQRETWNLSNHEKMKAVPVLHGEGNRLFKLGRYEEASSKYQEAIICLRNLQTKEKPWEVQWLKLEKMINTLILNYCQCLLKKEEYYEVLEHTSDILRHHPGIVKAYYVRARAHAEVWNEAEAKADLQKVLELEPSMQKAVRRELRLLENRMAEKQEEERLRCRNMLSQGATQPPAEPPTEPPAQSSTEPPAEPPTAPSAELSAGPPAEPATEPPPSPGHSLQH

SEQ ID NO: 89 - protein, 109 aa, Homo sapiens
  misc_feature: Incyte ID No LI813218.1.orf12000FEB01
GCEXIGXFMHYWXESEMVQSLWESLAVLQMVKRRATIWPSNSPYRHMPKIIENICSGLPSLGPLPLYGSSVFTLLSLASALFWSMFVKARAELLLAVHHCCLPPSQTRC

SEQ ID NO: 90 - protein, 94 aa, Homo sapiens
  misc_feature: Incyte ID No LI902522.3.orf22000FEB01
WSEFVAAFLSLVALLRRRRNEAEAERVKTKTAAKYGLSAQPRLVDIIAAVPPQYRKVLMPKLKAKPIRTASGIAVVAVMCKPHRCPHISFTGNI

SEQ ID NO: 91 - protein, 211 aa, Homo sapiens
  misc_feature: Incyte ID No LI474304.1.orf22000FEB01
EPSLATYHHIIRLFDQPGDPLKRSSFIIYDIMNELMGKRFSPKDPDDDKFFQSAMSICSSLRDLELAYQVHGLLKTGDNWKFIGPDQHRNLYYSKFFDLICLMEQIDVTLKWYEDLIPSAYFPHSQTMIHLLQALDVANRLEVIPKIWKGQFYFLCVRVHLTSISSTLIMNVLTLLLGMKTSILTEPNVLVEMYFKVMCSCCMLAIRRKFC

SEQ ID NO: 92 - protein, 290 aa, Homo sapiens
  misc_feature: Incyte ID No LI027320.1.orf32000FEB01
SDGGSDADFGGGSGEPDSDRGGERELRLRRGELGGRLLPRAAEEEMAGPNQLCIRRWTTKHVAVWLKDEGFFEYVDILCNKHRLDGITLLTLTEYDLRSPPLEIKVLGDIKRLMLSVRKLQKIHIDVLEEMGYNSDSPMGSMTPFISALQSTDWLCNGELSHDCDGPITDLNSDQYQYMNGKNKHSVRRLDPEYWKTILSCIYVFIVFGFTSFIMVIVHERVPDMQTYPPLPDIFLDSVPRIPWAFAMTEVCGMILCYIWLLVLLLHKHRYMAVYGRNYIEPLPFGVALV

SEQ ID NO: 93 - protein, 125 aa, Homo sapiens
  misc_feature: Incyte ID No LI228319.1.orf22000FEB01
EQAEEEKKPKDSTTPFESRLSQSRKFSWTEYLEATQTNAVPAKVFKMRLPHGFLPNMKLEVVDKRNPRLIRVATIVDVDDQRVKVHFDGWDHKYDYWVEADSPDIHPIGLCDVTGHPLEVPKRTK

SEQ ID NO: 94 - protein, 162 aa, Homo sapiens
  misc_feature: Incyte ID No LG197267.2.orf22000MAY19
AREAALPAAMREGQEMGPTPVPSNPLLHRSFPCWPRGWSHPVPSNPLLHRSFPCWPRGWSHPVPTRELLLEPAQPADLLPPAPTAGPCSLASWMLSQPGRGSQVKTGGTPTATAQDAEAPLPDCDLCLSPAPVGTWQPRAKAGWAGDPRNLSGNTFSPGWEQ

SEQ ID NO: 95 - protein, 181 aa, Homo sapiens
  misc_feature: Incyte ID No LG403332.1.orf22000MAY19
GGETMSKLSFRARALDAAKPLPIYRGKXMPDLNDCVSINRAVPQMPTGMEKEEESEHHLQRAISAQQVFREKKESMVIPVPEAESNVNYYNRLYKGEFKQPKQFIHIQRIWGHYQPETTLKFLLVCFVHLFLDHSISFNLGCRSAQGSVLRKIFCFSFLPKGKLRNTKFFAFPFCMANLFL

SEQ ID NO: 96 - protein, 198 aa, Homo sapiens [sequence omitted]

SEQ ID NO: 97 - protein, 349 aa, Homo sapiens
  misc_feature: Incyte ID No LG216612.3.orf32000MAY19
GTGPHGSAPRPPPVCCARGGLRSPGRRRAPGAGGEMGRYSGKTCRLLFMLVLTVAFFVAELVSGYLGNSIALLSDSFNMLSDLISLCVGLSAGYIARRPTRGFSATYGYARAEVVGALSNAVFLTALCFTIFVEAVLRLARPERIDDPELVLIVGVLGLLVNVVGLLIFQDCAAWFACCLRGRSRRLQQRQQLSEGCVPGAFGGPQGAEDPRRAADPTAPGSDSAVTLRGTSVERKREKGATVFANVAGDSFNTQNEPEDMMKKEKKSEALNIRGVLLHVMGDALGSVVVVIIILSSAFPLIKETAAILLQMVPKGVNMEELMSKLSAVPGISSVHEVHIWELVSGKII

SEQ ID NO: 98 - protein, 85 aa, Homo sapiens [sequence omitted]

SEQ ID NO: 99 - protein, 81 aa, Homo sapiens
  misc_feature: Incyte ID No LG093477.1.orf22000MAY19
VARTTGAHHHAWLSFVFLVEMGGFHHVGQTGLELLTSSDPPASASQSAGIIGVSHPTQPTTNINAVLMMYQTHKHTYLTRS

SEQ ID NO: 100 - protein, 433 aa, Homo sapiens
  misc_feature: Incyte ID No LG222880.1.orf22000MAY19
PPRRPCPRAPHPSALPAGPRDRACTCSGLSASPLRTSSWSSRHSSTRSRLGRGPGGGGRRSRGDASAERREGAGPGRQAAASAMNPRGLFQDFNPSKFLIYTCLLLFSVLLPLRLDGIIQWSYWAVFAPIWLWKLLVVAGASVGAGVWARNPRYRTEGEACVEFKALLIAVGIHLLLLMFEVLVCDRVERGTHFWLLVFMPLFFVSPVSVAACVWGFRHDRSLELEILCSVNILQFIFIALKLDRIIHWPWLVVFVPLWILMSFLCLVVLYYIVWSLLFLRSLDVVAEQRRTHVTMAISWITIVVPLLTFEVLLVHRLDGHNTFSYVSIFVPLWLSLLTLMATTFRRKGGNHWWFGIRRDFCQFLLEIFPFLREYGNISYDLHHEDSEDAEETSVPEAPKIAPIFGKKARVVITQSPGKYVPPPPKLNIDMPD

SEQ ID NO: 101 - protein, 419 aa, Homo sapiens
  misc_feature: Incyte ID No LG898320.3.orf32000MAY19
EVWDCPGNSRVSGPRPILGAMGVIGIQLVVTMVMASVMQKIIPHYSLARWLLCNGSLRWYQHPTEEELRILAGKQQKGKTKKDRKYNGHIESKPLTIPKDIDLHLETKSVTEVDTLALHYFPEYQWLVDFTVAATVVYLVTEVYYNFMKPTQEMNISLVWCLLVLSFAIKVLFSLTTHYFKVEDGGERSVCVTFGFFFFVKAMAVLIVTENYLEFGLETGFTNFSDSAMQFLEKQGLESQSPVSKLTFKFFLAIFCSFIGAFLTFPGLRLAQMHLDALNLATEKITQTLLHINFLAPLFMVLLWVKPITKDYIMNPPLGKESIPLMTEATFDTLRLWLIILLCALRLAMMRSHLQAYLNLAQKCVDQMKKEAGRISTVELQKMVIIPGVFIQNLSLPYQWIIVYCPILFTLNYHQLKGK

SEQ ID NO: 102 - protein, 127 aa, Homo sapiens
  misc_feature: Incyte ID No LG1327047.1.orf12000MAY19
GPPVTFGDHEGISKAMGNVPPKAETPLRCILENWDQLDSHMLRNKRLIFFCSTTWPRYPLQGGETWPPEGSINYNTSLQLHLFCRKEGIRSEVPYVQTFFSLRDNSQLCKKCDLCPTGSPRVYLPTL

SEQ ID NO: 103 - protein, 312 aa, Homo sapiens
  misc_feature: Incyte ID No LG235157.21.orf22000MAY19
STPEGIALAYGSLLLMALLPIFFGALRSVRCARGKNASDMPETITSRDAARFPIIASCTLLGLYLFFKIFSQEYINLLLSMYFFVLGILALSHTISPFMNKFFPASFPNRQYQLLFTQGSGENKEEIINYEFDTKDLVCLGLSSIVGVWYLLRKHWIANNLFGLAFSLNGVELLHLNNVSTGCILLGGLFIYDVFWVFGTNVMVTVAKSFEAPIKLVFPQDLLEKGLEANNFAMLGLGDVVIPGIFIALLLRFDISLKKNTHTYFYTSFAAYIFGLGLTIFIMHIFKHAQLRVLGGNPASYPEAHPLPHNIR

SEQ ID NO: 104 - protein, 477 aa, Homo sapiens
  misc_feature: Incyte ID No LG085713.1.orf12000MAY19
LELFVFSAFSMRTYVCHICSIAFTSLDMFRSHMQGSEHQIKESIVINLVKNSRKTQDSYQNECADYINVQKARGLEAKTCFRKMEESSLETRRYREVVDSRPRHRMFEQRLPFETFRTYAAPYNISQAMEKQLPHSKKTYDSFQDELEDYIKVQKARGLDPKTCFRKMRENSVDTHGYREMVDSGPRSRMCEQRFSHEASQTYQRPYHISPVESQLPQWLPTHSKRTYDSFQDELEDYIKVQKARGLEPKTCFRKIGDSSVETHRNREMVDVRPRHRMLEQKLPCETFQTYSGPYSISQVVENQLPHCLPAHDSKQRLDSISYCQLTRDCFPEKPVPLSLNQQENNSGSYSVESEVYKHLSSENNTADHQAGHKRKHQKRKRHLEEGKERPEKEQSKHKRKKSYEDTDLDKDKSIRQRKREEDRVKVSSGKLKHRKKKKSHDVPSEKEERKHRKEKKKSVEERTEEEMLWDESILGF

SEQ ID NO: 105 - protein, 105 aa, Homo sapiens
  misc_feature: Incyte ID No LG482421.1.orf32000MAY19
RENICSGLSPRPPSLHNFSSYKLLLAFINNSFLLEGLIRIFKEEENDSVIKGGMETVEGIFFKALLISFKLNFAKARDNIKEKLYLKLVIPNLSELYTHQRQYSY

SEQ ID NO: 106 - protein, 213 aa, Homo sapiens
  misc_feature: Incyte ID No LG330944.4.orf12000MAY19
QELNNITKLNEHFSKFGTIVNIQVAFKGDPEAALIQYLTNEEARKAISSTEAVLNNRFIRVLWHRENNEQPTLQSSAQLLLQQQQTLSHLSQQHHHLPQHLHQQQVLVAQSAPSTVHGGIQKMMSKPQTSGAYVLNKVPVKHRLGHAGGNQSDASHLLNQSGGAGEDCQIFSTPGHPKMIYSSSNLKTPSKLCSGSKSHDVQEVLKKKKQSGR

SEQ ID NO: 107 - protein, 150 aa, Homo sapiens
  misc_feature: Incyte ID No LI223060.1.orf32000MAY01
KNHQYFGIFATPFSVDIYMLPGNFEMECLELQSDVQLIEKFDFASLLDFCKTCLPNDKYPLLHNHTLFMSLLLGSTYICELLFSRMKNTKSKIRTKISDEHLENSLRIATTSIKPDTDGLRFSKTMSSTPLVLCCSLFFYNKKYQKSSEV

SEQ ID NO: 108 - protein, 298 aa, Homo sapiens
  misc_feature: Incyte ID No LI213087.1.orf22000MAY01
SRRSAAMAGGRGAPGRGRDEPPESYPQRQDHELQALEAIYGADFQDLRPDACGPVKEPPEINLVLYPQGLTGEEVYVKVDLRVKCPPTYPDVVPEIELKNAKGLSNESVNLLKSRLEELAKKHCGEVMIFELAYHVQSFLSEHNKPPPKSFHEEMLERRAQEEQQRLLEAKRKEEQEQREILHEIQRRKEEIKEEKXRKXMAKQERLEIASLSNQDHTSKKDPGGHRTAAILHGGSPDFVGNGKHRANSSGRSRRERQYSVCNSEDSPGSCEILYFNMGSPDQLMAPKGKCIGSDEQL

SEQ ID NO: 109 - protein, 89 aa, Homo sapiens
  misc_feature: Incyte ID No LI405330.1.orf32000MAY01
YKFLAESATLNYVADRQASTLQLGTKCFHCRISSRLKTERLVKTKCLKRVRCLGWACDVLSGLALLDGTLRSELLENLHLKGEAFRKQS

SEQ ID NO: 110 - protein, 771 aa, Homo sapiens
  misc_feature: Incyte ID No LI350243.2.orf22000MAY01
NREKIPFQEPKVSPVPLPLPSPHSKSTPSRQPPSLAASPGSSSGLTATVAQAMPYSPQLKPIQPIAHCYGRTFHSQPCLDSSQGQEKERQIIEGIFKGTCKSSDPWDTLVEQMVSHYSPFRESSGNGMKMEGLLNGSSDPHQSRLASIKAEADKIYSFTDNAPSPSIGGSSRLENTTPTQPLTPLHVVTQNGAEASSVKTNSPAYSDISDAGEDGEGKVDSVKSKDAEQLVKEGAKKTLFPPQPQSKDSPYYQGFESYYSPSYAQSSPGALNPSSQAGVESQALKTKRDEEPESIEGKVKNDICEEKKPELSSSSQQPSVIQQRPNMYMQSLYYNQYAYVPPYGYSDQSYHTHLLSTNTAYRQQYEEQQKRQSLEQQQRGVDKKAEMGLKEREAALKEEWKQKPSIPPTLTKAPSLTDLVKSGPGKAKEPGADPAKSVIIPKLDDSSKLPGQAPEGLKVKLSDASHLSKEASEAKTGAECGRQAEMDPILWYRQEAEPRMWTYVYPAKYSDIKSEDERWKEERDRKLKEERSRSKDSVPKEDGKESTSSDCKLPTSEESRLGSKEPRPSVHVPVSSPLTQHQSYIPYMHGYSYSQSYDPNHPSYRSMPAVMMQNYPGSYLPSSYSFSPYGSKVSGGEDADKARASPSVTCKSSSESKALDILQQHASHYKSKSPTISDKTSQERDRGGCGVVGGGGSCSSVGGASGGERSVDRPRTSPSQRLMSTHHHHHHLGYSLLPAQYNLPYAAGLSSTAIVASQQGSTPSLYPPPRR

SEQ ID NO: 111 - protein, 247 aa, Homo sapiens
  misc_feature: Incyte ID No LI445188.1.orf32000MAY01
VYSENMSTAIREVGVWRQTRTLLLKNYLIKCRTKKSSVQEILFPLFFLFWLILISMMHPNKKYEEVPNIELNPMDKFTLSNLILGYTPVTNITSSIMQKVSTDHLPDVIITEEYTNEKEMLTSSLSKPSNFVGVVFKDSMSYELRFFPDMIPVSSIYMDSRAGCSKSCEAAQYWSSGFTVLQASIDAAIIQLKTNVSLWKELESTKAVIMGETAVVEIDTFPRGVILIYLVIAFSPFGYFLAIHIVA

SEQ ID NO: 112 - protein, 98 aa, Homo sapiens
  misc_feature: Incyte ID No LI244378.1.orf12000MAY01
GLGSLNPGLWLLNGSRNSLGNFCDGQRKCSSVIFMDEIDSFGSSELEGDAGGNSEVQQMMLSRVALRPPRVSRTVYHGCEYEMRYLVTPAVFVAQGHH

SEQ ID NO: 113 - protein, 51 aa, Homo sapiens
  misc_feature: Incyte ID No LI236574.15.orf12000MAY01
DTLMKTWTIEWSRNSPLSLTITVAFLWSSSSPRIPKTAAEFIKIKFVNVTT

SEQ ID NO: 114 - protein, 94 aa, Homo sapiens [sequence omitted]

SEQ ID NO: 115 - protein, 64 aa, Homo sapiens
  misc_feature: Incyte ID No LI037940.6.orf12000MAY01
AHAEDSVMDHHFRKPANDITSQLEINFGDPWPPRTWRQGRTRWTWAWWAPKPWQQDRQVKCFCS

SEQ ID NO: 116 - protein, 238 aa, Homo sapiens
  misc_feature: Incyte ID No LI228550.3.orf22000MAY01
WMVLSLWRLCTCLFLAPSRQGGGSQALPSSMAPWPPGPSTVVVEPHQWPLGIRLNAGGTFCWPTLRAVMQRPFPQACVFTHLPSLAFAVTGREEPSRDPWWPTCGCHMLLFPACPATTHALHRVRWSHGGASFTQQPSNPDAVGRAGSLGYHHPCGCTAVPCSSGSGFPAVTSKPACPVCWGWLSFGLPITGRLVEKAVPRGRTRPRRQLPALPQKCQDVHQPLARARSRQSTVTGEC

SEQ ID NO: 117 - protein, 202 aa, Homo sapiens
  misc_feature: Incyte ID No LI027320.1.orf22000MAY01
RPGRCLRAVAPPRLHSGSPPRAPPPPLEALHSGEAGRAPDSDGGSDADFGGGSGEPDSDRGGERELRLRRGELGGRLLPRAAEEEMAGPNQLCIRRWTTKHVAVWLKDEGFFEYVDILCNKHRLDGITLLTLTEYDLRSPPLEIKVLGDIKRLMLSVRKLQKIHIDVLEEMGYNSDSTMGSMTPFISALQSTDWLCNGELSP

SEQ ID NO: 118 - protein, 172 aa, Homo sapiens
  misc_feature: Incyte ID No LI321475.1.orf22000MAY01
SLISKEDFKQMSPGIIQQLLSCSCHLPKDQQAKLPPTTLEKYGYSTVAVTLLTLGSMLGTALVLFHSCEENYRLILQLFVGLAVGTLSGDALPPPYPSGSCVYISRKPQNLGISMKAKVNIWKLMGLIGGIHGFVLDRTNVLFFLYHQMTSRACHWLMGTWVIPTILHSTLN

SEQ ID NO: 119 - protein, 166 aa, Homo sapiens
  misc_feature: Incyte ID No LI899552.5.orf12000MAY01
VNIKSPQPHLEGFHPARACLLLSTLLGVTYECHSCNSEVPTKPRALASSGVRPLFRVLRTAVVVQVTPKGMRRKGSMSSKAQGSQDSSPPFSPGTSREARGFTLCTFLLLLKCPAVCLPGQKFGATQAGLTMPQVLIWRAERSDAGTYKEERKEERLTLTRLLRRG

SEQ ID NO: 120 - protein, 194 aa, Homo sapiens [sequence omitted]

SEQ ID NO: 121 - protein, 313 aa, Homo sapiens
  misc_feature: Incyte ID No LI1072337.2.orf22000MAY01
LLLPAASAHQTATGPAEGFWGPPVLMLKRSKNFEPSACHGKNVVVTADCIVTQKRVPAARLGIKCQVSGSHVLSMWAASRDGCGNMAGRIEFNPIRVRTHYLHTIMKLELESKRQVSRPAAPDEEPSPTASCSLTGAQGSETQDFQEFIAENETAVMHLQSAEELERLKAEEDSSGSSASLDSSIESLGVCILEEPLAVPEELCPGLTAPILIQAQLPPGSSVLCFTENSDHPTASTVNSPSYLNSGPLVYYQVEQRPVLGVKGEPWYGRRLSLFPKGEGSECLLSPCYLTRGLVAPQTQLPSVNQRWGKHPP

SEQ ID NO: 122 - protein, 126 aa, Homo sapiens [sequence omitted]

SEQ ID NO: 123 - protein, 911 aa, Homo sapiens
  misc_feature: Incyte ID No LI902018.107.orf22000MAY01
TAWQCQGRVGVSPHCPASVGQPNTVCLVVCVHMCACVCPAEQQTAAPESCGAFPXYGSSFYALEEKHVGFSLDVGEIEKKGKGKKRRGRRSKKERRRGRKEGEEDQNPPCPRLSRELLDEKEPEVLQDSLDRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEDQRRKEEGEEKKGKKIKTHHAPGLSRELLDEKGPEVLQDSLDRCYSTPSGCLELTDSCQPYRSAFYILEQQRVGLAVDMDEIEKYQEVEEDQDPSCPRLSGELLDEKEPEVLQESLDRCYSTPSGCLELTDSCQPYRSAFYILEQQRVGLAVDMDEIEKYQEVEEDQDPSCPRLSRELLDEKEPEVLQDSLGRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRSKKERRRGRKEGEEDQNPPCPRLSRELLDEKGPEVLQDSLDRCYSTPSGCLELTDSCQPYRSAFYVLEQQRVGLAVDMDEIEKYKEVEEDQDPSCPRLSRELLDEKEPEVLQDSLDRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKIRRGRRSKKKRRRGRKEGEEDQNPPCPRLNSVLMEVEEPEVLQDSLDRCYSTPSMYCELRDSFQHYRSVFYSFEEQHISFALDMDNRFFTLTVTSLYLVFQMGVIFPQ

SEQ ID NO: 124 - protein, 198 aa, Homo sapiens
  misc_feature: Incyte ID No LI220495.1.orf12000MAY01
RFWLPHNVTWADLKNTEEATFPQAEDLYLAFPLAFCIFMVRLIFERFVAKPCAIALNIQANGPQIAPPNAILEKVFTAITKHPDEKRLEGLSKQLDWDVRSIQRWFRQRRNQEKPSTLTRFCESMWRFSFYLYVFTYGVRFLKKTPWLWNTRHCWYNYPYQPLTTDLHYYYILELSFYGSWMLFSVHWISXRIRTCGD

SEQ ID NO: 125 - protein, 205 aa, Homo sapiens
  misc_feature: Incyte ID No LI399478.1.orf32000MAY01
ADSRSPATAASPSAPALPVHSPQPPSAKPQRPLPSPMTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 126 - protein, 121 aa, Homo sapiens [sequence omitted]

SEQ ID NO: 127 - protein, 193 aa, Homo sapiens
  misc_feature: Incyte ID No LI025643.2.orf32000MAY01
LKWIPSSPGKLNEADHNGDLALRILAPLTTTGEVNCQPRWLVTKLMWTWWTKSGWSLVTPRGFQRRRSLVLPLSYINEWRPCVNAATLGAPGDTTCTFVGLCTSSTRNTQLDVMPEMAADCRGPSAGWVPTPNMQDSKGRTPVTCVPSMAAGYDICVSVSCSSRKQLDLELNRPRGQAQATVAWLVQHITSVF

SEQ ID NO: 128 - protein, 116 aa, Homo sapiens
  misc_feature: Incyte ID No LI233942.1.orf22000MAY01
DSQCMRSQPREHLFFTFLLKIHWGGIKFTPKKFAWAKSLQVPSENKILMIFFFFWLLGRDFQHSFTISQICPFSTHTQLSHLVRFILPKERCSPTLTLFQRATLSQPQQPCQEKAR

SEQ ID NO: 129 - protein, 186 aa, Homo sapiens
  misc_feature: Incyte ID No LI089158.1.orf12000MAY01
LSDFRLLHLHELGHKVPVMSNCQSGPSIXRSASPKXPXXENLPGAQAKXWVRTRTVACWYCRDAASVPSSPHRPRSISLSRVGLLLAATGILQPLIWLSSVSEISICRGSLGAQTVTSNWEVKCWWVKFTIVSTLLLHFLPSSQKMESQLLETLMTSSSRRLALPQTHDTSTTEPSSLDPHDFDRN

SEQ ID NO: 130 - protein, 90 aa, Homo sapiens
  misc_feature: Incyte ID No LI101046.1.orf22000MAY01
TYQPGFSLTKDNPRGSLDQPHLSGSPFSSSFKSPSFLQISSRLAALKDNPLLPAALLCLKGKGLAHIFSVWPPPPGHTAFFLAPFLTLLT

SEQ ID NO: 131 - protein, 167 aa, Homo sapiens
  misc_feature: Incyte ID No LI368676.2.orf12000MAY01
ESLCHRDPRKNIELYVRPMSLLGEVQLQPSTSLLPTLNRTFIWDVKAHKSIGLELQFSIPRMRQIGPVKSCPDGVTNSISGRIAATVVRIGTFCSNGTVSRIQDARRSENGLTPPMGSTPENVSGFSIETRSSIKRLCIIESVFEGEGSATLNVCQLPTKAPLRMSS

SEQ ID NO: 132 - protein, 249 aa, Homo sapiens
  misc_feature: Incyte ID No LI238713.1.orf22000MAY01
VKLKELFNRILRNQASVMQKRKTEKLKQEQKGQPRTVSPSTIRDGPSSAPATPTKAPYSPTTSKEKKIRITTNDGRQSMVTLKSSTNFFELQESIAREFNIPPYLQCIRYGFPPKELMPPQAGMEKEPVPLQHGDRITIEILKSKAEGGQSAAAHSAHTVKQEDIAVTGKLSSKELQEQAEKEMYSLCLLATLMGEDVWSYAKGLPHMFQQGGVFYSIMKKTMGMADGKHCTFPHLPGKTFVYNASEDR

SEQ ID NO: 133 - protein, 110 aa, Homo sapiens
  misc_feature: Incyte ID No LI720928.1.orf32000MAY01
TFTHMHIITGTQTFTHMHIITGTQTYTHMHILMGTQTFTRMHILMGTQTFTHMHMLMGTQIFTYMHKIMGTQTFTHMHMITGTQTYGSTGLWAHSLQHKHTDVPPIFLRV

SEQ ID NO: 134 - protein, 215 aa, Homo sapiens
  misc_feature: Incyte ID No LI221874.1.orf22000MAY01
IQPSVSKNDYQNGVCFVRLGTMSLSTEGEEETVTTLDYSHCSFRTSSGKRFLLLKKLLEELYLDANQIEELPKQLFNCQSLHKLSLPDNDLTTLPASIANLINLRGTGCQQEWNTGVSRKLSKIVKCLTIVEASVNPISKLPDGFSQLLNLTQLYLNDAFLEFLPANFGRLTKLQILELRENQLKMLPKTMNRLTQLERLDLGSNEFTGSALKVR

SEQ ID NO: 135 - protein, 198 aa, Homo sapiens [sequence omitted]

SEQ ID NO: 136 - protein, 147 aa, Homo sapiens
  misc_feature: Incyte ID No LI1143605.1.orf12000MAY01
TYSIWASSCKNKAECNELHPSVSVVQILAFILIRNIPGYARSVYSSFFAWFGKISLELFICQYHIWLAADTRGILVLIPGKPYAQHHCQHFHICLCGHMKFLRSLMILHRLLFAKDNSSLLKRLACIAAFFFCGLLILSSIQDKSKH

SEQ ID NO: 137 - protein, 153 aa, Homo sapiens
  misc_feature: Incyte ID No LI474069.7.orf12000MAY01
RSCVLNSDHMIHSHSPRYRSGSVNLNGCCEHRRALISTAVNPKLKDAVQARKRGIYLNTXLLIVVKLLFDGSAFAMKFPEKKSYEYKILKKSSTVLPLSESLFPFLDPPVMLCDYKFDDESAEEIRDHFMEMLDHTIQIEDLGNCRGNKHSLY

SEQ ID NO: 138 - protein, 279 aa, Homo sapiens
  misc_feature: Incyte ID No LI245193.3.orf12000MAY01
VRLAVATTEEEGRDAVEHGDRLSVMEGSGEQPGPQPQHPGDHRIRDGDFVVLKREDVFKAVQVQRRKKVTFEKQWFYLDNVIGHSYGTAFEVTSGGSLQPKKKREEPTAETKEAGTDNRNIVDDGKSQKLTQDDIKALKDKGIKGEEIVQQLIENSTTFRDKTEFAQDKYIKKKKKKYEAIITVVKPSTRILSIMYYAREPGKINHMRIRYTSPDVDVGKYPCWQQNDCDGNVCRLGAGCNDGTNGRFWLHYSAIPWRRTCSGSNSMFWISQIFSQWSL

SEQ ID NO: 139 - protein, 138 aa, Homo sapiens [sequence omitted]

SEQ ID NO: 140 - protein, 347 aa, Homo sapiens [sequence omitted]

SEQ ID NO: 141 - protein, 440 aa, Homo sapiens
  misc_feature: Incyte ID No LI337514.3.orf12000MAY01
SGWSWALAARGRRKLSSCTGGRVRAVGGSGACAVAMSVYARGPGPREAVPSGRPRPDTLTPPWVRQRAVTGTFCASWTPLRNRRAQRMATDMQRKRSSECLDGTLTPSDGQSMERTESPTPGMAQGMEPGAGQEGAMFVHARSYEDLTESEDGAASGDSHKEGTRGPPPLPTDMRQISQDFSELSTQLTGVARDLQEEMLPGSSEDWLEPPGAVGRPATEPPREGTTEGDEEDATEAWRLHQKHVFVLSEAGKPVYSRYGSEEALSSTMGVMVAPGVLPGGRQERHPLHPCRWLQGSIRAPEPAGASGGGSYAAVGTRAGAGAALHLLPDPKPSYRCAAAATSSSRSRTMICGAYSRAQSASPDNLLQLMARDPSFLMGAARCLPLAAAVRDTVSASLQQARARSLVFSILLGPQPARGTRAPKGPISAPHRPAPALQPH

SEQ ID NO: 142 - protein, 386 aa, Homo sapiens [sequence omitted]

SEQ ID NO: 143 - protein, 198 aa, Homo sapiens [sequence omitted]

SEQ ID NO: 144 - protein, 116 aa, Homo sapiens [sequence omitted]

SEQ ID NO: 145 - protein, 307 aa, Homo sapiens
  misc_feature: Incyte ID No LI197275.5.orf22000MAY01
SALQHLESLQKQYLFVSNKTGTLHEACEQLLKEQSELVDLAENIQQKLSYFNELETINTKLNSPTLSVNSDGFIPMLAKLDDCITYISSHPNFKDYPIYLLKFKQCLSKALHLMKTYTVNTLQTLTSQLLKRDPSSVPNADNAFTLFYVKFRAAAPKVRTLIEQIELRSEKIPEYQQLLNDIHQCYLDQRELLLGPSIACTVAELTSQNNRDHCALVRSGCAFMVHVCQDEHQLYNEFFTKPTSKLDELLEKLCVSLYDVFRPLIIHVIHLETLSELCGILKNEVLKIMCRTMLSNWGHPQLESSRC

SEQ ID NO: 146 - protein, 85 aa, Homo sapiens
  misc_feature: Incyte ID No LI336872.1.orf22000MAY01
SSLTGLDHLSLVKSKAYVHSKIPSCRRRLTSHSQPLSPLNCSHEDLRHTSLNTPFHMKILEAECPSICGFLFLFLRQDLSLSPSL

SEQ ID NO: 147 - protein, 155 aa, Homo sapiens
  misc_feature: Incyte ID No LI1092901.1.orf22000MAY01
DEHTFHMLSFKRPFVFKLALESQPTSFFFSGTFLHSLRITSLKNHDLIICHLVSVYIVMLVMAAELLSLDVFASQNFQNNFRCKAVFYIYKVMRGLLICTTSLLSMLQIITISPSTSLHSREIGRIPDKLSNHVFSAFLIQTQMVLLWQTLLVLF

SEQ ID NO: 148 - protein, 147 aa, Homo sapiens
  misc_feature: Incyte ID No LI022387.5.orf22000MAY01
IWVWPLLGPGLLGSTMASLLKALHPGADCGRPTGHGWALSQRACQHGHFHTQAGWEASYEVLKGPLCLQVAPSHTGEDRWASLVAKGTEVQSRECVAQCQHGLLWGQASCPGPKPPQAKACPWAHFLSRGLGFPISDVCERKACPLG

SEQ ID NO: 149 - protein, 110 aa, Homo sapiens
  misc_feature: Incyte ID No LI1188334.1.orf32000MAY01
FCIFFRTSSGKSSVINAMLWDKVLPSGIGHITNCFLSVEGTDGDKAYLMTEGSDEKKSVKTVNQLAHALHMDKDLKAGCLVRVFWPDHLQWLFDQVPDSYMLLQQNVILQ

SEQ ID NO: 150 - protein, 125 aa, Homo sapiens
  misc_feature: Incyte ID No LI1188664.1.orf32000MAY01
MLCFLHRSPMGKTSGKLFDSSDDDESDSEDDSNRFKIKPQFEGRAGQKLMDLQSHFGTDDRFRMDSRFLETDSEEEQEEVNEKKTAEEEELAEEKKKALNVVQSVLQINLSNSTNRGSVAAKKFK

SEQ ID NO: 151 - protein, 102 aa, Homo sapiens
  misc_feature: Incyte ID No LI247388.1.orf32000MAY01
NSPLQENGGQPGVVVVESVAPVGSGLSHRDCHLDPPALAPYLPWALRWPCFSGFASLAVSSSACTHPCPQHGLGAAETGGPAHPASDSCEVLSVACYGVLDP

SEQ ID NO: 152 - protein, 132 aa, Homo sapiens [sequence omitted]

SEQ ID NO: 153 - protein, 120 aa, Homo sapiens
  misc_feature: Incyte ID No LI1188967.1.orf12000MAY01
AGAKLAGFREPCGRSIYNPGGSYAMASRHLQATVTRATAKGPQRKYSSLGMNPWTFLAWPKICGALLGQVSMGPGTWLGILWGPVRDPFAQEHPKRPSAWDWSAHSQKDPRQSMGHEIQE

SEQ ID NO: 154 - protein, 288 aa, Homo sapiens
  misc_feature: Incyte ID No LI236230.3.orf12000MAY01
SKFIIHNVYSVSKQGVVILDDKSKELPHWVLSAMKCLANWPNCSDLKQPMYLGFEKDVFKTIADYYGHLKEPLLTFHLFDAFVSVLGLLQKEKVAVEAFQICCLLLPPENRRKLQLLMRMMARICLNKEMPPLCDGFGTRTLMVQTFSRCILCSKDEVDLDELLAARLVTFLMDNYQEILKVPLALQTSIEERVAHLRRVQIKYPGADMDITLSAPSFCRQISPEEFEYQRSYGLSGNLWAALLEEVITDAKLSNKEKKKKLKQFQKSYPEVYQERFPTPESAALSVS

SEQ ID NO: 155 - protein, 172 aa, Homo sapiens [sequence omitted]

SEQ ID NO: 156 - protein, 315 aa, Homo sapiens
  misc_feature: Incyte ID No LI1190057.1.orf22000MAY01
RRRPWPSVPCGLGMAFSQSHVMAARRHQHSRLIIEVDEYSSNPTQAFTFYNINQGRFQPPHVQMVDPVPHDAPKPPGYTRFVCVSDTHSRTDPIQMPYGDVLIHAGDFTELGLPSEVKKFNEWLGSLPYEYKIVIAGNHELTFDQEFMADLIKQDFYYFPSVSKLKPENYENVQSLLTNCIYLQDSEVTVRGFRIYGSPWQPWFYGWGFNLPRGQALLEKWNLIPEGVDILITHGPPLGFLDWVPKKMQRVGCVELLNTVQRRVQPRLHVFGHIHEGYGVMADGTTTYVNASVCTVNYQPVNPPIVIDLPTPRNS

SEQ ID NO: 157 - protein, 145 aa, Homo sapiens
  misc_feature: Incyte ID No LI221836.3.orf12000MAY01
VKNRWRNEEGSRNEMTTPSLDYSMPAPYRRVEAPVAYPEGENSHDKSSSERSTPPYLFPEYPEASKNTGQNREVSILYPGAKDQRQGSLLPEELEDQMPRLVAEESNRGSTTINKEEVNKGPFVAVVGVAKGVRDSGAPIQLDPL

SEQ ID NO: 158 - protein, 145 aa, Homo sapiens
  misc_feature: Incyte ID No LI221836.3.orf22000MAY01
LLWVLPKVLEIQELLFSLIPCNREELAERRKAVESWNPVPYSVASAAIPAAAIGEKARGYEESEGHNTPKLKNQRELEELKRTTEKLERVLAERNLFQQKVEELEQERNHWQSEFKKVQHELVIYSTQEAEGLYWSKKHSGLSPS

SEQ ID NO: 159 - protein, 108 aa, Homo sapiens
  misc_feature: Incyte ID No LI334047.3.orf12000MAY01
AKIHVKYTLPHTTSHLYLYFQTKSITAQKSAACTGLVTPRSVCSHQQHLKLVSVWQGHCWWPLXPSPLQWIQFGSPXEIHCSLTQKVVSFSMASSYHKIKQTKFSNRL

(19) United States
(12) Patent Application Publication
(10) Pub. No.: US 2003/0124569 A1
(43) Pub. Date: Jul. 3, 2003

(54) Secretory molecules

(76) Inventors: Scott R. Panzer, Sunnyvale, CA (US); Peter A. Spiro, Palo Alto, CA (US); Steven C. Banville, Palo Alto, CA (US); Purvi Shah, San Jose, CA (US); Michael S. Chalup, Sunnyvale, CA (US); Simon C. Chang, Mountain View, CA (US); Alice Chen, San Jose, CA (US); Steven D'sa, East Palo Alto, CA (US); Stefan Amshey, San Francisco, CA (US); Christopher R. Dahl, Fremont, CA (US); Tam C. Dam, San Jose, CA (US); Susan E. Daniels, Palo Alto, CA (US); Gerard E. Dufour, Castro Valley, CA (US); Vincent Flores, Union City, CA (US); Willy T. Fong, San Francisco, CA (US); Lila B. Greenawalt, San Jose, CA (US); Jennifer L. Hillman, Mountain View, CA (US); Anissa L. Jones, San Jose, CA (US); Tommy F. Liu, Daly City, CA (US); Ann M. Roseberry, Redwood City, CA (US); Bruce H. Rosen, Menlo Park, CA (US); Frank D. Russo, Sunnyvale, CA (US); Theresa K. Stockdreher, Sunnyvale, CA (US); Abel Daffo, San Jose, CA (US); Rachel J. Wright, Mountain View, CA (US); Pierre E. Yap, Lafayette, CA (US); Jimmy Y. Yu, Fremont, CA (US); Diana L. Bradley, Soquel, CA (US); Shawn R. Bratcher, Mountain View, CA (US); Wensheng Chen, Mountain View, CA (US); Howard J. Cohen, Palo Alto, CA (US); David M. Hodgson, Ann Arbor, MI (US); Stephen E. Lincoln, Redwood City, CA (US)

Correspondence Address: Incyte Genomics Inc, Legal Department, 3160 Porter Drive, Palo Alto, CA, 94304, US

(21) Appl. No.: 10/204,887

(22) PCT Filed: Feb. 1, 2001
(86) PCT No.: PCT/US01/03465

(51) Int. Cl. C12Q 1/68; C07H 21/02; C07H 21/04; C12P 19/34

(52) U.S. Cl. 435/6; 435/91.2; 536/23.1; 536/24.3

(57) ABSTRACT

The present invention provides purified secretory polynucleotides (sptm). Also encompassed are the polypeptides (SPTM) encoded by sptm. The invention also provides for the use of sptm, or complements, oligonucleotides, or fragments thereof in diagnostic assays. The invention further provides for vectors and host cells containing sptm for the expression of SPTM. The invention additionally provides for the use of isolated and purified SPTM to induce antibodies and to screen libraries of compounds and the use of anti-SPTM antibodies in diagnostic assays. Also provided are microarrays containing sptm and methods of use.

### TECHNICAL FIELD

The present invention relates to secretory molecules and to the use of these sequences in the diagnosis, study, prevention, and treatment of diseases associated with, as well as effects of exogenous compounds on, cell signaling and the expression of secretory molecules.

### BACKGROUND OF THE INVENTION

Protein transport and secretion are essential for cellular function. Protein transport is mediated by a signal peptide located at the amino terminus of the protein to be transported or secreted. The signal peptide is comprised of about ten to twenty hydrophobic amino acids which target the nascent protein from the ribosome to a particular membrane bound compartment such as the endoplasmic reticulum (ER). Proteins targeted to the ER may either proceed through the secretory pathway or remain in any of the secretory organelles such as the ER, Golgi apparatus, or lysosomes. Proteins that transit through the secretory pathway are either secreted into the extracellular space or retained in the plasma membrane. Proteins that are retained in the plasma membrane contain one or more transmembrane domains, each comprised of about 20 hydrophobic amino acid residues. Proteins that are secreted from the cell are generally synthesized as inactive precursors that are activated by post-translational processing events during transit through the secretory pathway. Such events include glycosylation, proteolysis, and removal of the signal peptide by a signal peptidase. Other events that may occur during protein transport include chaperone-dependent unfolding and folding of the nascent protein and interaction of the protein with a receptor or pore complex. Examples of secretory proteins with amino terminal signal peptides are discussed below and include proteins with important roles in cell-to-cell signaling. Such proteins include transmembrane receptors and cell surface markers, extracellular matrix molecules, cytokines, hormones, growth and differentiation factors, neuropeptides, vasomediators, ion channels, transporters/pumps, and proteases. (Reviewed in Alberts, B. et al. (1994) Molecular Biology of The Cell, Garland Publishing, New York N.Y., pp. 557-560, 582-592.)

G-protein coupled receptors (GPCRs) comprise a superfamily of integral membrane proteins which transduce extracellular signals. Not all GPCRs contain N-terminal signal peptides. GPCRs include receptors for biogenic amines such as dopamine, epinephrine, histamine, glutamate (metabotropic-type), acetylcholine (muscarinic-type), and serotonin; for lipid mediators of inflammation such as prostaglandins, platelet activating factor, and leukotrienes; for peptide hormones such as calcitonin, C5a anaphylatoxin, follicle stimulating hormone, gonadotropin releasing hormone, neurokinin, oxytocin, and thrombin; and for sensory signal mediators such as retinal photopigments and olfactory stimulatory molecules. The structure of these highly conserved receptors consists of seven hydrophobic transmembrane regions, cysteine disulfide bridges between the second and third extracellular loops, an extracellular N-terminus, and a cytoplasmic C-terminus. The N-terminus interacts with ligands, the disulfide bridges interact with agonists and antagonists, and the large third intracellular loop interacts with G proteins to activate second messengers such as cyclic AMP, phospholipase C, inositol triphosphate, or ion channels. (Reviewed in Watson, S. and Arkinstall, S. (1994) The G-protein Linked Receptor Facts Book, Academic Press, San Diego Calif., pp. 2-6; and Bolander, F. F. (1994) Molecular Endocrinology, Academic Press, San Diego Calif., pp. 162-176.)

Other types of receptors include cell surface antigens identified on leukocytic cells of the immune system. These antigens have been identified using systematic, monoclonal antibody (mAb)-based “shot gun” techniques. These techniques have resulted in the production of hundreds of mAbs directed against unknown cell surface leukocytic antigens. These antigens have been grouped into “clusters of differentiation” based on common immunocytochemical localization patterns in various differentiated and undifferentiated leukocytic cell types. Antigens in a given cluster are presumed to identify a single cell surface protein and are assigned a “cluster of differentiation” or “CD” designation. Some of the genes encoding proteins identified by CD antigens have been cloned and verified by standard molecular biology techniques. CD antigens have been characterized as both transmembrane proteins and cell surface proteins anchored to the plasma membrane via covalent attachment to fatty acid-containing glycolipids such as glycosylphosphatidylinositol (GPI). (Reviewed in Barclay, A. N. et al. (1995) The Leucocyte Antigen Facts Book, Academic Press, San Diego Calif., pp. 20 17-20.)

Matrix proteins (MPs) are transmembrane and extracellular proteins which function in formation, growth, remodeling, and maintenance of tissues and as important mediators and regulators of the inflammatory response. The expression and balance of MPs may be perturbed by biochemical changes that result from congenital, epigenetic, or infectious diseases. In addition, MPs affect leukocyte migration, proliferation, differentiation, and activation in the immune response. MPs are frequently characterized by the presence of one or more domains which may include collagen-like domains, EGF-like domains, immunoglobulin-like domains, and fibronectin-like domains. In addition, MPs may be heavily glycosylated and may contain an Arginine-Glycine-Aspartate (RGD) tripeptide motif which may play a role in adhesive interactions. MPs include extracellular proteins such as fibronectin, collagen, galectin, vitronectin and its proteolytic derivative somatomedin B; and cell adhesion receptors such as cell adhesion molecules (CAMs), cadherins, and integrins. (Reviewed in Ayad, S. et al. (1994) The Extracellular Matrix Facts Book, Academic Press, San Diego Calif., pp. 2-16;

Ruoslahti, E. (1997) Kidney Int. 51:1413-1417; Sjaastad, M. D. and Nelson, W. J. (1997) BioEssays 19:47-55.)

Cytokines are secreted by hematopoietic cells in response to injury or infection. Interleukins, neurotrophins, growth factors, interferons, and chemokines all define cytokine families that work in conjunction with cellular receptors to regulate cell proliferation and differentiation. In addition, cytokines effect activities such as leukocyte migration and function, hematopoietic cell proliferation, temperature regulation, acute response to infection, tissue remodeling, and apoptosis.

Chemokines, in particular, are small chemoattractant cytokines involved in inflammation, leukocyte proliferation and migration, angiogenesis and angiostasis, regulation of hematopoiesis, HIV infectivity, and stimulation of cytokine secretion. Chemokines generally contain 70-100 amino acids and are subdivided into four subfamilies based on the presence of conserved cysteine-based motifs. (Callard, R. and Gearing, A. (1994) The Cytokine Facts Book, Academic Press, New York N.Y., pp. 181-190, 210-213, 223-227.)

Growth and differentiation factors are secreted proteins which function in intercellular communication. Some factors require oligomerization or association with MPs for activity. Complex interactions among these factors and their receptors trigger intracellular signal transduction pathways that stimulate or inhibit cell division, cell differentiation, cell signaling, and cell motility. Most growth and differentiation factors act on cells in their local environment (paracrine signaling). There are three broad classes of growth and differentiation factors. The first class includes the large polypeptide growth factors such as epidermal growth factor, fibroblast growth factor, transforming growth factor, insulin-like growth factor, and platelet-derived growth factor. The second class includes the hematopoietic growth factors such as the colony stimulating factors (CSFs). Hematopoietic growth factors stimulate the proliferation and differentiation of blood cells such as B-lymphocytes, T-lymphocytes, erythrocytes, platelets, eosinophils, basophils, neutrophils, macrophages, and their stem cell precursors. The third class includes small peptide factors such as bombesin, vasopressin, oxytocin, endothelin, transferrin, angiotensin II, vasoactive intestinal peptide, and bradykinin which function as hormones to regulate cellular functions other than proliferation.

Growth and differentiation factors play critical roles in neoplastic transformation of cells in vitro and in tumor progression in vivo. Inappropriate expression of growth factors by tumor cells may contribute to vascularization and metastasis of tumors. During hematopoiesis, growth factor misregulation can result in anemias, leukemias, and lymphomas. Certain growth factors such as interferon are cytotoxic to tumor cells both in vivo and in vitro. Moreover, some growth factors and growth factor receptors are related both structurally and functionally to oncoproteins. In addition, growth factors affect transcriptional regulation of both proto-oncogenes and oncosuppressor genes. (Reviewed in Pimentel, E. (1994) Handbook of Growth Factors, CRC Press, Ann Arbor Mich., pp. 1-9.)

Proteolytic enzymes or proteases either activate or deactivate proteins by hydrolyzing peptide bonds. Proteases are found in the cytosol, in membrane-bound compartments, and in the extracellular space. The major families are the zinc, serine, cysteine, thiol, and carboxyl proteases.

Ion channels, ion pumps, and transport proteins mediate the transport of molecules across cellular membranes. Transport can occur by a passive, concentration-dependent mechanism or can be linked to an energy source such as ATP hydrolysis. Symporters and antiporters transport ions and small molecules such as amino acids, glucose, and drugs. Symporters transport molecules and ions unidirectionally, and antiporters transport molecules and ions bidirectionally. Transporter superfamilies include facilitative transporters and active ATP-binding cassette transporters which are involved in multiple-drug resistance and the targeting of antigenic peptides to MHC Class I molecules. These transporters bind to a specific ion or other molecule and undergo a conformational change in order to transfer the ion or molecule across the membrane. (Reviewed in Alberts, B. et al. (1994) Molecular Biology of The Cell, Garland Publishing, New York N.Y., pp. 523-546.)

Ion channels are formed by transmembrane proteins which create a lined passageway across the membrane through which water and ions, such as Na⁺, K⁺, Ca²⁺, and Cl⁻, enter and exit the cell. For example, chloride channels are involved in the regulation of the membrane electric potential as well as absorption and secretion of ions across the membrane. Chloride channels also regulate the internal pH of membrane-bound organelles.

Ion pumps are ATPases which actively maintain membrane gradients. Ion pumps are classified as P, V, or F according to their structure and function. All have one or more binding sites for ATP in their cytosolic domains. The P-class ion pumps include Ca²⁺ATPase and Na⁺/K⁺ATPase and function in transporting H⁺, Na⁺, K⁺, and Ca²⁺ ions. P-class pumps consist of two α and two β transmembrane subunits. The V- and F-class ion pumps have similar structures but transport only H⁺. F class H⁺ pumps mediate transport across the membranes of mitochondria and chloroplasts, while V-class H⁺ pumps regulate acidity inside lysosomes, endosomes, and plant vacuoles.

A family of structurally related intrinsic membrane proteins known as facilitative glucose transporters catalyze the movement of glucose and other selected sugars across the plasma membrane. The proteins in this family contain a highly conserved, large transmembrane domain comprised of 12 α-helices, and several weakly conserved, cytoplasmic and exoplasmic domains. (Pessin, J. E. and Bell, G. I. (1992) Annu. Rev. Physiol. 54:911-930.)

Amino acid transport is mediated by Na⁺ dependent amino acid transporters. These transporters are involved in gastrointestinal and renal uptake of dietary and cellular amino acids and in neuronal reuptake of neurotransmitters. Transport of cationic amino acids is mediated by the system y+ family and the cationic amino acid transporter (CAT) family. Members of the CAT family share a high degree of sequence homology, and each contains 12-14 putative transmembrane domains. (Ito, K. and Groudine, M. (1997) J. Biol. Chem. 272:26780-26786.)

Hormones are secreted molecules that travel through the circulation and bind to specific receptors on the surface of, or within, target cells. Although they have diverse biochemical compositions and mechanisms of action, hormones can be grouped into two categories. One category includes small lipophilic hormones that diffuse through the plasma membrane of target cells, bind to cytosolic or nuclear receptors, and form a complex that alters gene expression. Examples of these molecules include retinoic acid, thyroxine, and the cholesterol-derived steroid hormones such as progesterone, estrogen, testosterone, cortisol, and aldosterone. The second category includes hydrophilic hormones that function by binding to cell surface receptors that transduce signals across the plasma membrane. Examples of such hormones include amino acid derivatives such as catecholamines and peptide hormones such as glucagon, insulin, gastrin, secretin, cholecystokinin, adrenocorticotropic hormone, follicle stimulating hormone, luteinizing hormone, thyroid stimulating hormone, and vasopressin. (See, for example, Lodish et al. (1995) Molecular Cell Biology, Scientific American Books Inc., New York N.Y., pp. 856-864.)

Neuropeptides and vasomediators (NP/VM) comprise a large family of endogenous signaling molecules. Included in this family are neuropeptides and neuropeptide hormones such as bombesin, neuropeptide Y, neurotensin, neuromedin N, melanocortins, opioids, galanin, somatostatin, tachykinins, urotensin II and related peptides involved in smooth muscle stimulation, vasopressin, vasoactive intestinal peptide, and circulatory system-borne signaling molecules such as angiotensin, complement, calcitonin, endothelins, formyl-methionyl peptides, glucagon, cholecystokinin and gastrin. NP/VMs can transduce signals directly, modulate the activity or release of other neurotransmitters and hormones, and act as catalytic enzymes in cascades. The effects of NP/VMs range from extremely brief to long-lasting. (Reviewed in Martin, C. R. et al. (1985) Endocrine Physiology, Oxford University Press, New York, N.Y., pp. 57-62.)

The discovery of new secretory molecules satisfies a need in the art by providing new compositions which are useful in the diagnosis, study, prevention, and treatment of diseases associated with, as well as effects of exogenous compounds on, cell signaling and the expression of secretory molecules.

### SUMMARY OF THE INVENTION

The present invention relates to nucleic acid sequences comprising human polynucleotides encoding secretory polypeptides that contain signal peptides and/or transmembrane domains. These human polynucleotides (sptm) as presented in the Sequence Listing uniquely identify partial or full length genes encoding structural, functional, and regulatory polypeptides involved in cell signaling.

The invention provides an isolated polynucleotide comprising a polynucleotide sequence selected from the group consisting of a) a polynucleotide sequence selected from the group consisting of SEQ ID NO: 1-79; b) a naturally occurring polynucleotide sequence having at least 90% sequence identity to a polynucleotide sequence selected from the group consisting of SEQ ID NO: 1-79; c) a polynucleotide sequence complementary to a); d) a polynucleotide sequence complementary to b); and e) an RNA equivalent of a) through d). In one alternative, the polynucleotide comprises a polynucleotide sequence selected from the group consisting of SEQ ID NO: 1-79. In another alternative, the polynucleotide comprises at least 60 contiguous nucleotides of a polynucleotide sequence selected from the group consisting of a) a polynucleotide sequence selected from the group consisting of SEQ ID NO: 1-79; b) a naturally occurring polynucleotide sequence having at least 90% sequence identity to a polynucleotide sequence selected from the group consisting of SEQ ID NO: 1-79; c) a polynucleotide sequence complementary to a); d) a polynucleotide sequence complementary to b); and e) an RNA equivalent of a) through d). The invention further provides a composition for the detection of expression of secretory polynucleotides comprising at least one isolated polynucleotide comprising a polynucleotide sequence selected from the group consisting of a) a polynucleotide sequence selected from the group consisting of SEQ ID NO: 1-79; b) a naturally occurring polynucleotide sequence having at least 90% sequence identity to a polynucleotide sequence selected from the group consisting of SEQ ID NO: 1-79; c) a polynucleotide sequence complementary to a); d) a polynucleotide sequence complementary to b); and e) an RNA equivalent of a) through d); and a detectable label.

The invention also provides a method for detecting a target polynucleotide in a sample, said target polynucleotide comprising a polynucleotide sequence selected from the group consisting of a) a polynucleotide sequence selected from the group consisting of SEQ ID NO: 1-79; b) a naturally occurring polynucleotide sequence having at least 90% sequence identity to a polynucleotide sequence selected from the group consisting of SEQ ID NO: 1-79; c) a polynucleotide sequence complementary to a); d) a polynucleotide sequence complementary to b); and e) an RNA equivalent of a) through d). The method comprises a) amplifying said target polynucleotide or a fragment thereof using polymerase chain reaction amplification, and b) detecting the presence or absence of said amplified target polynucleotide or fragment thereof, and, optionally, if present, the amount thereof.

The invention also provides a method for detecting a target polynucleotide in a sample, said target polynucleotide comprising a polynucleotide sequence selected from the group consisting of a) a polynucleotide sequence selected from the group consisting of SEQ ID NO: 1-79; b) a naturally occurring polynucleotide sequence having at least 90% sequence identity to a polynucleotide sequence selected from the group consisting of SEQ ID NO: 1-79; c) a polynucleotide sequence complementary to a); d) a polynucleotide sequence complementary to b); and e) an RNA equivalent of a) through d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide, and b) detecting the presence or absence of said hybridization complex, and, optionally, if present, the amount thereof. In one alternative, the probe comprises at least 30 contiguous nucleotides. In another alternative, the probe comprises at least 60 contiguous nucleotides.

The invention further provides a recombinant polynucleotide comprising a promoter sequence operably linked to an isolated polynucleotide comprising a polynucleotide sequence selected from the group consisting of a) a polynucleotide sequence selected from the group consisting of SEQ ID NO: 1-79; b) a naturally occurring polynucleotide sequence having at least 90% sequence identity to a polynucleotide sequence selected from the group consisting of SEQ ID NO: 1-79; c) a polynucleotide sequence complementary to a); d) a polynucleotide sequence complementary to b); and e) an RNA equivalent of a) through d). In one alternative, the invention provides a cell transformed with the recombinant polynucleotide. In another alternative, the invention provides a transgenic organism comprising the recombinant polynucleotide. In a further alternative, the invention provides a method for producing a secretory polypeptide, the method comprising a) culturing a cell under conditions suitable for expression of the secretory polypeptide, wherein said cell is transformed with the recombinant polynucleotide, and b) recovering the secretory polypeptide so expressed.

The invention also provides a purified secretory polypeptide (SPTM) encoded by at least one polynucleotide comprising a polynucleotide sequence selected from the group consisting of SEQ ID NO: 1-79. Additionally, the invention provides an isolated antibody which specifically binds to the secretory polypeptide. The invention further provides a method of identifying a test compound which specifically binds to the secretory polypeptide, the method comprising the steps of a) providing a test compound; b) combining the secretory polypeptide with the test compound for a sufficient time and under suitable conditions for binding; and c) detecting binding of the secretory polypeptide to the test compound, thereby identifying the test compound which specifically binds the secretory polypeptide.

The invention further provides a microarray wherein at least one element of the microarray is an isolated polynucleotide comprising at least 60 contiguous nucleotides of a polynucleotide comprising a polynucleotide sequence selected from the group consisting of a) a polynucleotide sequence selected from the group consisting of SEQ ID NO: 1-79; b) a naturally occurring polynucleotide sequence having at least 90% sequence identity to a polynucleotide sequence selected from the group consisting of SEQ ID NO: 1-79; c) a polynucleotide sequence complementary to a); d) a polynucleotide sequence complementary to b); and e) an RNA equivalent of a) through d). The invention also provides a method for generating a transcript image of a sample which contains polynucleotides. The method comprises a) labeling the polynucleotides of the sample, b) contacting the elements of the microarray with the labeled polynucleotides of the sample under conditions suitable for the formation of a hybridization complex, and c) quantifying the expression of the polynucleotides in the sample.

Additionally, the invention provides a method for screening a compound for effectiveness in altering expression of a target polynucleotide, wherein said target polynucleotide comprises a polynucleotide sequence selected from the group consisting of a) a polynucleotide sequence selected from the group consisting of SEQ ID NO: 1-79; b) a naturally occurring polynucleotide sequence having at least 90% sequence identity to a polynucleotide sequence selected from the group consisting of SEQ ID NO: 1-79; c) a polynucleotide sequence complementary to a); d) a polynucleotide sequence s complementary to b); and e) an RNA equivalent of a) through d). The method comprises a) exposing a sample comprising the target polynucleotide to a compound, and b) detecting altered expression of the target polynucleotide, and c) comparing the expression of the target polynucleotide in the presence of varying amounts of the compound and in the absence of the compound.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide comprising a polynucleotide sequence selected from the group consisting of i) a polynucleotide sequence selected from the group consisting of SEQ ID NO: 1-79; ii) a naturally occurring polynucleotide sequence having at least 90% sequence identity to a polynucleotide sequence selected from the group consisting of SEQ ID NO: 1-79; iii) a polynucleotide sequence complementary to i), iv) a polynucleotide sequence complementary to ii), and v) an RNA equivalent of i)-iv). Hybridization occurs under conditions whereby a specific hybridization complex is formed between said probe and a target polynucleotide in the biological sample, said target polynucleotide comprising a polynucleotide sequence selected from the group consisting of i) a polynucleotide sequence selected from the group consisting of SEQ ID NO: 1-79; ii) a naturally occurring polynucleotide sequence having at least 90% sequence identity to a polynucleotide sequence selected from the group consisting of SEQ ID NO: 1-79; iii) a polynucleotide sequence complementary to i), iv) a polynucleotide sequence complementary to ii), and v) an RNA equivalent of i)-iv), and alternatively, the target polynucleotide comprises a fragment of a polynucleotide sequence selected from the group consisting of i-v above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### DESCRIPTION OF THE TABLES

Table 1 shows tile sequence identification numbers (SEQ ID NO:s) and template identification numbers (template IDs) corresponding to the polynucleotides of the present invention, along with polynucleotide segments of each template sequence as defined by the indicated “start” and “stop” nucleotide positions. The reading frames of the polynucleotide segments are shown, and the polypeptides encoded by the polynucleotide segments constitute either signal peptide (SP) or transmembrane (TM) domains, as indicated. The membrane topology of the encoded polypeptide sequence is indicated, the N-terminus (N) listed as being oriented to either the cytosolic (in) or non-cytosolic (out) side of the cell membrane or organelle.

Table 2 shows the sequence identification numbers (SEQ ID NO:s) corresponding to the polynucleotides of the present invention, along with component sequence identification numbers (component IDs) corresponding to each template. The component sequences, which were used to assemble the template sequences, are defined by the indicated “start” and “stop” nucleotide positions along each template.

Table 3 shows the tissue distribution profiles for the templates of the invention.

Table 4 summarizes the bioinformatics tools which are useful for analysis of the polynucleotides of the present invention. The first column of Table 4 lists analytical tools, programs, and algorithms, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score, the greater the homology between two sequences).

### DETAILED DESCRIPTION OF THE INVENTION

Before the nucleic acid sequences and methods are presented, it is to be understood that this invention is not limited to the particular machines, methods, and materials described. Although particular embodiments are described, machines, methods, and materials similar or equivalent to these embodiments may be used to practice the invention. The preferred machines, methods, and materials set forth are not intended to limit the scope of the invention which is limited only by the appended claims.

The singular forms “a”, “an”, and “the” include plural reference unless the context clearly dictates otherwise. All technical and scientific terms have the meanings commonly understood by one of ordinary skill in the art. All publications are incorporated by reference for the purpose of describing and disclosing the cell lines, vectors, and methodologies which are presented and which might be used in connection with the invention. Nothing in the specification is to be construed as an admission that the invention is not entitled to antedate such disclosure by virtue of prior invention.

Definitions

As used herein, the lower case “sptm” refers to a nucleic acid sequence, while the upper case “SPTM” refers to an amino acid sequence encoded by sptm. A “full-length” sptm refers to a nucleic acid sequence containing the entire coding region of a gene endogenously expressed in human tissue.

“Adjuvants” are materials such as Freund's adjuvant, mineral gels (aluminum hydroxide), and surface active substances (lysolecithin, pluronic polyols, polyanions, peptides, oil emulsions, keyhole limpet hemocyanin, and dinitrophenol) which may be administered to increase a host's immunological response.

“Allele” refers to an alternative form of a nucleic acid sequence. Alleles result from a “mutation,” a change or an alternative reading of the genetic code. Any given gene may have none, one, or many allelic forms. Mutations which give rise to alleles include deletions, additions, or substitutions of nucleotides. Each of these changes may occur alone, or in combination with the others, one or more times in a given nucleic acid sequence. The present invention encompasses allelic sptm.

“Amino acid sequence” refers to a peptide, a polypeptide, or a protein of either natural or synthetic origin. The amino acid sequence is not limited to the complete, endogenous amino acid sequence and may be a fragment, epitope, variant, or derivative of a protein expressed by a nucleic acid sequence.

“Amplification” refers to the production of additional copies of a sequence and is carried out using polymerase chain reaction (PCR) technologies well known in the art.

“Antibody” refers to intact molecules as well as to fragments thereof, such as Fab, F(ab′)₂, and Fv fragments, which are capable of binding the epitopic determinant. Antibodies that bind SPTM polypeptides can be prepared using intact polypeptides or using fragments containing small peptides of interest as the immunizing antigen. The polypeptide or peptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

“Antisense sequence” refers to a sequence capable of specifically hybridizing to a target sequence. The antisense sequence may include DNA, RNA, or any nucleic acid mimic or analog such as peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine.

“Antisense sequence” refers to a sequence capable of specifically hybridizing to a target sequence. The antisense sequence can be DNA, RNA, or any nucleic acid mimic or analog.

“Antisense technology” refers to any technology which relies on the specific hybridization of an antisense sequence to a target sequence.

A “bin” is a portion of computer memory space used by a computer program for storage of data, and bounded in such a manner that data stored in a bin may be retrieved by the program.

“Biologically active” refers to an amino acid sequence having a structural, regulatory, or biochemical function of a naturally occurring amino acid sequence.

“Clone joining” is a process for combining gene bins based upon the bins' containing sequence information from the same clone. The sequences may assemble into a primary gene transcript as well as one or more splice variants.

“Complementary” describes the relationship between two single-stranded nucleic acid sequences that anneal by base-pairing (5′-A-G-T-3′ pairs with its complement 3′-T-C-A-5′).

A “component sequence” is a nucleic acid sequence selected by a computer program such as PHRED and used to assemble a consensus or template sequence from one or more component sequences.

A “consensus sequence” or “template sequence” is a nucleic acid sequence which has been assembled from overlapping sequences, using a computer program for fragment assembly such as the GELVIEW fragment assembly system (Genetics Computer Group (GCG), Madison Wis.) or using a relational database management system (RDMS).

“Conservative amino acid substitutions” are those substitutions that, when made, least interfere with the properties of the original protein, i.e., the structure and especially the function of the protein is conserved and not significantly changed by such substitutions. The table below shows amino acids which may be substituted for an original amino acid in a protein and which are regarded as conservative substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Len, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative substitutions generally maintain (a) the structure of the polypeptide backbone in the area of the substitution, for example, as a beta sheet or alpha helical conformation, (b) the charge or hydrophobicity of the molecule at the target site, or (c) the bulk of the side chain.

“Deletion” refers to a change in either a nucleic or amino acid sequence in which at least one nucleotide or amino acid residue, respectively, is absent.

“Derivative” refers to the chemical modification of a nucleic acid sequence, such as by replacement of hydrogen by an alkyl, acyl, arino, hydroxyl, or other group.

The terms “element” and “array element” refer to a polynucleotide, polypeptide, or other chemical compound having a unique and defined position on a microarray.

“E-value” refers to the statistical probability that a match between two sequences occurred by chance.

A “fragment” is a unique portion of sptm or SPTM which is identical in sequence to but shorter in length than the parent sequence. A fragment may comprise up to the entire length of the defined sequence, minus one nucleotide/amino acid residue. For example, a fragment may comprise from 10 to 1000 contiguous amino acid residues or nucleotides. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous amino acid residues or nucleotides in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing and the figures, may be encompassed by the present embodiments.

A fragment of sptm comprises a region of unique polynucleotide sequence that specifically identifies sptm, for example, as distinct from any other sequence in the same genome. A fragment of sptm is useful, for example, in hybridization and amplification technologies and in analogous methods that distinguish sptm from related polynucleotide sequences. The precise length of a fragment of sptm and the region of sptm to which the fragment corresponds are routinely determinable by one of ordinary skill in the art based on the intended purpose for the fragment.

A fragment of SPTM is encoded by a fragment of sptm. A fragment of SPTM comprises a region of unique amino acid sequence that specifically identifies SPTM. For example, a fragment of SPTM is useful as an immunogenic peptide for the development of antibodies that specifically recognize SPTM. The precise length of a fragment of SPTM and the region of SPTM to which the fragment corresponds are routinely determinable by one of ordinary skill in the art based on the intended purpose for the fragment.

A “full length” nucleotide sequence is one containing at least a start site for translation to a protein sequence, followed by an open reading frame and a stop site, and encoding a “full length” polypeptide.

“Hit” refers to a sequence whose annotation will be used to describe a given template. Criteria for selecting the top hit are as follows: if the template has one or more exact nucleic acid matches, the top hit is the exact match with highest percent identity. If the template has no exact matches but has significant protein hits, the top hit is the protein hit with the lowest E-value. If the template has no significant protein hits, but does have significant non-exact nucleotide hits, the top hit is the nucleotide hit with the lowest E-value.

“Homology” refers to sequence similarity either between a reference nucleic acid sequence and at least a fragment of an sptm or between a reference amino acid sequence and a fragment of an SPTM.

“Hybridization” refers to the process by which a strand of nucleotides anneals with a complementary strand through base pairing. Specific hybridization is an indication that two nucleic acid sequences share a high degree of identity. Specific hybridization complexes form under defined annealing conditions, and remain hybridized after the “washing” step. The defined hybridization conditions include the annealing conditions and the washing step(s), the latter of which is particularly important in determining the stringency of the hybridization process, with more stringent conditions allowing less non-specific binding, i.e., binding between pairs of nucleic acid probes that are not perfectly matched. Permissive conditions for annealing of nucleic acid sequences are routinely determinable and may be consistent among hybridization experiments, whereas wash conditions may be varied among experiments to achieve the desired stringency.

Generally, stringency of hybridization is expressed with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization is well known and can be found in Sambrook et al., 1989, Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., or 55° C. may be used. SSC concentration may be varied from about 0.2 to 2×SSC, with SDS being present at about 0.1%. Typically, blocking reagents are used to block non-specific hybridization. Such blocking reagents include, for instance, denatured salmon sperm DNA at about 100-200 μg/ml. Useful variations on these conditions will be readily apparent to those skilled in the art. Hybridization, particularly under high stringency conditions, may be suggestive of evolutionary similarity between the nucleotides. Such similarity is strongly indicative of a similar role for the nucleotides and their resultant proteins.

Other parameters, such as temperature, salt concentration, and detergent concentration may be varied to achieve the desired stringency. Denaturants, such as formamide at a concentration of about 35-50% v/v, may also be used under particular circumstances, such as RNA:DNA hybridizations. Appropriate hybridization conditions are routinely determinable by one of ordinary skill in the art.

“Immunogenic” describes the potential for a natural, recombinant, or synthetic peptide, epitope, polypeptide, or protein to induce antibody production in appropriate animals, cells, or cell lines.

“Insertion” or “addition” refers to a change in either a nucleic or amino acid sequence in which at least one nucleotide or residue, respectively, is added to the sequence.

“Labeling” refers to the covalent or noncovalent joining of a polynucleotide, polypeptide, or antibody with a reporter molecule capable of producing a detectable or measurable signal.

“Microarray” is any arrangement of nucleic acids, amino acids, antibodies, etc., on a substrate. The substrate may be a solid support such as beads, glass, paper, nitrocellulose, nylon, or an appropriate membrane.

“Linkers” are short stretches of nucleotide sequence which may be added to a vector or an sptm to create restriction endonuclease sites to facilitate cloning. “Polylinkers” are engineered to incorporate multiple restriction enzyme sites and to provide for the use of enzymes which leave 5′ or 3′ overhangs (e.g., BamHI, EcoRI, and HindIII) and those which provide blunt ends (e.g., EcoRV, SnaBI, and StuI).

“Naturally occurring” refers to an endogenous polynucleotide or polypeptide that may be isolated from viruses or prokaryotic or eukaryotic cells.

“Nucleic acid sequence” refers to the specific order of nucleotides joined by phosphodiester bonds in a linear, polymeric arrangement. Depending on the number of nucleotides, the nucleic acid sequence can be considered an oligomer, oligonucleotide, or polynucleotide. The nucleic acid can be DNA, RNA, or any nucleic acid analog, such as PNA, may be of genomic or synthetic origin, may be either double-stranded or single-stranded, and can represent either the sense or antisense (complementary) strand.

“Oligomer” refers to a nucleic acid sequence of at least about 6 nucleotides and as many as about 60 nucleotides, preferably about 15 to 40 nucleotides, and most preferably between about 20 and 30 nucleotides, that may be used in hybridization or amplification technologies. Oligomers may be used as, e.g., primers for PCR, and are usually chemically synthesized.

“Operably linked” refers to the situation in which a first nucleic acid sequence is placed in a functional relationship with the second nucleic acid sequence. For instance, a promoter is operably linked to a coding sequence if the promoter affects the transcription or expression of the coding sequence. Generally, operably linked DNA sequences may be in close proximity or contiguous and, where necessary to join two protein coding regions, in the same reading frame.

“Peptide nucleic acid” (PNA) refers to a DNA mimic in which nucleotide bases are attached to a pseudopeptide backbone to increase stability. PNAs, also designated antigene agents, can prevent gene expression by targeting complementary messenger RNA.

The phrases “percent identity” and “% identity”, as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and Sharp, P. M. (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polyniucleotide sequence pairs.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to determine alignment between a known polynucleotide sequence and other sequences on a variety of databases. Also available is a tool called “BLAST 2 Sequences” that is used for direct pairwise comparison of two nucleotide sequences. “BLAST 2 Sequences” can be accessed and used interactively at http://www.ncbi.rlm nih.gov/gorf/bl2/. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.9 (May 7, 1999) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in figures or Sequence Listings, may be used to describe a length over which percentage identity may be measured.

Nucleic acid sequences that do not show a high degree of identity may nevertheless encode similar amino acid sequences due to the degeneracy of the genetic code. It is understood that changes in nucleic acid sequence can be made using this degeneracy to produce multiple nucleic acid sequences that all encode substantially the same protein.

The phrases “percent identity” and “% identity”, as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the hydrophobicity and acidity of the substituted residue, thus preserving the structure (and therefore function) of the folded polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.9 (May 7, 1999) with blastp set at default parameters. Such default parameters may be, for example:

Matrix: BLOSUM62

Open Gap: 11 and Extension Gap: 1 penalty

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in figures or Sequence Listings, may be used to describe a length over which percentage identity may be measured.

“Post-translational modification” of an SPTM may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu and the SPTM.

“Probe” refers to sptm or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the figures and Sequence Listing, may be used.

Methods for preparing and using probes and primers are described in the references, for example Sambrook et al., 1989, Molecular Cloning: A Laboratory Manual, 2^{nd} ed., vol. 1-3, Cold 30 Spring Harbor Press, Plainview N.Y.; Ausubel et al., 1987, Current Protocols in Molecular Biology, Greene Publ. Assoc. & Wiley-Intersciences, New York N.Y.; Innis et al., 1990, PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

“Purified” refers to molecules, either polynucleotides or polypeptides that are isolated or separated from their natural environment and are at least 60% free, preferably at least 75% free, and most preferably at least 90% free from other compounds with which they are naturally associated.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

“Regulatory element” refers to a nucleic acid sequence from nontranslated regions of a gene, and includes enhancers, promoters, introns, and 3+ untranslated regions, which interact with host proteins to carry out or regulate transcription or translation.

“Reporter” molecules are chemical or biochemical moieties used for labeling a nucleic acid, an amino acid, or an antibody. They include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

“Sample” is used in its broadest sense. Samples may contain nucleic or amino acids, antibodies, or other materials, and may be derived from any source (e.g., bodily fluids including, but not limited to, saliva, blood, and urine; chromosome(s), organelles, or membranes isolated from a cell; genomic DNA, RNA, or cDNA in solution or bound to a substrate; and cleared cells or tissues or blots or imprints from such cells or tissues).

“Specific binding” or “specifically binding” refers to the interaction between a protein or peptide and its agonist, antibody, antagonist, or other binding partner. The interaction is dependent upon the presence of a particular structure of the protein, e.g., the antigenic determinant or epitope, recognized by the binding molecule. For example, if an antibody is specific for epitope “A,” the presence of a polypeptide containing epitope A, or the presence of free unlabeled A, in a reaction containing free labeled A and the antibody will reduce the amount of labeled A that binds to the antibody.

“Substitution” refers to the replacement of at least one nucleotide or amino acid by a different nucleotide or amino acid.

“Substrate” refers to any suitable rigid or semi-rigid support including, e.g., membranes, filters, chips, slides, wafers, fibers, magnetic or nonmagnetic beads, gels, tubing, plates, polymers, microparticles or capillaries. The substrate can have a variety of surface forms, such as wells, trenches, pins, channels and pores, to which polynucleotides or polypeptides are bound.

A “transcript image” refers to the collective pattern of gene expression by a particular tissue or cell type under given conditions at a given time.

“Transformation” refers to a process by which exogenous DNA enters a recipient cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed.

“Transformants” include stably transformed cells in which the inserted DNA is capable of replication either as an autonomously replicating plasmid or as part of the host chromosome, as well as cells which transiently express inserted DNA or RNA.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, and plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 25% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 30%, at least 50%, at least 60%, at least 70%, at least 80%, at least 90%, at least 95% or even at least 98% or greater sequence identity over a certain defined length. The variant may result in “conservative” amino acid changes which do not affect structural and/or chemical properties. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

In an alternative, variants of the polynucleotides of the present invention may be generated through recombinant methods. One possible method is a DNA shuffling technique such as MOLECULTARBREEDING (Maxygen Inc., Santa Clara Calif.; described in U.S. Pat. No. 5,837,458; Chang, C. -C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of SPTM, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 95%, or at least 98% or greater sequence identity over a certain defined length of one of the polypeptides.

### THE INVENTION

In a particular embodiment, cDNA sequences derived from human tissues and cell lines were aligned based on nucleotide sequence identity and assembled into “consensus” or “template” sequences which are designated by the template identification numbers (template IDs) in column 2 of Table 1. The sequence identification numbers (SEQ ID NO:s) corresponding to the template IDs are shown in column 1. Segments of the template sequences are defined by the “start” and “stop” nucleotide positions listed in columns 3 and 4. These segments, when translated in the reading frames indicated in column 5, have similarity to signal peptide (SP) or transmembrane (TM) domain consensus sequences, as indicated in column 6.

The invention incorporates the nucleic acid sequences of these templates as disclosed in the Sequence Listing and the use of these sequences in the diagnosis and treatment of disease states characterized by defects in cell signaling. The invention further utilizes these sequences in hybridization and amplification technologies, and in particular, in technologies which assess gene expression patterns correlated with specific cells or tissues and their responses in vivo or in vitro to pharmaceutical agents, toxins, and other treatments. In this manner, the sequences of the present invention are used to develop a transcript image for a particular cell or tissue.

Derivation of Nucleic Acid Sequences

cDNA was isolated from libraries constructed using RNA derived from normal and diseased human tissues and cell lines. The human tissues and cell lines used for cDNA library construction were selected from a broad range of sources to provide a diverse population of cDNAs representative of gene transcription throughout the human body. Descriptions of the human tissues and cell lines used for cDNA library construction are provided in the LIFESEQ database (Incyte Genomics, Inc. (Incyte), Palo Alto Calif.). Human tissues were broadly selected from, for example, cardiovascular, dermatologic, endocrine, gastrointestinal, hematopoietic/immune system, musculoskeletal, neural, reproductive, and urologic sources.

Cell lines used for cDNA library construction were derived from, for example, leukemic cells, teratocarcinomas, neuroepitheliomas, cervical carcinoma, lung fibroblasts, and endothelial cells. Such cell lines include, for example, THP-1, Jurkat, HUVEC, hNT2, WI38, HeLa, and other cell lines commonly used and available from public depositories (American Type Culture Collection, Manassas Va.). Prior to mRNA isolation, cell lines were untreated, treated with a pharmaceutical agent such as 5′-aza-2′-deoxycytidine, treated with an activating agent such as lipopolysaccharide in the case of leukocytic cell lines, or, in the case of endothelial cell lines, subjected to shear stress.

Sequencing of the cDNAs

Methods for DNA sequencing are well known in the art. Conventional enzymatic methods employ the Klenow fragment of DNA polymerase I, SEQUENASE DNA polymerase (U.S. Biochemical Corporation, Cleveland Ohio), Taq polymerase (Applied Biosystems, Foster City Calif.), thermostable T7 polymerase (Amersham Pharmacia Biotech, Inc. (Amersham Pharmacia Biotech), Piscataway N.J.), or combinations of polymerases and proofreading exonucleases such as those found in the ELONGASE amplification system (Life Technologies Inc. (Life Technologies), Gaithersburg Md.), to extend the nucleic acid sequence from an oligonucleotide primer annealed to the DNA template of interest. Methods have been developed for the use of both single-stranded and double-stranded templates. Chain termination reaction products may be electrophoresed on urea-polyacrylamide gels and detected either by autoradiography (for radioisotope-labeled nucleotides) or by fluorescence (for fluorophore-labeled nucleotides). Automated methods for mechanized reaction preparation, sequencing, and analysis using fluorescence detection methods have been developed. Machines used to prepare cDNAs for sequencing can include the MICROLAB 2200 liquid transfer system (Hamilton Company (Hamilton), Reno Nev.), Peltier thermal cycler (PTC200; MJ Research, Inc. (MJ Research), Watertown Mass.), and ABI CATALYST 800 thermal cycler (Applied Biosystems). Sequencing can be carried out using, for example, the ABI 373 or 377 (Applied Biosystems) or MEGABACE 1000 (Molecular Dynamics, Inc. (Molecular Dynamics), Sunnyvale Calif.) DNA sequencing systems, or other automated and manual sequencing systems well known in the art.

The nucleotide sequences of the Sequence Listing have been prepared by current, state-of-the-art, automated methods and, as such, may contain occasional sequencing errors or unidentified nucleotides. Such unidentified nucleotides are designated by an N. These infrequent unidentified bases do not represent a hindrance to practicing the invention for those skilled in the art. Several methods employing standard recombinant techniques may be used to correct errors and complete the missing sequence information. (See, e.g., those described in Ausubel, F. M. et al. (1997) Short Protocols in Molecular Biology, John Wiley & Sons, New York N.Y.; and Sambrook, J. et al. (1989) Molecular Cloning A Laboratory Manual, Cold Spring Harbor Press, Plainview N.Y.)

Assembly of cDNA Sequences

Human polynucleotide sequences may be assembled using programs or algorithms well known in the art. Sequences to be assembled are related, wholly or in part, and may be derived from a single or many different transcripts. Assembly of the sequences can be performed using such programs as PHRAP (Phils Revised Assembly Program) and the GELVIEW fragment assembly system (GCG), or other methods known in the art.

Alternatively, cDNA sequences are used as “component” sequences that are assembled into “template” or “consensus” sequences as follows. Sequence chromatograms are processed, verified, and quality scores are obtained using PHRED. Raw sequences are edited using an editing pathway known as Block 1 (See, e.g., the LIFESEQ Assembled User Guide, Incyte Genomics, Palo Alto, Calif.). A series of BLAST comparisons is performed and low-information segments and repetitive elements (e.g., dinucleotide repeats, Alu repeats, etc.) are replaced by “n's”, or masked, to prevent spurious matches. Mitochondrial and ribosomal RNA sequences are also removed. The processed sequences are then loaded into a relational database management system (RDMS) which assigns edited sequences to existing templates, if available. When additional sequences are added into the RDMS, a process is initiated which modifies existing templates or creates new templates from works in progress (i.e., nonfinal assembled sequences) containing queued sequences or the sequences themselves. After the new sequences have been assigned to templates, the templates can be merged into bins. If multiple templates exist in one bin, the bin can be split and the templates reannotated.

Once gene bins have been generated based upon sequence alignments, bins are “clone joined” based upon clone information. Clone joining occurs when the 5′ sequence of one clone is present in one bin and the 3′ sequence from the same clone is present in a different bin, indicating that the two bins should be merged into a single bin. Only bins which share at least two different clones are merged.

A resultant template sequence may contain either a partial or a full length open reading frame, or all or part of a genetic regulatory element. This variation is due in part to the fact that the full length cDNAs of many genes are several hundred, and sometimes several thousand, bases in length. With current technology, cDNAs comprising the coding regions of large genes cannot be cloned because of vector limitations, incomplete reverse transcription of the mRNA, or incomplete “second strand” synthesis. Template sequences may be extended to include additional contiguous sequences derived from the parent RNA transcript using a variety of methods known to those of skill in the art. Extension may thus be used to achieve the full length coding sequence of a gene.

Analysis of the cDNA Sequences

The cDNA sequences are analyzed using a variety of programs and algorithms which are well known in the art. (See, e.g., Ausubel, 1997, supra, Chapter 7.7; Meyers, R. A. (Ed.) (1995) Molecular Biology and Biotechnology, Wiley VCH, New York N.Y., pp. 856-853; and Table 4.) These analyses comprise both reading frame determinations, e.g., based on triplet codon periodicity for particular organisms (Fickett, J. W. (1982) Nucleic Acids Res. 10:5303-5318); analyses of potential start and stop codons; and homology searches.

Computer programs known to those of skill in the art for performing computer-assisted searches for amino acid and nucleic acid sequence similarity, include, for example, Basic Local Alignment Search Tool (BLAST; Altschul, S. F. (1993) J. Mol. Evol. 36:290-300; Altschul, S. F. et al. (1990) J. Mol. Biol. 215:403-410). BLAST is especially useful in determining exact matches and comparing two sequence fragments of arbitrary but equal lengths, whose alignment is locally maximal and for which the alignment score meets or exceeds a threshold or cutoff score set by the user (Karlin, S. et al. (1988) Proc. Natl. Acad. Sci. USA 85:841-845). Using an appropriate search tool (e.g., BLAST or HMM), GenBank, SwissProt, BLOCKS, PFAM and other databases may be searched for sequences containing regions of homology to a query sptm or SPTM of the present invention.

Other approaches to the identification, assembly, storage, and display of nucleotide and polypeptide sequences are provided in “Relational Database for Storing Biomolecule Information,” U.S. Ser. No. 08/947,845, filed Oct. 9, 1997; “Project-Based Full-Length Biomolecular Sequence Database,” U.S. Ser. No. 08/811,758, filed Mar. 6, 1997; and “Relational Database and System for Storing Information Relating to Biomolecular Sequences,” U.S. Ser. No. 09/034,807, filed Mar. 4, 1998, all of which are incorporated by reference herein in their entirety.

Protein hierarchies can be assigned to the putative encoded polypeptide based on, e.g., motif, BLAST, or biological analysis. Methods for assigning these hierarchies are described, for example, in “Database System Employing Protein Function Hierarchies for Viewing Biomolecular Sequence Data,” U.S. Ser. No. 08/812,290, filed Mar. 6, 1997, incorporated herein by reference.

Human Secretory Sequences

The sptm of the present invention may be used for a variety of diagnostic and therapeutic purposes. For example, an sptm may be used to diagnose a particular condition, disease, or disorder associated with cell signaling. Such conditions, diseases, and disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an immune system disorder such as such as inflammation, actinic keratosis, acquired immunodeficiency syndrome (AIDS), Addison's disease, adult respiratory distress syndrome, allergies, ankylosing spondylitis, amyloidosis, anemia, arteriosclerosis, asthma, atherosclerosis, autoimmune hemolytic anemia, autoimmune thyroiditis, bronchitis, bursitis, cholecystitis, cirrhosis, contact dermatitis, Crohn's disease, atopic dermatitis, dermatomyositis, diabetes mellitus, emphysema, erythroblastosis fetalis, erythema nodosum, atrophic gastritis, glomerulonephritis, Goodpasture's syndrome, gout, Graves' disease, Hashimoto's thyroiditis, paroxysmal nocturnal hemoglobinuria, hepatitis, hypereosinophilia, irritable bowel syndrome, episodic lymphopenia with lymphocytotoxins, mixed connective tissue disease (MCTD), multiple sclerosis, myasthenia gravis, myocardial or pericardial inflammation, myelofibrosis, osteoarthritis, osteoporosis, pancreatitis, polycythemia vera, polymyositis, psoriasis, Reiter's syndrome, rheumatoid artritis, scleroderma, Sjögren's syndrome, systemic anaphylaxis, systemic lupus erythematosus, systemic sclerosis, primary thrombocythemia, thrombocytopenic purpura, ulcerative colitis, uveitis, Werner syndrome, complications of cancer, hemodialysis, and extracorporeal circulation, trauma, and hematopoietic cancer including lymphoma, leukemia, and myeloma; and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorder of the central nervous system, cerebral palsy, a neuroskeletal disorder, an autonomic nervous system disorder, a cranial nerve disorder, a spinal cord disease, muscular dystrophy and other neuromuscular disorder, a peripheral nervous system disorder, dermatomyositis and polymyositis, inherited, metabolic, endocrine, and toxic myopathy, myasthenia gravis, periodic paralysis, a mental disorder including mood, anxiety, and schizophrenic disorder, seasonal affective disorder (SAD), akathesia, amnesia, catatonia, diabetic neuropathy, tardive dyskinesia, dystonias, paranoid psychoses, postherpetic neuralgia, and Tourette's disorder. The sptm can be used to detect the presence of, or to quantify the amount of, an sptm-related polynucleotide in a sample. This information is then compared to information obtained from appropriate reference samples, and a diagnosis is established. Alternatively, a polynucleotide complementary to a given sptm can inhibit or inactivate a therapeutically relevant gene related to the sptm.

Analysis of sptm Expression Patterns

The expression of sptm may be routinely assessed by hybridization-based methods to determine, for example, the tissue-specificity, disease-specificity, or developmental stage-specificity of sptm expression. For example, the level of expression of sptm may be compared among different cell types or tissues, among diseased and normal cell types or tissues, among cell types or tissues at different developmental stages, or among cell types or tissues undergoing various treatments. This type of analysis is useful, for example, to assess the relative levels of sptm expression in fully or partially differentiated cells or tissues, to determine if changes in sptm expression levels are correlated with the development or progression of specific disease states, and to assess the response of a cell or tissue to a specific therapy, for example, in pharmacological or toxicological studies. Methods for the analysis of sptm expression are based on hybridization and amplification technologies and include membrane-based procedures such as northern blot analysis, high-throughput procedures that utilize, for example, microarrays, and PCR-based procedures.

Hybridization and Genetic Analysis

The sptm, their fragments, or complementary sequences, may be used to identify the presence of and/or to determine the degree of similarity between two (or more) nucleic acid sequences. The sptm may be hybridized to naturally occurring or recombinant nucleic acid sequences under appropriately selected temperatures and salt concentrations. Hybridization with a probe based on the nucleic acid sequence of at least one of the sptm allows for the detection of nucleic acid sequences, including genomic sequences, which are identical or related to the sptm of the Sequence Listing. Probes may be selected from non-conserved or unique regions of at least one of the polynucleotides of SEQ ID NO: 1-79 and tested for their ability to identify or amplify the target nucleic acid sequence using standard protocols.

Polynucleotide sequences that are capable of hybridizing, in particular, to those shown in SEQ ID NO: 1-79 and fragments thereof, can be identified using various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions are discussed in “Definitions.”

A probe for use in Southern or northern hybridization may be derived from a fragment of an sptm sequence, or its complement, that is up to several hundred nucleotides in length and is either single-stranded or double-stranded. Such probes may be hybridized in solution to biological materials such as plasmids, bacterial, yeast, or human artificial chromosomes, cleared or sectioned tissues, or to artificial substrates containing sptm. Microarrays are particularly suitable for identifying the presence of and detecting the level of expression for multiple genes of interest by examining gene expression correlated with, e.g., various stages of development, treatment with a drug or compound, or disease progression. An array analogous to a dot or slot blot may be used to arrange and link polynucleotides to the surface of a substrate using one or more of the following: mechanical (vacuum), chemical, thermal, or UV bonding procedures. Such an array may contain any number of sptm and may be produced by hand or by using available devices, materials, and machines.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.)

Probes may be labeled by either PCR or enzymatic techniques using a variety of commercially available reporter molecules. For example, commercial kits are available for radioactive and chemiluminescent labeling (Amersham Pharmacia Biotech) and for alkaline phosphatase labeling (Life Technologies). Alternatively, sptm may be cloned into commercially available vectors for the production of RNA probes. Such probes may be transcribed in the presence of at least one labeled nucleotide (e.g., ³²P-ATP, Amersham Pharmacia Biotech).

Additionally the polynucleotides of SEQ ID NO: 1-79 or suitable fragments thereof can be used to isolate full length cDNA sequences utilizing hybridization and/or amplification procedures well known in the art, e.g., cDNA library screening, PCR amplification, etc. The molecular cloning of such fall length cDNA sequences may employ the method of cDNA library screening with probes using the hybridization, stringency, washing, and probing strategies described above and in Ausubel, supra, Chapters 3, 5, and 6. These procedures may also be employed with genomic libraries to isolate genomic sequences of sptm in order to analyze, e.g., regulatory elements.

Genetic Mapping

Gene identification and mapping are important in the investigation and treatment of almost all conditions, diseases, and disorders. Cancer, cardiovascular disease, Alzheimer's disease, arthritis, diabetes, and mental illnesses are of particular interest. Each of these conditions is more complex than the single gene defects of sickle cell anemia or cystic fibrosis, with select groups of genes being predictive of predisposition for a particular condition, disease, or disorder. For example, cardiovascular disease may result from malfunctioning receptor molecules that fail to clear cholesterol from the bloodstream, and diabetes may result when a particular individual's immune system is activated by an infection and attacks the insulin-producing cells of the pancreas. In some studies, Alzheimer's disease has been linked to a gene on chromosome 21; other studies predict a different gene and location. Mapping of disease genes is a complex and reiterative process and generally proceeds from genetic linkage analysis to physical mapping.

As a condition is noted among members of a family, a genetic linkage map traces parts of chromosomes that are inherited in the same pattern as the condition. Statistics link the inheritance of particular conditions to particular regions of chromosomes, as defined by RFLP or other markers. (See, for example, Lander, E. S. and Botstein, D. (1986) Proc. Natl. Acad. Sci. USA 83:7353-7357.) Occasionally, genetic markers and their locations are known from previous studies. More often, however, the markers are simply stretches of DNA that differ among individuals. Examples of genetic linkage maps can be found in various scientific journals or at the Online Mendelian Inheritance in Man (OMIM) World Wide Web site.

In another embodiment of the invention, sptm sequences may be used to generate hybridization probes useful in chromosomal mapping of naturally occurring genomic sequences. Either coding or noncoding sequences of sptm may be used, and in some instances, noncoding sequences may be preferable over coding sequences. For example, conservation of an sptm coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data. (See, e.g., Meyers, supra, pp. 965-968.) Correlation between the location of sptm on a physical chromosomal map and a specific disorder, or a predisposition to a specific disorder, may help define the region of DNA associated with that disorder. The sptm sequences may also be used to detect polymorphisms that are genetically linked to the inheritance of a particular condition, disease, or disorder.

In situ hybridization of chromosomal preparations and genetic mapping techniques, such as linkage analysis using established chromosomal markers, may be used for extending existing genetic maps. Often the placement of a gene on the chromosome of another mammalian species, such as mouse, may reveal associated markers even if the number or arm of the corresponding human chromosome is not known. These new marker sequences can be mapped to human chromosomes and may provide valuable information to investigators searching for disease genes using positional cloning or other gene discovery techniques. Once a disease or syndrome has been crudely correlated by genetic linkage with a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequences of the subject invention may also be used to detect differences in chromosomal architecture due to translocation, inversion, etc., among normal, carrier, or affected individuals.

Once a disease-associated gene is mapped to a chromosomal region, the gene must be cloned in order to identify mutations or other alterations (e.g., translocations or inversions) that may be correlated with disease. This process requires a physical map of the chromosomal region containing the disease-gene of interest along with associated markers. A physical map is necessary for determining the nucleotide sequence of and order of marker genes on a particular chromosomal region. Physical mapping techniques are well known in the art and require the generation of overlapping sets of cloned DNA fragments from a particular organelle, chromosome, or genome. These clones are analyzed to reconstruct and catalog their order. Once the position of a marker is determined, the DNA from that region is obtained by consulting the catalog and selecting clones from that region. The gene of interest is located through positional cloning techniques using hybridization or similar methods.

Diagnostic Uses

The sptm of the present invention may be used to design probes useful in diagnostic assays. Such assays, well known to those skilled in the art, may be used to detect or confirm conditions, disorders, or diseases associated with abnormal levels of sptm expression. Labeled probes developed from sptm sequences are added to a sample under hybridizing conditions of desired stringency. In some instances, sptm, or fragments or oligonucleotides derived from sptm, may be used as primers in amplification steps prior to hybridization. The amount of hybridization complex formed is quantified and compared with standards for that cell or tissue. If sptm expression varies significantly from the standard, the assay indicates the presence of the condition, disorder, or disease. Qualitative or quantitative diagnostic methods may include northern, dot blot, or other membrane or dip-stick based technologies or multiple-sample format technologies such as PCR, enzyme-linked immunosorbent assay (ELISA)-like, pin, or chip-based assays.

The probes described above may also be used to monitor the progress of conditions, disorders, or diseases associated with abnormal levels of sptm expression, or to evaluate the efficacy of a particular therapeutic treatment. The candidate probe may be identified from the sptm that are specific to a given human tissue and have not been observed in GenBank or other genome databases. Such a probe may be used in animal studies, preclinical tests, clinical trials, or in monitoring the treatment of an individual patient. In a typical process, standard expression is established by methods well known in the art for use as a basis of comparison, samples from patients affected by the disorder or disease are combined with the probe to evaluate any deviation from the standard profile, and a therapeutic agent is administered and effects are monitored to generate a treatment profile. Efficacy is evaluated by determining whether the expression progresses toward or returns to the standard normal pattern. Treatment profiles may be generated over a period of several days or several months. Statistical methods well known to those skilled in the art may be use to determine the significance of such therapeutic agents.

The polynucleotides are also useful for identifying individuals from minute biological samples, for example, by matching the RFLP pattern of a sample's DNA to that of an individual's DNA. The polynucleotides of the present invention can also be used to determine the actual base-by-base DNA sequence of selected portions of an individual's genome. These sequences can be used to prepare PCR primers for amplifying and isolating such selected DNA, which can then be sequenced. Using this technique, an individual can be identified through a unique set of DNA sequences. Once a unique ID database is established for an individual, positive identification of that individual can be made from extremely small tissue samples.

In a particular aspect, oligonucleotide primers derived from the sptm of the invention may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from sptm are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequences of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

DNA-based identification techniques are critical in forensic technology. DNA sequences taken from very small biological samples such as tissues, e.g., hair or skin, or body fluids, e.g., blood, saliva, semen, etc., can be amplified using, e.g., PCR, to identify individuals. (See, e.g., Erlich, H. (1992) PCR Technology, Freeman and Co., New York, N.Y.). Similarly, polynucleotides of the present invention can be used as polymorphic markers.

There is also a need for reagents capable of identifying the source of a particular tissue. Appropriate reagents can comprise, for example, DNA probes or primers prepared from the sequences of the present invention that are specific for particular tissues. Panels of such reagents can identify tissue by species and/or by organ type. In a similar fashion, these reagents can be used to screen tissue cultures for contamination.

The polynucleotides of the present invention can also be used as molecular weight markers on nucleic acid gels or Southern blots, as diagnostic probes for the presence of a specific mRNA in a particular cell type, in the creation of subtracted cDNA libraries which aid in the discovery of novel polynucleotides, in selection and synthesis of oligomers for attachment to an array or other support, and as an antigen to elicit an immune response.

Disease Model Systems Using sptm

The polynucleotides encoding SPTM or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capeechi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

The polynucleotides encoding SPTM may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

The polynucleotides encoding SPTM of the invention can also be used to create “knockin” humanized animals (pigs) or transgenic animals (mice or rats) to model human disease. With knockin technology, a region of sptm is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress sptm, resulting, e.g., in the secretion of SPTM in its milk, may also serve as a convenient source of that protein (Janne, J. et al. (1998) Biotechnol. Annu. Rev. 4:55-74).

Screening Assays

SPTM encoded by polynucleotides of the present invention may be used to screen for molecules that bind to or are bound by the encoded polypeptides. The binding of the polypeptide and the molecule may activate (agonist), increase, inhibit (antagonist), or decrease activity of the polypeptide or the bound molecule. Examples of such molecules include antibodies, oligonucleotides, proteins (e.g., receptors), or small molecules.

Preferably, the molecule is closely related to the natural ligand of the polypeptide, e.g., a ligand or fragment thereof, a natural substrate, or a structural or functional mimetic. (See, Coligan et al., (1991) Current Protocols in Immunology 1(2): Chapter 5.) Similarly, the molecule can be closely related to the natural receptor to which the polypeptide binds, or to at least a fragment of the receptor, e.g., the active site. In either case, the molecule can be rationally designed using known techniques. Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide or cell membrane fractions which contain the expressed polypeptide are then contacted with a test compound and binding, stimulation, or inhibition of activity of either the polypeptide or the molecule is analyzed.

An assay may simply test binding of a candidate compound to the polypeptide, wherein binding is detected by a fluorophore, radioisotope, enzyme conjugate, or other detectable label. Alternatively, the assay may assess binding in the presence of a labeled competitor.

Additionally, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay using, e.g., a monoclonal or polyclonal antibody, can measure polypeptide level in a sample. The antibody can measure polypeptide level by either binding, directly or indirectly, to the polypeptide or by competing with the polypeptide for a substrate.

All of the above assays can be used in a diagnostic or prognostic context. The molecules discovered using these assays can be used to treat disease or to bring about a particular result in a patient (e.g., blood vessel growth) by activating or inhibiting the polypeptide/molecule. Moreover, the assays can discover agents which may inhibit or enhance the production of the polypeptide from suitably manipulated cells or tissues.

Transcript Imaging and Toxicological Testing

Another embodiment relates to the use of sptm to develop a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,940,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity pertaining to cell signaling.

Transcript images which profile sptm expression may be generated using transcripts isolated from tissues, cell lines, biopsies, or other biological samples. The transcript image may thus reflect sptm expression in vivo, as in the case of a tissue or biopsy sample, or in vitro, as in the case of a cell line.

Transcript images which profile sptm expression may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and Anderson, N. L. (2000) Toxicol. Lett. 112-113:467-71, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of SPTM encoded by polynucleotides of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomnic profile may also be generated using antibodies specific for SPTM to quantify the levels of SPTM expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-11; Mendoze, L. G. et al. (1999) Biotechniques 27:778-88). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and Seilhamer, J. (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the SPTM encoded by polynucleotides of the present invention.

In another embodiment, the toxicity of a test compound is assessed by treating a biological sample containing proteins with the test compound. Proteins from the biological sample are incubated with antibodies specific to the SPTM encoded by polynucleotides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Transcript images may be used to profile sptm expression in distinct tissue types. This process can be used to determine cell signaling activity in a particular tissue type relative to this activity in a different tissue type. Transcript images may be used to generate a profile of sptm expression characteristic of diseased tissue. Transcript images of tissues before and after treatment may be used for diagnostic purposes, to monitor the progression of disease, and to monitor the efficacy of drug treatments for diseases which affect cell signaling activity.

Transcript images of cell lines can be used to assess cell signaling activity and/or to identify cell lines that lack or misregulate this activity. Such cell lines may then be treated with pharmaceutical agents, and a transcript image following treatment may indicate the efficacy of these agents in restoring desired levels of this activity. A similar approach may be used to assess the toxicity of pharmaceutical agents as reflected by undesirable changes in cell signaling activity. Candidate pharmaceutical agents may be evaluated by comparing their associated transcript images with those of pharmaceutical agents of known effectiveness.

Antisense Molecules

The polynucleotides of the present invention are useful in antisense technology. Antisense technology or therapy relies on the modulation of expression of a target protein through the specific binding of an antisense sequence to a target sequence encoding the target protein or directing its expression. (See, e.g., Agrawal, S., ed. (1996) Antisense TheraDeutics, Humana Press Inc., Totawa N.J.; Alama, A. et al. (1997) Pharmacol. Res. 36(3):171-178; Crooke, S. T. (1997) Adv. Pharmacol. 40:1-49; Sharma, H. W. and R. Narayanan (1995) Bioessays 17(12):1055-1063; and Lavrosky, Y. et al. (1997) Biochem. Mol. Med. 62(l):11-22.) An antisense sequence is a polynucleotide sequence capable of specifically hybridizing to at least a portion of the target sequence. Antisense sequences bind to cellular mRNA and/or genomic DNA, affecting translation and/or transcription. Antisense sequences can be DNA, RNA, or nucleic acid mimics and analogs. (See, e.g., Rossi, J. J. et al. (1991) Antisense Res. Dev. 1(3):285-288; Lee, R. et al. (1998) Biochemistry 37(3):900-1010; Pardridge, W. M. et al. (1995) Proc. Natl. Acad. Sci. USA 92(12):5592-5596; and Nielsen, P. E. and Haaima, G. (1997) Chem. Soc. Rev. 96:73-78.) Typically, the binding which results in modulation of expression occurs through hybridization or binding of complementary base pairs. Antisense sequences can also bind to DNA duplexes through specific interactions in the major groove of the double helix.

The polynucleotides of the present invention and fragments thereof can be used as antisense sequences to modify the expression of the polypeptide encoded by sptm. The antisense sequences can be produced ex vivo, such as by using any of the ABI nucleic acid synthesizer series (Applied Biosystems) or other automated systems known in the art. Antisense sequences can also be produced biologically, such as by transforming an appropriate host cell with an expression vector containing the sequence of interest. (See, e.g., Agrawal, supra.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E., et al. (1998) J. Allergy Clin. Immunol. 102(3):469-475; and Scanlon, K. J., et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, F. M. et al. (1995) Current Protocols in Molecular Biology, John Wiley & Sons, New York N.Y.; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

Expression

In order to express a biologically active SPTM, the nucleotide sequences encoding SPTM or fragments thereof may be inserted into an appropriate expression vector, i.e., a vector which contains the necessary elements for transcriptional and translational control of the inserted coding sequence in a suitable host. Methods which are well known to those skilled in the art may be used to construct expression vectors containing sequences encoding SPTM and appropriate transcriptional and translational control elements. These methods include in vitro recombinant DNA techniques, synthetic techniques, and in vivo genetic recombination. (See, e.g., Sambrook, supra, Chapters 4, 8, 16, and 17; and Ausubel, supra, Chapters 9, 10, 13, and 16.)

A variety of expression vector/host systems may be utilized to contain and express sequences encoding SPTM. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal (mammalian) cell systems. (See, e.g., Sambrook, supra; Ausubel, 1995, supra, Van Heece, G. and S. M. Schuster (1989) J. Biol. Chem. 264:5503-5509; Bitter, G. A. et al. (1987) Methods Enzymol. 153:516-544; Scorer, C. A. et al. (1994) Bio/Technology 12:181-184; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; Coruzzi, G. et al. (1984) EMBO J. 3:1671-1680; Broglie, R. et al. (1984) Science 224:838-843; Winter, J. et al. (1991) Results Probl. Cell Differ. 17:85-105; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al., (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

For long term production of recombinant proteins in mammalian systems, stable expression of SPTM in cell lines is preferred. For example, sequences encoding SPTM can be transformed into cell lines using expression vectors which may contain viral origins of replication and/or endogenous expression elements and a selectable marker gene on the same or on a separate vector. Any number of selection systems may be used to recover transformed cell lines. (See, e.g., Wigler, M. et al. (1977) Cell 11:223-232; Lowy, I. et al. (1980) Cell 22:817-823.; Wigler, M. et al. (1980) Proc. Natl. Acad. Sci. USA 77:3567-3570; Colbere-Garapin, F. et al. (1981) J. Mol. Biol. 150:1-14; Hartman, S. C. and R. C. Mulligan (1988) Proc. Natl. Acad. Sci. USA 85:8047-8051; Rhodes, C. A. (1995) Methods Mol. Biol. 55:121-131.)

Therapeutic Uses of sptm

The polynucleotides encoding SPTM of the invention may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassemias, familial hypercholesterolemia, and hemophilia resulting from Factor VIII or Factor IX deficiencies (Crystal, R. G. (1995) Science 270:404-410; Verma, I. M. and Somia, N. (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA. 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in sptm expression or regulation causes disease, the expression of sptm from an appropriate population of transduced cells may alleviate the clinical manifestations caused by the genetic deficiency.

In a further embodiment of the invention, diseases or disorders caused by deficiencies in sptm are treated by constructing mammalian expression vectors comprising sptm and introducing these vectors by mechanical means into sptm-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and Anderson, W. F. (1993) Annu. Rev. Biochem. 62:191-217;

Ivics, Z. (1997) Cell 91:501-510; Boulay, J -L. and Récipon, H. (1998) Curr. Opin. Biotechnol. 9:445-450).

Expression vectors that may be effective for the expression of sptm include, but are not limited to, the PCDNA 3.1, EPITAG, PRCCMV2, PREP, PVAX vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). The sptm of the invention may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and Bujard, H. (1992) Proc. Natl. Acad. Sci.

U.S.A. 89:5547-5551; Gossen, M. et al., (1995) Science 268:1766-1769; Rossi, F. M. V. and Blau, H. M. (1998) Curr. Opin. Biotechnol. 9:451456), commercially available in the T-REX plasmid (Invitrogen); the ecdysone-inducible promoter (available in the plasmids PVGRXR and PIND;

Invitrogen); the FK506/rapamycin inducible promoter; or the RU486/mifepristone inducible promoter (Rossi, F. M. V. and Blau, H. M. supra), or (iii) a tissue-specific promoter or the native promoter of the endogenous gene encoding SPTM from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and Eb, A. J. (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to sptm expression are treated by constructing a retrovirus vector consisting of (i) sptm under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. U.S.A. 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and Miller, A. D. (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. U.S.A. 95:1201-1206; Su, L. (1997) Blood 89:2283-2290).

In the alternative, an adenovirus-based gene therapy delivery system is used to deliver sptm to cells which have one or more genetic abnormalities with respect to the expression of sptm. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and Somia, N. (1997) Nature 18:389:239-242, both incorporated by reference herein.

In another alternative, a herpes-based, gene therapy delivery system is used to deliver sptm to target cells which have one or more genetic abnormalities with respect to the expression of sptm. The use of herpes simplex virus (HSV)-based vectors may be especially valuable for introducing sptm to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res.169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. 1999 J. Virol. 73:519-532 and Xu, H. et al., (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver sptm to target cells. The biology of the prototypic alphavirus, Semliki Forest Virus (SFV), has been studied extensively and gene transfer vectors have been based on the SFV genome (Garoff, H. and Li, K -J. (1998) Curr. Opin. Biotech. 9:464469). During alphavirus RNA replication, a subgenomic RNA is generated that normally encodes the viral capsid proteins. This subgenomic RNA replicates to higher levels than the full-length genomic RNA, resulting in the overproduction of capsid proteins relative to the viral proteins with enzymatic activity (e.g., protease and polymerase). Similarly, inserting sptm into the alphavirus genome in place of the capsid-coding region results in the production of a large number of sptm RNAs and the synthesis of high levels of SPTM in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphavirus can be altered to suit the needs of the gene therapy application (Dryga, S. A. et al. (1997) Virology 228:74-83). The wide host range of alphaviruses will allow the introduction of sptm into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Antibodies

Anti-SPTM antibodies may be used to analyze protein expression levels. Such antibodies include, but are not limited to, polyclonal, monoclonal, chimeric, single chain, and Fab fragments. For descriptions of and protocols of antibody technologies, see, e.g., Pound J. D. (1998) Immunochemical Protocols, Humana Press, Totowa, N.J.

The amino acid sequence encoded by the sptm of the Sequence Listing may be analyzed by appropriate software (e.g., LASERGENE NAVIGATOR software, DNASTAR) to determine regions of high immunogenicity. The optimal sequences for immunization are selected from the C-terminus, the N-terminus, and those intervening, hydrophilic regions of the polypeptide which are likely to be exposed to the external environment when the polypeptide is in its natural conformation. Analysis used to select appropriate epitopes is also described by Ausubel (1997, supra, Chapter 11.7). Peptides used for antibody induction do not need to have biological activity; however, they must be antigenic. Peptides used to induce specific antibodies may have an amino acid sequence consisting of at least five amino acids, preferably at least 10 amino acids, and most preferably at least 15 amino acids. A peptide which mimics an antigenic fragment of the natural polypeptide may be fused with another protein such as keyhole limpet hemocyanin (KLH; Sigma, St. Louis Mo.) for antibody production. A peptide encompassing an antigenic region may be expressed from an sptm, synthesized as described above, or purified from human cells.

Procedures well known in the art may be used for the production of antibodies. Various hosts including mice, goats, and rabbits, may be immunized by injection with a peptide. Depending on the host species, various adjuvants may be used to increase immunological response.

In one procedure, peptides about 15 residues in length may be synthesized using an ABI 431A peptide synthesizer (Applied Biosystems) using fmoc-chemistry and coupled to KLH (Sigma) by reaction with M-maleimidobenzoyl-N-hydroxysuccinimide ester (Ausubel, 1995, supra). Rabbits are immunized with the peptide-KLH complex in complete Freund's adjuvant. The resulting antisera are tested for antipeptide activity by binding the peptide to plastic, blocking with 1% bovine serum albumin (BSA), reacting with rabbit antisera, washing, and reacting with radioiodinated goat anti-rabbit IgG. Antisera with antipeptide activity are tested for anti-SPTM activity using protocols well known in the art, including ELISA, radioimmunoassay (RIA), and immunoblotting.

In another procedure, isolated and purified peptide may be used to immunize mice (about 100 μg of peptide) or rabbits (about 1 mg of peptide). Subsequently, the peptide is radioiodinated and used to screen the immunized animals' B-lymphocytes for production of antipeptide antibodies. Positive cells are then used to produce hybridomas using standard techniques. About 20 mg of peptide is sufficient for labeling and screening several thousand clones. Hybridomas of interest are detected by screening with radioiodinated peptide to identify those fusions producing peptide-specific monoclonal antibody. In a typical protocol, wells of a multi-well plate (FAST, Becton-Dickinson, Palo Alto, Calif.) are coated with affinity-purified, specific rabbit-anti-mouse (or suitable anti-species IgG) antibodies at 10 mg/ml. The coated wells are blocked with 1% BSA and washed and exposed to supernatants from hybridomas. After incubation, the wells are exposed to radiolabeled peptide at 1 mg/ml.

Clones producing antibodies bind a quantity of labeled peptide that is detectable above background. Such clones are expanded and subjected to 2 cycles of cloning. Cloned hybridomas are injected into pristane-treated mice to produce ascites, and monoclonal antibody is purified from the ascitic fluid by affinity chromatography on protein A (Amersham Pharmacia Biotech). Several procedures for the production of monoclonal antibodies, including in vitro production, are described in Pound (supra). Monoclonal antibodies with antipeptide activity are tested for anti-SPTM activity using protocols well known in the art, including ELISA, RIA, and immunoblotting.

Antibody fragments containing specific binding sites for an epitope may also be generated. For example, such fragments include, but are not limited to, the F(ab′)2 fragments produced by pepsin digestion of the antibody molecule, and the Fab fragments generated by reducing the disulfide bridges of the F(ab′)2 fragments. Alternatively, construction of Fab expression libraries in filamentous bacteriophage allows rapid and easy identification of monoclonal fragments with desired specificity (Pound, supra, Chaps. 45-47). Antibodies generated against polypeptide encoded by sptm can be used to purify and characterize full-length SPTM protein and its activity, binding partners, etc.

Assays Using Antibodies

Anti-SPTM antibodies may be used in assays to quantify the amount of SPTM found in a particular human cell. Such assays include methods utilizing the antibody and a label to detect expression level under normal or disease conditions. The peptides and antibodies of the invention may be used with or without modification or labeled by joining them, either covalently or noncovalently, with a reporter molecule.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the SPTM and its specific antibody and the measurement of such complexes. These and other assays are described in Pound (supra).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, including U.S. Ser. No. 60/205,287, U.S. Ser. No. 60/205,324, U.S. Ser. No. 60/205,286, U.S. Ser. No. 60/205,323, U.S. Ser. No. 60/185,215, U.S. Ser. No. 60/185,216, and U.S. Ser. No. 60/205,232, are hereby expressly incorporated by reference.

### EXAMPLES

I. Construction of cDNA Libraries

RNA was purchased from CLONTECH Laboratories, Inc. (Palo Alto Calif. ) or isolated from various tissues. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated with either isopropanol or sodium acetate and ethanol, or by other routine methods.

Phenol extraction and precipitation of RNA were repeated as necessary to increase RNA purity. In most cases, RNA was treated with DNase. For most libraries, poly(A+) RNA was isolated using oligo d(T)-coupled paramagnetic particles (Promega Corporation (Promega), Madison Wis.), OLIGOTEX latex particles (QIAGEN, Inc. (QIAGEN), Valencia Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Inc., Austin Tex.).

In some cases, Stratagene was provided with RNA and constructed the corresponding cDNA libraries. Otherwise, cDNA was synthesized and cDNA libraries were constructed with the UNIZAP vector system (Stratagene Cloning Systems, Inc. (Stratagene), La Jolla Calif.) or SUPERSCRIPT plasmid system (Life Technologies), using the recommended procedures or similar methods known in the art. (See, e.g., Ausubel, 1997, supra, Chapters 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL SI 000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PCDNA2.1 plasmid (Invitrogen, Carlsbad Calif.), PBK-CMV plasmid (Stratagene), or pINCY (Incyte Genomics, Palo Alto Calif.), or derivatives thereof. Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids were recovered from host cells by in vivo excision using the UNIZAP vector system (Stratagene) or by cell lysis. Plasmids were purified using at least one of the following: the Magic or WIZARD Minipreps DNA purification system (Promega); the AGTC Miniprep purification kit (Edge BioSystems, Gaithersburg Md.); and the QIAWELL 8, QIAWELL 8 Plus, and QIAWELL 8 Ultra plasmid purification systems or the R.E.A.L. PREP 96 plasmid purification kit (QIAGEN). Following precipitation, plasmids were resuspended in 0.1 ml of distilled water and stored, with or without lyophilization, at 4° C.

Alternatively, plasmid DNA was amplified from host cell lysates using direct link PCR in a high-throughput format. (Rao, V. B. (1994) Anal. Biochem. 216:1-14.) Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Inc. (Molecular Probes), Eugene Oreg.) and a FLUOROSKAN II fluorescence scanner (Labsystems Oy, Helsinki, Finland).

III. Sequencing and Analysis

cDNA sequencing reactions were processed using standard methods or high-throughput instrumentation such as the ABI CATALYST 800 thermal cycler (Applied Biosystems) or the PTC-200 thermal cycler (MJ Research) in conjunction with the HYDRA microdispenser (Robbins Scientific Corp., Sunnyvale Calif.) or the MICROLAB 2200 liquid transfer system (Hamilton). cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, Chapter 7.7). Some of the cDNA sequences were selected for extension using the techniques disclosed in Example VIII.

IV. Assembly and Analysis of Sequences

Component sequences from chromatograms were subject to PHRED analysis and assigned a quality score. The sequences having at least a required quality score were subject to various pre-processing editing pathways to eliminate, e.g., low quality 3′ ends, vector and linker sequences, polyA tails, Alu repeats, mitochondrial and ribosomal sequences, bacterial contamination sequences, and sequences smaller than 50 base pairs. In particular, low-information sequences and repetitive elements (e.g., dinucleotide repeats, Alu repeats, etc.) were replaced by “n's”, or masked, to prevent spurious matches.

Processed sequences were then subject to assembly procedures in which the sequences were assigned to gene bins (bins). Each sequence could only belong to one bin. Sequences in each gene bin were assembled to produce consensus sequences (templates). Subsequent new sequences were added to existing bins using BLASTn (v. 1.4 WashU) and CROSSMATCH. Candidate pairs were identified as all BLAST hits having a quality score greater than or equal to 150. Alignments of at least 82% local identity were accepted into the bin. The component sequences from each bin were assembled using a version of PHRAP. Bins with several overlapping component sequences were assembled using DEEP PHRAP. The orientation (sense or antisense) of each assembled template was determined based on the number and orientation of its component sequences. Template sequences as disclosed in the sequence listing correspond to sense strand sequences (the “forward” reading frames), to the best determination. The complementary (antisense) strands are inherently disclosed herein. The component sequences which were used to assemble each template consensus sequence are listed in Table 2 along with their positions along the template nucleotide sequences.

Bins were compared against each other and those having local similarity of at least 82% were combined and reassembled. Reassembled bins having templates of insufficient overlap (less than 95% local identity) were re-split. Assembled templates were also subject to analysis by STITCHER/EXON MAPPER algorithms which analyze the probabilities of the presence of splice variants, alternatively spliced exons, splice junctions, differential expression of alternative spliced genes across tissue types or disease states, etc. These resulting bins were subject to several rounds of the above assembly procedures.

Once gene bins were generated based upon sequence alignments, bins were clone joined based upon clone information. If the 5′ sequence of one clone was present in one bin and the 3′ sequence from the same clone was present in a different bin, it was likely that the two bins actually belonged together in a single bin. The resulting combined bins underwent assembly procedures to regenerate the consensus sequences.

The final assembled templates were subsequently annotated using the following procedure. Template sequences were analyzed using BLASTn (v2.0, NCBI) versus gbpri (GenBank version 120). “Hits” were defined as an exact match having from 95% local identity over 200 base pairs through 100% local identity over 100 base pairs, or a homolog match having an E-value, i.e. a probability score, of ≦1×10⁻⁸. The hits were subject to frameshift FASTx versus GENPEPT (GenBank version 120). (See Table 4). In this analysis, a homolog match was defined as having an E-value of ≦1×10⁻⁸. The assembly method used above was described in “System and Methods for Analyzing Biomolecular Sequences,” U.S. Ser. No. 09/276,534, filed Mar. 25, 1999, and the LIFESEQ Gold user manual (Incyte) both incorporated by reference herein.

Following assembly, template sequences were subjected to motif, BLAST, and functional analyses, and categorized in protein hierarchies using methods described in, e.g., “Database System Employing Protein Function Hierarchies for Viewing Biomolecular Sequence Data,” U.S. Ser. No. 08/812,290, filed Mar. 6, 1997; “Relational Database for Storing Biomolecule Information,” U.S. Ser. No. 08/947,845, filed Oct. 9, 1997; “Project-Based Full-Length Biomolecular Sequence Database,” U.S. Ser. No. 08/811,758, filed Mar. 6, 1997; and “Relational Database and System for Storing Information Relating to Biomolecular Sequences,” U.S. Ser. No. 09/034,807, filed Mar. 4, 1998, all of which are incorporated by reference herein.

The template sequences were further analyzed by translating each template in all three forward reading frames and searching each translation against the Pfam database of hidden Markov model-based protein families and domains using the HMMER software package (available to the public from Washington University School of Medicine, St. Louis Mo.). (See also World Wide Web site http://pfam.wustl.edu/ for detailed descriptions of Pfam protein domains and families.)

Additionally, the template sequences were translated in all three forward reading frames, and each translation was searched against hidden Markov models for signal peptides using the HMMER software package. Construction of hidden Markov models and their usage in sequence analysis has been described. (See, for example, Eddy, S. R. (1996) Curr. Opin. Str. Biol. 6:361-365.) Only those signal peptide hits with a cutoff score of 11 bits or greater are reported. A cutoff score of 11 bits or greater corresponds to at least about 91-94% true-positives in signal peptide prediction. Template sequences were also translated in all three forward reading frames, and each translation was searched against TMAP, a program that uses weight matrices to delineate transmembrane segments on protein sequences and determine orientation, with respect to the cell cytosol (Persson, B. and Argos, P. (1994) J. Mol. Biol. 237:182-192, and Persson, B. and Argos, P. (1996) Protein Sci. 5:363-371.) Regions of templates which, when translated, contain similarity to signal peptide or transmembrane consensus sequences are reported in Table 1.

Template sequences are further analyzed using the bioinformatics tools listed in Table 4, or using sequence analysis software known in the art such as MACDNASIS PRO software (Hitachi Software Engineering, South San Francisco Calif.) and LASERGENE software (DNASTAR). Template sequences may be further queried against public databases such as the GenBank rodent, mammalian, vertebrate, prokaryote, and eukaryote databases.

V. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel, 1995, supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotide sequences encoding SPTM are analyzed with respect to the tissue sources from which they were derived. Polynucleotide sequences encoding SPTM were assembled, at least in part, with overlapping Incyte cDNA sequences. Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category for each polynucleotide sequence encoding SPTM is counted and divided by the total number of libraries across all categories for each polynucleotide sequence encoding SPTM. Similarly, each human tissue is classified into one of the following disease/condition categories: cancer, cell line, developmental, inflammation, neurological, trauma, cardiovascular, pooled, and other, and the number of libraries in each category for each polynucleotide sequence encoding SPTM is counted and divided by the total number of libraries across all categories for each polynucleotide sequence encoding SPTM. The resulting percentages reflect the tissue-specific and disease-specific expression of cDNA encoding SPTM. Percentage values of tissue-specific expression are reported in Table 3. cDNA sequences and cDNA library/tissue information are found in the LIFESEQ GOLD database (Incyte Genomics, Palo Alto Calif.).

VI. Tissue Distribution Profiling

A tissue distribution profile is determined for each template by compiling the cDNA library tissue classifications of its component cDNA sequences. Each component sequence, is derived from a cDNA library constructed from a human tissue. Each human tissue is classified into one of the following categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. Template sequences, component sequences, and cDNA library/tissue information are found in the LIFESEQ GOLD database (Incyte Genomics, Palo Alto Calif.).

Table 3 shows the tissue distribution profile for the templates of the invention. For each template, the three most frequently observed tissue categories are shown in column 2, along with the percentage of component sequences belonging to each category. Only tissue categories with percentage values of ≧10% are shown. A tissue distribution of “widely distributed” in column 2 indicates percentage values of <10% in all tissue categories.

VII. Transcript Image Analysis

Transcript images are generated as described in Seilhamer et al., “Comparative Gene Transcript Analysis,” U.S. Pat. No. 5,840,484, incorporated herein by reference.

VIII. Extension of Polynucleotide Sequences and Isolation of a Full-length cDNA

Oligonucleotide primers designed using an sptm of the Sequence Listing are used to extend the nucleic acid sequence. One primer is synthesized to initiate 5′ extension of the template, and the other primer, to initiate 3′ extension of the template. The initial primers may be designed using OLIGO 4.06 software (National Biosciences, Inc. (National Biosciences), Plymouth Minn.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations are avoided. Selected human cDNA libraries are used to extend the sequence. If more than one extension is necessary or desired, additional or nested sets of primers are designed.

High fidelity amplification is obtained by PCR using methods well known in the art. PCR is performed in 96-well plates using the PTC-200 thermal cycler (MJ Research). The reaction mix contains DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2,3, and4 repeated 20 times; Step 6: 68° C., 5 min; Step 7: storage at 4° C. In the alternative, the parameters for primer pair T7 and SK+ are as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well is determined by dispensing 100 μl PICOGREEN quantitation reagent (0.25% (v/v); Molecular Probes) dissolved in 1×Tris-EDTA (TE) and 0.5 μl of undiluted PCR product into each well of an opaque fluorimeter plate (Corning Incorporated (Corning), Corning N.Y.), allowing the DNA to bind to the reagent. The plate is scanned in a FLUOROSKAN II (Labsystems Oy) to measure the fluorescence of the sample and to quantify the concentration of DNA. A 5 μl to 10 μl aliquot of the reaction mixture is analyzed by electrophoresis on a 1% agarose mini-gel to determine which reactions are successful in extending the sequence.

The extended nucleotides are desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides are separated on low concentration (0.6 to 0.8%) agarose 25 gels, fragments are excised, and agar digested with AGAR ACE (Promega). Extended clones are religated using T4 ligase (New England Biolabs, Inc., Beverly Mass.) into pUC 18 vector (Amersham Pharmacia Biotech), treated with Pfu DNA polymerase (Stratagene) to fill-in restriction site overhangs, and transfected into competent E. coli cells. Transformed cells are selected on antibiotic-containing media, individual colonies are picked and cultured overnight at 37° C. in 384-well plates in LB/2×carbenicillin liquid media.

The cells are lysed, and DNA is amplified by PCR using Taq DNA polymerase (Amershain Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA is quantified by PICOGREEN reagent (Molecular Probes) as described above. Samples with low DNA recoveries are reamplified using the same conditions as described above. Samples are diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, the sptm is used to obtain regulatory sequences (promoters, introns, and enhancers) using the procedure above, oligonucleotides designed for such extension, and an appropriate genomic library.

IX. Labeling of Probes and Southern Hybridization Analyses

Hybridization probes derived from the sptm of the Sequence Listing are employed for screening cDNAs, mRNAs, or genomic DNA. The labeling of probe nucleotides between 100 and 1000 nucleotides in length is specifically described, but essentially the same procedure may be used with larger cDNA fragments. Probe sequences are labeled at room temperature for 30 minutes using a T4 polynucleotide kinase, γ³²P-ATP, and 0.5×One-Phor-All Plus (Amersham Pharmacia Biotech) buffer and purified using a ProbeQuant G-50 Microcolumn (Amersham Pharmacia Biotech). The probe mixture is diluted to 10⁷ dpm/μg/ml hybridization buffer and used in a typical membrane-based hybridization analysis.

The DNA is digested with a restriction endonuclease such as Eco RV and is electrophoresed through a 0.7% agarose gel. The DNA fragments are transferred from the agarose to nylon membrane (NYTRAN Plus, Schleicher & Schuell, Inc., Keene N.H.) using procedures specified by the manufacturer of the membrane. Prehybridization is carried out for three or more hours at 68° C., and hybridization is carried out overnight at 68° C. To remove non-specific signals, blots are sequentially washed at room temperature under increasingly stringent conditions, up to 0.1×saline sodium citrate (SSC) and 0.5% sodium dodecyl sulfate. After the blots are placed in a PHOSPHORIMAGER cassette (Molecular Dynamics) or are exposed to autoradiography film, hybridization patterns of standard and experimental lanes are compared. Essentially the same procedure is employed when screening RNA.

X. Chromosome Mapping of sptm

The cDNA sequences which were used to assemble SEQ ID NO: 1-79 are compared with sequences from the Incyte LIFESEQ database and public domain databases using BLAST and other implementations of the Smith-Waterman algorithm. Sequences from these databases that match SEQ ID NO: 1-79 are assembled into clusters of contiguous and overlapping sequences using assembly algorithms such as PHRAP (Table 4). Radiation hybrid and genetic mapping data available from public resources such as the Stanford Human Genome Center (SHGC), Whitehead Institute for Genome Research (WIGR), and Genethon are used to determine if any of the clustered sequences have been previously mapped. Inclusion of a mapped sequence in a cluster will result in the assignment of all sequences of that cluster, including its particular SEQ ID NO:, to that map location. The genetic map locations of SEQ ID NO: 1-79 are described as ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Gendthon which provide boundaries for radiation hybrid markers whose sequences were included in each of the clusters.

XI. Microarray Analysis

Probe Preparation from Tissue or Cell Samples

Total RNA is isolated from tissue samples using the guanidinium thiocyanate method and polyA⁺ RNA is purified using the oligo (dT) cellulose method. Each polyA⁺ RNA sample is reverse transcribed using MMLV reverse-transcriptase, 0.05 pg/μl oligo-dT primer (21 mer), 1×first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng polyA⁺ RNA with GEMBRIGHT kits (Incyte). Specific control polyA⁺ RNAs are synthesized by in vitro transcription from non-coding yeast genomic DNA (W. Lei, unpublished). As quantitative controls, the control mRNAs at 0.002 ng, 0.02 ng, 0.2 ng, and 2 ng are diluted into reverse transcription reaction at ratios of 1:100,000, 1:10,000, 1:1000, 1:100 (w/w) to sample mRNA respectively. The control mRNAs are diluted into reverse transcription reaction at ratios of 1:3, 3:1,1:10, 10:1,1:25, 25:1 (w/w) to sample mRNA differential expression patterns. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Probes are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The probe is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester, Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 ml of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford, Mass.) for 30 minutes at 60° C. followed by washes in 0.2% SDS and distilled water as before.

Hybridization

Hybridization reactions contain 9 μl of probe mixture consisting of 0.2 μg each of Cy3 and Cy5 labeled cDNA synthesis products in 5×SSC, 0.2% SDS hybridization buffer. The probe mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20×microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the probe mix at a known concentration. A specific location on the array contains a complementary DNA sequence, allowing the intensity of the signal at that location to be correlated with a weight ratio of hybridizing species of 1:100,000. When two probes from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (AID) conversion board (Analog Devices, Inc., Norwood, Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

XII. Complementary Nucleic Acids

Sequences complementary to the sptm are used to detect, decrease, or inhibit expression of the naturally occurring nucleotide. The use of oligonucleotides comprising from about 15 to 30 base pairs is typical in the art. However, smaller or larger sequence fragments can also be used. Appropriate oligonucleotides are designed from the sptm using OLIGO 4.06 software (National Biosciences) or other appropriate programs and are synthesized using methods standard in the art or ordered from a commercial supplier. To inhibit transcription, a complementary oligonucleotide is designed from the most unique 5′ sequence and used to prevent transcription factor binding to the promoter sequence. To inhibit translation, a complementary oligonucleotide is designed to prevent ribosomal binding and processing of the transcript.

XIII. Expression of SPTM

Expression and purification of SPTM is accomplished using bacterial or virus-based expression systems. For expression of SPTM in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express SPTM upon induction with isopropyl beta-D-thiogalactopyranoside (IPTG). Expression of SPTM in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding SPTM by either homologous recombination or bacterial-mediated transposition involving transfer plasmid intermediates. Viral infectivity is maintained and the strong polyhedrin promoter drives high levels of cDNA transcription. Recombinant baculovirus is used to infect Snodoptera frugiperda (Sf9) insect cells in most cases, or human hepatocytes, in some cases. Infection of the latter requires additional genetic modifications to baculovirus. (See e.g., Engelhard, supra; and Sandig, supra.)

In most expression systems, SPTM is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from SPTM at specifically engineered sites. FLAG, an 8-amino acid peptide, enables immunoaffinity purification using commercially available monoclonal and polyclonal anti-FLAG antibodies (Eastman Kodak Company, Rochester N.Y.). 6-His, a stretch of six consecutive histidine residues, enables purification on metal-chelate resins (QIAGEN). Methods for protein expression and purification are discussed in Ausubel (1995, supra, Chapters 10 and 16). Purified SPTM obtained by these methods can be used directly in the following activity assay.

XIV. Demonstration of SPTM Activity

An assay for SPTM activity measures the expression of SPTM on the cell surface. cDNA encoding SPTM is subcloned into an appropriate mammalian expression vector suitable for high levels of cDNA expression. The resulting construct is transfected into a nonhuman cell line such as NIH3T3. Cell surface proteins are labeled with biotin using methods known in the art. Immunoprecipitations are performed using SPTM-specific antibodies, and immunoprecipitated samples are analyzed using SDS-PAGE and immunoblotting techniques. The ratio of labeled immunoprecipitant to unlabeled immunoprecipitant is proportional to the amount of SPTM expressed on the cell surface.

Alternatively, an assay for SPTM activity measures the amount of SPTM in secretory, membrane-bound organelles. Transfected cells as described above are harvested and lysed. The lysate is fractionated using methods known to those of skill in the art, for example, sucrose gradient ultracentrifugation. Such methods allow the isolation of subcellular components such as the Golgi apparatus, ER, small membrane-bound vesicles, and other secretory organelles. Immunoprecipitations from fractionated and total cell lysates are performed using SPTM-specific antibodies, and immunoprecipitated samples are analyzed using SDS-PAGE and immunoblotting techniques. The concentration of SPTM in secretory organelles relative to SPTM in total cell lysate is proportional to the amount of SPTM in transit through the secretory pathway.

XV. Functional Assays

SPTM function is assessed by expressing sptm at physiologically elevated levels in mammalian cell culture systems. cDNA is subcloned into a mammalian expression vector containing a strong promoter that drives high levels of cDNA expression. Vectors of choice include pCMV SPORT (Life Technologies) and pCR3.1 (Invitrogen Corporation, Carlsbad Calif.), both of which contain the cytomegalovirus promoter. 5-10 μg of recombinant vector are transiently transfected into a human cell line, preferably of endothelial or hematopoietic origin, using either liposonme formulations or electroporation. 1-2 μg of an additional plasmid containing sequences encoding a marker protein are co-transfected.

Expression of a marker protein provides a means to distinguish transfected cells from nontransfected cells and is a reliable predictor of cDNA expression from the recombinant vector. Marker proteins of choice include, e.g., Green Fluorescent Protein (GFP; CLONTECH), CD64, or a CD64-GFP fusion protein. Flow cytometry (FCM), an automated laser optics-based technique, is used to identity transfected cells expressing GFP or CD64-GFP and to evaluate the apoptotic state of the cells and other cellular properties.

FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of SPTM on gene expression can be assessed using highly purified populations of cells transfected with sequences encoding SPTM and either CD64 or CD64-GFP. CD64 and CD64-GFP are expressed on the surface of transfected cells and bind to conserved regions of human immunoglobulin G (IgG). Transfected cells ate efficiently separated from nontransfected cells using magnetic beads coated with either human IgG or antibody against CD64 (DYNAL, Inc., Lake Success N.Y.). mRNA can be purified from the cells using methods well known by those of skill in the art. Expression of mRNA encoding SPTM and other genes of interest can be analyzed by northern analysis or microarray techniques.

XVI. Production of Antibodies

SPTM substantially purified using polyacrylamide gel electrophoresis (PAGE; see, e.g., Harrington, M. G. (1990) Methods Enzymol. 182:488-495), or other purification techniques, is used to immunize rabbits and to produce antibodies using standard protocols.

Alternatively, the SPTM amino acid sequence is analyzed using LASERGENE software (DNASTAR) to determine regions of high immunogenicity, and a corresponding peptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, Chapter 11.)

Typically, peptides 15 residues in length are synthesized using an ABI 431A peptide synthesizer (Applied Biosystems) using fmoc-chemistry and coupled to KLH (Sigma) by reaction with N-maleimidobenzoyl-N-hydroxysuccinimide ester (MBS) to increase immunogenicity. (See, e.g., Ausubel, supra.) Rabbits are immunized with the peptide-KLH complex in complete Freund's adjuvant. Resulting antisera are tested for antipeptide activity by, for example, binding the peptide to plastic, blocking with 1% BSA, reacting with rabbit antisera, washing, and reacting with radio-iodinated goat anti-rabbit IgG. Antisera with antipeptide activity are tested for anti-SPTM activity using protocols well known in the art, including ELISA, RIA, and immunoblotting.

XVII. Purification of Naturally Occurring SPTM Using Specific Antibodies

Naturally occurring or recombinant SPTM is substantially purified by immunoaffinity chromatography using antibodies specific for SPTM. An immunoaffinity column is constructed by covalently coupling anti-SPTM antibody to an activated chromatographic resin, such as CNBr-activated SEPHAROSE (Amersham Pharmacia Biotech). After the coupling, the resin is blocked and washed according to the manufacturer's instructions.

Media containing SPTM are passed over the immunoaffinity column, and the column is washed under conditions that allow the preferential absorbance of SPTM (e.g., high ionic strength buffers in the presence of detergent). The column is eluted under conditions that disrupt antibody/SPTM binding (e.g., a buffer of pH 2 to pH 3, or a high concentration of a chaotrope, such as urea or thiocyanate ion), and SPTM is collected.

XVIII. Identification of Molecules Which Interact with SPTM

SPTM, or biologically active fragments thereof, are labeled with 125I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled SPTM, washed, and any wells with labeled SPTM complex are assayed. Data obtained using different concentrations of SPTM are used to calculate values for the number, affinity, and association of SPTM with the candidate molecules.

Alternatively, molecules interacting with SPTM are analyzed using the yeast two-hybrid system as described in Fields, S. and O. Song (1989) Nature 340:245-246, or using commercially available kits based on the two-hybrid system, such as the MATCHMAKER system (CLONTECH). SPTM may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

TABLE 1

| SEQ | | | | | Domain | |
|---|---|---|---|---|---|---|
| ID NO: | Template ID | Start | Stop | Frame | Type | Topology |
| 1 | LG:223939.1:2000FEB18 | 202 | 288 | forward 1 | TM | N in |
| 2 | LG:397140.1:2000FEB18 | 508 | 588 | forward 1 | TM | |
| 2 | LG:397140.1:2000FEB18 | 236 | 319 | forward 2 | TM | N out |
| 2 | LG:397140.1:2000FEB18 | 377 | 463 | forward 2 | TM | N out |
| 2 | LG:397140.1:2000FEB18 | 288 | 374 | forward 3 | TM | |
| 2 | LG:397140.1:2000FEB18 | 480 | 566 | forward 3 | TM | |
| 3 | LG:1094205.1:2000FEB18 | 826 | 912 | forward 1 | TM | N in |
| 3 | LG:1094205.1:2000FEB18 | 867 | 953 | forward 3 | TM | N out |
| 4 | LG:481361.5:2000FEB18 | 115 | 201 | forward 1 | TM | N out |
| 4 | LG:481361.5:2000FEB18 | 295 | 354 | forward 1 | TM | N out |
| 4 | LG:481361.5:2000FEB18 | 373 | 459 | forward 1 | TM | N out |
| 4 | LG:481361.5:2000FEB18 | 101 | 187 | forward 2 | TM | N out |
| 4 | LG:481361.5:2000FEB18 | 369 | 443 | forward 3 | TM | N out |
| 5 | LG:981170.1:2000FEB18 | 10 | 78 | forward 1 | TM | N out |
| 5 | LG:981170.1:2000FEB18 | 598 | 648 | forward 1 | TM | N out |
| 5 | LG:981170.1:2000FEB18 | 790 | 876 | forward 1 | TM | N out |
| 5 | LG:981170.1:2000FEB18 | 1057 | 1143 | forward 1 | TM | N out |
| 5 | LG:981170.1:2000FEB18 | 1372 | 1458 | forward 1 | TM | N out |
| 5 | LG:981170.1:2000FEB18 | 1678 | 1764 | forward 1 | TM | N out |
| 5 | LG:981170.1:2000FEB18 | 1885 | 1959 | forward 1 | TM | N out |
| 5 | LG:981170.1:2000FEB18 | 11 | 82 | forward 2 | TM | N out |
| 5 | LG:981170.1:2000FEB18 | 731 | 817 | forward 2 | TM | N out |
| 5 | LG:981170.1:2000FEB18 | 1025 | 1105 | forward 2 | TM | N out |
| 5 | LG:981170.1:2000FEB18 | 1364 | 1450 | forward 2 | TM | N out |
| 5 | LG:981170.1:2000FEB18 | 1643 | 1696 | forward 2 | TM | N out |
| 5 | LG:981170.1:2000FEB18 | 1895 | 1954 | forward 2 | TM | N out |
| 5 | LG:981170.1:2000FEB18 | 24 | 110 | forward 3 | TM | N out |
| 5 | LG:981170.1:2000FEB18 | 843 | 899 | forward 3 | TM | N out |
| 5 | LG:981170.1:2000FEB18 | 1068 | 1136 | forward 3 | TM | N out |
| 5 | LG:981170.1:2000FEB18 | 1599 | 1655 | forward 3 | TM | N out |
| 5 | LG:981170.1:2000FEB18 | 1707 | 1793 | forward 3 | TM | N out |
| 5 | LG:981170.1:2000FEB18 | 1875 | 1961 | forward 3 | TM | N out |
| 6 | LI:197613.1:2000FEB01 | 140 | 226 | forward 2 | TM | N out |
| 6 | LI:197613.1:2000FEB01 | 269 | 355 | forward 2 | TM | N out |
| 7 | LI:902682.1:2000FEB01 | 225 | 311 | forward 3 | TM | N out |
| 8 | LI:212029.1:2000FEB01 | 2087 | 2149 | forward 2 | TM | N out |
| 8 | LI:212029.1:2000FEB01 | 2162 | 2224 | forward 2 | TM | N out |
| 9 | LI:249170.1:2000FEB01 | 208 | 282 | forward 1 | TM | N in |
| 10 | LI:813218.1:2000FEB01 | 466 | 552 | forward 1 | TM | N out |
| 10 | LI:813218.1:2000FEB01 | 137 | 202 | forward 2 | TM | N out |
| 10 | LI:813218.1:2000FEB01 | 356 | 436 | forward 2 | TM | N out |
| 10 | LI:813218.1:2000FEB01 | 452 | 520 | forward 2 | TM | N out |
| 11 | LI:902522.3:2000FEB01 | 99 | 158 | forward 3 | TM | N out |
| 12 | LI:474304.1:2000FEB01 | 64 | 147 | forward 1 | TM | N in |
| 12 | LI:474304.1:2000FEB01 | 217 | 285 | forward 1 | TM | N in |
| 12 | LI:474304.1:2000FEB01 | 298 | 372 | forward 1 | TM | N in |
| 12 | LI:474304.1:2000FEB01 | 26 | 88 | forward 2 | TM | N out |
| 12 | LI:474304.1:2000FEB01 | 110 | 172 | forward 2 | TM | N out |
| 12 | LI:474304.1:2000FEB01 | 200 | 271 | forward 2 | TM | N out |
| 12 | LI:474304.1:2000FEB01 | 332 | 418 | forward 2 | TM | N out |
| 12 | LI:474304.1:2000FEB01 | 890 | 952 | forward 2 | TM | N out |
| 12 | LI:474304.1:2000FEB01 | 72 | 158 | forward 3 | TM | N out |
| 12 | LI:474304.1:2000FEB01 | 273 | 332 | forward 3 | TM | N out |
| 12 | LI:474304.1:2000FEB01 | 330 | 383 | forward 3 | TM | N out |
| 12 | LI:474304.1:2000FEB01 | 606 | 674 | forward 3 | TM | N out |
| 13 | LI:027320.1:2000FEB01 | 544 | 603 | forward 1 | TM | N out |
| 13 | LI:027320.1:2000FEB01 | 694 | 768 | forward 1 | TM | N out |
| 13 | LI:027320.1:2000FEB01 | 1102 | 1182 | forward 1 | TM | N out |
| 13 | LI:027320.1:2000FEB01 | 1180 | 1236 | forward 1 | TM | N out |
| 13 | LI:027320.1:2000FEB01 | 545 | 604 | forward 2 | TM | |
| 13 | LI:027320.1:2000FEB01 | 851 | 913 | forward 2 | TM | |
| 13 | LI:027320.1:2000FEB01 | 932 | 994 | forward 2 | TM | |
| 13 | LI:027320.1:2000FEB01 | 1013 | 1075 | forward 2 | TM | |
| 13 | LI:027320.1:2000FEB01 | 1172 | 1228 | forward 2 | TM | |
| 13 | LI:027320.1:2000FEB01 | 579 | 665 | forward 3 | TM | N in |
| 13 | LI:027320.1:2000FEB01 | 720 | 806 | forward 3 | TM | N in |
| 13 | LI:027320.1:2000FEB01 | 828 | 908 | forward 3 | TM | N in |
| 13 | LI:027320.1:2000FEB01 | 1020 | 1103 | forward 3 | TM | N in |
| 13 | LI:027320.1:2000FEB01 | 1116 | 1202 | forward 3 | TM | N in |
| 14 | LI:228319.1:2000FEB01 | 76 | 162 | forward 1 | TM | N out |
| 14 | LI:228319.1:2000FEB01 | 119 | 187 | forward 2 | TM | N in |
| 14 | LI:228319.1:2000FEB01 | 506 | 592 | forward 2 | TM | N in |
| 14 | LI:228319.1:2000FEB01 | 63 | 149 | forward 3 | TM | N in |
| 14 | LI:228319.1:2000FEB01 | 369 | 455 | forward 3 | TM | N in |
| 14 | LI:228319.1:2000FEB01 | 708 | 770 | forward 3 | TM | N in |
| 14 | LI:228319.1:2000FEB01 | 786 | 848 | forward 3 | TM | N in |
| 15 | LG:197267.2:2000MAY19 | 11 | 88 | forward 2 | TM | N in |
| 15 | LG:197267.2:2000MAY19 | 200 | 271 | forward 2 | TM | N in |
| 16 | LG:403332.1:2000MAY19 | 352 | 417 | forward 1 | TM | N in |
| 16 | LG:403332.1:2000MAY19 | 490 | 552 | forward 1 | TM | N in |
| 16 | LG:403332.1:2000MAY19 | 562 | 624 | forward 1 | TM | N in |
| 16 | LG:403332.1:2000MAY19 | 721 | 783 | forward 1 | TM | N in |
| 16 | LG:403332.1:2000MAY19 | 796 | 858 | forward 1 | TM | N in |
| 16 | LG:403332.1:2000MAY19 | 871 | 948 | forward 1 | TM | N in |
| 16 | LG:403332.1:2000MAY19 | 1012 | 1098 | forward 1 | TM | N in |
| 16 | LG:403332.1:2000MAY19 | 1138 | 1188 | forward 1 | TM | N in |
| 16 | LG:403332.1:2000MAY19 | 1192 | 1278 | forward 1 | TM | N in |
| 16 | LG:403332.1:2000MAY19 | 1453 | 1530 | forward 1 | TM | N in |
| 16 | LG:403332.1:2000MAY19 | 365 | 427 | forward 2 | TM | N in |
| 16 | LG:403332.1:2000MAY19 | 521 | 607 | forward 2 | TM | N in |
| 16 | LG:403332.1:2000MAY19 | 644 | 727 | forward 2 | TM | N in |
| 16 | LG:403332.1:2000MAY19 | 800 | 886 | forward 2 | TM | N in |
| 16 | LC:403332.1:2000MAY19 | 911 | 973 | forward 2 | TM | N in |
| 16 | LG:403332.1:2000MAY19 | 986 | 1048 | forward 2 | TM | N in |
| 16 | LG:403332.1:2000MAY19 | 1211 | 1297 | forward 2 | TM | N in |
| 16 | LG:403332.1:2000MAY19 | 1445 | 1504 | forward 2 | TM | N in |
| 16 | LG:403332.1:2000MAY19 | 375 | 461 | forward 3 | TM | |
| 16 | LG:403332.1:2000MAY19 | 495 | 557 | forward 3 | TM | |
| 16 | LG:403332.1:2000MAY19 | 594 | 656 | forward 3 | TM | |
| 16 | LG:403332.1:2000MAY19 | 681 | 767 | forward 3 | TM | |
| 16 | LG:403332.1:2000MAY19 | 990 | 1052 | forward 3 | TM | |
| 16 | LG:403332.1:2000MAY19 | 1077 | 1139 | forward 3 | TM | |
| 16 | LG:403332.1:2000MAY19 | 1203 | 1274 | forward 3 | TM | |
| 16 | LG:403332.1:2000MAY19 | 1332 | 1385 | forward 3 | TM | |
| 17 | LG:983076.3:2000MAY19 | 479 | 565 | forward 2 | TM | N in |
| 17 | LG:983076.3:2000MAY19 | 704 | 790 | forward 2 | TM | N in |
| 17 | LG:983076.3:2000MAY19 | 114 | 200 | forward 3 | TM | N out |
| 17 | LG:983076.3:2000MAY19 | 261 | 317 | forward 3 | TM | N out |
| 17 | LG:983076.3:2000MAY19 | 501 | 587 | forward 3 | TM | N out |
| 17 | LG:983076.3:2000MAY19 | 738 | 794 | forward 3 | TM | N out |
| 18 | LG:216612.3:2000MAY19 | 120 | 206 | forward 3 | TM | N in |
| 18 | LG:216612.3:2000MAY19 | 234 | 284 | forward 3 | TM | N in |
| 18 | LG:216612.3:2000MAY19 | 327 | 413 | forward 3 | TM | N in |
| 18 | LG:216612.3:2000MAY19 | 444 | 530 | forward 3 | TM | N in |
| 18 | LG:216612.3:2000MAY19 | 810 | 887 | forward 3 | TM | N in |
| 19 | LG:322465.1:2000MAY19 | 239 | 319 | forward 2 | TM | N out |
| 19 | LG:322465.1:2000MAY19 | 276 | 329 | forward 3 | TM | N out |
| 20 | LG:093477.1:2000MAY19 | 25 | 111 | forward 1 | TM | N out |
| 20 | LG:093477.1:2000MAY19 | 11 | 85 | forward 2 | TM | N out |
| 20 | LG:093477.1:2000MAY19 | 242 | 295 | forward 2 | TM | N out |
| 20 | LG:093477.1:2000MAY19 | 24 | 110 | forward 3 | TM | N in |
| 20 | LG:093477.1:2000MAY19 | 657 | 719 | forward 3 | TM | N in |
| 21 | LG:222880.1:2000MAY19 | 211 | 297 | forward 1 | TM | |
| 21 | LG:222880.1:2000MAY19 | 1663 | 1749 | forward 1 | TM | |
| 21 | LG:222880.1:2000MAY19 | 1801 | 1863 | forward 1 | TM | |
| 21 | LG:222880.1:2000MAY19 | 1906 | 1968 | forward 1 | TM | |
| 21 | LG:222880.1:2000MAY19 | 2269 | 2316 | forward 1 | TM | |
| 21 | LG:222880.1:2000MAY19 | 425 | 487 | forward 2 | TM | N in |
| 21 | LG:222880.1:2000MAY19 | 506 | 568 | forward 2 | TM | N in |
| 21 | LG:222880.1:2000MAY19 | 611 | 691 | forward 2 | TM | N in |
| 21 | LG:222880.1:2000MAY19 | 698 | 784 | forward 2 | TM | N in |
| 21 | LG:222880.1:2000MAY19 | 875 | 961 | forward 2 | TM | N in |
| 21 | LG:222880.1:2000MAY19 | 1016 | 1078 | forward 2 | TM | N in |
| 21 | LG:222880.1:2000MAY19 | 1106 | 1168 | forward 2 | TM | N in |
| 21 | LG:222880.1:2000MAY19 | 1544 | 1630 | forward 2 | TM | N in |
| 21 | LG:222880.1:2000MAY19 | 1691 | 1774 | forward 2 | TM | N in |
| 21 | LG:222880.1:2000MAY19 | 1940 | 2026 | forward 2 | TM | N in |
| 21 | LG:222880.1:2000MAY19 | 2315 | 2395 | forward 2 | TM | N in |
| 21 | LG:222880.1:2000MAY19 | 1710 | 1784 | forward 3 | TM | N out |
| 21 | LG:222880.1:2000MAY19 | 1809 | 1895 | forward 3 | TM | N out |
| 21 | LG:222880.1:2000MAY19 | 1926 | 2009 | forward 3 | TM | N out |
| 21 | LG:222880.1:2000MAY19 | 2064 | 2129 | forward 3 | TM | N out |
| 22 | LG:898320.3:2000MAY19 | 151 | 237 | forward 1 | TM | N out |
| 22 | LG:898320.3:2000MAY19 | 478 | 534 | forward 1 | TM | N out |
| 22 | LG:898320.3:2000MAY19 | 736 | 822 | forward 1 | TM | N out |
| 22 | LG:898320.3:2000MAY19 | 1528 | 1599 | forward 1 | TM | N out |
| 22 | LG:898320.3:2000MAY19 | 1663 | 1710 | forward 1 | TM | N out |
| 22 | LG:898320.3:2000MAY19 | 2017 | 2088 | forward 1 | TM | N out |
| 22 | LG:898320.3:2000MAY19 | 2131 | 2184 | forward 1 | TM | N out |
| 22 | LG:898320.3:2000MAY19 | 719 | 796 | forward 2 | TM | N in |
| 22 | LG:898320.3:2000MAY19 | 1334 | 1420 | forward 2 | TM | N in |
| 22 | LG:898320.3:2000MAY19 | 1598 | 1681 | forward 2 | TM | N in |
| 22 | LG:898320.3:2000MAY19 | 1733 | 1819 | forward 2 | TM | N in |
| 22 | LG:898320.3:2000MAY19 | 1919 | 1984 | forward 2 | TM | N in |
| 22 | LG:898320.3:2000MAY19 | 2078 | 2140 | forward 2 | TM | N in |
| 22 | LG:898320.3:2000MAY19 | 2159 | 2245 | forward 2 | TM | N in |
| 22 | LG:898320.3:2000MAY19 | 90 | 176 | forward 3 | TM | N out |
| 22 | LG:898320.3:2000MAY19 | 411 | 485 | forward 3 | TM | N out |
| 22 | LG:898320.3:2000MAY19 | 501 | 578 | forward 3 | TM | N out |
| 22 | LG:898320.3:2000MAY19 | 600 | 686 | forward 3 | TM | N out |
| 22 | LG:898320.3:2000MAY19 | 783 | 854 | forward 3 | TM | N out |
| 22 | LG:898320.3:2000MAY19 | 912 | 989 | forward 3 | TM | N out |
| 22 | LG:898320.3:2000MAY19 | 1023 | 1097 | forward 3 | TM | N out |
| 22 | LG:898320.3:2000MAY19 | 1164 | 1226 | forward 3 | TM | N out |
| 22 | LG:898320.3:2000MAY19 | 1236 | 1298 | forward 3 | TM | N out |
| 22 | LG:898320.3:2000MAY19 | 1335 | 1421 | forward 3 | TM | N out |
| 22 | LG:898320.3:2000MAY19 | 2016 | 2093 | forward 3 | TM | N out |
| 22 | LG:898320.3:2000MAY19 | 2151 | 2237 | forward 3 | TM | N out |
| 23 | LG:1327047.1:2000MAY19 | 466 | 552 | forward 1 | TM | N out |
| 23 | LG:1327047.1:2000MAY19 | 137 | 202 | forward 2 | TM | N out |
| 23 | LG:1327047.1:2000MAY19 | 356 | 436 | forward 2 | TM | N out |
| 23 | LG:1327047.1:2000MAY19 | 452 | 520 | forward 2 | TM | N out |
| 23 | LG:1327047.1:2000MAY19 | 1131 | 1217 | forward 3 | TM | |
| 24 | LG:235157.21:2000MAY19 | 11 | 91 | forward 2 | TM | N out |
| 24 | LG:235157.21:2000MAY19 | 161 | 247 | forward 2 | TM | N out |
| 24 | LG:235157.21:2000MAY19 | 467 | 529 | forward 2 | TM | N out |
| 24 | LG:235157.21:2000MAY19 | 551 | 613 | forward 2 | TM | N out |
| 24 | LG:235157.21:2000MAY19 | 686 | 760 | forward 2 | TM | N out |
| 24 | LG:235157.21:2000MAY19 | 785 | 862 | forward 2 | TM | N out |
| 25 | LG:085713.1:2000MAY19 | 97 | 183 | forward 1 | TM | N out |
| 25 | LG:085713.1:2000MAY19 | 1489 | 1575 | forward 1 | TM | N out |
| 25 | LG:085713.1:2000MAY19 | 1786 | 1854 | forward 1 | TM | N out |
| 25 | LG:085713.1:2000MAY19 | 2275 | 2361 | forward 1 | TM | N out |
| 25 | LG:085713.1:2000MAY19 | 2407 | 2493 | forward 1 | TM | N out |
| 25 | LG:085713.1:2000MAY19 | 134 | 211 | forward 2 | TM | N in |
| 25 | LG:085713.1:2000MAY19 | 1481 | 1528 | forward 2 | TM | N in |
| 25 | LG:085713.1:2000MAY19 | 2099 | 2164 | forward 2 | TM | N in |
| 25 | LG:085713.1:2000MAY19 | 2387 | 2449 | forward 2 | TM | N in |
| 25 | LG:085713.1:2000MAY19 | 2474 | 2536 | forward 2 | TM | N in |
| 25 | LG:085713.1:2000MAY19 | 72 | 158 | forward 3 | TM | N out |
| 25 | LG:085713.1:2000MAY19 | 1488 | 1574 | forward 3 | TM | N out |
| 25 | LG:085713.1:2000MAY19 | 2037 | 2108 | forward 3 | TM | N out |
| 25 | LG:085713.1:2000MAY19 | 2184 | 2270 | forward 3 | TM | N out |
| 25 | LG:085713.1:2000MAY19 | 2346 | 2432 | forward 3 | TM | N out |
| 26 | LG:482421.1:2000MAY19 | 385 | 456 | forward 1 | TM | N out |
| 26 | LG:482421.1:2000MAY19 | 715 | 777 | forward 1 | TM | N out |
| 26 | LG:482421.1:2000MAY19 | 841 | 909 | forward 1 | TM | N out |
| 26 | LG:482421.1:2000MAY19 | 9701 | 1056 | forward 1 | TM | N out |
| 26 | LG:482421.1:2000MAY19 | 1222 | 1284 | forward 1 | TM | N out |
| 26 | LG:482421.1:2000MAY19 | 1423 | 1491 | forward 1 | TM | N out |
| 26 | LG:482421.1:2000MAY19 | 2185 | 2271 | forward 1 | TM | N out |
| 26 | LG:482421.1:2000MAY19 | 2518 | 2604 | forward 1 | TM | N out |
| 26 | LG:482421.1:2000MAY19 | 2674 | 2760 | forward 1 | TM | N out |
| 26 | LG:482421.1:2000MAY19 | 11 | 64 | forward 2 | TM | N out |
| 26 | LG:482421.1:2000MAY19 | 260 | 346 | forward 2 | TM | N out |
| 26 | LG:482421.1:2000MAY19 | 416 | 502 | forward 2 | TM | N out |
| 26 | LG:482421.1:2000MAY19 | 506 | 586 | forward 2 | TM | N out |
| 26 | LG:482421.1:2000MAY19 | 857 | 943 | forward 2 | TM | N out |
| 26 | LG:482421.1:2000MAY19 | 965 | 1021 | forward 2 | TM | N out |
| 26 | LG:482421.1:2000MAY19 | 1934 | 1987 | forward 2 | TM | N out |
| 26 | LG:482421.1:2000MAY19 | 2120 | 2206 | forward 2 | TM | N out |
| 26 | LG:482421.1:2000MAY19 | 2549 | 2635 | forward 2 | TM | N out |
| 26 | LG:482421.1:2000MAY19 | 2693 | 2773 | forward 2 | TM | N out |
| 26 | LG:482421.1:2000MAY19 | 12 | 65 | forward 3 | TM | N out |
| 26 | LG:482421.1:2000MAY19 | 258 | 332 | forward 3 | TM | N out |
| 26 | LG:482421.1:2000MAY19 | 603 | 689 | forward 3 | TM | N out |
| 26 | LG:482421.1:2000MAY19 | 690 | 773 | forward 3 | TM | N out |
| 26 | LG:482421.1:2000MAY19 | 822 | 905 | forward 3 | TM | N out |
| 26 | LG:482421.1:2000MAY19 | 972 | 1028 | forward 3 | TM | N out |
| 26 | LG:482421.1:2000MAY19 | 1074 | 1130 | forward 3 | TM | N out |
| 26 | LG:482421.1:2000MAY19 | 2403 | 2489 | forward 3 | TM | N out |
| 26 | LG:482421.1:2000MAY19 | 2550 | 2636 | forward 3 | TM | N out |
| 26 | LG:482421.1:2000MAY19 | 2673 | 2753 | forward 3 | TM | N out |
| 27 | LG:330944.4:2000MAY19 | 389 | 475 | forward 2 | TM | N out |
| 28 | LI:223060.1:2000MAY01 | 433 | 519 | forward 1 | TM | N in |
| 28 | LI:223060.1:2000MAY01 | 1267 | 1353 | forward 1 | TM | N in |
| 28 | LI:223060.1:2000MAY01 | 1639 | 1704 | forward 1 | TM | N in |
| 28 | LI:223060.1:2000MAY01 | 1759 | 1842 | forward 1 | TM | N in |
| 28 | LI:223060.1:2000MAY01 | 260 | 334 | forward 2 | TM | N out |
| 28 | LI:223060.1:2000MAY01 | 938 | 1003 | forward 2 | TM | N out |
| 28 | LI:223060.1:2000MAY01 | 1553 | 1639 | forward 2 | TM | N out |
| 28 | LI:223060.1:2000MAY01 | 1428 | 1514 | forward 3 | TM | N out |
| 29 | LI:213087.1:2000MAY01 | 732 | 818 | forward 3 | TM | N in |
| 30 | LI:405330.1:2000MAY01 | 103 | 165 | forward 1 | TM | |
| 30 | LI:405330.1:2000MAY01 | 406 | 492 | forward 1 | TM | |
| 30 | LI:405330.1:2000MAY01 | 1276 | 1362 | forward 1 | TM | |
| 30 | LI:405330.1:2000MAY01 | 1396 | 1449 | forward 1 | TM | |
| 30 | LI:405330.1:2000MAY01 | 1615 | 1674 | forward 1 | TM | |
| 30 | LI:405330.1:2000MAY01 | 1864 | 1917 | forward 1 | TM | |
| 30 | LI:405330.1:2000MAY01 | 1966 | 2016 | forward 1 | TM | |
| 30 | LI:405330.1:2000MAY01 | 2017 | 2097 | forward 1 | TM | |
| 30 | LI:405330.1:2000MAY01 | 2257 | 2319 | forward 1 | TM | |
| 30 | LI:405330.1:2000MAY01 | 2329 | 2397 | forward 1 | TM | |
| 30 | LI:405330.1:2000MAY01 | 2416 | 2478 | forward 1 | TM | |
| 30 | LI:405330.1:2000MAY01 | 485 | 550 | forward 2 | TM | N out |
| 30 | LI:405330.1:2000MAY01 | 671 | 757 | forward 2 | TM | N out |
| 30 | LI:405330.1:2000MAY01 | 1283 | 1357 | forward 2 | TM | N out |
| 30 | LI:405330.1:2000MAY01 | 1511 | 1579 | forward 2 | TM | N out |
| 30 | LI:405330.1:2000MAY01 | 1877 | 1933 | forward 2 | TM | N out |
| 30 | LI:405330.1:2000MAY01 | 2339 | 2395 | forward 2 | TM | N out |
| 30 | LI:405330.1:2000MAY01 | 2411 | 2473 | forward 2 | TM | N out |
| 30 | LI:405330.1:2000MAY01 | 249 | 320 | forward 3 | TM | N in |
| 30 | LI:405330.1:2000MAY01 | 678 | 764 | forward 3 | TM | N in |
| 30 | LI:405330.1:2000MAY01 | 990 | 1070 | forward 3 | TM | N in |
| 30 | LI:405330.1:2000MAY01 | 1650 | 1721 | forward 3 | TM | N in |
| 30 | LI:405330.1:2000MAY01 | 2265 | 2351 | forward 3 | TM | N in |
| 30 | LI:405330.1:2000MAY01 | 2433 | 2513 | forward 3 | TM | N in |
| 31 | LI:350243.2:2000MAY01 | 3799 | 3852 | forward 1 | TM | |
| 31 | LI:350243.2:2000MAY01 | 4453 | 4533 | forward 1 | TM | |
| 31 | LI:350243.2:2000MAY01 | 5392 | 5460 | forward 1 | TM | |
| 31 | LI:350243.2:2000MAY01 | 5944 | 6030 | forward 1 | TM | |
| 31 | LI:350243.2:2000MAY01 | 6256 | 6333 | forward 1 | TM | |
| 31 | LI:350243.2:2000MAY01 | 7003 | 7071 | forward 1 | TM | |
| 31 | LI:350243.2:2000MAY01 | 7333 | 7398 | forward 1 | TM | |
| 31 | LI:350243.2:2000MAY01 | 7552 | 7626 | forward 1 | TM | |
| 31 | LI:350243.2:2000MAY01 | 7780 | 7845 | forward 1 | TM | |
| 31 | LI:350243.2:2000MAY01 | 7867 | 7923 | forward 1 | TM | |
| 31 | LI:350243.2:2000MAY01 | 7954 | 8025 | forward 1 | TM | |
| 31 | LI:350243.2:2000MAY01 | 8407 | 8490 | forward 1 | TM | |
| 31 | LI:350243.2:2000MAY01 | 4361 | 4447 | forward 2 | TM | N out |
| 31 | LI:350243.2:2000MAY01 | 4724 | 4807 | forward 2 | TM | N out |
| 31 | LI:350243.2:2000MAY01 | 5402 | 5488 | forward 2 | TM | N out |
| 31 | LI:350243.2:2000MAY01 | 5618 | 5668 | forward 2 | TM | N out |
| 31 | LI:350243.2:2000MAY01 | 5687 | 5767 | forward 2 | TM | N out |
| 31 | LI:350243.2:2000MAY01 | 6263 | 6334 | forward 2 | TM | N out |
| 31 | LI:350243.2:2000MAY01 | 6488 | 6574 | forward 2 | TM | N out |
| 31 | LI:350243.2:2000MAY01 | 7610 | 7696 | forward 2 | TM | N out |
| 31 | LI:350243.2:2000MAY01 | 7814 | 7900 | forward 2 | TM | N out |
| 31 | LI:350243.2:2000MAY01 | 7991 | 8047 | forward 2 | TM | N out |
| 31 | LI:350243.2:2000MAY01 | 8501 | 8587 | forward 2 | TM | N out |
| 31 | LI:350243.2:2000MAY01 | 8699 | 8785 | forward 2 | TM | N out |
| 31 | LI:350243.2:2000MAY01 | 3975 | 4052 | forward 3 | TM | N in |
| 31 | LI:350243.2:2000MAY01 | 5037 | 5108 | forward 3 | TM | N in |
| 31 | LI:350243.2:2000MAY01 | 6090 | 6155 | forward 3 | TM | N in |
| 31 | LI:350243.2:2000MAY01 | 6276 | 6338 | forward 3 | TM | N in |
| 31 | LI:350243.2:2000MAY01 | 6357 | 6419 | forward 3 | TM | N in |
| 31 | LI:350243.2:2000MAY01 | 6492 | 6557 | forward 3 | TM | N in |
| 31 | LI:350243.2:2000MAY01 | 7005 | 7055 | forward 3 | TM | N in |
| 31 | LI:350243.2:2000MAY01 | 7185 | 7271 | forward 3 | TM | N in |
| 31 | LI:350243.2:2000MAY01 | 7365 | 7439 | forward 3 | TM | N in |
| 31 | LI:350243.2:2000MAY01 | 7650 | 7727 | forward 3 | TM | N in |
| 31 | LI:350243.2:2000MAY01 | 7818 | 7868 | forward 3 | TM | N in |
| 31 | LI:350243.2:2000MAY01 | 7881 | 7949 | forward 3 | TM | N in |
| 31 | LI:350243.2:2000MAY01 | 8445 | 8507 | forward 3 | TM | N in |
| 31 | LI:350243.2:2000MAY01 | 8526 | 8585 | forward 3 | TM | N in |
| 32 | LI:445188.1:2000MAY01 | 10 | 63 | forward 1 | TM | N in |
| 32 | LI:445188.1:2000MAY01 | 169 | 240 | forward 1 | TM | N in |
| 32 | LI:445188.1:2000MAY01 | 337 | 408 | forward 1 | TM | N in |
| 32 | LI:445188.1:2000MAY01 | 406 | 471 | forward 1 | TM | N in |
| 32 | LI:445188.1:2000MAY01 | 796 | 861 | forward 1 | TM | N in |
| 32 | LI:445188.1:2000MAY01 | 967 | 1041 | forward 1 | TM | N in |
| 32 | LI:445188.1:2000MAY01 | 1135 | 1209 | forward 1 | TM | N in |
| 32 | LI:445188.1:2000MAY01 | 1228 | 1314 | forward 1 | TM | N in |
| 32 | LI:445188.1:2000MAY01 | 1396 | 1473 | forward 1 | TM | N in |
| 32 | LI:445188.1:2000MAY01 | 131 | 217 | forward 2 | TM | N in |
| 32 | LI:445188.1:2000MAY01 | 335 | 421 | forward 2 | TM | N in |
| 32 | LI:445188.1:2000MAY01 | 980 | 1042 | forward 2 | TM | N in |
| 32 | LI:445188.1:2000MAY01 | 1136 | 1189 | forward 2 | TM | N in |
| 32 | LI:445188.1:2000MAY01 | 1445 | 1522 | forward 2 | TM | N in |
| 32 | LI:445188.1:2000MAY01 | 12 | 59 | forward 3 | TM | N out |
| 32 | LI:445188.1:2000MAY01 | 135 | 221 | forward 3 | TM | N out |
| 32 | LI:445188.1:2000MAY01 | 258 | 344 | forward 3 | TM | N out |
| 32 | LI:445188.1:2000MAY01 | 351 | 431 | forward 3 | TM | N out |
| 32 | LI:445188.1:2000MAY01 | 573 | 650 | forward 3 | TM | N out |
| 32 | LI:445188.1:2000MAY01 | 819 | 893 | forward 3 | TM | N out |
| 32 | LI:445188.1:2000MAY01 | 1008 | 1094 | forward 3 | TM | N out |
| 32 | LI:445188.1:2000MAY01 | 1149 | 1235 | forward 3 | TM | N out |
| 32 | LI:445188.1:2000MAY01 | 1695 | 1763 | forward 3 | TM | N out |
| 33 | LI:244378.1:2000MAY01 | 28 | 114 | forward 1 | TM | N out |
| 33 | LI:244378.1:2000MAY01 | 403 | 462 | forward 1 | TM | N out |
| 33 | LI:244378.1:2000MAY01 | 466 | 549 | forward 1 | TM | N out |
| 33 | LI:244378.1:2000MAY01 | 1972 | 2058 | forward 1 | TM | N out |
| 33 | LI:244378.1:2000MAY01 | 11 | 76 | forward 2 | TM | N in |
| 33 | LI:244378.1:2000MAY01 | 401 | 487 | forward 2 | TM | N in |
| 33 | LI:244378.1:2000MAY01 | 533 | 619 | forward 2 | TM | N in |
| 33 | LI:244378.1:2000MAY01 | 1313 | 1369 | forward 2 | TM | N in |
| 33 | LI:244378.1:2000MAY01 | 1985 | 2035 | forward 2 | TM | N in |
| 33 | LI:244378.1:2000MAY01 | 24 | 86 | forward 3 | TM | N out |
| 33 | LI:244378.1:2000MAY01 | 108 | 170 | forward 3 | TM | N out |
| 33 | LI:244378.1:2000MAY01 | 1980 | 2045 | forward 3 | TM | N out |
| 34 | LI:236574.15:2000MAY01 | 1 | 81 | forward 1 | TM | N out |
| 34 | LI:236574.15:2000MAY01 | 97 | 183 | forward 1 | TM | N out |
| 34 | LI:236574.15:2000MAY01 | 95 | 166 | forward 2 | TM | N out |
| 34 | LI:236574.15:2000MAY01 | 239 | 325 | forward 2 | TM | N out |
| 34 | LI:236574.15:2000MAY01 | 93 | 155 | forward 3 | TM | N in |
| 35 | LI:010100.20:2000MAY01 | 43 | 96 | forward 1 | TM | N in |
| 35 | LI:010100.20:2000MAY01 | 169 | 255 | forward 1 | TM | N in |
| 35 | LI:010100.20:2000MAY01 | 328 | 399 | forward 1 | TM | N in |
| 35 | LI:010100.20:2000MAY01 | 658 | 720 | forward 1 | TM | N in |
| 35 | LI:010100.20:2000MAY01 | 742 | 804 | forward 1 | TM | N in |
| 35 | LI:010100.20:2000MAY01 | 910 | 996 | forward 1 | TM | N in |
| 35 | LI:010100.20:2000MAY01 | 1117 | 1203 | forward 1 | TM | N in |
| 35 | LI:010100.20:2000MAY01 | 1240 | 1317 | forward 1 | TM | N in |
| 35 | LI:010100.20:2000MAY01 | 1528 | 1611 | forward 1 | TM | N in |
| 35 | LI:010100.20:2000MAY01 | 1822 | 1908 | forward 1 | TM | N in |
| 35 | LI:010100.20:2000MAY01 | 2206 | 2280 | forward 1 | TM | N in |
| 35 | LI:010100.20:2000MAY01 | 2671 | 2754 | forward 1 | TM | N in |
| 35 | LI:010100.20:2000MAY01 | 2899 | 2973 | forward 1 | TM | N in |
| 35 | LI:010100.20:2000MAY01 | 3037 | 3123 | forward 1 | TM | N in |
| 35 | LI:010100.20:2000MAY01 | 3349 | 3435 | forward 1 | TM | N in |
| 35 | LI:010100.20:2000MAY01 | 26 | 109 | forward 2 | TM | N in |
| 35 | LI:010100.20:2000MAY01 | 146 | 232 | forward 2 | TM | N in |
| 35 | LI:010100.20:2000MAY01 | 530 | 592 | forward 2 | TM | N in |
| 35 | LI:010100.20:2000MAY01 | 743 | 796 | forward 2 | TM | N in |
| 35 | LI:010100.20:2000MAY01 | 953 | 1039 | forward 2 | TM | N in |
| 35 | LI:010100.20:2000MAY01 | 1154 | 1207 | forward 2 | TM | N in |
| 35 | LI:010100.20:2000MAY01 | 1274 | 1360 | forward 2 | TM | N in |
| 35 | LI:010100.20:2000MAY01 | 1364 | 1435 | forward 2 | TM | N in |
| 35 | LI:010100.20:2000MAY01 | 1544 | 1630 | forward 2 | TM | N in |
| 35 | LI:010100.20:2000MAY01 | 1748 | 1822 | forward 2 | TM | N in |
| 35 | LI:010100.20:2000MAY01 | 1922 | 2008 | forward 2 | TM | N in |
| 35 | LI:010100.20:2000MAY01 | 2354 | 2440 | forward 2 | TM | N in |
| 35 | LI:010100.20:2000MAY01 | 2663 | 2731 | forward 2 | TM | N in |
| 35 | LI:010100.20:2000MAY01 | 2732 | 2818 | forward 2 | TM | N in |
| 35 | LI:010100.20:2000MAY01 | 3287 | 3373 | forward 2 | TM | N in |
| 35 | LI:010100.20:2000MAY01 | 3386 | 3472 | forward 2 | TM | N in |
| 35 | LI:010100.20:2000MAY01 | 141 | 227 | forward 3 | TM | |
| 35 | LI:010100.20:2000MAY01 | 375 | 452 | forward 3 | TM | |
| 35 | LI:010100.20:2000MAY01 | 768 | 824 | forward 3 | TM | |
| 35 | LI:010100.20:2000MAY01 | 969 | 1049 | forward 3 | TM | |
| 35 | LI:010100.20:2000MAY01 | 1146 | 1214 | forward 3 | TM | |
| 35 | LI:010100.20:2000MAY01 | 1281 | 1367 | forward 3 | TM | |
| 35 | LI:010100.20:2000MAY01 | 1590 | 1652 | forward 3 | TM | |
| 35 | LI:010100.20:2000MAY01 | 1704 | 1766 | forward 3 | TM | |
| 35 | LI:010100.20:2000MAY01 | 1908 | 1976 | forward 3 | TM | |
| 35 | LI:010100.20:2000MAY01 | 2208 | 2294 | forward 3 | TM | |
| 35 | LI:010100.20:2000MAY01 | 2700 | 2762 | forward 3 | TM | |
| 35 | LI:010100.20:2000MAY01 | 2781 | 2843 | forward 3 | TM | |
| 35 | LI:010100.20:2000MAY01 | 2862 | 2924 | forward 3 | TM | |
| 35 | LI:010100.20:2000MAY01 | 2946 | 3020 | forward 3 | TM | |
| 35 | LI:010100.20:2000MAY01 | 3051 | 3131 | forward 3 | TM | |
| 35 | LI:010100.20:2000MAY01 | 3135 | 3206 | forward 3 | TM | |
| 35 | LI:010100.20:2000MAY01 | 3324 | 3389 | forward 3 | TM | |
| 36 | LI:037940.6:2000MAY01 | 163 | 249 | forward 1 | TM | N in |
| 36 | LI:037940.6:2000MAY01 | 14 | 100 | forward 2 | TM | |
| 36 | LI:037940.6:2000MAY01 | 161 | 247 | forward 2 | TM | |
| 36 | LI:037940.6:2000MAY01 | 365 | 451 | forward 2 | TM | |
| 36 | LI:037940.6:2000MAY01 | 12 | 92 | forward 3 | TM | N out |
| 36 | LI:037940.6:2000MAY01 | 387 | 473 | forward 3 | TM | N out |
| 37 | LI:228550.3:2000MAY01 | 1537 | 1602 | forward 1 | TM | N in |
| 37 | LI:228550.3:2000MAY01 | 1603 | 1680 | forward 1 | TM | N in |
| 37 | LI:228550.3:2000MAY01 | 1846 | 1899 | forward 1 | TM | N in |
| 37 | LI:228550.3:2000MAY01 | 4165 | 4239 | forward 1 | TM | N in |
| 37 | LI:228550.3:2000MAY01 | 4549 | 4629 | forward 1 | TM | N in |
| 37 | LI:228550.3:2000MAY01 | 4651 | 4737 | forward 1 | TM | N in |
| 37 | LI:228550.3:2000MAY01 | 5371 | 5430 | forward 1 | TM | N in |
| 37 | LI:228550.3:2000MAY01 | 7300 | 7362 | forward 1 | TM | N in |
| 37 | LI:228550.3:2000MAY01 | 7408 | 7470 | forward 1 | TM | N in |
| 37 | LI:228550.3:2000MAY01 | 7582 | 7659 | forward 1 | TM | N in |
| 37 | LI:228550.3:2000MAY01 | 1622 | 1678 | forward 2 | TM | N in |
| 37 | LI:228550.3:2000MAY01 | 1718 | 1804 | forward 2 | TM | N in |
| 37 | LI:228550.3:2000MAY01 | 1841 | 1927 | forward 2 | TM | N in |
| 37 | LI:228550.3:2000MAY01 | 1976 | 2038 | forward 2 | TM | N in |
| 37 | LI:228550.3:2000MAY01 | 2066 | 2128 | forward 2 | TM | N in |
| 37 | LI:228550.3:2000MAY01 | 2150 | 2236 | forward 2 | TM | N in |
| 37 | LI:228550.3:2000MAY01 | 2546 | 2608 | forward 2 | TM | N in |
| 37 | LI:228550.3:2000MAY01 | 3746 | 3829 | forward 2 | TM | N in |
| 37 | LI:228550.3:2000MAY01 | 4457 | 4543 | forward 2 | TM | N in |
| 37 | LI:228550.3:2000MAY01 | 4724 | 4810 | forward 2 | TM | N in |
| 37 | LI:228550.3:2000MAY01 | 7229 | 7303 | forward 2 | TM | N in |
| 37 | LI:228550.3:2000MAY01 | 7637 | 7723 | forward 2 | TM | N in |
| 37 | LI:228550.3:2000MAY01 | 3978 | 4031 | forward 3 | TM | N in |
| 37 | LI:228550.3:2000MAY01 | 4362 | 4424 | forward 3 | TM | N in |
| 37 | LI:228550.3:2000MAY01 | 4440 | 4502 | forward 3 | TM | N in |
| 37 | LI:228550.3:2000MAY01 | 4575 | 4637 | forward 3 | TM | N in |
| 37 | LI:228550.3:2000MAY01 | 4671 | 4733 | forward 3 | TM | N in |
| 37 | LI:228550.3:2000MAY01 | 5070 | 5135 | forward 3 | TM | N in |
| 37 | LI:228550.3:2000MAY01 | 6963 | 7049 | forward 3 | TM | N in |
| 37 | LI:228550.3:2000MAY01 | 7308 | 7361 | forward 3 | TM | N in |
| 37 | LI:228550.3:2000MAY01 | 7482 | 7535 | forward 3 | TM | N in |
| 37 | LI:228550.3:2000MAY01 | 7617 | 7703 | forward 3 | TM | N in |
| 38 | LI:027320.1:2000MAY01 | 637 | 723 | forward 1 | TM | N in |
| 38 | LI:027320.1:2000MAY01 | 796 | 882 | forward 1 | TM | N in |
| 38 | LI:027320.1:2000MAY01 | 970 | 1050 | forward 1 | TM | N in |
| 38 | LI:027320.1:2000MAY01 | 1081 | 1167 | forward 1 | TM | N in |
| 38 | LI:027320.1:2000MAY01 | 692 | 778 | forward 2 | TM | N in |
| 38 | LI:027320.1:2000MAY01 | 857 | 928 | forward 2 | TM | N in |
| 38 | LI:027320.1:2000MAY01 | 932 | 1012 | forward 2 | TM | N in |
| 38 | LI:027320.1:2000MAY01 | 1079 | 1165 | forward 2 | TM | N in |
| 38 | LI:027320.1:2000MAY01 | 1220 | 1306 | forward 2 | TM | N in |
| 38 | LI:027320.1:2000MAY01 | 1032 | 1118 | forward 3 | TM | N out |
| 38 | LI:027320.1:2000MAY01 | 1128 | 1190 | forward 3 | TM | N out |
| 38 | LI:027320.1:2000MAY01 | 1242 | 1292 | forward 3 | TM | N out |
| 39 | LI:321475.1:2000MAY01 | 610 | 681 | forward 1 | TM | N out |
| 39 | LI:321475.1:2000MAY01 | 1081 | 1167 | forward 1 | TM | N out |
| 39 | LI:321475.1:2000MAY01 | 1207 | 1284 | forward 1 | TM | N out |
| 39 | LI:321475.1:2000MAY01 | 125 | 187 | forward 2 | TM | N out |
| 39 | LI:321475.1:2000MAY01 | 200 | 262 | forward 2 | TM | N out |
| 39 | LI:321475.1:2000MAY01 | 356 | 442 | forward 2 | TM | N out |
| 39 | LI:321475.1:2000MAY01 | 641 | 727 | forward 2 | TM | N out |
| 39 | LI:321475.1:2000MAY01 | 791 | 877 | forward 2 | TM | N out |
| 39 | LI:321475.1:2000MAY01 | 920 | 982 | forward 2 | TM | N out |
| 39 | LI:321475.1:2000MAY01 | 1013 | 1075 | forward 2 | TM | N out |
| 39 | LI:321475.1:2000MAY01 | 1124 | 1186 | forward 2 | TM | N out |
| 39 | LI:321475.1:2000MAY01 | 1202 | 1264 | forward 2 | TM | N out |
| 39 | LI:321475.1:2000MAY01 | 1409 | 1495 | forward 2 | TM | N out |
| 39 | LI:321475.1:2000MAY01 | 822 | 908 | forward 3 | TM | N in |
| 39 | LI:321475.1:2000MAY01 | 1125 | 1208 | forward 3 | TM | N in |
| 40 | LI:899552.5:2000MAY01 | 286 | 351 | forward 1 | TM | N in |
| 40 | LI:899552.5:2000MAY01 | 970 | 1056 | forward 1 | TM | N in |
| 40 | LI:899552.5:2000MAY01 | 1114 | 1176 | forward 1 | TM | N in |
| 40 | LI:899552.5:2000MAY01 | 1900 | 1947 | forward 1 | TM | N in |
| 40 | LI:899552.5:2000MAY01 | 1079 | 1165 | forward 2 | TM | N in |
| 40 | LI:899552.5:2000MAY01 | 1691 | 1765 | forward 2 | TM | N in |
| 40 | LI:899552.5:2000MAY01 | 834 | 896 | forward 3 | TM | N in |
| 40 | LI:899552.5:2000MAY01 | 915 | 977 | forward 3 | TM | N in |
| 40 | LI:899552.5:2000MAY01 | 996 | 1058 | forward 3 | TM | N in |
| 41 | LI:1071848.1:2000MAY01 | 961 | 1020 | forward 1 | TM | N out |
| 41 | LI:1071848.1:2000MAY01 | 1117 | 1194 | forward 1 | TM | N out |
| 41 | LI:1071848.1:2000MAY01 | 1058 | 1144 | forward 2 | TM | N out |
| 41 | LI:1071848.1:2000MAY01 | 996 | 1067 | forward 3 | TM | N in |
| 41 | LI:1071848.1:2000MAY01 | 1080 | 1142 | forward 3 | TM | N in |
| 41 | LI:1071848.1:2000MAY01 | 1155 | 1217 | forward 3 | TM | N in |
| 42 | LI:1072337.2:2000MAY01 | 49 | 135 | forward 1 | TM | N out |
| 42 | LI:1072337.2:2000MAY01 | 463 | 525 | forward 1 | TM | N out |
| 42 | LI:1072337.2:2000MAY01 | 652 | 726 | forward 1 | TM | N out |
| 42 | LI:1072337.2:2000MAY01 | 2344 | 2397 | forward 1 | TM | N out |
| 42 | LI:1072337.2:2000MAY01 | 2518 | 2589 | forward 1 | TM | N out |
| 42 | LI:1072337.2:2000MAY01 | 2614 | 2667 | forward 1 | TM | N out |
| 42 | LI:1072337.2:2000MAY01 | 2923 | 3000 | forward 1 | TM | N out |
| 42 | LI:1072337.2:2000MAY01 | 3181 | 3252 | forward 1 | TM | N out |
| 42 | LI:1072337.2:2000MAY01 | 3577 | 3657 | forward 1 | TM | N out |
| 42 | LI:1072337.2:2000MAY01 | 4264 | 4344 | forward 1 | TM | N out |
| 42 | LI:1072337.2:2000MAY01 | 77 | 136 | forward 2 | TM | N out |
| 42 | LI:1072337.2:2000MAY01 | 230 | 316 | forward 2 | TM | N out |
| 42 | LI:1072337.2:2000MAY01 | 1997 | 2047 | forward 2 | TM | N out |
| 42 | LI:1072337.2:2000MAY01 | 2057 | 2125 | forward 2 | TM | N out |
| 42 | LI:1072337.2:2000MAY01 | 2609 | 2686 | forward 2 | TM | N out |
| 42 | LI:1072337.2:2000MAY01 | 2861 | 2914 | forward 2 | TM | N out |
| 42 | LI:1072337.2:2000MAY01 | 3242 | 3328 | forward 2 | TM | N out |
| 42 | LI:1072337.2:2000MAY01 | 3440 | 3496 | forward 2 | TM | N out |
| 42 | LI:1072337.2:2000MAY01 | 3587 | 3673 | forward 2 | TM | N out |
| 42 | LI:1072337.2:2000MAY01 | 4391 | 4468 | forward 2 | TM | N out |
| 42 | LI:1072337.2:2000MAY01 | 2382 | 2435 | forward 3 | TM | N in |
| 42 | LI:1072337.2:2000MAY01 | 2529 | 2594 | forward 3 | TM | N in |
| 42 | LI:1072337.2:2000MAY01 | 2877 | 2948 | forward 3 | TM | N in |
| 42 | LI:1072337.2:2000MAY01 | 2994 | 3065 | forward 3 | TM | N in |
| 42 | LI:1072337.2:2000MAY01 | 3618 | 3695 | forward 3 | TM | N in |
| 42 | LI:1072337.2:2000MAY01 | 4386 | 4460 | forward 3 | TM | N in |
| 43 | LI:251489.5:2000MAY01 | 31 | 117 | forward 1 | TM | N out |
| 43 | LI:251489.5:2000MAY01 | 44 | 130 | forward 2 | TM | N out |
| 44 | LI:902018.107:2000MAY01 | 595 | 681 | forward 1 | TM | N out |
| 44 | LI:902018.107:2000MAY01 | 1429 | 1515 | forward 1 | TM | N out |
| 44 | LI:902018.107:2000MAY01 | 2098 | 2184 | forward 1 | TM | N out |
| 44 | LI:902018.107:2000MAY01 | 2341 | 2409 | forward 1 | TM | N out |
| 44 | LI:902018.107:2000MAY01 | 2833 | 2895 | forward 1 | TM | N out |
| 44 | LI:902018.107:2000MAY01 | 2926 | 2988 | forward 1 | TM | N out |
| 44 | LI:902018.107:2000MAY01 | 3616 | 3666 | forward 1 | TM | N out |
| 44 | LI:902018.107:2000MAY01 | 3766 | 3837 | forward 1 | TM | N out |
| 44 | LI:902018.107:2000MAY01 | 3937 | 3990 | forward 1 | TM | N out |
| 44 | LI:902018.107:2000MAY01 | 2675 | 2761 | forward 2 | TM | N in |
| 44 | LI:902018.107:2000MAY01 | 3611 | 3697 | forward 2 | TM | N in |
| 44 | LI:902018.107:2000MAY01 | 4043 | 4117 | forward 2 | TM | N in |
| 44 | LI:902018.107:2000MAY01 | 4151 | 4237 | forward 2 | TM | N in |
| 44 | LI:902018.107:2000MAY01 | 3057 | 3143 | forward 3 | TM | N in |
| 44 | LI:902018.107:2000MAY01 | 3555 | 3638 | forward 3 | TM | N in |
| 44 | LI:902018.107:2000MAY01 | 3681 | 3734 | forward 3 | TM | N in |
| 44 | LI:902018.107:2000MAY01 | 3915 | 3977 | forward 3 | TM | N in |
| 44 | LI:902018.107:2000MAY01 | 4002 | 4064 | forward 3 | TM | N in |
| 44 | LI:902018.107:2000MAY01 | 4080 | 4160 | forward 3 | TM | N in |
| 44 | LI:902018.107:2000MAY01 | 4170 | 4226 | forward 3 | TM | N in |
| 45 | LI:220495.1:2000MAY01 | 91 | 150 | forward 1 | TM | N out |
| 45 | LI:220495.1:2000MAY01 | 205 | 291 | forward 1 | TM | N out |
| 45 | LI:220495.1:2000MAY01 | 499 | 579 | forward 1 | TM | N out |
| 45 | LI:220495.1:2000MAY01 | 637 | 699 | forward 1 | TM | N out |
| 45 | LI:220495.1:2000MAY01 | 718 | 780 | forward 1 | TM | N out |
| 45 | LI:220495.1:2000MAY01 | 853 | 936 | forward 1 | TM | N out |
| 45 | LI:220495.1:2000MAY01 | 994 | 1080 | forward 1 | TM | N out |
| 45 | LI:220495.1:2000MAY01 | 1513 | 1590 | forward 1 | TM | N out |
| 45 | LI:220495.1:2000MAY01 | 1591 | 1671 | forward 1 | TM | N out |
| 45 | LI:220495.1:2000MAY01 | 1702 | 1788 | forward 1 | TM | N out |
| 45 | LI:220495.1:2000MAY01 | 1939 | 2022 | forward 1 | TM | N out |
| 45 | LI:220495.1:2000MAY01 | 2344 | 2430 | forward 1 | TM | N out |
| 45 | LI:220495.1:2000MAY01 | 2542 | 2592 | forward 1 | TM | N out |
| 45 | LI:220495.1:2000MAY01 | 779 | 832 | forward 2 | TM | N in |
| 45 | LI:220495.1:2000MAY01 | 851 | 916 | forward 2 | TM | N in |
| 45 | LI:220495.1:2000MAY01 | 1418 | 1504 | forward 2 | TM | N in |
| 45 | LI:220495.1:2000MAY01 | 1589 | 1642 | forward 2 | TM | N in |
| 45 | LI:220495.1:2000MAY01 | 1682 | 1768 | forward 2 | TM | N in |
| 45 | LI:220495.1:2000MAY01 | 1823 | 1885 | forward 2 | TM | N in |
| 45 | LI:220495.1:2000MAY01 | 1898 | 1960 | forward 2 | TM | N in |
| 45 | LI:220495.1:2000MAY01 | 2894 | 2974 | forward 2 | TM | N in |
| 45 | LI:220495.1:2000MAY01 | 486 | 536 | forward 3 | TM | N in |
| 45 | LI:220495.1:2000MAY01 | 618 | 677 | forward 3 | TM | N in |
| 45 | LI:220495.1:2000MAY01 | 960 | 1034 | forward 3 | TM | N in |
| 45 | LI:220495.1:2000MAY01 | 1692 | 1751 | forward 3 | TM | N in |
| 45 | LI:220495.1:2000MAY01 | 1812 | 1895 | forward 3 | TM | N in |
| 45 | LI:220495.1:2000MAY01 | 1932 | 1991 | forward 3 | TM | N in |
| 45 | LI:220495.1:2000MAY01 | 2307 | 2366 | forward 3 | TM | N in |
| 45 | LI:220495.1:2000MAY01 | 2391 | 2462 | forward 3 | TM | N in |
| 45 | LI:220495.1:2000MAY01 | 2835 | 2921 | forward 3 | TM | N in |
| 45 | LI:220495.1:2000MAY01 | 2970 | 3056 | forward 3 | TM | N in |
| 46 | LI:399478.1:2000MAY01 | 823 | 885 | forward 1 | TM | N in |
| 46 | LI:399478.1:2000MAY01 | 982 | 1059 | forward 1 | TM | N in |
| 46 | LI:399478.1:2000MAY01 | 1087 | 1173 | forward 1 | TM | N in |
| 46 | LI:399478.1:2000MAY01 | 1330 | 1416 | forward 1 | TM | N in |
| 46 | LI:399478.1:2000MAY01 | 1639 | 1716 | forward 1 | TM | N in |
| 46 | LI:399478.1:2000MAY01 | 1756 | 1836 | forward 1 | TM | N in |
| 46 | LI:399478.1:2000MAY01 | 1849 | 1899 | forward 1 | TM | N in |
| 46 | LI:399478.1:2000MAY01 | 2497 | 2574 | forward 1 | TM | N in |
| 46 | LI:399478.1:2000MAY01 | 2716 | 2772 | forward 1 | TM | N in |
| 46 | LI:399478.1:2000MAY01 | 107 | 178 | forward 2 | TM | N out |
| 46 | LI:399478.1:2000MAY01 | 1025 | 1087 | forward 2 | TM | N out |
| 46 | LI:399478.1:2000MAY01 | 1103 | 1165 | forward 2 | TM | N out |
| 46 | LI:399478.1:2000MAY01 | 1640 | 1702 | forward 2 | TM | N out |
| 46 | LI:399478.1:2000MAY01 | 1718 | 1780 | forward 2 | TM | N out |
| 46 | LI:399478.1:2000MAY01 | 2063 | 2149 | forward 2 | TM | N out |
| 46 | LI:399478.1:2000MAY01 | 519 | 605 | forward 3 | TM | N in |
| 46 | LI:399478.1:2000MAY01 | 834 | 914 | forward 3 | TM | N in |
| 46 | LI:399478.1:2000MAY01 | 1014 | 1076 | forward 3 | TM | N in |
| 46 | LI:399478.1:2000MAY01 | 1101 | 1163 | forward 3 | TM | N in |
| 46 | LI:399478.1:2000MAY01 | 1386 | 1439 | forward 3 | TM | N in |
| 46 | LI:399478.1:2000MAY01 | 1509 | 1595 | forward 3 | TM | N in |
| 46 | LI:399478.1:2000MAY01 | 1659 | 1745 | forward 3 | TM | N in |
| 46 | LI:399478.1:2000MAY01 | 1803 | 1886 | forward 3 | TM | N in |
| 46 | LI:399478.1:2000MAY01 | 2583 | 2669 | forward 3 | TM | N in |
| 47 | LI:229648.2:2000MAY01 | 556 | 606 | forward 1 | TM | |
| 47 | LI:229648.2:2000MAY01 | 1009 | 1095 | forward 1 | TM | |
| 47 | LI:229648.2:2000MAY01 | 1324 | 1410 | forward 1 | TM | |
| 47 | LI:229648.2:2000MAY01 | 1633 | 1719 | forward 1 | TM | |
| 47 | LI:229648.2:2000MAY01 | 1840 | 1914 | forward 1 | TM | |
| 47 | LI:229648.2:2000MAY01 | 776 | 832 | forward 2 | TM | N in |
| 47 | LI:229648.2:2000MAY01 | 983 | 1069 | forward 2 | TM | N in |
| 47 | LI:229648.2:2000MAY01 | 1610 | 1663 | forward 2 | TM | N in |
| 47 | LI:229648.2:2000MAY01 | 1862 | 1921 | forward 2 | TM | N in |
| 47 | LI:229648.2:2000MAY01 | 726 | 812 | forward 3 | TM | N in |
| 47 | LI:229648.2:2000MAY01 | 1002 | 1088 | forward 3 | TM | N in |
| 47 | LI:229648.2:2000MAY01 | 1533 | 1589 | forward 3 | TM | N in |
| 47 | LI:229648.2:2000MAY01 | 1641 | 1727 | forward 3 | TM | N in |
| 47 | LI:229648.2:2000MAY01 | 1809 | 1895 | forward 3 | TM | N in |
| 48 | LI:025643.2:2000MAY01 | 1526 | 1591 | forward 2 | TM | |
| 48 | LI:025643.2:2000MAY01 | 1542 | 1598 | forward 3 | TM | N out |
| 49 | LI:233942.1:2000MAY01 | 184 | 270 | forward 1 | TM | N out |
| 49 | LI:233942.1:2000MAY01 | 541 | 627 | forward 1 | TM | N out |
| 49 | LI:233942.1:2000MAY01 | 671 | 745 | forward 2 | TM | N out |
| 49 | LI:233942.1:2000MAY01 | 1352 | 1438 | forward 2 | TM | N out |
| 49 | LI:233942.1:2000MAY01 | 1715 | 1768 | forward 2 | TM | N out |
| 49 | LI:233942.1:2000MAY01 | 2021 | 2104 | forward 2 | TM | N out |
| 49 | LI:233942.1:2000MAY01 | 138 | 221 | forward 3 | TM | N in |
| 49 | LI:233942.1:2000MAY01 | 558 | 614 | forward 3 | TM | N in |
| 49 | LI:233942.1:2000MAY01 | 684 | 758 | forward 3 | TM | N in |
| 49 | LI:233942.1:2000MAY01 | 1179 | 1256 | forward 3 | TM | N in |
| 49 | LI:233942.1:2000MAY01 | 1986 | 2057 | forward 3 | TM | N in |
| 50 | LI:089158.1:2000MAY01 | 2443 | 2526 | forward 1 | TM | N out |
| 50 | LI:089158.1:2000MAY01 | 2602 | 2652 | forward 1 | TM | N out |
| 50 | LI:089158.1:2000MAY01 | 3772 | 3858 | forward 1 | TM | N out |
| 50 | LI:089158.1:2000MAY01 | 1052 | 1120 | forward 2 | TM | N in |
| 50 | LI:089158.1:2000MAY01 | 1523 | 1597 | forward 2 | TM | N in |
| 50 | LI:089158.1:2000MAY01 | 1643 | 1729 | forward 2 | TM | N in |
| 50 | LI:089158.1:2000MAY01 | 2396 | 2476 | forward 2 | TM | N in |
| 50 | LI:089158.1:2000MAY01 | 3335 | 3421 | forward 2 | TM | N in |
| 50 | LI:089158.1:2000MAY01 | 1020 | 1100 | forward 3 | TM | N in |
| 50 | LI:089158.1:2000MAY01 | 1497 | 1583 | forward 3 | TM | N in |
| 50 | LI:089158.1:2000MAY01 | 1722 | 1790 | forward 3 | TM | N in |
| 50 | LI:089158.1:2000MAY01 | 3069 | 3146 | forward 3 | TM | N in |
| 51 | LI:101046.1:2000MAY01 | 292 | 378 | forward 1 | TM | N out |
| 51 | LI:101046.1:2000MAY01 | 811 | 897 | forward 1 | TM | N out |
| 51 | LI:101046.1:2000MAY01 | 1261 | 1308 | forward 1 | TM | N out |
| 51 | LI:101046.1:2000MAY01 | 368 | 451 | forward 2 | TM | N in |
| 51 | LI:101046.1:2000MAY01 | 827 | 913 | forward 2 | TM | N in |
| 51 | LI:101046.1:2000MAY01 | 2102 | 2188 | forward 2 | TM | N in |
| 51 | LI:101046.1:2000MAY01 | 711 | 785 | forward 3 | TM | N out |
| 51 | LI:101046.1:2000MAY01 | 873 | 932 | forward 3 | TM | N out |
| 51 | LI:101046.1:2000MAY01 | 1002 | 1079 | forward 3 | TM | N out |
| 52 | LI:368676.2:2000MAY01 | 2077 | 2163 | forward 1 | TM | |
| 52 | LI:368676.2:2000MAY01 | 3079 | 3165 | forward 1 | TM | |
| 52 | LI:368676.2:2000MAY01 | 2984 | 3058 | forward 2 | TM | N in |
| 52 | LI:368676.2:2000MAY01 | 2937 | 3023 | forward 3 | TM | |
| 53 | LI:238713.1:2000MAY01 | 844 | 930 | forward 1 | TM | |
| 53 | LI:238713.1:2000MAY01 | 110 | 184 | forward 2 | TM | N out |
| 53 | LI:238713.1:2000MAY01 | 194 | 256 | forward 2 | TM | N out |
| 53 | LI:238713.1:2000MAY01 | 728 | 814 | forward 2 | TM | N out |
| 53 | LI:238713.1:2000MAY01 | 297 | 383 | forward 3 | TM | N out |
| 53 | LI:238713.1:2000MAY01 | 423 | 485 | forward 3 | TM | N out |
| 53 | LI:238713.1:2000MAY01 | 570 | 656 | forward 3 | TM | N out |
| 53 | LI:238713.1:2000MAY01 | 696 | 782 | forward 3 | TM | N out |
| 53 | LI:238713.1:2000MAY01 | 1101 | 1154 | forward 3 | TM | N out |
| 53 | LI:238713.1:2000MAY01 | 1635 | 1721 | forward 3 | TM | N out |
| 54 | LI:720928.1:2000MAY01 | 208 | 264 | forward 1 | TM | N out |
| 54 | LI:720928.1:2000MAY01 | 637 | 693 | forward 1 | TM | N out |
| 55 | LI:221874.1:2000MAY01 | 877 | 954 | forward 1 | TM | N out |
| 55 | LI:221874.1:2000MAY01 | 2086 | 2172 | forward 1 | TM | N out |
| 55 | LI:221874.1:2000MAY01 | 1187 | 1258 | forward 2 | TM | N out |
| 55 | LI:221874.1:2000MAY01 | 2171 | 2230 | forward 2 | TM | N out |
| 55 | LI:221874.1:2000MAY01 | 348 | 434 | forward 3 | TM | N in |
| 55 | LI:221874.1:2000MAY01 | 1116 | 1190 | forward 3 | TM | N in |
| 56 | LI:1143545.3:2000MAY01 | 458 | 523 | forward 2 | TM | |
| 56 | LI:1143545.3:2000MAY01 | 2144 | 2230 | forward 2 | TM | |
| 57 | LI:1143605.1:2000MAY01 | 349 | 435 | forward 1 | TM | N in |
| 57 | LI:1143605.1:2000MAY01 | 445 | 528 | forward 1 | TM | N in |
| 57 | LI:1143605.1:2000MAY01 | 544 | 624 | forward 1 | TM | N in |
| 57 | LI:1143605.1:2000MAY01 | 775 | 861 | forward 1 | TM | N in |
| 57 | LI:1143605.1:2000MAY01 | 1102 | 1188 | forward 1 | TM | N in |
| 57 | LI:1143605.1:2000MAY01 | 1240 | 1302 | forward 1 | TM | N in |
| 57 | LI:1143605.1:2000MAY01 | 1378 | 1464 | forward 1 | TM | N in |
| 57 | LI:1143605.1:2000MAY01 | 1726 | 1800 | forward 1 | TM | N in |
| 57 | LI:1143605.1:2000MAY01 | 1828 | 1890 | forward 1 | TM | N in |
| 57 | LI:1143605.1:2000MAY01 | 1918 | 1980 | forward 1 | TM | N in |
| 57 | LI:1143605.1:2000MAY01 | 17 | 82 | forward 2 | TM | N out |
| 57 | LI:1143605.1:2000MAY01 | 368 | 445 | forward 2 | TM | N out |
| 57 | LI:1143605.1:2000MAY01 | 632 | 718 | forward 2 | TM | N out |
| 57 | LI:1143605.1:2000MAY01 | 899 | 958 | forward 2 | TM | N out |
| 57 | LI:1143605.1:2000MAY01 | 1043 | 1102 | forward 2 | TM | N out |
| 57 | LI:1143605.1:2000MAY01 | 1157 | 1243 | forward 2 | TM | N out |
| 57 | LI:1143605.1:2000MAY01 | 1607 | 1681 | forward 2 | TM | N out |
| 57 | LI:1143605.1:2000MAY01 | 1766 | 1849 | forward 2 | TM | N out |
| 57 | LI:1143605.1:2000MAY01 | 1859 | 1927 | forward 2 | TM | N out |
| 57 | LI:1143605.1:2000MAY01 | 1940 | 1999 | forward 2 | TM | N out |
| 57 | LI:1143605.1:2000MAY01 | 351 | 437 | forward 3 | TM | N out |
| 57 | LI:1143605.1:2000MAY01 | 513 | 584 | forward 3 | TM | N out |
| 57 | LI:1143605.1:2000MAY01 | 735 | 821 | forward 3 | TM | N out |
| 57 | LI:1143605.1:2000MAY01 | 1326 | 1412 | forward 3 | TM | N out |
| 57 | LI:1143605.1:2000MAY01 | 1446 | 1526 | forward 3 | TM | N out |
| 57 | LI:1143605.1:2000MAY01 | 1605 | 1688 | forward 3 | TM | N out |
| 57 | LI:1143605.1:2000MAY01 | 1806 | 1868 | forward 3 | TM | N out |
| 57 | LI:1143605.1:2000MAY01 | 1893 | 1955 | forward 3 | TM | N out |
| 58 | LI:474069.7:2000MAY01 | 706 | 783 | forward 1 | TM | |
| 58 | LI:474069.7:2000MAY01 | 1366 | 1428 | forward 1 | TM | |
| 58 | LI:474069.7:2000MAY01 | 1738 | 1824 | forward 1 | TM | |
| 58 | LI:474069.7:2000MAY01 | 698 | 748 | forward 2 | TM | N in |
| 58 | LI:474069.7:2000MAY01 | 965 | 1051 | forward 2 | TM | N in |
| 58 | LI:474069.7:2000MAY01 | 1241 | 1300 | forward 2 | TM | N in |
| 58 | LI:474069.7:2000MAY01 | 1487 | 1537 | forward 2 | TM | N in |
| 58 | LI:474069.7:2000MAY01 | 2030 | 2107 | forward 2 | TM | N in |
| 58 | LI:474069.7:2000MAY01 | 27 | 92 | forward 3 | TM | N in |
| 58 | LI:474069.7:2000MAY01 | 699 | 785 | forward 3 | TM | N in |
| 58 | LI:474069.7:2000MAY01 | 936 | 1001 | forward 3 | TM | N in |
| 58 | LI:474069.7:2000MAY01 | 1929 | 2003 | forward 3 | TM | N in |
| 59 | LI:245193.3:2000MAY01 | 1195 | 1275 | forward 1 | TM | N in |
| 59 | LI:245193.3:2000MAY01 | 1579 | 1656 | forward 1 | TM | N in |
| 59 | LI:245193.3:2000MAY01 | 1789 | 1860 | forward 1 | TM | N in |
| 59 | LI:245193.3:2000MAY01 | 2029 | 2091 | forward 1 | TM | N in |
| 59 | LI:245193.3:2000MAY01 | 1562 | 1621 | forward 2 | TM | N in |
| 59 | LI:245193.3:2000MAY01 | 1880 | 1957 | forward 2 | TM | N in |
| 59 | LI:245193.3:2000MAY01 | 2156 | 2206 | forward 2 | TM | N in |
| 59 | LI:245193.3:2000MAY01 | 2513 | 2575 | forward 2 | TM | N in |
| 59 | LI:245193.3:2000MAY01 | 2591 | 2653 | forward 2 | TM | N in |
| 59 | LI:245193.3:2000MAY01 | 2669 | 2731 | forward 2 | TM | N in |
| 59 | LI:245193.3:2000MAY01 | 1548 | 1634 | forward 3 | TM | N in |
| 59 | LI:245193.3:2000MAY01 | 1659 | 1745 | forward 3 | TM | N in |
| 59 | LI:245193.3:2000MAY01 | 2154 | 2231 | forward 3 | TM | N in |
| 59 | LI:245193.3:2000MAY01 | 2364 | 2450 | forward 3 | TM | N in |
| 59 | LI:245193.3:2000MAY01 | 2571 | 2618 | forward 3 | TM | N in |
| 59 | LI:245193.3:2000MAY01 | 2649 | 2735 | forward 3 | TM | N in |
| 60 | LI:403872.1:2000MAY01 | 562 | 624 | forward 1 | TM | |
| 60 | LI:403872.1:2000MAY01 | 730 | 780 | forward 1 | TM | |
| 60 | LI:403872.1:2000MAY01 | 1498 | 1575 | forward 1 | TM | |
| 60 | LI:403872.1:2000MAY01 | 1648 | 1722 | forward 1 | TM | |
| 60 | LI:403872.1:2000MAY01 | 2359 | 2433 | forward 1 | TM | |
| 60 | LI:403872.1:2000MAY01 | 371 | 454 | forward 2 | TM | N in |
| 60 | LI:403872.1:2000MAY01 | 731 | 781 | forward 2 | TM | N in |
| 60 | LI:403872.1:2000MAY01 | 956 | 1012 | forward 2 | TM | N in |
| 60 | LI:403872.1:2000MAY01 | 1106 | 1192 | forward 2 | TM | N in |
| 60 | LI:403872.1:2000MAY01 | 1541 | 1600 | forward 2 | TM | N in |
| 60 | LI:403872.1:2000MAY01 | 1661 | 1747 | forward 2 | TM | N in |
| 60 | LI:403872.1:2000MAY01 | 1994 | 2050 | forward 2 | TM | N in |
| 60 | LI:403872.1:2000MAY01 | 2261 | 2332 | forward 2 | TM | N in |
| 60 | LI:403872.1:2000MAY01 | 2357 | 2443 | forward 2 | TM | N in |
| 60 | LI:403872.1:2000MAY01 | 543 | 629 | forward 3 | TM | N out |
| 60 | LI:403872.1:2000MAY01 | 708 | 782 | forward 3 | TM | N out |
| 60 | LI:403872.1:2000MAY01 | 1530 | 1601 | forward 3 | TM | N out |
| 60 | LI:403872.1:2000MAY01 | 1656 | 1715 | forward 3 | TM | N out |
| 60 | LI:403872.1:2000MAY01 | 1983 | 2069 | forward 3 | TM | N out |
| 60 | LI:403872.1:2000MAY01 | 2145 | 2225 | forward 3 | TM | N out |
| 60 | LI:403872.1:2000MAY01 | 2226 | 2309 | forward 3 | TM | N out |
| 60 | LI:403872.1:2000MAY01 | 2349 | 2426 | forward 3 | TM | N out |
| 61 | LI:1086294.1:2000MAY01 | 748 | 834 | forward 1 | TM | N out |
| 61 | LI:1086294.1:2000MAY01 | 2278 | 2352 | forward 1 | TM | N out |
| 61 | LI:1086294.1:2000MAY01 | 2306 | 2356 | forward 2 | TM | N out |
| 62 | LI:337514.3:2000MAY01 | 1906 | 1992 | forward 1 | TM | N in |
| 62 | LI:337514.3:2000MAY01 | 1512 | 1598 | forward 3 | TM | N out |
| 63 | LI:230711.1:2000MAY01 | 898 | 978 | forward 1 | TM | N out |
| 63 | LI:230711.1:2000MAY01 | 1327 | 1389 | forward 1 | TM | N out |
| 63 | LI:230711.1:2000MAY01 | 2059 | 2145 | forward 1 | TM | N out |
| 63 | LI:230711.1:2000MAY01 | 134 | 220 | forward 2 | TM | N out |
| 63 | LI:230711.1:2000MAY01 | 1517 | 1591 | forward 2 | TM | N out |
| 63 | LI:230711.1:2000MAY01 | 1631 | 1708 | forward 2 | TM | N out |
| 63 | LI:230711.1:2000MAY01 | 2033 | 2119 | forward 2 | TM | N out |
| 63 | LI:230711.1:2000MAY01 | 114 | 188 | forward 3 | TM | N in |
| 63 | LI:230711.1:2000MAY01 | 570 | 620 | forward 3 | TM | N in |
| 63 | LI:230711.1:2000MAY01 | 648 | 716 | forward 3 | TM | N in |
| 63 | LI:230711.1:2000MAY01 | 1101 | 1160 | forward 3 | TM | N in |
| 64 | LI:040338.2:2000MAY01 | 794 | 880 | forward 2 | TM | N in |
| 64 | LI:040338.2:2000MAY01 | 162 | 233 | forward 3 | TM | N out |
| 64 | LI:040338.2:2000MAY01 | 708 | 791 | forward 3 | TM | N out |
| 65 | LI:399174.2:2000MAY01 | 622 | 693 | forward 1 | TM | |
| 65 | LI:399174.2:2000MAY01 | 958 | 1008 | forward 1 | TM | |
| 65 | LI:399174.2:2000MAY01 | 1027 | 1080 | forward 1 | TM | |
| 65 | LI:399174.2:2000MAY01 | 1303 | 1377 | forward 1 | TM | |
| 65 | LI:399174.2:2000MAY01 | 965 | 1018 | forward 2 | TM | N in |
| 65 | LI:399174.2:2000MAY01 | 126 | 200 | forward 3 | TM | N out |
| 66 | LI:197275.5:2000MAY01 | 211 | 285 | forward 1 | TM | N out |
| 66 | LI:197275.5:2000MAY01 | 761 | 811 | forward 2 | TM | N out |
| 66 | LI:197275.5:2000MAY01 | 210 | 281 | forward 3 | TM | N out |
| 66 | LI:197275.5:2000MAY01 | 537 | 623 | forward 3 | TM | N out |
| 67 | LI:336872.1:2000MAY01 | 175 | 231 | forward 1 | TM | N out |
| 67 | LI:336872.1:2000MAY01 | 1099 | 1185 | forward 1 | TM | N out |
| 67 | LI:336872.1:2000MAY01 | 1450 | 1536 | forward 1 | TM | N out |
| 67 | LI:336872.1:2000MAY01 | 182 | 247 | forward 2 | TM | N in |
| 67 | LI:336872.1:2000MAY01 | 1001 | 1072 | forward 2 | TM | N in |
| 67 | LI:336872.1:2000MAY01 | 1169 | 1246 | forward 2 | TM | N in |
| 67 | LI:336872.1:2000MAY01 | 1373 | 1447 | forward 2 | TM | N in |
| 67 | LI:336872.1:2000MAY01 | 171 | 224 | forward 3 | TM | N out |
| 67 | LI:336872.1:2000MAY01 | 1089 | 1175 | forward 3 | TM | N out |
| 67 | LI:336872.1:2000MAY01 | 1302 | 1364 | forward 3 | TM | N out |
| 68 | LI:1092901.1:2000MAY01 | 172 | 258 | forward 1 | TM | N out |
| 68 | LI:1092901.1:2000MAY01 | 299 | 379 | forward 2 | TM | N out |
| 68 | LI:1092901.1:2000MAY01 | 425 | 511 | forward 2 | TM | N out |
| 69 | LI:022387.5:2000MAY01 | 1195 | 1281 | forward 1 | TM | N out |
| 69 | LI:022387.5:2000MAY01 | 710 | 796 | forward 2 | TM | N out |
| 69 | LI:022387.5:2000MAY01 | 1214 | 1276 | forward 2 | TM | N out |
| 69 | LI:022387.5:2000MAY01 | 675 | 743 | forward 3 | TM | N out |
| 69 | LI:022387.5:2000MAY01 | 1092 | 1175 | forward 3 | TM | N out |
| 69 | LI:022387.5:2000MAY01 | 1215 | 1301 | forward 3 | TM | N out |
| 70 | LI:1188334.1:2000MAY01 | 208 | 294 | forward 1 | TM | N out |
| 70 | LI:1188334.1:2000MAY01 | 370 | 456 | forward 1 | TM | N out |
| 70 | LI:1188334.1:2000MAY01 | 11 | 82 | forward 2 | TM | N out |
| 70 | LI:1188334.1:2000MAY01 | 131 | 190 | forward 2 | TM | N out |
| 70 | LI:1188334.1:2000MAY01 | 12 | 80 | forward 3 | TM | N in |
| 70 | LI:1188334.1:2000MAY01 | 123 | 194 | forward 3 | TM | N in |
| 71 | LI:1188664.1:2000MAY01 | 274 | 348 | forward 1 | TM | N in |
| 71 | LI:1188664.1:2000MAY01 | 607 | 657 | forward 1 | TM | N in |
| 71 | LI:1188664.1:2000MAY01 | 254 | 304 | forward 2 | TM | N in |
| 71 | LI:1188664.1:2000MAY01 | 494 | 580 | forward 2 | TM | N in |
| 72 | LI:247388.1:2000MAY01 | 1126 | 1212 | forward 1 | TM | N out |
| 72 | LI:247388.1:2000MAY01 | 134 | 220 | forward 2 | TM | N in |
| 72 | LI:247388.1:2000MAY01 | 389 | 448 | forward 2 | TM | N in |
| 72 | LI:247388.1:2000MAY01 | 968 | 1033 | forward 2 | TM | N in |
| 72 | LI:247388.1:2000MAY01 | 741 | 812 | forward 3 | TM | N in |
| 72 | LI:247388.1:2000MAY01 | 1200 | 1277 | forward 3 | TM | N in |
| 73 | LI:816339.4:2000MAY01 | 466 | 534 | forward 1 | TM | N in |
| 73 | LI:816339.4:2000MAY01 | 562 | 648 | forward 1 | TM | N in |
| 73 | LI:816339.4:2000MAY01 | 937 | 1017 | forward 1 | TM | N in |
| 73 | LI:816339.4:2000MAY01 | 1162 | 1248 | forward 1 | TM | N in |
| 73 | LI:816339.4:2000MAY01 | 1306 | 1368 | forward 1 | TM | N in |
| 73 | LI:816339.4:2000MAY01 | 1390 | 1452 | forward 1 | TM | N in |
| 73 | LI:816339.4:2000MAY01 | 407 | 487 | forward 2 | TM | N out |
| 73 | LI:816339.4:2000MAY01 | 575 | 652 | forward 2 | TM | N out |
| 73 | LI:816339.4:2000MAY01 | 698 | 784 | forward 2 | TM | N out |
| 73 | LI:816339.4:2000MAY01 | 950 | 1012 | forward 2 | TM | N out |
| 73 | LI:816339.4:2000MAY01 | 1043 | 1105 | forward 2 | TM | N out |
| 73 | LI:816339.4:2000MAY01 | 1136 | 1198 | forward 2 | TM | N out |
| 73 | LI:816339.4:2000MAY01 | 1244 | 1306 | forward 2 | TM | N out |
| 73 | LI:816339.4:2000MAY01 | 1334 | 1396 | forward 2 | TM | N out |
| 73 | LI:816339.4:2000MAY01 | 1424 | 1486 | forward 2 | TM | N out |
| 73 | LI:816339.4:2000MAY01 | 468 | 554 | forward 3 | TM | N out |
| 73 | LI:816339.4:2000MAY01 | 591 | 644 | forward 3 | TM | N out |
| 73 | LI:816339.4:2000MAY01 | 933 | 1010 | forward 3 | TM | N out |
| 73 | LI:816339.4:2000MAY01 | 1182 | 1244 | forward 3 | TM | N out |
| 73 | LI:816339.4:2000MAY01 | 1257 | 1319 | forward 3 | TM | N out |
| 74 | LI:1188967.1:2000MAY01 | 14 | 67 | forward 2 | TM | N out |
| 74 | LI:1188967.1:2000MAY01 | 272 | 325 | forward 2 | TM | N out |
| 75 | LI:236230.3:2000MAY01 | 1081 | 1149 | forward 1 | TM | N in |
| 75 | LI:236230.3:2000MAY01 | 1195 | 1281 | forward 1 | TM | N in |
| 75 | LI:236230.3:2000MAY01 | 1330 | 1416 | forward 1 | TM | N in |
| 75 | LI:236230.3:2000MAY01 | 182 | 253 | forward 2 | TM | N out |
| 75 | LI:236230.3:2000MAY01 | 1016 | 1102 | forward 2 | TM | N out |
| 75 | LI:236230.3:2000MAY01 | 1154 | 1240 | forward 2 | TM | N out |
| 75 | LI:236230.3:2000MAY01 | 1346 | 1432 | forward 2 | TM | N out |
| 75 | LI:236230.3:2000MAY01 | 63 | 149 | forward 3 | TM | N out |
| 75 | LI:236230.3:2000MAY01 | 396 | 449 | forward 3 | TM | N out |
| 75 | LI:236230.3:2000MAY01 | 567 | 644 | forward 3 | TM | N out |
| 75 | LI:236230.3:2000MAY01 | 1107 | 1181 | forward 3 | TM | N out |
| 75 | LI:236230.3:2000MAY01 | 1320 | 1382 | forward 3 | TM | N out |
| 75 | LI:236230.3:2000MAY01 | 1410 | 1472 | forward 3 | TM | N out |
| 76 | LI:246728.3:2000MAY01 | 1633 | 1695 | forward 1 | TM | N out |
| 76 | LI:246728.3:2000MAY01 | 1783 | 1854 | forward 1 | TM | N out |
| 76 | LI:246728.3:2000MAY01 | 2492 | 2566 | forward 2 | TM | N out |
| 76 | LI:246728.3:2000MAY01 | 792 | 854 | forward 3 | TM | N in |
| 76 | LI:246728.3:2000MAY01 | 2325 | 2375 | forward 3 | TM | N in |
| 77 | LI:1190057.1:2000MAY01 | 646 | 696 | forward 1 | TM | N in |
| 77 | LI:1190057.1:2000MAY01 | 1195 | 1263 | forward 1 | TM | N in |
| 77 | LI:1190057.1:2000MAY01 | 1591 | 1668 | forward 1 | TM | N in |
| 77 | LI:1190057.1:2000MAY01 | 2104 | 2169 | forward 1 | TM | N in |
| 77 | LI:1190057.1:2000MAY01 | 2233 | 2295 | forward 1 | TM | N in |
| 77 | LI:1190057.1:2000MAY01 | 2320 | 2382 | forward 1 | TM | N in |
| 77 | LI:1190057.1:2000MAY01 | 2623 | 2703 | forward 1 | TM | N in |
| 77 | LI:1190057.1:2000MAY01 | 3124 | 3207 | forward 1 | TM | N in |
| 77 | LI:1190057.1:2000MAY01 | 1217 | 1270 | forward 2 | TM | N out |
| 77 | LI:1190057.1:2000MAY01 | 1595 | 1681 | forward 2 | TM | N out |
| 77 | LI:1190057.1:2000MAY01 | 2267 | 2353 | forward 2 | TM | N out |
| 77 | LI:1190057.1:2000MAY01 | 2420 | 2491 | forward 2 | TM | N out |
| 77 | LI:1190057.1:2000MAY01 | 2624 | 2698 | forward 2 | TM | N out |
| 77 | LI:1190057.1:2000MAY01 | 2720 | 2785 | forward 2 | TM | N out |
| 77 | LI:1190057.1:2000MAY01 | 3014 | 3067 | forward 2 | TM | N out |
| 77 | LI:1190057.1:2000MAY01 | 3116 | 3202 | forward 2 | TM | N out |
| 77 | LI:1190057.1:2000MAY01 | 1905 | 1991 | forward 3 | TM | N in |
| 77 | LI:1190057.1:2000MAY01 | 2217 | 2297 | forward 3 | TM | N in |
| 77 | LI:1190057.1:2000MAY01 | 2601 | 2657 | forward 3 | TM | N in |
| 77 | LI:1190057.1:2000MAY01 | 3072 | 3143 | forward 3 | TM | N in |
| 78 | LI:221836.3:2000MAY01 | 775 | 846 | forward 1 | TM | N out |
| 78 | LI:221836.3:2000MAY01 | 2050 | 2106 | forward 1 | TM | N out |
| 78 | LI:221836.3:2000MAY01 | 2197 | 2265 | forward 1 | TM | N out |
| 78 | LI:221836.3:2000MAY01 | 2539 | 2613 | forward 1 | TM | N out |
| 78 | LI:221836.3:2000MAY01 | 2216 | 2302 | forward 2 | TM | |
| 78 | LI:221836.3:2000MAY01 | 2582 | 2668 | forward 2 | TM | |
| 78 | LI:221836.3:2000MAY01 | 1899 | 1976 | forward 3 | TM | N out |
| 78 | LI:221836.3:2000MAY01 | 2034 | 2120 | forward 3 | TM | N out |
| 78 | LI:221836.3:2000MAY01 | 2199 | 2261 | forward 3 | TM | N out |
| 78 | LI:221836.3:2000MAY01 | 2283 | 2345 | forward 3 | TM | N out |
| 78 | LI:221836.3:2000MAY01 | 2490 | 2564 | forward 3 | TM | N out |
| 79 | LI:334047.3:2000MAY01 | 115 | 201 | forward 1 | TM | N out |
| 79 | LI:334047.3:2000MAY01 | 56 | 142 | forward 2 | TM | N in |
| 79 | LI:334047.3:2000MAY01 | 497 | 577 | forward 2 | TM | N in |

TABLE 2

| SEQ | | | |
|---|---|---|---|
| ID NO: | Component ID | Start | Stop |
| 1 | 2079155F6 | 1 | 474 |
| 1 | 2079155H1 | 1 | 287 |
| 1 | 6485024H1 | 30 | 531 |
| 1 | 269086H1 | 55 | 416 |
| 1 | g4194935 | 215 | 662 |
| 1 | g3174230 | 294 | 608 |
| 2 | g3405927 | 511 | 865 |
| 2 | 4061048T8 | 213 | 755 |
| 2 | 5961372H1 | 1 | 539 |
| 3 | 4318348F6 | 361 | 482 |
| 3 | g4739876 | 483 | 760 |
| 3 | g5637692 | 483 | 760 |
| 3 | g5392945 | 603 | 1049 |
| 3 | 5338818H1 | 827 | 1078 |
| 3 | 6484158H1 | 1 | 544 |
| 3 | 5792010H1 | 36 | 340 |
| 3 | 5785018H1 | 36 | 336 |
| 3 | 5793390H1 | 36 | 306 |
| 3 | 6555681H1 | 125 | 694 |
| 3 | 3903879H1 | 156 | 435 |
| 3 | g4850881 | 306 | 759 |
| 4 | g2411092 | 1 | 241 |
| 4 | 7104605H1 | 1 | 533 |
| 4 | 4699688T6 | 146 | 533 |
| 5 | 6440110H1 | 1 | 71 |
| 5 | 5406681H1 | 1 | 122 |
| 5 | 6953084H1 | 63 | 615 |
| 5 | 5373405H1 | 63 | 285 |
| 5 | 5373405F8 | 83 | 602 |
| 5 | 3942701H1 | 190 | 481 |
| 5 | 4340882H1 | 392 | 586 |
| 5 | 4340882F6 | 392 | 865 |
| 5 | 3323337H1 | 449 | 716 |
| 5 | 1479486F6 | 453 | 867 |
| 5 | 1479486H1 | 453 | 688 |
| 5 | 4774836H1 | 1896 | 2149 |
| 5 | 4229676H1 | 1940 | 2149 |
| 5 | 4254348H1 | 2050 | 2148 |
| 5 | 1993010H1 | 483 | 763 |
| 5 | 3471007H1 | 681 | 951 |
| 5 | 3915086H1 | 718 | 875 |
| 5 | 2708491F6 | 775 | 1111 |
| 5 | 2708491H1 | 776 | 1069 |
| 5 | 6307323H1 | 845 | 1181 |
| 5 | 551890H1 | 939 | 1121 |
| 5 | 4492287H1 | 940 | 1315 |
| 5 | 3783242H1 | 1095 | 1382 |
| 5 | 1479372H1 | 1229 | 1476 |
| 5 | 2761062H1 | 1232 | 1497 |
| 5 | 2761062R6 | 1232 | 1489 |
| 5 | 6001120H1 | 1289 | 1774 |
| 5 | 6977932H1 | 1468 | 1665 |
| 5 | 7033269H1 | 1506 | 2033 |
| 5 | 3466972H1 | 1516 | 1770 |
| 5 | 2788543H1 | 1517 | 1748 |
| 5 | 5858122H1 | 1588 | 1848 |
| 5 | 2705868F6 | 1617 | 1980 |
| 5 | 5022301H1 | 1617 | 1884 |
| 5 | 2705868H1 | 1618 | 1886 |
| 5 | 5373405T6 | 1620 | 2144 |
| 5 | 1479486T6 | 1623 | 2109 |
| 5 | 2761062T6 | 1622 | 2108 |
| 5 | 2705868T6 | 1635 | 2107 |
| 5 | 3468165H1 | 1631 | 1895 |
| 5 | 1993688T6 | 1696 | 2108 |
| 5 | 1993688F6 | 1703 | 2147 |
| 5 | 1993688H1 | 1703 | 1950 |
| 5 | 666099H1 | 1721 | 1946 |
| 5 | g3423957 | 1776 | 2148 |
| 5 | g3241592 | 1821 | 2154 |
| 5 | 4340882T6 | 1846 | 2111 |
| 5 | 5016835H1 | 1893 | 2144 |
| 6 | 6206710H1 | 1 | 518 |
| 6 | 1632421H1 | 231 | 457 |
| 6 | 3255825H1 | 242 | 491 |
| 6 | 1951956T6 | 1 | 423 |
| 6 | g1099892 | 61 | 470 |
| 6 | 1356903H1 | 545 | 744 |
| 6 | 4987735H1 | 279 | 444 |
| 6 | 4987835H1 | 279 | 444 |
| 7 | 3170506F6 | 176 | 241 |
| 7 | 6387831H1 | 334 | 463 |
| 7 | 5607909H1 | 1 | 243 |
| 7 | 3558469F6 | 14 | 444 |
| 7 | 3558469H1 | 14 | 293 |
| 7 | g2006934 | 14 | 114 |
| 7 | 6109819H1 | 33 | 325 |
| 7 | g2464233 | 46 | 103 |
| 7 | 3170506H1 | 176 | 452 |
| 8 | g2069455 | 1 | 404 |
| 8 | 1597036H1 | 1 | 218 |
| 8 | 6429759H1 | 68 | 471 |
| 8 | 4013148H1 | 178 | 462 |
| 8 | g2064597 | 256 | 604 |
| 8 | 3296932F6 | 312 | 950 |
| 8 | 3296932H1 | 312 | 563 |
| 8 | 5839392H1 | 315 | 545 |
| 8 | 1254055H1 | 421 | 639 |
| 8 | 3527901H1 | 434 | 704 |
| 8 | 3402996H1 | 549 | 697 |
| 8 | 3503954F6 | 615 | 1002 |
| 8 | 3503954H1 | 616 | 915 |
| 8 | 3985708H1 | 623 | 812 |
| 8 | 6117978H1 | 729 | 1018 |
| 8 | 3620240H1 | 767 | 976 |
| 8 | 5912238H1 | 872 | 1066 |
| 8 | 1961296H1 | 965 | 1237 |
| 8 | 3406891F6 | 1026 | 1582 |
| 8 | 3406891H1 | 1026 | 1266 |
| 8 | 6196163H1 | 1193 | 1793 |
| 8 | 6196263H1 | 1193 | 1660 |
| 8 | 6059105H1 | 1200 | 1336 |
| 8 | 3296932T6 | 1249 | 1867 |
| 8 | 4367370H2 | 1253 | 1496 |
| 8 | 1908946F6 | 1257 | 1607 |
| 8 | 1908946H1 | 1257 | 1499 |
| 8 | g1958480 | 1311 | 1530 |
| 8 | 2270984R6 | 1377 | 1831 |
| 8 | 2270975H1 | 1377 | 1613 |
| 8 | 2270984H1 | 1377 | 1605 |
| 8 | g850966 | 1497 | 1759 |
| 8 | 5028068H1 | 1499 | 1786 |
| 8 | 1808792T6 | 1564 | 2151 |
| 8 | 987831H1 | 1575 | 1772 |
| 8 | 3406891T6 | 1593 | 2149 |
| 8 | 3410491H1 | 1602 | 1683 |
| 8 | 3503954T6 | 1606 | 2156 |
| 8 | 1808792F6 | 1606 | 2061 |
| 8 | 1808792H1 | 1606 | 1852 |
| 8 | 2270984T6 | 1688 | 2209 |
| 8 | g3231619 | 1764 | 2196 |
| 8 | 1908946T6 | 1856 | 2139 |
| 8 | g851209 | 1874 | 2177 |
| 8 | g3765675 | 1877 | 2174 |
| 8 | g3755028 | 1885 | 2189 |
| 8 | g1153884 | 1912 | 2174 |
| 8 | 5511650H1 | 2088 | 2271 |
| 9 | g2016674 | 1 | 372 |
| 9 | 6147645H1 | 830 | 1263 |
| 9 | 6338012H1 | 1 | 401 |
| 9 | 6336313H1 | 22 | 565 |
| 9 | 6577916H1 | 22 | 400 |
| 9 | g907436 | 1 | 324 |
| 9 | 6146280H1 | 39 | 573 |
| 9 | g1556832 | 14 | 174 |
| 9 | 6334269H1 | 187 | 736 |
| 9 | 6147106H1 | 189 | 743 |
| 9 | g2180097 | 1 | 381 |
| 9 | 6145722H1 | 314 | 826 |
| 9 | g1969671 | 44 | 388 |
| 9 | 6147235H1 | 444 | 987 |
| 9 | g1109940 | 188 | 316 |
| 9 | 6146408H1 | 530 | 954 |
| 9 | 6149660H1 | 543 | 1006 |
| 9 | 6144394H1 | 586 | 1189 |
| 9 | g1970587 | 1 | 235 |
| 10 | g993188 | 793 | 1107 |
| 10 | g3752938 | 851 | 1199 |
| 10 | 3070204H1 | 595 | 886 |
| 10 | 4420032H1 | 906 | 1146 |
| 10 | 3448504T6 | 187 | 819 |
| 10 | 4401644H1 | 1029 | 1282 |
| 10 | 5092611H1 | 1 | 214 |
| 10 | 5092611F6 | 1 | 621 |
| 10 | 1667402H1 | 159 | 214 |
| 10 | 3410836H1 | 533 | 781 |
| 10 | 3448604H1 | 535 | 785 |
| 10 | 3448504R6 | 535 | 884 |
| 10 | 5955967H1 | 566 | 794 |
| 10 | g2367695 | 570 | 959 |
| 10 | 5499560R6 | 579 | 1009 |
| 10 | g2629724 | 594 | 1046 |
| 10 | g4089777 | 675 | 993 |
| 10 | g1440904 | 708 | 1042 |
| 10 | g1941334 | 715 | 940 |
| 10 | g2078942 | 715 | 957 |
| 10 | g2901697 | 748 | 1048 |
| 11 | 2967428H1 | 1 | 284 |
| 12 | 3002696H1 | 1 | 285 |
| 12 | 3002696F6 | 1 | 394 |
| 12 | 1356974F6 | 109 | 495 |
| 12 | 1356974H1 | 109 | 353 |
| 12 | 3694703H1 | 215 | 496 |
| 12 | 1241180H1 | 253 | 311 |
| 12 | 5432511H1 | 253 | 362 |
| 12 | 5864554H1 | 391 | 666 |
| 12 | 4959207H1 | 409 | 524 |
| 12 | 5641442H1 | 415 | 654 |
| 12 | g2163443 | 423 | 741 |
| 12 | 2923379H1 | 430 | 686 |
| 12 | 4699556H1 | 430 | 683 |
| 12 | 040379H1 | 431 | 592 |
| 12 | 3032206H1 | 430 | 716 |
| 12 | 2509521H1 | 434 | 662 |
| 12 | 2509521F6 | 434 | 665 |
| 12 | 1703965H1 | 437 | 682 |
| 12 | 5605693H1 | 437 | 697 |
| 12 | 5546287H1 | 474 | 665 |
| 12 | 3801120H1 | 653 | 948 |
| 12 | 4878230H1 | 661 | 910 |
| 12 | 60135211B1 | 810 | 1213 |
| 12 | 60135113V1 | 834 | 1227 |
| 12 | 60135211V1 | 834 | 1227 |
| 13 | 70319065D1 | 190 | 589 |
| 13 | 70320239D1 | 212 | 793 |
| 13 | 70320883D1 | 212 | 645 |
| 13 | 70318969D1 | 213 | 774 |
| 13 | 70317969D1 | 398 | 791 |
| 13 | 70318139D1 | 398 | 931 |
| 13 | 70317613D1 | 398 | 857 |
| 13 | 70318020D1 | 398 | 856 |
| 13 | 70317863D1 | 398 | 791 |
| 13 | 70319554D1 | 398 | 791 |
| 13 | 70318409D1 | 398 | 695 |
| 13 | 70319562D1 | 398 | 741 |
| 13 | 70317555D1 | 398 | 635 |
| 13 | 70318235D1 | 399 | 928 |
| 13 | 70320237D1 | 399 | 852 |
| 13 | 70318920D1 | 399 | 812 |
| 13 | 70320035D1 | 411 | 741 |
| 13 | 70318273D1 | 417 | 856 |
| 13 | 4120208H1 | 332 | 571 |
| 13 | 70320719D1 | 447 | 856 |
| 13 | 3882715H1 | 1 | 312 |
| 13 | 2797803F6 | 1 | 452 |
| 13 | 2797803H1 | 1 | 245 |
| 13 | 2788120H1 | 4 | 256 |
| 13 | 70317458D1 | 481 | 876 |
| 13 | 70318194D1 | 481 | 856 |
| 13 | 70320117D1 | 521 | 856 |
| 13 | 70319709D1 | 559 | 988 |
| 13 | 70319736D1 | 639 | 1133 |
| 13 | 70319869D1 | 715 | 856 |
| 13 | 2797803T6 | 97 | 633 |
| 13 | 70320906D1 | 743 | 1133 |
| 13 | 70320282D1 | 743 | 856 |
| 13 | 3873636H1 | 291 | 586 |
| 13 | 70319282D1 | 877 | 1310 |
| 13 | 70319385D1 | 904 | 1133 |
| 13 | g2329469 | 121 | 408 |
| 13 | 3873636T6 | 1 | 336 |
| 13 | 70321152D1 | 466 | 931 |
| 13 | 70317918D1 | 466 | 930 |
| 13 | 70320713D1 | 466 | 856 |
| 13 | 70320635D1 | 466 | 856 |
| 13 | 70320227D1 | 466 | 855 |
| 13 | 70317509D1 | 466 | 856 |
| 13 | 70320434D1 | 466 | 876 |
| 13 | 70320694D1 | 468 | 856 |
| 13 | 70320790D1 | 466 | 838 |
| 13 | 70318744D1 | 466 | 880 |
| 13 | 70318259D1 | 467 | 930 |
| 13 | 70318517D1 | 481 | 931 |
| 14 | g982879 | 1 | 359 |
| 14 | g1039681 | 62 | 330 |
| 14 | 1850733F6 | 165 | 655 |
| 14 | 1850733H1 | 165 | 450 |
| 14 | 5962837H1 | 290 | 797 |
| 14 | g1040085 | 419 | 732 |
| 14 | g982880 | 468 | 732 |
| 14 | 70060347V1 | 660 | 1048 |
| 15 | g1855688 | 706 | 918 |
| 15 | g2464352 | 752 | 910 |
| 15 | g1471158 | 757 | 906 |
| 15 | 2127070H1 | 769 | 901 |
| 15 | g2836064 | 779 | 901 |
| 15 | g3665793 | 787 | 901 |
| 15 | g2969091 | 800 | 901 |
| 15 | 1473808H1 | 1 | 110 |
| 15 | g1548716 | 7 | 330 |
| 15 | g1940204 | 1 | 464 |
| 15 | 6298845H1 | 40 | 208 |
| 15 | 2204071H1 | 40 | 222 |
| 15 | g1859154 | 40 | 370 |
| 15 | 6736659H1 | 65 | 581 |
| 15 | 1494078H1 | 66 | 277 |
| 15 | 1803588H1 | 72 | 335 |
| 15 | 2277953H1 | 119 | 392 |
| 15 | 2500164H1 | 126 | 380 |
| 15 | 2500349F6 | 126 | 614 |
| 15 | 2500349H1 | 126 | 376 |
| 15 | 3886423H1 | 209 | 462 |
| 15 | 2604501H1 | 221 | 478 |
| 15 | 3899701H1 | 225 | 501 |
| 15 | 2717183H1 | 231 | 467 |
| 15 | 7058162H1 | 192 | 582 |
| 15 | 3614668H1 | 134 | 417 |
| 15 | 3986769H1 | 264 | 521 |
| 15 | 3246169H1 | 221 | 454 |
| 15 | 6916455H1 | 221 | 496 |
| 15 | 4856582H1 | 213 | 492 |
| 15 | 319110R6 | 228 | 742 |
| 15 | 319110H1 | 228 | 610 |
| 15 | 4651030H1 | 279 | 521 |
| 15 | g1856065 | 227 | 715 |
| 15 | 5313986H1 | 245 | 479 |
| 15 | 817768H1 | 254 | 505 |
| 15 | 3984239H1 | 265 | 539 |
| 15 | g810072 | 267 | 601 |
| 15 | g2064405 | 265 | 723 |
| 15 | 3374148H1 | 269 | 527 |
| 15 | g1025985 | 271 | 535 |
| 15 | 6548704H1 | 274 | 571 |
| 15 | g896704 | 276 | 572 |
| 15 | 2501178H1 | 276 | 513 |
| 15 | g842912 | 278 | 529 |
| 15 | g920007 | 323 | 566 |
| 15 | 4772879H1 | 286 | 566 |
| 15 | 4240190H1 | 296 | 541 |
| 15 | 2751946H1 | 326 | 585 |
| 15 | 2500349T6 | 331 | 858 |
| 15 | g1166133 | 332 | 498 |
| 15 | g1471157 | 339 | 531 |
| 15 | 319110T6 | 345 | 865 |
| 15 | 5213230H1 | 356 | 581 |
| 15 | 977791H1 | 357 | 574 |
| 15 | 977791R1 | 357 | 785 |
| 15 | 2771133H1 | 361 | 606 |
| 15 | 2132792R6 | 373 | 512 |
| 15 | 1699741H1 | 374 | 602 |
| 15 | 1927601H1 | 386 | 626 |
| 15 | 1803588T6 | 396 | 863 |
| 15 | 2413149H1 | 403 | 565 |
| 15 | 612807H1 | 427 | 521 |
| 15 | 5122537T6 | 413 | 870 |
| 15 | 5028542H1 | 423 | 679 |
| 15 | 4365757H1 | 421 | 681 |
| 15 | 6736648H1 | 428 | 901 |
| 15 | g2537655 | 437 | 871 |
| 15 | 1348978H1 | 438 | 680 |
| 15 | 955324R1 | 438 | 901 |
| 15 | 2132792H1 | 436 | 685 |
| 15 | 1348899H1 | 438 | 697 |
| 15 | 955324H1 | 438 | 680 |
| 15 | 1616045H1 | 441 | 659 |
| 15 | 1637850H1 | 450 | 654 |
| 15 | 1635340H1 | 450 | 654 |
| 15 | 4835330H1 | 453 | 722 |
| 15 | 3267673H1 | 453 | 707 |
| 15 | 1621983H1 | 453 | 665 |
| 15 | g5110236 | 456 | 914 |
| 15 | g3254447 | 461 | 913 |
| 15 | g3961279 | 466 | 901 |
| 15 | 2872292H1 | 466 | 733 |
| 15 | 4958730H1 | 469 | 733 |
| 15 | g5878660 | 470 | 907 |
| 15 | g5544770 | 475 | 901 |
| 15 | 4567732H1 | 489 | 759 |
| 15 | 374248H1 | 498 | 720 |
| 15 | g4005355 | 504 | 901 |
| 15 | g5365013 | 508 | 913 |
| 15 | 5992634H1 | 507 | 803 |
| 15 | g3988037 | 521 | 919 |
| 15 | 906113H1 | 541 | 796 |
| 15 | 906113R1 | 541 | 901 |
| 15 | 537271H1 | 542 | 794 |
| 15 | 905790T1 | 541 | 858 |
| 15 | 2751121H1 | 551 | 642 |
| 15 | 611349H1 | 563 | 824 |
| 15 | 2491963H1 | 563 | 818 |
| 15 | g3232145 | 568 | 912 |
| 15 | g3802367 | 572 | 924 |
| 15 | g1548831 | 584 | 877 |
| 15 | 5164893H1 | 599 | 862 |
| 15 | g3770372 | 602 | 901 |
| 15 | 1473995H1 | 616 | 805 |
| 15 | 1473995T1 | 616 | 861 |
| 15 | 5986850H1 | 620 | 901 |
| 15 | g4511097 | 621 | 901 |
| 15 | 6212219H1 | 625 | 901 |
| 15 | g4332172 | 627 | 914 |
| 15 | 2132792T6 | 636 | 862 |
| 15 | 2687592H1 | 650 | 886 |
| 15 | 5083760H1 | 677 | 931 |
| 15 | 2204071T6 | 678 | 858 |
| 15 | g809963 | 684 | 901 |
| 15 | 2103287H1 | 690 | 912 |
| 15 | g896705 | 691 | 905 |
| 15 | g3601113 | 695 | 908 |
| 15 | g1114641 | 695 | 877 |
| 15 | 5551489H1 | 706 | 900 |
| 16 | 2738293F6 | 1 | 355 |
| 16 | 2738293H1 | 1 | 169 |
| 16 | 1301651F6 | 1104 | 1404 |
| 16 | 1301651H1 | 1104 | 1232 |
| 16 | 2130054R6 | 1113 | 1391 |
| 16 | 2130054H1 | 1113 | 1285 |
| 16 | 1232594H1 | 1113 | 1253 |
| 16 | 2738293T6 | 1129 | 1578 |
| 16 | 412176F1 | 1153 | 1615 |
| 16 | g4312034 | 1174 | 1553 |
| 16 | g776598 | 1269 | 1557 |
| 16 | g787388 | 1308 | 1554 |
| 16 | g5541568 | 1355 | 1615 |
| 16 | 2562775H1 | 471 | 729 |
| 16 | 5290883H1 | 544 | 798 |
| 16 | 4014888H1 | 1 | 215 |
| 16 | 3632619T6 | 662 | 1050 |
| 16 | g5177519 | 10 | 495 |
| 16 | g776684 | 711 | 998 |
| 16 | g786907 | 711 | 959 |
| 16 | 6950344H1 | 146 | 736 |
| 16 | 1232594F1 | 727 | 1253 |
| 16 | g4223240 | 748 | 1170 |
| 16 | 418633R6 | 158 | 575 |
| 16 | 1220942H1 | 772 | 1035 |
| 16 | 412176R1 | 158 | 612 |
| 16 | 416149H1 | 158 | 363 |
| 16 | 412176H1 | 158 | 344 |
| 16 | 3441272H1 | 802 | 1039 |
| 16 | 2645796T6 | 875 | 1050 |
| 16 | g834367 | 200 | 342 |
| 16 | 2645796H1 | 884 | 1050 |
| 16 | 2645796F6 | 884 | 1175 |
| 16 | 4730205H1 | 244 | 413 |
| 16 | 418633T6 | 1004 | 1574 |
| 16 | 3873188H1 | 361 | 619 |
| 16 | 1301651T6 | 1104 | 1576 |
| 17 | 6816661H1 | 1 | 424 |
| 17 | 6816661J1 | 303 | 880 |
| 18 | 6765038H1 | 1 | 356 |
| 18 | 6823189H1 | 1 | 515 |
| 18 | 6823189J1 | 268 | 821 |
| 18 | 6927865H1 | 598 | 1049 |
| 18 | g1012738 | 705 | 947 |
| 19 | 6485634R9 | 1 | 494 |
| 19 | 6488342R9 | 1 | 385 |
| 19 | 4347981T9 | 299 | 607 |
| 19 | 4347981F8 | 359 | 958 |
| 19 | 4347981H1 | 855 | 958 |
| 20 | 4187505H1 | 1 | 114 |
| 20 | 4187505F8 | 1 | 618 |
| 20 | 4187505F9 | 1 | 583 |
| 20 | 6064542T8 | 300 | 655 |
| 20 | 4187505T8 | 386 | 774 |
| 20 | 4187505T9 | 409 | 774 |
| 20 | 2721392H1 | 600 | 830 |
| 21 | 1930289T6 | 1293 | 1817 |
| 21 | 5784355T6 | 1318 | 1728 |
| 21 | 3659618T6 | 1332 | 1820 |
| 21 | 2762471H1 | 1353 | 1596 |
| 21 | 3400826H1 | 1373 | 1600 |
| 21 | g3647867 | 1385 | 1792 |
| 21 | g4302533 | 1430 | 1884 |
| 21 | 954028H1 | 1436 | 1704 |
| 21 | 7081513H1 | 1 | 524 |
| 21 | 2509720H1 | 252 | 479 |
| 21 | 3242083H1 | 367 | 616 |
| 21 | 3644604F6 | 369 | 626 |
| 21 | 3644604H1 | 370 | 665 |
| 21 | 5741818H1 | 406 | 601 |
| 21 | 7082116H1 | 437 | 980 |
| 21 | 2060770H1 | 625 | 869 |
| 21 | 5113293H1 | 708 | 973 |
| 21 | g1891059 | 713 | 1017 |
| 21 | 6479360H1 | 821 | 1302 |
| 21 | 5784455H1 | 850 | 1085 |
| 21 | 5784355H1 | 850 | 1112 |
| 21 | 5794459H1 | 850 | 1145 |
| 21 | 5784355F6 | 855 | 1390 |
| 21 | 6832370H1 | 873 | 1476 |
| 21 | 3734122H1 | 873 | 1159 |
| 21 | 6836219H1 | 945 | 1256 |
| 21 | 5104159H1 | 959 | 1198 |
| 21 | 4512707H1 | 1004 | 1259 |
| 21 | 1836548H1 | 1054 | 1110 |
| 21 | 1006724H1 | 1060 | 1307 |
| 21 | 523339H1 | 1124 | 1369 |
| 21 | 630148H1 | 1154 | 1436 |
| 21 | 5897974H1 | 1196 | 1465 |
| 21 | 459407H1 | 1219 | 1502 |
| 21 | 1930289F6 | 1289 | 1794 |
| 21 | 1930289H1 | 1289 | 1559 |
| 21 | g6398856 | 1470 | 1886 |
| 21 | g5368595 | 1474 | 1928 |
| 21 | g5662921 | 1484 | 1926 |
| 21 | g2279246 | 1491 | 1886 |
| 21 | g3432804 | 1495 | 1928 |
| 21 | g3927268 | 1499 | 1890 |
| 21 | g3751389 | 1532 | 1885 |
| 21 | 3028225H1 | 1536 | 1817 |
| 21 | g3244749 | 1546 | 1891 |
| 21 | 1696241T6 | 1547 | 1867 |
| 21 | 1696241F6 | 1554 | 1907 |
| 21 | 1696241H1 | 1554 | 1797 |
| 21 | g6471216 | 1557 | 1864 |
| 21 | g5110284 | 1565 | 2014 |
| 21 | g4373194 | 1572 | 1928 |
| 21 | g5664143 | 1577 | 1926 |
| 21 | 2293651H1 | 1576 | 1806 |
| 21 | g3869595 | 1585 | 1927 |
| 21 | 725947H1 | 1585 | 1828 |
| 21 | g2401363 | 1589 | 1886 |
| 21 | 6773275J1 | 1592 | 2142 |
| 21 | 91156118 | 1606 | 1886 |
| 21 | g1892265 | 1659 | 1925 |
| 21 | g4242906 | 1677 | 1860 |
| 21 | 5399680H1 | 1684 | 1881 |
| 21 | g764351 | 1720 | 1939 |
| 21 | g2716497 | 1721 | 2098 |
| 21 | g2804847 | 1743 | 2084 |
| 21 | 957645T1 | 1747 | 1843 |
| 21 | 957645R6 | 1747 | 1886 |
| 21 | 957645T6 | 1747 | 1843 |
| 21 | 957645H1 | 1747 | 1852 |
| 21 | g2930525 | 1756 | 2091 |
| 21 | 825718H1 | 1892 | 2187 |
| 21 | 3169276F6 | 1905 | 2315 |
| 21 | 3169276H1 | 1906 | 2174 |
| 21 | 4880539H1 | 2029 | 2262 |
| 21 | 551186H1 | 2088 | 2334 |
| 21 | 5223525H1 | 2143 | 2294 |
| 21 | g1959837 | 2258 | 2561 |
| 21 | 3314476F6 | 2280 | 2591 |
| 21 | 3314476H1 | 2280 | 2530 |
| 21 | 6773275H1 | 2281 | 2746 |
| 21 | 2134443H1 | 2500 | 2591 |
| 21 | 3169276T6 | 2500 | 2591 |
| 22 | 478871H1 | 2084 | 2377 |
| 22 | g3931712 | 2092 | 2342 |
| 22 | g4533567 | 2097 | 2481 |
| 22 | g5856043 | 2081 | 2480 |
| 22 | g3932376 | 2082 | 2484 |
| 22 | g4073477 | 2082 | 2485 |
| 22 | g4900706 | 2083 | 2480 |
| 22 | g6198867 | 2084 | 2488 |
| 22 | 2466234H1 | 2106 | 2334 |
| 22 | 4824519F6 | 939 | 1471 |
| 22 | g2444680 | 949 | 1228 |
| 22 | 3960643H2 | 962 | 1234 |
| 22 | g2816130 | 1111 | 1586 |
| 22 | g5395011 | 1122 | 1577 |
| 22 | g1812285 | 1238 | 1577 |
| 22 | g1194260 | 1297 | 1389 |
| 22 | g2818085 | 1298 | 1586 |
| 22 | 3248605H1 | 1302 | 1611 |
| 22 | g6472165 | 1303 | 1565 |
| 22 | 1781932R6 | 1312 | 1806 |
| 22 | 1781932H1 | 1312 | 1579 |
| 22 | g2819140 | 1345 | 1570 |
| 22 | 4365019H1 | 1372 | 1640 |
| 22 | 6521348H1 | 1423 | 1950 |
| 22 | 1454503F6 | 1596 | 2079 |
| 22 | 1454503H1 | 1596 | 1797 |
| 22 | 1454503F1 | 1596 | 2144 |
| 22 | 031646H1 | 1628 | 1864 |
| 22 | 1698275H1 | 1730 | 1954 |
| 22 | 2477996H1 | 1833 | 2038 |
| 22 | g1088089 | 1838 | 2109 |
| 22 | 6051243H1 | 1859 | 2290 |
| 22 | 6051243J1 | 1859 | 2290 |
| 22 | 719445H1 | 1906 | 2117 |
| 22 | 1454503T6 | 1941 | 2444 |
| 22 | 1753732H1 | 1969 | 2210 |
| 22 | 1753732F6 | 1969 | 2336 |
| 22 | g6040701 | 2013 | 2480 |
| 22 | g2779360 | 2014 | 2292 |
| 22 | g5879420 | 2024 | 2486 |
| 22 | g5848637 | 2028 | 2490 |
| 22 | g4989391 | 2035 | 2488 |
| 22 | g4136876 | 2036 | 2488 |
| 22 | g4734230 | 2036 | 2481 |
| 22 | 3011670T6 | 2038 | 2442 |
| 22 | 1306758T6 | 2045 | 2444 |
| 22 | 3698556H1 | 2046 | 2293 |
| 22 | g6132228 | 2080 | 2481 |
| 22 | g5813048 | 2080 | 2480 |
| 22 | g5595187 | 2080 | 2481 |
| 22 | 919527H1 | 2154 | 2367 |
| 22 | 2288878H1 | 2216 | 2464 |
| 22 | 043873H1 | 2227 | 2474 |
| 22 | g4332802 | 2257 | 2480 |
| 22 | 2929901H1 | 2277 | 2381 |
| 22 | 2929910H1 | 2277 | 2496 |
| 22 | 2130537H1 | 2284 | 2487 |
| 22 | g659717 | 2325 | 2492 |
| 22 | 4594675H1 | 2398 | 2486 |
| 22 | g856894 | 1 | 320 |
| 22 | 2493973H1 | 1 | 312 |
| 22 | 1306758H1 | 1 | 206 |
| 22 | 1306758F6 | 1 | 245 |
| 22 | 2892162H1 | 3 | 128 |
| 22 | 6460769H2 | 27 | 92 |
| 22 | 6464146H2 | 27 | 600 |
| 22 | 6784388H1 | 281 | 734 |
| 22 | 3297069H1 | 291 | 401 |
| 22 | 3011670F6 | 367 | 436 |
| 22 | 3011670H1 | 367 | 674 |
| 22 | 4571406H1 | 659 | 940 |
| 22 | 4571406F9 | 660 | 1290 |
| 22 | 7161658H1 | 663 | 1132 |
| 22 | 2731281H1 | 894 | 1128 |
| 22 | 4824519H1 | 939 | 1194 |
| 23 | 5092611H1 | 1 | 214 |
| 23 | 5092611F6 | 1 | 621 |
| 23 | 1667402H1 | 159 | 214 |
| 23 | 3410836H1 | 533 | 781 |
| 23 | 3448604H1 | 535 | 785 |
| 23 | 3448504R6 | 535 | 884 |
| 23 | g1099119 | 641 | 962 |
| 23 | 2752130H1 | 776 | 960 |
| 23 | g1099240 | 776 | 1080 |
| 23 | g1018781 | 866 | 1119 |
| 23 | 219949R6 | 875 | 1273 |
| 23 | 219949H1 | 875 | 1016 |
| 23 | 215836H1 | 875 | 1112 |
| 23 | 219949T6 | 875 | 1237 |
| 23 | 2752130T6 | 913 | 1077 |
| 23 | 2017342H1 | 1100 | 1334 |
| 24 | g6038562 | 1 | 431 |
| 24 | g6398073 | 1 | 423 |
| 24 | 2114662H1 | 226 | 409 |
| 24 | 4802387F8 | 403 | 845 |
| 24 | 3814249H1 | 637 | 864 |
| 24 | g3764669 | 22 | 458 |
| 24 | g5595942 | 31 | 436 |
| 24 | g4526293 | 31 | 392 |
| 24 | g2359335 | 39 | 217 |
| 24 | 3426470H1 | 38 | 296 |
| 24 | 3751121H1 | 64 | 360 |
| 24 | 5102466H1 | 67 | 320 |
| 24 | g5543678 | 70 | 341 |
| 24 | g5857639 | 70 | 492 |
| 24 | g1483656 | 70 | 494 |
| 24 | g1687609 | 70 | 310 |
| 24 | g2240940 | 70 | 414 |
| 24 | g1728565 | 70 | 496 |
| 24 | g2191816 | 70 | 412 |
| 24 | g2914811 | 70 | 480 |
| 24 | g2910290 | 70 | 370 |
| 24 | g3051051 | 70 | 382 |
| 24 | g2206444 | 70 | 408 |
| 24 | g2912492 | 70 | 325 |
| 24 | g1516143 | 71 | 240 |
| 24 | 3250685H1 | 79 | 373 |
| 24 | 720142H1 | 79 | 209 |
| 24 | 4567816H1 | 121 | 383 |
| 24 | 7059154H1 | 135 | 293 |
| 24 | 1671750H1 | 174 | 387 |
| 24 | 1511456H1 | 174 | 374 |
| 24 | 1746566H1 | 177 | 451 |
| 24 | 1746566F6 | 177 | 633 |
| 24 | 5163064H1 | 194 | 458 |
| 24 | 4401870H1 | 194 | 456 |
| 24 | 2850494H1 | 194 | 474 |
| 24 | 620602H1 | 195 | 454 |
| 24 | 6317553H1 | 202 | 488 |
| 24 | 6317552H1 | 202 | 488 |
| 24 | 4543261F6 | 217 | 615 |
| 24 | 4543261H1 | 217 | 479 |
| 24 | 2886956H1 | 219 | 490 |
| 24 | g3116861 | 220 | 535 |
| 24 | 2298804H1 | 223 | 477 |
| 24 | 3530239H1 | 224 | 499 |
| 24 | 6043133H1 | 223 | 484 |
| 24 | 4986150H1 | 239 | 517 |
| 24 | 3506149H1 | 238 | 548 |
| 24 | g1165727 | 258 | 501 |
| 24 | 1747695H1 | 261 | 510 |
| 24 | 1747316H1 | 261 | 523 |
| 24 | 1231741H1 | 276 | 527 |
| 24 | 3221695H1 | 279 | 600 |
| 24 | g2184331 | 285 | 732 |
| 24 | 1692992H1 | 306 | 516 |
| 24 | 1692983H1 | 306 | 516 |
| 24 | 551035H1 | 318 | 578 |
| 24 | 1398934H1 | 318 | 577 |
| 24 | 033465H1 | 318 | 595 |
| 24 | 2483413H1 | 329 | 583 |
| 24 | 4567659H1 | 334 | 601 |
| 24 | g919123 | 349 | 531 |
| 24 | g892506 | 347 | 650 |
| 24 | 1696018H1 | 364 | 566 |
| 24 | 4996050H1 | 377 | 649 |
| 24 | 4249442H1 | 380 | 586 |
| 24 | 524140H1 | 374 | 618 |
| 24 | 5290888H1 | 385 | 616 |
| 24 | 1876529H1 | 389 | 658 |
| 24 | 749418H1 | 402 | 648 |
| 24 | 4802787H1 | 403 | 660 |
| 24 | 070211H1 | 404 | 562 |
| 24 | 3488984H1 | 404 | 625 |
| 24 | g856452 | 405 | 698 |
| 24 | g777504 | 405 | 703 |
| 24 | 2410459H1 | 407 | 651 |
| 24 | g759872 | 415 | 726 |
| 24 | 179723H1 | 419 | 544 |
| 24 | 2701594H1 | 423 | 676 |
| 24 | 5432663H1 | 427 | 693 |
| 24 | 1319180H1 | 432 | 542 |
| 24 | 3324294H1 | 509 | 779 |
| 24 | 2303707H1 | 510 | 635 |
| 24 | 5020923H1 | 514 | 790 |
| 24 | 2298247H1 | 518 | 638 |
| 24 | 3614560H1 | 547 | 781 |
| 24 | 6741535H1 | 680 | 937 |
| 24 | g6397955 | 22 | 223 |
| 24 | 6754236J1 | 1 | 584 |
| 24 | 6754123J1 | 1 | 509 |
| 24 | 6757969J1 | 1 | 550 |
| 24 | 6203321H1 | 1 | 292 |
| 24 | 6758815J1 | 1 | 591 |
| 24 | 6757686J1 | 1 | 479 |
| 24 | g3594017 | 10 | 463 |
| 24 | g6399041 | 22 | 486 |
| 25 | g3077101 | 1343 | 1721 |
| 25 | 2552885T6 | 1025 | 1390 |
| 25 | g2594419 | 1042 | 1432 |
| 25 | g1647965 | 1086 | 1408 |
| 25 | g4451382 | 1116 | 1433 |
| 25 | 3919118T8 | 1167 | 1620 |
| 25 | g316154 | 1190 | 1474 |
| 25 | g2958693 | 1358 | 1720 |
| 25 | 2902677H1 | 1204 | 1500 |
| 25 | 5821366H1 | 1235 | 1554 |
| 25 | 5819630H1 | 1235 | 1546 |
| 25 | 5816778H1 | 1235 | 1549 |
| 25 | 3640567H1 | 1374 | 1579 |
| 25 | 5814634H1 | 1235 | 1560 |
| 25 | 5817645H1 | 1235 | 1562 |
| 25 | 5822469H1 | 1234 | 1486 |
| 25 | 5813545H1 | 1235 | 1493 |
| 25 | 5812917H1 | 1235 | 1509 |
| 25 | 5818829H1 | 1235 | 1458 |
| 25 | 5817650H1 | 1235 | 1402 |
| 25 | g4295078 | 1242 | 1433 |
| 25 | g5934094 | 1284 | 1720 |
| 25 | g5741261 | 1288 | 1723 |
| 25 | 2463542T6 | 1288 | 1769 |
| 25 | g1982163 | 1341 | 1571 |
| 25 | 2655535F7 | 226 | 649 |
| 25 | 2655535F6 | 226 | 655 |
| 25 | 6887246J1 | 370 | 475 |
| 25 | 6887246H1 | 379 | 482 |
| 25 | 1902994H1 | 381 | 630 |
| 25 | 4096762H1 | 416 | 721 |
| 25 | 4510286H1 | 455 | 705 |
| 25 | 007733H1 | 530 | 799 |
| 25 | 5910362H1 | 646 | 948 |
| 25 | 2857263H1 | 653 | 766 |
| 25 | 2857263F6 | 653 | 1118 |
| 25 | 6212439H1 | 655 | 938 |
| 25 | 2869957H1 | 667 | 946 |
| 25 | 2869957F6 | 667 | 1228 |
| 25 | g942739 | 676 | 955 |
| 25 | g1477126 | 687 | 1120 |
| 25 | g1527541 | 687 | 1069 |
| 25 | g969342 | 687 | 806 |
| 25 | g2184408 | 725 | 1175 |
| 25 | 5307452H1 | 767 | 1019 |
| 25 | 2655535T6 | 768 | 1384 |
| 25 | 6518401H1 | 806 | 1348 |
| 25 | 3919118F8 | 847 | 1404 |
| 25 | 2869957T6 | 850 | 1394 |
| 25 | g2037747 | 890 | 1199 |
| 25 | 6321277H1 | 969 | 1213 |
| 25 | 2463542F6 | 969 | 1516 |
| 25 | 2463542H1 | 969 | 1188 |
| 25 | 552697H1 | 979 | 1207 |
| 25 | 6815232H1 | 996 | 1477 |
| 25 | 3484912H1 | 1007 | 1342 |
| 25 | 7040095H1 | 1020 | 1549 |
| 25 | 6743438H1 | 1 | 343 |
| 25 | 5959701H1 | 1 | 541 |
| 25 | 7199644H1 | 39 | 460 |
| 25 | 3801333H1 | 46 | 327 |
| 25 | 6146685H1 | 131 | 608 |
| 25 | 3021452H1 | 203 | 499 |
| 25 | 2655535H1 | 226 | 520 |
| 25 | g867230 | 2483 | 2783 |
| 25 | g1477035 | 2519 | 2783 |
| 25 | 6158466H1 | 2530 | 2692 |
| 25 | 646057H1 | 2530 | 2784 |
| 25 | 3535428H1 | 2550 | 2782 |
| 25 | g1479354 | 2562 | 2781 |
| 25 | g3095823 | 2573 | 2706 |
| 25 | 2878073H1 | 2577 | 2784 |
| 25 | g944173 | 2582 | 2679 |
| 25 | 3702759H1 | 2594 | 2782 |
| 25 | g5325988 | 2594 | 2782 |
| 25 | g969343 | 2630 | 2782 |
| 25 | 2043940H1 | 2651 | 2782 |
| 25 | 661586H1 | 2658 | 2813 |
| 25 | 5056769H1 | 2702 | 2789 |
| 25 | 6332343H1 | 1960 | 2520 |
| 25 | 5327933H1 | 1964 | 2225 |
| 25 | g3658839 | 1983 | 2394 |
| 25 | g39274l4 | 1988 | 2423 |
| 25 | 5275308H1 | 2005 | 2238 |
| 25 | 2673389H1 | 2047 | 2295 |
| 25 | g274328 | 2457 | 2782 |
| 25 | 7237473H1 | 2048 | 2569 |
| 25 | 5333974H1 | 2054 | 2302 |
| 25 | 1982603H1 | 2091 | 2315 |
| 25 | 2044369H1 | 2192 | 2427 |
| 25 | g4196655 | 2192 | 2327 |
| 25 | 4726834H1 | 2192 | 2469 |
| 25 | 1772731H1 | 2244 | 2461 |
| 25 | 4266001H1 | 2279 | 2468 |
| 25 | 4730142H1 | 2304 | 2558 |
| 25 | g2555947 | 2309 | 2616 |
| 25 | 840781H1 | 2310 | 2582 |
| 25 | 4492313H1 | 2310 | 2757 |
| 25 | 4593969H1 | 2310 | 2579 |
| 25 | 4492214H1 | 2315 | 2782 |
| 25 | g5109339 | 2326 | 2784 |
| 25 | 613645H1 | 2337 | 2571 |
| 25 | 2132842H1 | 2376 | 2617 |
| 25 | g4073710 | 2384 | 2785 |
| 25 | g1527498 | 2413 | 2783 |
| 25 | g2184179 | 2453 | 2785 |
| 25 | 2721848H1 | 2456 | 2707 |
| 25 | 2637714H1 | 2456 | 2709 |
| 25 | g2569732 | 1382 | 1794 |
| 25 | 3042074H1 | 1471 | 1786 |
| 25 | 4714518H1 | 1907 | 1991 |
| 25 | 4261452H1 | 1474 | 1733 |
| 25 | 2773951H1 | 1488 | 1742 |
| 25 | g3050704 | 1508 | 1703 |
| 25 | 6023537H1 | 1527 | 1763 |
| 25 | 2653867H1 | 1534 | 1820 |
| 25 | g4522739 | 1927 | 2326 |
| 25 | g3960542 | 1580 | 1841 |
| 25 | g1119058 | 1591 | 1716 |
| 25 | 6411539H1 | 1616 | 2116 |
| 25 | g5369593 | 1952 | 2423 |
| 25 | 2097957H1 | 1626 | 1902 |
| 25 | 3769037H1 | 1634 | 1888 |
| 25 | 3509114H1 | 1638 | 1908 |
| 25 | 3927329H1 | 1656 | 1922 |
| 25 | 2593752H1 | 1679 | 1869 |
| 25 | g1981200 | 1681 | 1968 |
| 25 | 1683135H1 | 1757 | 1897 |
| 25 | 5832853H1 | 1792 | 2069 |
| 25 | 806178H1 | 1796 | 2022 |
| 25 | 3558496H1 | 1804 | 2073 |
| 25 | 2795155H1 | 1843 | 2089 |
| 25 | 1982603R6 | 1867 | 2315 |
| 25 | 2876519H1 | 1883 | 2138 |
| 25 | g3804453 | 1895 | 2327 |
| 25 | g5812417 | 1896 | 2308 |
| 25 | 5563230H1 | 1959 | 2187 |
| 26 | 3719577H1 | 1538 | 1698 |
| 26 | g2166727 | 1572 | 1672 |
| 26 | g1259102 | 2007 | 2205 |
| 26 | 693434H1 | 2012 | 2270 |
| 26 | 6021316H1 | 2013 | 2538 |
| 26 | 2605880H1 | 2014 | 2246 |
| 26 | 2605880F6 | 2014 | 2595 |
| 26 | 4132607F6 | 1614 | 2100 |
| 26 | g2166556 | 1633 | 1729 |
| 26 | g2013024 | 1983 | 2258 |
| 26 | g1321421 | 2014 | 2246 |
| 26 | 1258355F6 | 2015 | 2225 |
| 26 | 1258355F1 | 2015 | 2511 |
| 26 | 1258355H1 | 2015 | 2216 |
| 26 | 4584658H1 | 2019 | 2164 |
| 26 | 2211736H1 | 2025 | 2282 |
| 26 | 2956194H1 | 2026 | 2103 |
| 26 | 5853223H1 | 2026 | 2227 |
| 26 | 3597053H1 | 1993 | 2297 |
| 26 | 6718635H1 | 2026 | 2279 |
| 26 | 4728680H1 | 2026 | 2159 |
| 26 | 3210462H1 | 2026 | 2225 |
| 26 | g707728 | 2026 | 2234 |
| 26 | g727700 | 2038 | 2283 |
| 26 | 2228104H1 | 2043 | 2111 |
| 26 | 3597053F7 | 1997 | 2600 |
| 26 | 2839674H2 | 2043 | 2159 |
| 26 | 2932162H1 | 2043 | 2135 |
| 26 | 6096855H1 | 2043 | 2204 |
| 26 | 3717718H1 | 2043 | 2155 |
| 26 | g1294859 | 2046 | 2640 |
| 26 | 3448635H1 | 2048 | 2289 |
| 26 | 3900947R8 | 2055 | 2678 |
| 26 | 2814661H1 | 1998 | 2286 |
| 26 | 3901994H1 | 2055 | 2321 |
| 26 | 2604117H1 | 2069 | 2314 |
| 26 | 4538547H1 | 2069 | 2325 |
| 26 | g789232 | 2070 | 2294 |
| 26 | g1677967 | 2093 | 2316 |
| 26 | g1783925 | 2101 | 2388 |
| 26 | g4690089 | 2125 | 2583 |
| 26 | g4685239 | 2125 | 2332 |
| 26 | g858499 | 2138 | 2459 |
| 26 | 5725860H2 | 2176 | 2244 |
| 26 | 123421H1 | 2177 | 2318 |
| 26 | g777923 | 2177 | 2289 |
| 26 | g789198 | 2203 | 2454 |
| 26 | g781495 | 2204 | 2455 |
| 26 | 2667384H1 | 2205 | 2433 |
| 26 | g1697403 | 2230 | 2531 |
| 26 | g3933029 | 2237 | 2737 |
| 26 | 2939915H1 | 2235 | 2393 |
| 26 | 4643005H1 | 2235 | 2484 |
| 26 | 4704174F9 | 1 | 569 |
| 26 | 3122989H1 | 273 | 555 |
| 26 | 4704174H1 | 322 | 569 |
| 26 | 4777379H1 | 348 | 606 |
| 26 | 2834749F6 | 405 | 694 |
| 26 | 2834749H1 | 420 | 694 |
| 26 | 5911946H1 | 436 | 722 |
| 26 | 4721035H1 | 446 | 695 |
| 26 | 1601324H1 | 453 | 599 |
| 26 | g4876168 | 456 | 887 |
| 26 | 5372175H1 | 456 | 681 |
| 26 | 1992918H1 | 466 | 560 |
| 26 | 2782119H1 | 516 | 746 |
| 26 | g1628776 | 669 | 1024 |
| 26 | 6256994H1 | 670 | 928 |
| 26 | g1312588 | 674 | 886 |
| 26 | 2101267H1 | 697 | 941 |
| 26 | g1948843 | 762 | 1091 |
| 26 | 7076736H1 | 791 | 1279 |
| 26 | 2135134F6 | 816 | 1213 |
| 26 | 2135134H1 | 816 | 1077 |
| 26 | 2792758H1 | 907 | 1221 |
| 26 | 5151110H1 | 1175 | 1353 |
| 26 | 6529106H1 | 1196 | 1624 |
| 26 | 3136230H1 | 1206 | 1482 |
| 26 | 3136230F6 | 1206 | 1635 |
| 26 | 7041019H1 | 1218 | 1660 |
| 26 | 178010H1 | 1249 | 1493 |
| 26 | 4109878H1 | 1348 | 1617 |
| 26 | 2135134T6 | 1388 | 1727 |
| 26 | g434268 | 1404 | 1671 |
| 26 | 2809378T6 | 1477 | 1727 |
| 26 | g707734 | 1513 | 1698 |
| 26 | 2900195H1 | 1510 | 1714 |
| 26 | 7088770H1 | 2241 | 2784 |
| 26 | 2688875H1 | 2289 | 2543 |
| 26 | g1688483 | 2301 | 2695 |
| 26 | 3126776H1 | 2302 | 2574 |
| 26 | 4132607T6 | 2348 | 2933 |
| 26 | 3900947T8 | 2378 | 2822 |
| 26 | g3924091 | 2375 | 2772 |
| 26 | 764214H1 | 2380 | 2661 |
| 26 | g1697306 | 2396 | 2775 |
| 26 | g5589502 | 2398 | 2775 |
| 26 | g2210147 | 2404 | 2787 |
| 26 | 2900195T6 | 2417 | 2907 |
| 26 | 1262991R1 | 2417 | 2917 |
| 26 | 1262991H1 | 2417 | 2655 |
| 26 | 4978404H1 | 2446 | 2697 |
| 26 | 4508011H1 | 2457 | 2720 |
| 26 | 3735710H1 | 2461 | 2698 |
| 26 | 6217351H1 | 2472 | 2933 |
| 26 | g2524340 | 2476 | 2948 |
| 26 | 2117643T6 | 2489 | 2846 |
| 26 | g3645213 | 2494 | 2955 |
| 26 | g4372648 | 2495 | 2775 |
| 26 | g3596913 | 2496 | 2958 |
| 26 | g4372672 | 2500 | 2951 |
| 26 | g3674408 | 2514 | 2955 |
| 26 | g4310744 | 2515 | 2963 |
| 26 | 723560H1 | 2516 | 2722 |
| 26 | g3595253 | 2521 | 2964 |
| 26 | g2525244 | 2522 | 2770 |
| 26 | 4160050T6 | 2527 | 2934 |
| 26 | g4665502 | 2536 | 2964 |
| 26 | 2743458H1 | 2544 | 2802 |
| 26 | 2738862H1 | 2544 | 2790 |
| 26 | 2743458F7 | 2544 | 2949 |
| 26 | g5637396 | 2548 | 2955 |
| 26 | g3432798 | 2549 | 2950 |
| 26 | g5369924 | 2550 | 2979 |
| 26 | g4310271 | 2552 | 2949 |
| 26 | g3841661 | 2554 | 2953 |
| 26 | g4888429 | 2557 | 2949 |
| 26 | 4620145H1 | 2558 | 2805 |
| 26 | 2605880T6 | 2563 | 2848 |
| 26 | 3207727H1 | 2565 | 2825 |
| 26 | g1783745 | 2570 | 2949 |
| 26 | g1677968 | 2571 | 2950 |
| 26 | 3980476H1 | 2573 | 2751 |
| 26 | 3966076H1 | 2572 | 2704 |
| 26 | 3966076F7 | 2572 | 2963 |
| 26 | 3966076T7 | 2572 | 2859 |
| 26 | g5445604 | 2583 | 2959 |
| 26 | 6494640H1 | 2583 | 2945 |
| 26 | g4622298 | 2586 | 2949 |
| 26 | g6439089 | 2592 | 2960 |
| 26 | 1258355T6 | 2592 | 2910 |
| 26 | g5540648 | 2607 | 2960 |
| 26 | 2273680H1 | 2614 | 2887 |
| 26 | g4620011 | 2619 | 2949 |
| 26 | 1711022H1 | 2630 | 2844 |
| 26 | 6587024H1 | 2633 | 2749 |
| 26 | 1472231H1 | 2634 | 2836 |
| 26 | 1472231T1 | 2634 | 2906 |
| 26 | 5177089H1 | 2643 | 2915 |
| 26 | 1711022F7 | 2646 | 2964 |
| 26 | g1860279 | 2645 | 2949 |
| 26 | g3837895 | 2646 | 2958 |
| 26 | g1644779 | 2650 | 2945 |
| 26 | 5645429H1 | 2650 | 2902 |
| 26 | g858397 | 2651 | 2926 |
| 26 | g4107761 | 2656 | 2949 |
| 26 | g839957 | 2659 | 2950 |
| 26 | g657066 | 2662 | 2963 |
| 26 | g5325849 | 2682 | 2956 |
| 26 | g727611 | 2687 | 2950 |
| 26 | 1413430H1 | 2685 | 2938 |
| 26 | 2887585H1 | 2686 | 2946 |
| 26 | g1242496 | 2689 | 2949 |
| 26 | 1796886H1 | 2696 | 2945 |
| 26 | g1688397 | 2697 | 2945 |
| 26 | 1711022T7 | 2706 | 2842 |
| 26 | g2941734 | 2711 | 2965 |
| 26 | 3022288H1 | 2725 | 2949 |
| 26 | g777818 | 2734 | 2959 |
| 26 | g434265 | 2732 | 2949 |
| 26 | g789233 | 2736 | 2941 |
| 26 | g789199 | 2741 | 2954 |
| 26 | g3144224 | 2745 | 2949 |
| 26 | 2737665H1 | 2751 | 2945 |
| 26 | g4195279 | 2774 | 2953 |
| 26 | 4718419H1 | 2784 | 2980 |
| 26 | g1948844 | 2803 | 2958 |
| 26 | g2524323 | 2860 | 2949 |
| 26 | g2106662 | 2878 | 2948 |
| 27 | 6506033H1 | 273 | 426 |
| 27 | 6506133H1 | 273 | 434 |
| 27 | 5742229H1 | 319 | 613 |
| 27 | 6334334H1 | 237 | 643 |
| 27 | 3388613F6 | 1 | 485 |
| 27 | 3388613H1 | 1 | 291 |
| 27 | g1815060 | 33 | 488 |
| 27 | 6941841H1 | 79 | 525 |
| 28 | 5372512H1 | 1 | 156 |
| 28 | 3481951H1 | 1 | 300 |
| 28 | 086514H1 | 116 | 324 |
| 28 | 6773036J1 | 136 | 255 |
| 28 | 3228141H1 | 256 | 535 |
| 28 | 469821H1 | 427 | 664 |
| 28 | 4323062H1 | 454 | 579 |
| 28 | g6131888 | 498 | 919 |
| 28 | 2997736H1 | 585 | 872 |
| 28 | 648674H1 | 723 | 978 |
| 28 | g6474252 | 755 | 1175 |
| 28 | 2720552F6 | 824 | 1376 |
| 28 | 2720552H1 | 824 | 1055 |
| 28 | 3731554H1 | 848 | 1141 |
| 28 | g1294839 | 880 | 1372 |
| 28 | g1294842 | 880 | 1336 |
| 28 | 5837915H1 | 1008 | 1273 |
| 28 | 5598427H1 | 1063 | 1333 |
| 28 | 7060462H1 | 1093 | 1625 |
| 28 | 2925401H1 | 1115 | 1340 |
| 28 | 3147672H1 | 1212 | 1519 |
| 28 | 7353404H1 | 1216 | 1753 |
| 28 | g2018589 | 1262 | 1572 |
| 28 | 5290315H1 | 1344 | 1593 |
| 28 | 2720552T6 | 1402 | 1736 |
| 28 | g570734 | 1437 | 1695 |
| 28 | 999643H1 | 1449 | 1694 |
| 28 | 6805084J1 | 1475 | 1968 |
| 28 | 1631088F6 | 1521 | 1966 |
| 28 | 2092882H1 | 1521 | 1721 |
| 28 | g1242302 | 1550 | 1775 |
| 28 | g2955269 | 1564 | 1775 |
| 28 | 139470H1 | 1620 | 1736 |
| 28 | 2072462H1 | 1639 | 1906 |
| 28 | 4286166H1 | 1641 | 1911 |
| 28 | g1242305 | 1660 | 1775 |
| 28 | 085524H1 | 1714 | 1911 |
| 28 | g1210407 | 1752 | 1950 |
| 28 | g1196175 | 1753 | 1950 |
| 28 | 4287958H1 | 1768 | 1911 |
| 28 | 1631088H1 | 1789 | 1966 |
| 29 | g5446946 | 632 | 897 |
| 29 | g3075841 | 221 | 576 |
| 29 | g2767512 | 248 | 577 |
| 29 | g2818989 | 248 | 593 |
| 29 | 3394790H1 | 353 | 636 |
| 29 | g2901002 | 453 | 708 |
| 29 | 804254H1 | 569 | 849 |
| 29 | 2906845H1 | 1 | 78 |
| 29 | 2741409T6 | 1 | 548 |
| 29 | 2741409F6 | 7 | 488 |
| 29 | 2741409H1 | 7 | 269 |
| 29 | 658133H1 | 12 | 268 |
| 29 | 3836585H1 | 31 | 316 |
| 29 | 4796210H1 | 129 | 412 |
| 29 | 6205209H1 | 154 | 370 |
| 29 | 1447527H1 | 218 | 457 |
| 30 | 7189828H2 | 1 | 573 |
| 30 | 7031283H1 | 176 | 699 |
| 30 | g4406653 | 255 | 2784 |
| 30 | 5594735H1 | 403 | 643 |
| 30 | 4858522H1 | 468 | 592 |
| 30 | 5151418H1 | 659 | 780 |
| 30 | 6440827H1 | 673 | 1152 |
| 30 | 3817033H1 | 949 | 1206 |
| 30 | 5007439H1 | 954 | 1182 |
| 30 | 5187677H1 | 997 | 1255 |
| 30 | 5003069H1 | 1121 | 1381 |
| 30 | 6838881H1 | 1261 | 1723 |
| 30 | g772660 | 1266 | 1605 |
| 30 | g769304 | 1267 | 1535 |
| 30 | g564397 | 1267 | 1459 |
| 30 | g900136 | 1342 | 1684 |
| 30 | 5077690H1 | 1399 | 1642 |
| 30 | 6742827H1 | 1434 | 1983 |
| 30 | 1398653H1 | 1574 | 1839 |
| 30 | 1398653F6 | 1574 | 1819 |
| 30 | 5085840H1 | 1578 | 1764 |
| 30 | 5188226H1 | 1630 | 1896 |
| 30 | 1429039H1 | 1640 | 1909 |
| 30 | 5390274H1 | 1752 | 2017 |
| 30 | 5719068H1 | 1774 | 2172 |
| 30 | 1926364H1 | 1778 | 2016 |
| 30 | 4987108F8 | 2070 | 2609 |
| 30 | 4986308H1 | 2070 | 2366 |
| 30 | 4643979H1 | 2083 | 2337 |
| 30 | 4987108T8 | 2105 | 2606 |
| 30 | 4987108T9 | 2118 | 2684 |
| 30 | 7351533H1 | 2126 | 2615 |
| 30 | 2073148T6 | 2288 | 2745 |
| 30 | 607997H1 | 2302 | 2551 |
| 30 | 1625327T6 | 2305 | 2743 |
| 30 | 1398653T6 | 2312 | 2756 |
| 30 | 1625327F6 | 2312 | 2753 |
| 30 | 1625327H1 | 2312 | 2521 |
| 30 | 2073148F6 | 2312 | 2787 |
| 30 | g5235046 | 2312 | 2790 |
| 30 | 2073148H1 | 2312 | 2565 |
| 30 | g4988528 | 2313 | 2787 |
| 30 | g3238921 | 2317 | 2785 |
| 30 | g5850283 | 2319 | 2784 |
| 30 | g5233757 | 2323 | 2787 |
| 30 | g4688102 | 2337 | 2784 |
| 30 | 2606717H1 | 2337 | 2597 |
| 30 | g5451665 | 2363 | 2784 |
| 30 | g6473523 | 2372 | 2789 |
| 30 | 1689007H1 | 2390 | 2605 |
| 30 | g2883211 | 2398 | 2787 |
| 30 | g6471739 | 2401 | 2788 |
| 30 | g3765856 | 2401 | 2784 |
| 30 | 2840911H1 | 2418 | 2684 |
| 30 | g5445474 | 2428 | 2784 |
| 30 | g6199495 | 2432 | 2784 |
| 30 | g2817078 | 2436 | 2793 |
| 30 | g5367496 | 2437 | 2784 |
| 30 | g900137 | 2445 | 2791 |
| 30 | g3254684 | 2451 | 2790 |
| 30 | g821905 | 2459 | 2792 |
| 30 | g816772 | 2543 | 2792 |
| 30 | g560418 | 2544 | 2784 |
| 30 | g3051927 | 2582 | 2790 |
| 30 | g2358712 | 2597 | 2784 |
| 30 | g796532 | 2689 | 2791 |
| 31 | 6111576H1 | 9012 | 9325 |
| 31 | 5158913H1 | 9018 | 9313 |
| 31 | 5159112H1 | 9018 | 9178 |
| 31 | 6100031H1 | 9056 | 9341 |
| 31 | 70405514D1 | 9077 | 9213 |
| 31 | 70333057D1 | 9083 | 9540 |
| 31 | 70321671D1 | 9083 | 9258 |
| 31 | 70404961D1 | 9124 | 9213 |
| 31 | 6112811H1 | 9200 | 9519 |
| 31 | 70337581D1 | 9209 | 9540 |
| 31 | 70337708D1 | 9209 | 9540 |
| 31 | 70347673D1 | 9209 | 9540 |
| 31 | 70337784D1 | 9215 | 9521 |
| 31 | 6107901H1 | 9218 | 9540 |
| 31 | 6116727H1 | 9225 | 9517 |
| 31 | 5442124H1 | 9226 | 9473 |
| 31 | 70337937D1 | 9235 | 9540 |
| 31 | 70339709D1 | 9243 | 9540 |
| 31 | 70322912D1 | 9243 | 9521 |
| 31 | 70346838D1 | 9243 | 9361 |
| 31 | 70346734D1 | 9243 | 9361 |
| 31 | 70338406D1 | 9258 | 9657 |
| 31 | 70346872D1 | 9262 | 9521 |
| 31 | g4325928 | 8675 | 8909 |
| 31 | 2874640H1 | 8718 | 8904 |
| 31 | 6019242H1 | 8727 | 8917 |
| 31 | 70404974D1 | 8821 | 9213 |
| 31 | 3387504H1 | 8840 | 8897 |
| 31 | 6299464H1 | 8898 | 9207 |
| 31 | 6300602H1 | 8898 | 9216 |
| 31 | 6343373H1 | 8905 | 9205 |
| 31 | 4570393H1 | 8939 | 9203 |
| 31 | 70340823D1 | 8943 | 9202 |
| 31 | 6602501H1 | 8954 | 9535 |
| 31 | 6080143H1 | 8956 | 9450 |
| 31 | 70406005D1 | 8957 | 9207 |
| 31 | 3903264H1 | 8957 | 9192 |
| 31 | 70405554D1 | 8957 | 9122 |
| 31 | 3171665H1 | 8957 | 9054 |
| 31 | 6576424H1 | 8962 | 9187 |
| 31 | 6731345H1 | 8964 | 9514 |
| 31 | 5177674H2 | 8964 | 9233 |
| 31 | 5605333H1 | 8964 | 9106 |
| 31 | 6823919H1 | 8969 | 9510 |
| 31 | 6823919J1 | 8969 | 9505 |
| 31 | 6859476H1 | 8985 | 9421 |
| 31 | 6855538H1 | 8985 | 9515 |
| 31 | 5752336H1 | 8989 | 9520 |
| 31 | 5756348H1 | 8990 | 9549 |
| 31 | 6604143H1 | 8989 | 9547 |
| 31 | 5296001H1 | 8992 | 9145 |
| 31 | 7220264H1 | 9007 | 9505 |
| 31 | 7313036H1 | 9006 | 9503 |
| 31 | g3399851 | 8504 | 8903 |
| 31 | g1939318 | 8504 | 8895 |
| 31 | g3150717 | 8508 | 8904 |
| 31 | 1978921H1 | 8533 | 8804 |
| 31 | g3427618 | 8559 | 8904 |
| 31 | g1885652 | 8614 | 8904 |
| 31 | 2917437F6 | 8632 | 9171 |
| 31 | 2917437H1 | 8633 | 8895 |
| 31 | 1242917H1 | 7364 | 7579 |
| 31 | 1887279F6 | 7369 | 7953 |
| 31 | 1887279H1 | 7369 | 7448 |
| 31 | 6873590H1 | 7384 | 7978 |
| 31 | 1904771H1 | 7463 | 7736 |
| 31 | 1594587T6 | 7476 | 7994 |
| 31 | 2158441H1 | 7477 | 7745 |
| 31 | 1434154H1 | 7497 | 7745 |
| 31 | 388880H1 | 7499 | 7757 |
| 31 | 347519H1 | 7502 | 7737 |
| 31 | 1922153T6 | 7506 | 7994 |
| 31 | 038495H1 | 7515 | 7757 |
| 31 | 038806H1 | 7515 | 7797 |
| 31 | 1922153R6 | 7530 | 7991 |
| 31 | 039364H1 | 7529 | 7762 |
| 31 | 1922153H1 | 7530 | 7801 |
| 31 | 1645260H1 | 7572 | 7680 |
| 31 | g3052905 | 7573 | 8094 |
| 31 | 3714030H1 | 7598 | 7910 |
| 31 | g3146667 | 7603 | 8097 |
| 31 | 2880781T6 | 7616 | 7992 |
| 31 | g4187707 | 7634 | 8100 |
| 31 | 1887279T6 | 7636 | 7992 |
| 31 | g3897482 | 7653 | 8095 |
| 31 | g4969820 | 7654 | 8097 |
| 31 | g4689698 | 7657 | 8097 |
| 31 | g2718258 | 7659 | 8095 |
| 31 | g4260707 | 7661 | 8100 |
| 31 | g6450348 | 7669 | 8101 |
| 31 | 1384216F6 | 7675 | 8102 |
| 31 | 1384216H1 | 7675 | 7953 |
| 31 | 1383157H1 | 7675 | 7890 |
| 31 | 1384216T6 | 7677 | 7992 |
| 31 | g5742420 | 7680 | 8097 |
| 31 | g4435778 | 7689 | 8098 |
| 31 | g5590793 | 7708 | 8098 |
| 31 | 2312710H1 | 7709 | 7944 |
| 31 | 3465757H1 | 7722 | 8000 |
| 31 | g6138571 | 7718 | 8097 |
| 31 | g853538 | 7740 | 7992 |
| 31 | 3861860H1 | 7754 | 7939 |
| 31 | g1277259 | 7774 | 8101 |
| 31 | g6503686 | 7774 | 8091 |
| 31 | g6044658 | 7779 | 8097 |
| 31 | g2023638 | 7782 | 8105 |
| 31 | 408644H1 | 7784 | 7992 |
| 31 | g843313 | 7788 | 8091 |
| 31 | g2952680 | 7805 | 8104 |
| 31 | g2229584 | 7806 | 8097 |
| 31 | 3122773H1 | 7863 | 8172 |
| 31 | g1963933 | 7882 | 8014 |
| 31 | g3058765 | 7903 | 8097 |
| 31 | 4295759H1 | 7935 | 8174 |
| 31 | 3296620H1 | 7949 | 8097 |
| 31 | 1252602T6 | 7949 | 8061 |
| 31 | 4179145H1 | 8046 | 8317 |
| 31 | g1859398 | 8055 | 8246 |
| 31 | 2950731F6 | 8069 | 8580 |
| 31 | 2950731H1 | 8069 | 8345 |
| 31 | 6028146H1 | 8097 | 8380 |
| 31 | 5486417H1 | 8097 | 8370 |
| 31 | 5486517H1 | 8097 | 8304 |
| 31 | 3214839H1 | 8129 | 8371 |
| 31 | 961014H1 | 8139 | 8419 |
| 31 | g1939317 | 8141 | 8602 |
| 31 | 2026064R6 | 8157 | 8553 |
| 31 | 2026064H1 | 8157 | 8432 |
| 31 | 6833043H1 | 8221 | 8813 |
| 31 | 6582104T1 | 8245 | 8801 |
| 31 | 2957010H1 | 8248 | 8528 |
| 31 | 3188576H1 | 8270 | 8606 |
| 31 | 6160420H1 | 8289 | 8582 |
| 31 | 4358203H1 | 8289 | 8572 |
| 31 | 273134H1 | 8307 | 8488 |
| 31 | 4461544H1 | 8325 | 8508 |
| 31 | 2950731T6 | 8348 | 8863 |
| 31 | 2026064T6 | 8348 | 8866 |
| 31 | 3731083H1 | 8401 | 8571 |
| 31 | 4627722H1 | 8422 | 8688 |
| 31 | g3897842 | 8446 | 8904 |
| 31 | 3527823H1 | 8448 | 8639 |
| 31 | g5878132 | 8463 | 8908 |
| 31 | g6476346 | 8474 | 8905 |
| 31 | g3841840 | 8481 | 8904 |
| 31 | g3432813 | 8495 | 8904 |
| 31 | g848912 | 2041 | 2367 |
| 31 | 6155860H1 | 2079 | 2380 |
| 31 | 5160887H2 | 2077 | 2339 |
| 31 | 1730365H1 | 2137 | 2253 |
| 31 | 7289495H1 | 2498 | 2716 |
| 31 | g5839114 | 2563 | 3016 |
| 31 | g6139732 | 2641 | 3042 |
| 31 | 360481H1 | 2850 | 3061 |
| 31 | 7086425H1 | 3497 | 3853 |
| 31 | 3825611H1 | 3604 | 3881 |
| 31 | 7280555H1 | 3778 | 4288 |
| 31 | 638057H1 | 4220 | 4486 |
| 31 | 6972901H1 | 4316 | 4865 |
| 31 | 1912997H1 | 4535 | 4745 |
| 31 | 5519751H1 | 4653 | 4868 |
| 31 | 4285086F6 | 4774 | 5061 |
| 31 | 71059434V1 | 4774 | 5377 |
| 31 | 71250506V1 | 4774 | 5349 |
| 31 | 71249863V1 | 4774 | 5433 |
| 31 | 4285086H1 | 4776 | 5097 |
| 31 | 71249286V1 | 4781 | 5353 |
| 31 | 71063350V1 | 4930 | 5441 |
| 31 | 4248781H1 | 4989 | 5249 |
| 31 | 71064013V1 | 5073 | 5658 |
| 31 | 71250536V1 | 5100 | 5657 |
| 31 | 71065578V1 | 5254 | 5657 |
| 31 | 71112003V1 | 5355 | 5678 |
| 31 | 6093082H1 | 5376 | 5664 |
| 31 | 71114663V1 | 5379 | 5655 |
| 31 | 5506319H1 | 5391 | 5647 |
| 31 | g5675608 | 5471 | 5935 |
| 31 | 1233336H1 | 5529 | 5670 |
| 31 | 1233340H1 | 5529 | 5669 |
| 31 | 1233275F1 | 5529 | 6093 |
| 31 | 1233275H1 | 5529 | 5771 |
| 31 | 5350013H1 | 5593 | 5663 |
| 31 | 1350122F1 | 5617 | 6131 |
| 31 | 1350122H1 | 5617 | 5864 |
| 31 | 6582104H1 | 5749 | 6350 |
| 31 | 1272111F1 | 5749 | 6328 |
| 31 | 1272111H1 | 5749 | 5966 |
| 31 | 1268160F1 | 5837 | 6376 |
| 31 | 1268160H1 | 5837 | 5970 |
| 31 | 7001273H1 | 5888 | 6459 |
| 31 | 2880781F6 | 5919 | 6392 |
| 31 | 2880781H1 | 5919 | 6235 |
| 31 | 087051H1 | 5952 | 6255 |
| 31 | 6983691H1 | 5984 | 6152 |
| 31 | 6201190H1 | 6031 | 6471 |
| 31 | 6201790H1 | 6042 | 6471 |
| 31 | g1959108 | 6053 | 6610 |
| 31 | 7080464H1 | 6067 | 6583 |
| 31 | g2191498 | 6075 | 6507 |
| 31 | 2534081H1 | 6250 | 6492 |
| 31 | 5926420H1 | 6346 | 6644 |
| 31 | 4846224H2 | 6368 | 6676 |
| 31 | 6782312H1 | 6413 | 7032 |
| 31 | 7162675H1 | 6428 | 6832 |
| 31 | 2483472H1 | 6560 | 6820 |
| 31 | 6774735J1 | 6564 | 7115 |
| 31 | 3350377H1 | 6564 | 6849 |
| 31 | 3374950H1 | 6564 | 6842 |
| 31 | 6432475H1 | 6598 | 7078 |
| 31 | 6782312J1 | 6592 | 7292 |
| 31 | 6436285H1 | 6598 | 7058 |
| 31 | 705724H1 | 6672 | 6920 |
| 31 | g573365 | 6676 | 7044 |
| 31 | 1594587F6 | 6687 | 7069 |
| 31 | 1594587H1 | 6687 | 6915 |
| 31 | 6774735H1 | 6732 | 7173 |
| 31 | 2541992H1 | 6721 | 6923 |
| 31 | 4541545H1 | 6758 | 7002 |
| 31 | 755175R1 | 6768 | 7321 |
| 31 | 755175H1 | 6768 | 6985 |
| 31 | 756456H1 | 6768 | 7031 |
| 31 | 1252602H1 | 6783 | 6920 |
| 31 | 1252602F6 | 6783 | 6978 |
| 31 | 2150018H1 | 6786 | 7056 |
| 31 | 2490476H1 | 6812 | 7065 |
| 31 | 1655265H1 | 6866 | 7129 |
| 31 | 6997276H1 | 6919 | 7496 |
| 31 | g843312 | 6925 | 7278 |
| 31 | g853537 | 6925 | 7255 |
| 31 | g2115300 | 6929 | 7315 |
| 31 | 6307352H1 | 6936 | 7393 |
| 31 | 1889141H1 | 6950 | 7270 |
| 31 | 3388377H1 | 7017 | 7341 |
| 31 | 7360267H1 | 7024 | 7239 |
| 31 | g1986541 | 7052 | 7349 |
| 31 | 3414787H1 | 7098 | 7329 |
| 31 | 4750579H2 | 7106 | 7385 |
| 31 | 4576939H1 | 7113 | 7253 |
| 31 | 2573123H1 | 7128 | 7366 |
| 31 | 6501709H1 | 7132 | 7450 |
| 31 | 837427H1 | 7133 | 7372 |
| 31 | 6837032H1 | 7133 | 7387 |
| 31 | 2742578H1 | 7170 | 7414 |
| 31 | g923876 | 7189 | 7484 |
| 31 | 3230227H1 | 7210 | 7362 |
| 31 | 103247H1 | 7254 | 7488 |
| 31 | g1312411 | 7271 | 7738 |
| 31 | 5509393H1 | 7289 | 7520 |
| 31 | 4402891H1 | 7309 | 7388 |
| 31 | 3256579H1 | 7314 | 7559 |
| 31 | 3254592H1 | 7314 | 7550 |
| 31 | 1661957H1 | 7330 | 7555 |
| 31 | 5285340H1 | 7345 | 7413 |
| 31 | 7281173H1 | 1 | 510 |
| 31 | 6622925J1 | 63 | 589 |
| 31 | 6911740H1 | 63 | 480 |
| 31 | 6850369H1 | 187 | 771 |
| 31 | 6852822H1 | 187 | 514 |
| 31 | 6538053H1 | 253 | 621 |
| 31 | 70682475V1 | 297 | 917 |
| 31 | 70683802V1 | 297 | 796 |
| 31 | 1518525F6 | 297 | 749 |
| 31 | 1518525H1 | 297 | 485 |
| 31 | 6476129H1 | 308 | 901 |
| 31 | 2450348H1 | 346 | 615 |
| 31 | 4661820H2 | 361 | 420 |
| 31 | 70683210V1 | 385 | 1052 |
| 31 | 6331467H1 | 425 | 996 |
| 31 | 6911740J1 | 483 | 1063 |
| 31 | 70682237V1 | 528 | 1146 |
| 31 | 5546278H1 | 537 | 759 |
| 31 | 5546278F8 | 537 | 1007 |
| 31 | 7104540H1 | 555 | 1088 |
| 31 | 6084311H1 | 575 | 1119 |
| 31 | 7040029H1 | 606 | 1128 |
| 31 | 7039822H1 | 606 | 1246 |
| 31 | 7103540H1 | 607 | 993 |
| 31 | 4559288H1 | 695 | 888 |
| 31 | 4559288F8 | 699 | 1263 |
| 31 | 70708922V1 | 703 | 964 |
| 31 | 2137232F6 | 709 | 1120 |
| 31 | 2137232H1 | 709 | 997 |
| 31 | 7343723H1 | 721 | 1319 |
| 31 | 70683497V1 | 740 | 1394 |
| 31 | 70680391V1 | 747 | 1268 |
| 31 | 5808779H2 | 755 | 992 |
| 31 | 6433283H1 | 841 | 1387 |
| 31 | 7359432H1 | 845 | 1364 |
| 31 | 7196090H1 | 851 | 1329 |
| 31 | 70046830V1 | 863 | 1263 |
| 31 | 70569476V1 | 878 | 1120 |
| 31 | 70569482V1 | 877 | 1456 |
| 31 | 2171346F6 | 883 | 1372 |
| 31 | 2171346H1 | 883 | 1121 |
| 31 | 2170895H1 | 883 | 1117 |
| 31 | 70679948V1 | 928 | 1514 |
| 31 | 70683916V1 | 930 | 1509 |
| 31 | 7237350H1 | 933 | 1413 |
| 31 | 7237380H1 | 933 | 1397 |
| 31 | 1813175H1 | 936 | 1204 |
| 31 | 70569167V1 | 940 | 1120 |
| 31 | 5547274H1 | 949 | 1151 |
| 31 | 5547274F8 | 949 | 1018 |
| 31 | 6717279H1 | 952 | 1536 |
| 31 | 70682430V1 | 971 | 1509 |
| 31 | 7203232H1 | 981 | 1352 |
| 31 | 70684182V1 | 1013 | 1517 |
| 31 | 70681054V1 | 1034 | 1633 |
| 31 | 70681099V1 | 1034 | 1633 |
| 31 | 2207642H1 | 1046 | 1286 |
| 31 | 70570849V1 | 1056 | 1126 |
| 31 | 70684761V1 | 1095 | 1515 |
| 31 | 70679678V1 | 1100 | 1650 |
| 31 | 70679671V1 | 1135 | 1520 |
| 31 | 70684064V1 | 1135 | 1732 |
| 31 | 70682692V1 | 1170 | 1671 |
| 31 | 70687134V1 | 1215 | 1435 |
| 31 | 70680881V1 | 1229 | 1685 |
| 31 | 70680492V1 | 1277 | 1923 |
| 31 | 6345849H1 | 1279 | 1540 |
| 31 | 70680243V1 | 1282 | 1652 |
| 31 | 70684419V1 | 1321 | 1825 |
| 31 | 70684171V1 | 1321 | 1825 |
| 31 | 70685150V1 | 1325 | 1818 |
| 31 | 2013142R6 | 1336 | 1776 |
| 31 | 2013142H1 | 1336 | 1518 |
| 31 | 4257159F7 | 1340 | 1952 |
| 31 | 4257159H1 | 1340 | 1514 |
| 31 | 6879724H1 | 1358 | 1902 |
| 31 | 6779454J1 | 1374 | 1931 |
| 31 | 70047152V1 | 1374 | 1888 |
| 31 | 7284681H1 | 1382 | 1855 |
| 31 | 70680875V1 | 1399 | 1941 |
| 31 | g2224530 | 1461 | 8904 |
| 31 | 5546278T8 | 1485 | 2054 |
| 31 | 2013142T6 | 1492 | 2039 |
| 31 | 70047143V1 | 1501 | 1932 |
| 31 | 70704799V1 | 1537 | 1725 |
| 31 | 6842915H1 | 1547 | 2041 |
| 31 | 5680223H1 | 1582 | 1832 |
| 31 | g4533055 | 1617 | 2079 |
| 31 | g5512485 | 1621 | 2079 |
| 31 | 7282247H1 | 1629 | 2142 |
| 31 | g4568745 | 1637 | 2079 |
| 31 | g317862 | 1662 | 2037 |
| 31 | g3039050 | 1670 | 2078 |
| 31 | 4257159T7 | 1676 | 2261 |
| 31 | 3520727R6 | 1685 | 2079 |
| 31 | 3520727T6 | 1685 | 2020 |
| 31 | 3520727H1 | 1685 | 1970 |
| 31 | 70689194V1 | 1689 | 1909 |
| 31 | 4527468H1 | 1728 | 1976 |
| 31 | 1518525T6 | 1744 | 2324 |
| 31 | g848911 | 1748 | 2062 |
| 31 | g897000 | 1748 | 2092 |
| 31 | g846844 | 1748 | 1979 |
| 31 | 5948631H1 | 1767 | 2077 |
| 31 | 4947717H1 | 1816 | 2060 |
| 31 | g4328051 | 1836 | 2079 |
| 31 | 1407176T6 | 1887 | 2312 |
| 31 | g6397504 | 1899 | 2369 |
| 31 | g2675405 | 1913 | 2390 |
| 31 | 1904789T6 | 1914 | 2329 |
| 31 | 70686880V1 | 1918 | 2138 |
| 31 | 6879724J1 | 1922 | 2510 |
| 31 | 1904789H1 | 1921 | 2184 |
| 31 | 1904789F6 | 1921 | 2366 |
| 31 | g4312534 | 1952 | 2364 |
| 31 | 6955654H1 | 1972 | 2379 |
| 31 | 6880650J1 | 1978 | 2213 |
| 31 | 5498289H1 | 1990 | 2241 |
| 31 | g4534624 | 1991 | 2369 |
| 32 | 3427795F6 | 1 | 425 |
| 32 | 3427795H1 | 1 | 125 |
| 32 | 6413185H1 | 1 | 199 |
| 32 | 2798241F6 | 4 | 507 |
| 32 | 2798241H1 | 4 | 190 |
| 32 | g1063751 | 13 | 303 |
| 32 | 890219H1 | 20 | 279 |
| 32 | 4755289H1 | 60 | 323 |
| 32 | 6908205J1 | 358 | 968 |
| 32 | g1064447 | 401 | 679 |
| 32 | 6744456H1 | 421 | 990 |
| 32 | 7100413H1 | 477 | 1038 |
| 32 | 3074435H1 | 562 | 828 |
| 32 | 3894976H1 | 626 | 934 |
| 32 | 3894976F6 | 633 | 1124 |
| 32 | 182014H1 | 678 | 871 |
| 32 | 5374989H1 | 761 | 997 |
| 32 | 3990437H1 | 826 | 1119 |
| 32 | 4073773H1 | 832 | 1124 |
| 32 | 4073881F6 | 832 | 1310 |
| 32 | 4073773F6 | 832 | 1251 |
| 32 | 4073249H1 | 832 | 1045 |
| 32 | g3803176 | 860 | 1322 |
| 32 | g3016772 | 860 | 1149 |
| 32 | g3677768 | 860 | 1186 |
| 32 | 6570162H1 | 907 | 1178 |
| 32 | 6265054H1 | 907 | 1175 |
| 32 | 6264218H1 | 907 | 1175 |
| 32 | g4532299 | 979 | 1419 |
| 32 | 6586921H1 | 1010 | 1531 |
| 32 | 5331958H1 | 1030 | 1159 |
| 32 | g5112058 | 1065 | 1520 |
| 32 | g2100884 | 1065 | 1539 |
| 32 | 5565058H1 | 1065 | 1297 |
| 32 | 6563309H1 | 1225 | 1789 |
| 32 | 6563209H1 | 1225 | 1773 |
| 32 | g3174367 | 1230 | 1354 |
| 32 | 6563509H1 | 1264 | 1773 |
| 32 | 2798241T6 | 1278 | 1780 |
| 32 | 4073773T6 | 1314 | 1782 |
| 32 | 1003401H1 | 1326 | 1569 |
| 32 | 3894976T6 | 1329 | 1780 |
| 32 | g2101001 | 1364 | 1680 |
| 32 | 6342025H1 | 1412 | 1818 |
| 32 | g5593969 | 1422 | 1818 |
| 32 | g2357643 | 1509 | 1825 |
| 32 | g822002 | 1552 | 1825 |
| 32 | 6322537H1 | 1570 | 1816 |
| 33 | 1634654H1 | 1610 | 1839 |
| 33 | 60117794D3 | 1 | 562 |
| 33 | 60117794D1 | 1 | 460 |
| 33 | 70158065V1 | 1 | 384 |
| 33 | g4309459 | 2 | 310 |
| 33 | 60211518U1 | 18 | 510 |
| 33 | 60211520U1 | 18 | 304 |
| 33 | 777324R6 | 60 | 552 |
| 33 | 777324H1 | 60 | 303 |
| 33 | 70159276V1 | 75 | 428 |
| 33 | 60211517U1 | 85 | 162 |
| 33 | 961319H1 | 87 | 308 |
| 33 | 961184T1 | 87 | 271 |
| 33 | 2275142H1 | 96 | 359 |
| 33 | 70160733V1 | 113 | 620 |
| 33 | 5085664H1 | 131 | 306 |
| 33 | 70157316V1 | 168 | 682 |
| 33 | 70159504V1 | 185 | 551 |
| 33 | 70159696V1 | 185 | 655 |
| 33 | 564074H1 | 223 | 436 |
| 33 | 70159471V1 | 309 | 826 |
| 33 | g1147353 | 351 | 769 |
| 33 | 60211524U1 | 377 | 847 |
| 33 | 70159585V1 | 380 | 935 |
| 33 | g1148991 | 452 | 799 |
| 33 | 1561109F6 | 518 | 999 |
| 33 | 1561109H1 | 518 | 734 |
| 33 | 70158015V1 | 522 | 1007 |
| 33 | 70161128V1 | 578 | 1105 |
| 33 | 60211519U1 | 586 | 1130 |
| 33 | 3807313H1 | 607 | 849 |
| 33 | 70455557V1 | 618 | 1130 |
| 33 | 70157813V1 | 629 | 1216 |
| 33 | 70159960V1 | 636 | 1144 |
| 33 | 3495050H1 | 646 | 942 |
| 33 | 70277446V1 | 656 | 941 |
| 33 | 60211521U1 | 658 | 1131 |
| 33 | 689505H1 | 669 | 909 |
| 33 | 70159857V1 | 700 | 1193 |
| 33 | 70272966V1 | 727 | 1249 |
| 33 | 2132279R6 | 753 | 1363 |
| 33 | 2132279H1 | 753 | 1001 |
| 33 | 70157717V1 | 762 | 1216 |
| 33 | 70157909V1 | 773 | 1216 |
| 33 | 70456567V1 | 795 | 1370 |
| 33 | 4741196H1 | 828 | 1088 |
| 33 | 4588447H1 | 828 | 994 |
| 33 | 71191743V1 | 832 | 1483 |
| 33 | 71189175V1 | 837 | 1294 |
| 33 | 70457596V1 | 863 | 1238 |
| 33 | 70456909V1 | 882 | 1428 |
| 33 | 71191729V1 | 881 | 1404 |
| 33 | 1580127H1 | 886 | 1072 |
| 33 | 70457420V1 | 915 | 1479 |
| 33 | 1600238H1 | 928 | 1161 |
| 33 | 70454132V1 | 949 | 1490 |
| 33 | 70454972V1 | 949 | 1611 |
| 33 | 70455589V1 | 954 | 1447 |
| 33 | 1563424H1 | 958 | 1224 |
| 33 | 4106652H1 | 984 | 1142 |
| 33 | 70158516V1 | 988 | 1257 |
| 33 | 4959017H1 | 990 | 1264 |
| 33 | 71188204V1 | 994 | 1446 |
| 33 | 60211523U1 | 1037 | 1544 |
| 33 | 70454512V1 | 1039 | 1519 |
| 33 | 70454364V1 | 1051 | 1604 |
| 33 | 70454390V1 | 1051 | 1603 |
| 33 | 70456889V1 | 1070 | 1701 |
| 33 | 70452566V1 | 1097 | 1581 |
| 33 | 71190479V1 | 1107 | 1395 |
| 33 | 70272589V1 | 1127 | 1424 |
| 33 | 70456244V1 | 1127 | 1681 |
| 33 | g2183587 | 1145 | 1416 |
| 33 | 3088034H1 | 1157 | 1443 |
| 33 | 70456004V1 | 1169 | 1806 |
| 33 | 3245093H1 | 1179 | 1437 |
| 33 | 70159879V1 | 1196 | 1700 |
| 33 | 6034154H1 | 1199 | 1788 |
| 33 | 70159176V1 | 1226 | 1777 |
| 33 | g1886430 | 1247 | 1678 |
| 33 | 71128906V1 | 1247 | 1507 |
| 33 | 71188432V1 | 1254 | 1589 |
| 33 | 71191660V1 | 1255 | 1810 |
| 33 | 1662925F6 | 1290 | 1775 |
| 33 | 1663527F6 | 1290 | 1621 |
| 33 | 1663527H1 | 1290 | 1524 |
| 33 | 1662925H1 | 1290 | 1411 |
| 33 | 5658206H1 | 1301 | 1535 |
| 33 | 70457670V1 | 1313 | 1838 |
| 33 | g2902092 | 1320 | 1660 |
| 33 | 70455019V1 | 1343 | 1884 |
| 33 | 70454189V1 | 1351 | 1752 |
| 33 | 70455783V1 | 1376 | 1882 |
| 33 | 777324T6 | 1381 | 2019 |
| 33 | 70456596V1 | 1407 | 2011 |
| 33 | 3987203H1 | 1406 | 1705 |
| 33 | 70157664V1 | 1410 | 1852 |
| 33 | 2132279T6 | 1411 | 2028 |
| 33 | g761771 | 1447 | 1605 |
| 33 | 2148914H1 | 1468 | 1724 |
| 33 | 60117794B1 | 1486 | 2036 |
| 33 | g2882479 | 1488 | 1831 |
| 33 | 5434158H1 | 1500 | 1741 |
| 33 | 1354153H1 | 1503 | 1772 |
| 33 | 714828H1 | 1536 | 1699 |
| 33 | 70277464V1 | 1544 | 1934 |
| 33 | 910569H1 | 1543 | 1843 |
| 33 | 2276506R6 | 1548 | 2003 |
| 33 | 2276506H1 | 1548 | 1658 |
| 33 | 70160695V1 | 1558 | 2009 |
| 33 | 70273430V1 | 1578 | 2068 |
| 33 | 60117794B2 | 1581 | 2055 |
| 33 | 6321907H1 | 1597 | 1884 |
| 33 | 2276506T6 | 1599 | 2026 |
| 33 | 70158895V1 | 1606 | 2066 |
| 33 | 70454932V1 | 1651 | 2061 |
| 33 | 1561109T6 | 1647 | 2029 |
| 33 | g3173670 | 1668 | 2072 |
| 33 | g2718341 | 1670 | 2067 |
| 33 | 506569H1 | 1707 | 1938 |
| 33 | g6029335 | 1710 | 2067 |
| 33 | g5366970 | 1765 | 2074 |
| 33 | 70457179V1 | 1781 | 1980 |
| 33 | g5444610 | 1790 | 2067 |
| 33 | g2554086 | 1793 | 2052 |
| 33 | 1638111F6 | 1809 | 2198 |
| 33 | 71156557V1 | 1820 | 1960 |
| 33 | g765481 | 1866 | 2070 |
| 33 | g2742608 | 1869 | 2074 |
| 33 | g3679767 | 1900 | 2198 |
| 33 | g6197210 | 1917 | 2072 |
| 33 | g3805642 | 1940 | 2070 |
| 33 | g2368950 | 1945 | 2069 |
| 33 | g2183586 | 1957 | 2020 |
| 33 | g2541498 | 1994 | 2067 |
| 33 | 1638111H1 | 1999 | 2198 |
| 33 | 2756125H1 | 2094 | 2198 |
| 33 | 1635764H1 | 2099 | 2198 |
| 33 | 1632232H1 | 1610 | 1822 |
| 33 | g6200220 | 1636 | 2068 |
| 34 | 5804093H1 | 1 | 177 |
| 34 | 2684902H1 | 8 | 257 |
| 34 | 5833363H1 | 27 | 136 |
| 34 | g3280141 | 29 | 431 |
| 34 | 4203938H1 | 100 | 374 |
| 34 | 2563955H1 | 179 | 402 |
| 35 | 70408909D1 | 1832 | 2372 |
| 35 | 70403746D1 | 1839 | 2338 |
| 35 | 70403011D1 | 1835 | 2338 |
| 35 | 70401407D1 | 1845 | 2338 |
| 35 | 70401227D1 | 1839 | 2339 |
| 35 | 70402036D1 | 1840 | 2338 |
| 35 | 70409006D1 | 1848 | 2338 |
| 35 | 70408788D1 | 1854 | 2338 |
| 35 | 70403814D1 | 1854 | 2338 |
| 35 | 70408844D1 | 1865 | 2338 |
| 35 | 70409308D1 | 1865 | 2337 |
| 35 | 70403074D1 | 1869 | 2338 |
| 35 | 70400541D1 | 1870 | 2338 |
| 35 | 70409808D1 | 1872 | 2337 |
| 35 | 70401172D1 | 1879 | 2338 |
| 35 | 70401503D1 | 1883 | 2338 |
| 35 | 70408973D1 | 1899 | 2338 |
| 35 | 70401272D1 | 1906 | 2338 |
| 35 | 70409425D1 | 1903 | 2338 |
| 35 | 70401367D1 | 1909 | 2338 |
| 35 | 70402747D1 | 1908 | 2338 |
| 35 | 70408459D1 | 1908 | 2338 |
| 35 | 70409112D1 | 1911 | 2427 |
| 35 | 70401329D1 | 1910 | 2427 |
| 35 | 70402712D1 | 1917 | 2338 |
| 35 | 70408374D1 | 1919 | 2338 |
| 35 | 70403748D1 | 1923 | 2338 |
| 35 | 70400493D1 | 1925 | 2338 |
| 35 | 3021378H1 | 1931 | 2239 |
| 35 | 70400784D1 | 1935 | 2338 |
| 35 | 70410219D1 | 1938 | 2338 |
| 35 | 70403042D1 | 1941 | 2338 |
| 35 | 70401079D1 | 1942 | 2338 |
| 35 | 70409409D1 | 1942 | 2337 |
| 35 | 70403961D1 | 1943 | 2338 |
| 35 | 70409163D1 | 1947 | 2427 |
| 35 | 70402796D1 | 1950 | 2338 |
| 35 | 3700809H1 | 1951 | 2251 |
| 35 | 70402339D1 | 1963 | 2338 |
| 35 | 3475377H1 | 1965 | 2116 |
| 35 | 5640458H1 | 1965 | 2103 |
| 35 | 70400757D1 | 1967 | 2338 |
| 35 | 70403528D1 | 1969 | 2338 |
| 35 | 70403542D1 | 1974 | 2426 |
| 35 | 70408826D1 | 1978 | 2337 |
| 35 | 70400431D1 | 1993 | 2654 |
| 35 | 70403190D1 | 1999 | 2427 |
| 35 | 70408542D1 | 2001 | 2337 |
| 35 | 70410141D1 | 2005 | 2338 |
| 35 | 70401284D1 | 2002 | 2338 |
| 35 | 71220096V1 | 2021 | 2742 |
| 35 | 7262840H1 | 2031 | 2527 |
| 35 | 3277490H1 | 2025 | 2282 |
| 35 | 4806444H1 | 2027 | 2305 |
| 35 | 5464475H1 | 2039 | 2303 |
| 35 | 70410172D1 | 2029 | 2427 |
| 35 | 4725604H1 | 2044 | 2327 |
| 35 | g1010563 | 2062 | 2431 |
| 35 | 2744790H1 | 2075 | 2336 |
| 35 | g1766452 | 2086 | 2417 |
| 35 | 70401307D1 | 2099 | 2441 |
| 35 | 6507755H1 | 2109 | 2637 |
| 35 | 70831953V1 | 2115 | 2668 |
| 35 | 3987571H1 | 2119 | 2384 |
| 35 | 70401747D1 | 2124 | 2360 |
| 35 | 3426754H1 | 2130 | 2401 |
| 35 | 485095H1 | 2143 | 2396 |
| 35 | 3492396H1 | 2150 | 2434 |
| 35 | 3931556H1 | 2156 | 2448 |
| 35 | 3930720H1 | 2156 | 2466 |
| 35 | 3934202H1 | 2158 | 2442 |
| 35 | 4889738H1 | 2172 | 2465 |
| 35 | 4906569H2 | 2195 | 2471 |
| 35 | 7272031H1 | 2200 | 2720 |
| 35 | 71219448V1 | 2202 | 2828 |
| 35 | 5468686H1 | 2207 | 2384 |
| 35 | 4914352H1 | 2208 | 2502 |
| 35 | g1636142 | 2209 | 2466 |
| 35 | 6160218H1 | 2219 | 2480 |
| 35 | 3873766H1 | 2234 | 2528 |
| 35 | 70403069D1 | 2234 | 2337 |
| 35 | 5425426H1 | 2245 | 2514 |
| 35 | 3643322H1 | 2257 | 2528 |
| 35 | 2123362F6 | 2257 | 2701 |
| 35 | 2123362H1 | 2257 | 2585 |
| 35 | 033931H1 | 2257 | 2335 |
| 35 | 4615229H1 | 2260 | 2522 |
| 35 | 3212960H1 | 2260 | 2557 |
| 35 | 6729709H1 | 2268 | 2832 |
| 35 | 2821789H1 | 2283 | 2592 |
| 35 | 6815772J1 | 2289 | 2737 |
| 35 | 6815772H1 | 2289 | 2738 |
| 35 | 4199572H1 | 2313 | 2480 |
| 35 | 2795091H1 | 2323 | 2571 |
| 35 | 1389192H1 | 2325 | 2575 |
| 35 | g1628772 | 2328 | 2690 |
| 35 | 70403666D1 | 2340 | 2804 |
| 35 | 2501716H1 | 2341 | 2567 |
| 35 | 3562675H1 | 2344 | 2655 |
| 35 | 3229405H1 | 2346 | 2449 |
| 35 | 4807968H1 | 2346 | 2604 |
| 35 | 2207651H1 | 2350 | 2546 |
| 35 | 5921344H1 | 2352 | 2625 |
| 35 | 6553789H1 | 2361 | 2831 |
| 35 | 3370548H1 | 2359 | 2635 |
| 35 | 6553802H1 | 2361 | 2837 |
| 35 | 5203637H1 | 2361 | 2624 |
| 35 | 2360538H1 | 2370 | 2620 |
| 35 | 5512063H1 | 2374 | 2618 |
| 35 | 466927H1 | 2384 | 2614 |
| 35 | 2867737H1 | 2404 | 2706 |
| 35 | 5022887H1 | 2406 | 2691 |
| 35 | 6361886H1 | 2430 | 2806 |
| 35 | 1750069H1 | 2444 | 2698 |
| 35 | 70409940D1 | 2447 | 2654 |
| 35 | 1459993H1 | 2451 | 2698 |
| 35 | 1591570H1 | 2457 | 2667 |
| 35 | 70400763D1 | 2458 | 2659 |
| 35 | 70402011D1 | 1804 | 2338 |
| 35 | 70402022D1 | 1804 | 2338 |
| 35 | 70401984D1 | 1804 | 2338 |
| 35 | 70403362D1 | 1804 | 2344 |
| 35 | 70409507D1 | 1803 | 2338 |
| 35 | 70400560D1 | 1803 | 2337 |
| 35 | 70408612D1 | 1803 | 2338 |
| 35 | 70408741D1 | 1803 | 2338 |
| 35 | 70403513D1 | 1803 | 2321 |
| 35 | 70409234D1 | 1803 | 2338 |
| 35 | 70409561D1 | 1803 | 2336 |
| 35 | 70402084D1 | 1803 | 2304 |
| 35 | 70402132D1 | 1803 | 2338 |
| 35 | 70404004D1 | 1803 | 2338 |
| 35 | 70402821D1 | 1803 | 2337 |
| 35 | 70403962D1 | 1803 | 2295 |
| 35 | 70402206D1 | 1803 | 2338 |
| 35 | 70409421D1 | 1804 | 2338 |
| 35 | 70402038D1 | 1803 | 2304 |
| 35 | 70403722D1 | 1803 | 2277 |
| 35 | 70410122D1 | 1803 | 2338 |
| 35 | 70408553D1 | 1803 | 2296 |
| 35 | 70401060D1 | 1803 | 2304 |
| 35 | 70409920D1 | 1805 | 2338 |
| 35 | 70409860D1 | 1803 | 2266 |
| 35 | 70409739D1 | 1803 | 2336 |
| 35 | 70402741D1 | 1803 | 2324 |
| 35 | 70403761D1 | 1803 | 2338 |
| 35 | 70401941D1 | 1803 | 2337 |
| 35 | 70402900D1 | 1803 | 2251 |
| 35 | 70403535D1 | 1803 | 2252 |
| 35 | 70401221D1 | 1803 | 2241 |
| 35 | 70402260D1 | 1803 | 2238 |
| 35 | 70402886D1 | 1803 | 2251 |
| 35 | 70403149D1 | 1803 | 2277 |
| 35 | 70409015D1 | 1803 | 2254 |
| 35 | 70403254D1 | 1803 | 2226 |
| 35 | 70401797D1 | 1803 | 2338 |
| 35 | 70401877D1 | 1803 | 2194 |
| 35 | 70402928D1 | 1803 | 2194 |
| 35 | 70402401D1 | 1803 | 2251 |
| 35 | 70401108D1 | 1803 | 2157 |
| 35 | 70403940D1 | 1803 | 2339 |
| 35 | 70409514D1 | 1803 | 2338 |
| 35 | 70400487D1 | 1803 | 2162 |
| 35 | 70409135D1 | 1804 | 2341 |
| 35 | 70401488D1 | 1804 | 2338 |
| 35 | 70410083D1 | 1803 | 2338 |
| 35 | 70401410D1 | 1803 | 2338 |
| 35 | 70401822D1 | 1804 | 2427 |
| 35 | 70409405D1 | 1804 | 2338 |
| 35 | 70402730D1 | 1803 | 2362 |
| 35 | 70400849D1 | 1804 | 2338 |
| 35 | 70404129D1 | 1804 | 2338 |
| 35 | 70408998D1 | 1804 | 2338 |
| 35 | 70408367D1 | 1803 | 2338 |
| 35 | 70402838D1 | 1803 | 2338 |
| 35 | 70403713D1 | 1803 | 2359 |
| 35 | 70402007D1 | 1804 | 2336 |
| 35 | 70409325D1 | 1804 | 2337 |
| 35 | 70409816D1 | 1803 | 2338 |
| 35 | 70408486D1 | 1803 | 2337 |
| 35 | 70401965D1 | 1804 | 2320 |
| 35 | 70403142D1 | 1804 | 2339 |
| 35 | 70400962D1 | 1803 | 2338 |
| 35 | 70408422D1 | 1803 | 2320 |
| 35 | 70408820D1 | 1804 | 2337 |
| 35 | 70400582D1 | 1803 | 2336 |
| 35 | 70410053D1 | 1803 | 2336 |
| 35 | 70401113D1 | 1803 | 2319 |
| 35 | 70409463D1 | 1804 | 2334 |
| 35 | 70402134D1 | 1803 | 2398 |
| 35 | 70401254D1 | 1803 | 2313 |
| 35 | 70401763D1 | 1803 | 2349 |
| 35 | 70402564D1 | 1803 | 2338 |
| 35 | 70401261D1 | 1803 | 2318 |
| 35 | 70400898D1 | 1803 | 2284 |
| 35 | 70403285D1 | 1803 | 2340 |
| 35 | 70403790D1 | 1803 | 2278 |
| 35 | 70402785D1 | 1803 | 2315 |
| 35 | 70409925D1 | 1803 | 2330 |
| 35 | 70410196D1 | 1804 | 2299 |
| 35 | 70409675D1 | 1803 | 2302 |
| 35 | 70409070D1 | 1804 | 2367 |
| 35 | 70401207D1 | 1804 | 2240 |
| 35 | 70409958D1 | 1803 | 2252 |
| 35 | 70401936D1 | 1804 | 2252 |
| 35 | 70404095D1 | 1804 | 2233 |
| 35 | 70402994D1 | 1803 | 2249 |
| 35 | 70400636D1 | 1804 | 2338 |
| 35 | 70403096D1 | 1804 | 2210 |
| 35 | 70409831D1 | 1804 | 2252 |
| 35 | 70403804D1 | 1803 | 2302 |
| 35 | 70409643D1 | 1804 | 2313 |
| 35 | 70402932D1 | 1803 | 2254 |
| 35 | 70402629D1 | 1804 | 2317 |
| 35 | 70408768D1 | 1803 | 2208 |
| 35 | 70403063D1 | 1803 | 2109 |
| 35 | 70404055D1 | 1803 | 2108 |
| 35 | 70402323D1 | 1803 | 2110 |
| 35 | 70408812D1 | 1805 | 2338 |
| 35 | 70409104D1 | 1805 | 2337 |
| 35 | 70408438D1 | 1805 | 2337 |
| 35 | 70401075D1 | 1803 | 2319 |
| 35 | 70403544D1 | 1804 | 2198 |
| 35 | 70408402D1 | 1806 | 2338 |
| 35 | 70400531D1 | 1805 | 2337 |
| 35 | 70402219D1 | 1804 | 2291 |
| 35 | 70402089D1 | 1807 | 2338 |
| 35 | 70401825D1 | 1807 | 2338 |
| 35 | 1954602F6 | 1809 | 2353 |
| 35 | 70403675D1 | 1807 | 2337 |
| 35 | 70403886D1 | 1806 | 2338 |
| 35 | 70402238D1 | 1809 | 2338 |
| 35 | 1954602H1 | 1809 | 2131 |
| 35 | 70404096D1 | 1816 | 2338 |
| 35 | 70409147D1 | 1818 | 2338 |
| 35 | 5858068H1 | 2465 | 2736 |
| 35 | 3713316H1 | 2484 | 2679 |
| 35 | 721887H1 | 2502 | 2694 |
| 35 | g959590 | 2512 | 2820 |
| 35 | 2596901H1 | 2588 | 2671 |
| 35 | 3989566H1 | 2601 | 2867 |
| 35 | 6944124H1 | 2626 | 2802 |
| 35 | 3400559H1 | 2673 | 2919 |
| 35 | 097934H1 | 2682 | 2866 |
| 35 | 6804789J1 | 2681 | 3271 |
| 35 | 5274591H1 | 2704 | 2955 |
| 35 | 5760373H1 | 2710 | 2981 |
| 35 | 2897406H1 | 2720 | 3008 |
| 35 | 3395763H1 | 2725 | 3003 |
| 35 | 5844479H1 | 2750 | 2990 |
| 35 | 3739173H1 | 2768 | 2968 |
| 35 | 793322H1 | 2782 | 2978 |
| 35 | g1186329 | 2822 | 3147 |
| 35 | 627833H1 | 2950 | 3186 |
| 35 | 1365903H1 | 2960 | 3198 |
| 35 | 1880754H1 | 2998 | 3282 |
| 35 | 4318269H1 | 2998 | 3264 |
| 35 | 856560R1 | 3000 | 3571 |
| 35 | 856560H1 | 3000 | 3186 |
| 35 | 4370060H1 | 3034 | 3306 |
| 35 | 5714869H1 | 3036 | 3301 |
| 35 | 5522327H1 | 3060 | 3290 |
| 35 | 2918451H1 | 3179 | 3459 |
| 35 | g2567520 | 3239 | 3726 |
| 35 | g2344517 | 3268 | 3725 |
| 35 | 6373991H1 | 3274 | 3536 |
| 35 | g5545977 | 3324 | 3722 |
| 35 | g6046858 | 3332 | 3730 |
| 35 | g1162523 | 3346 | 3716 |
| 35 | g694395 | 3474 | 3700 |
| 35 | 5574617H1 | 3485 | 3724 |
| 35 | 6594032J1 | 1 | 551 |
| 35 | 6445080H1 | 23 | 407 |
| 35 | 2719866H1 | 66 | 321 |
| 35 | g1118206 | 183 | 545 |
| 35 | 4588942H1 | 237 | 476 |
| 35 | 5621363H1 | 268 | 564 |
| 35 | 4456238R6 | 282 | 551 |
| 35 | 4456238F6 | 282 | 551 |
| 35 | 4456238H1 | 282 | 553 |
| 35 | 6471492H1 | 387 | 942 |
| 35 | 5713424H1 | 440 | 752 |
| 35 | 7213908H1 | 512 | 984 |
| 35 | 6757054J1 | 584 | 1183 |
| 35 | 5742313H1 | 626 | 906 |
| 35 | 6611978H1 | 665 | 788 |
| 35 | 3070545H1 | 686 | 962 |
| 35 | 4741922H1 | 749 | 1030 |
| 35 | 7189954H1 | 752 | 1208 |
| 35 | 6926389H1 | 1008 | 1571 |
| 35 | 4776088H1 | 1134 | 1387 |
| 35 | 2214468F6 | 1174 | 1631 |
| 35 | 2214468H1 | 1174 | 1420 |
| 35 | 2212841H1 | 1174 | 1399 |
| 35 | 6989363H1 | 1174 | 1639 |
| 35 | 7007113H1 | 1298 | 1772 |
| 35 | 7007165H1 | 1298 | 1834 |
| 35 | 7266085H1 | 1330 | 1885 |
| 35 | 6450746H1 | 1384 | 1895 |
| 35 | 2944529H1 | 1458 | 1560 |
| 35 | 7286863H1 | 1603 | 2193 |
| 35 | 6757054H1 | 1619 | 2276 |
| 35 | 7264414H1 | 1685 | 2348 |
| 35 | 6825784H1 | 1686 | 1922 |
| 35 | 6825784J1 | 1686 | 1922 |
| 35 | 7361696H1 | 1704 | 2248 |
| 35 | 4797422H1 | 1724 | 2008 |
| 35 | 70400437D1 | 1754 | 2337 |
| 35 | 70401090D1 | 1761 | 2338 |
| 35 | 70409807D1 | 1774 | 2338 |
| 35 | 70409354D1 | 1775 | 2338 |
| 35 | 70409253D1 | 1791 | 2337 |
| 35 | 70409150D1 | 1796 | 2337 |
| 35 | 70401695D1 | 1803 | 2338 |
| 35 | 70400797D1 | 1815 | 2337 |
| 35 | 70409128D1 | 1797 | 2338 |
| 35 | 70402543D1 | 1800 | 2337 |
| 35 | 70402249D1 | 1798 | 2338 |
| 35 | 70402246D1 | 1800 | 2338 |
| 35 | 70408860D1 | 1807 | 2336 |
| 35 | 3398388H1 | 1798 | 2050 |
| 35 | 70404125D1 | 1801 | 2338 |
| 35 | 70402651D1 | 1803 | 2195 |
| 35 | 70403707D1 | 1803 | 2338 |
| 35 | 70402959D1 | 1802 | 2338 |
| 35 | 70408464D1 | 1803 | 2301 |
| 35 | 70408915D1 | 1803 | 2338 |
| 35 | 70410162D1 | 1803 | 2292 |
| 35 | 70410097D1 | 1802 | 2338 |
| 35 | 70401266D1 | 1803 | 2224 |
| 35 | 70402758D1 | 1803 | 2338 |
| 35 | 70408969D1 | 1803 | 2270 |
| 35 | 70402907D1 | 1803 | 2140 |
| 35 | 70400530D1 | 1803 | 2338 |
| 35 | 70410094D1 | 1803 | 2338 |
| 35 | 70409720D1 | 1803 | 2338 |
| 35 | 70402087D1 | 1803 | 2338 |
| 35 | 70401876D1 | 1803 | 2338 |
| 35 | 70401160D1 | 1803 | 2338 |
| 35 | 70401129D1 | 1803 | 2338 |
| 35 | 70401829D1 | 1803 | 2423 |
| 35 | 70402085D1 | 1803 | 2418 |
| 35 | 70400655D1 | 1803 | 2426 |
| 35 | 70403265D1 | 1803 | 2347 |
| 35 | 70400589D1 | 1803 | 2328 |
| 35 | 70404011D1 | 1803 | 2320 |
| 35 | 70401668D1 | 1803 | 2338 |
| 35 | 70400417D1 | 1803 | 2337 |
| 35 | 70403763D1 | 1803 | 2342 |
| 36 | g1713124 | 1 | 225 |
| 36 | 542565R6 | 1 | 574 |
| 36 | 542565H1 | 1 | 270 |
| 36 | 544741H1 | 1 | 223 |
| 36 | 6785188H1 | 58 | 523 |
| 36 | 4623103H1 | 157 | 318 |
| 36 | g2704045 | 274 | 790 |
| 36 | 3033590H1 | 376 | 650 |
| 37 | g1276226 | 5072 | 5610 |
| 37 | 70882057V1 | 5076 | 5658 |
| 37 | 70617362V1 | 5079 | 5552 |
| 37 | 1981909H1 | 5086 | 5337 |
| 37 | 7170527H1 | 5086 | 5710 |
| 37 | 70616182V1 | 5094 | 5578 |
| 37 | 70618269V1 | 5139 | 5670 |
| 37 | 70621136V1 | 5131 | 5648 |
| 37 | 6894475J1 | 5133 | 5663 |
| 37 | 70619097V1 | 5143 | 5540 |
| 37 | 70616216V1 | 5159 | 5538 |
| 37 | 2485114H1 | 5160 | 5405 |
| 37 | 70620234V1 | 5178 | 5658 |
| 37 | 70879023V1 | 5182 | 5658 |
| 37 | 70618332V1 | 5184 | 5669 |
| 37 | 70620057V1 | 5141 | 5754 |
| 37 | 70619914V1 | 5206 | 5686 |
| 37 | 4261932H1 | 5214 | 5489 |
| 37 | 70650034V1 | 5223 | 5581 |
| 37 | g273566 | 5225 | 5652 |
| 37 | 70880890V1 | 5181 | 5772 |
| 37 | 70624446V1 | 5194 | 5760 |
| 37 | 5483092H1 | 5248 | 5549 |
| 37 | 5477863H1 | 5246 | 5515 |
| 37 | 70617479V1 | 5207 | 5803 |
| 37 | 5726773H1 | 5253 | 5918 |
| 37 | 70879133V1 | 5262 | 5658 |
| 37 | 70620636V1 | 5213 | 5857 |
| 37 | 70616839V1 | 5223 | 5881 |
| 37 | 70619815V1 | 5225 | 5879 |
| 37 | 70879948V1 | 5274 | 5520 |
| 37 | 70621903V1 | 5359 | 5757 |
| 37 | 70618382V1 | 5315 | 5905 |
| 37 | 1890857F6 | 5303 | 5840 |
| 37 | 1890857H1 | 5303 | 5541 |
| 37 | 70504360V1 | 5308 | 5498 |
| 37 | 3040285H1 | 5393 | 5663 |
| 37 | 70619930V1 | 5350 | 5885 |
| 37 | 70619070V1 | 5415 | 5743 |
| 37 | 70620328V1 | 5360 | 5985 |
| 37 | 70496941V1 | 5416 | 5680 |
| 37 | 70498283V1 | 5416 | 5694 |
| 37 | 70618563V1 | 5290 | 5529 |
| 37 | 3785328H1 | 5356 | 5662 |
| 37 | 70618812V1 | 5372 | 5746 |
| 37 | 70503581V1 | 5358 | 5593 |
| 37 | 70617418V1 | 5376 | 6105 |
| 37 | 5318827H1 | 5423 | 5719 |
| 37 | 4696141H1 | 5364 | 5605 |
| 37 | 3559054H1 | 5362 | 5656 |
| 37 | 862836H1 | 5364 | 5626 |
| 37 | 70621195V1 | 5405 | 5842 |
| 37 | 70620447V1 | 5403 | 6074 |
| 37 | 70616133V1 | 5404 | 5955 |
| 37 | 70880367V1 | 5417 | 5985 |
| 37 | 70615666V1 | 5408 | 5883 |
| 37 | 70615899V1 | 5411 | 6074 |
| 37 | 70615906V1 | 5420 | 5883 |
| 37 | 70620788V1 | 5422 | 6011 |
| 37 | 70619384V1 | 5423 | 6095 |
| 37 | 7352079H1 | 5485 | 5748 |
| 37 | 70618060V1 | 5452 | 5977 |
| 37 | 6247388H1 | 5422 | 6026 |
| 37 | 2528121H1 | 5422 | 5746 |
| 37 | 70620749V1 | 5443 | 6099 |
| 37 | 70498315V1 | 5453 | 5700 |
| 37 | 70620969V1 | 5454 | 5992 |
| 37 | 70618329V1 | 5465 | 6017 |
| 37 | 70620484V1 | 5472 | 6145 |
| 37 | 70616943V1 | 5480 | 5964 |
| 37 | 70616995V1 | 5486 | 5993 |
| 37 | 3865123H1 | 5486 | 5687 |
| 37 | 70618253V1 | 5507 | 5998 |
| 37 | 70619738V1 | 5509 | 6115 |
| 37 | 70619181V1 | 5504 | 6230 |
| 37 | 70620918V1 | 5521 | 6074 |
| 37 | 6262661H1 | 5507 | 5608 |
| 37 | 70888391V1 | 5563 | 6008 |
| 37 | 70617133V1 | 5533 | 6012 |
| 37 | 70879158V1 | 5595 | 5759 |
| 37 | 70619674V1 | 5548 | 6011 |
| 37 | 70620880V1 | 5570 | 6190 |
| 37 | 70619138V1 | 5593 | 6082 |
| 37 | 70620826V1 | 5579 | 5690 |
| 37 | 70618435V1 | 5616 | 6301 |
| 37 | 70619862V1 | 5613 | 6059 |
| 37 | 70620613V1 | 5615 | 6328 |
| 37 | 1516536H1 | 5596 | 5840 |
| 37 | 70619476V1 | 5631 | 6020 |
| 37 | 6935004H1 | 5623 | 6174 |
| 37 | 2640363H1 | 5629 | 5912 |
| 37 | 3604521H1 | 5635 | 5746 |
| 37 | 70619382V1 | 5713 | 6146 |
| 37 | 70818695V1 | 5659 | 6204 |
| 37 | 70620844V1 | 5664 | 6228 |
| 37 | 3760875H1 | 5641 | 5966 |
| 37 | 3943020H1 | 5645 | 5969 |
| 37 | 70620559V1 | 5673 | 6335 |
| 37 | 70616094V1 | 5672 | 6266 |
| 37 | 4201144H1 | 5656 | 5973 |
| 37 | 4201180H1 | 5656 | 5972 |
| 37 | 70881782V1 | 5733 | 6187 |
| 37 | 4902889H1 | 5668 | 5767 |
| 37 | 7218613H1 | 5666 | 6233 |
| 37 | 70620784V1 | 5742 | 6067 |
| 37 | 1478381H1 | 5702 | 5967 |
| 37 | 3472002H1 | 5757 | 6011 |
| 37 | 70882025V1 | 5814 | 6382 |
| 37 | 70620923V1 | 5854 | 6402 |
| 37 | 70616260V1 | 5818 | 6436 |
| 37 | 70880143V1 | 5867 | 6322 |
| 37 | 70616268V1 | 5819 | 6421 |
| 37 | 6307739H1 | 5814 | 6207 |
| 37 | 7215905H1 | 5846 | 6309 |
| 37 | 70615731V1 | 5872 | 6442 |
| 37 | 70618948V1 | 5844 | 6363 |
| 37 | 1483044F6 | 5842 | 6402 |
| 37 | 1483044H1 | 5842 | 6067 |
| 37 | g5689372 | 1 | 6921 |
| 37 | 6764731J1 | 177 | 746 |
| 37 | 71225106V1 | 380 | 591 |
| 37 | 71225845V1 | 648 | 1232 |
| 37 | 71224962V1 | 708 | 1268 |
| 37 | 70857749V1 | 759 | 1308 |
| 37 | 70858791V1 | 781 | 1316 |
| 37 | 70854894V1 | 817 | 1388 |
| 37 | 70857824V1 | 834 | 1385 |
| 37 | 70855972V1 | 920 | 1308 |
| 37 | 70857266V1 | 994 | 1308 |
| 37 | 6772619H1 | 1598 | 1776 |
| 37 | 7185296H1 | 1642 | 2055 |
| 37 | 7073243H1 | 3204 | 3547 |
| 37 | 6766490H1 | 3385 | 3817 |
| 37 | 7017748H1 | 3445 | 3942 |
| 37 | 6114505F6 | 3540 | 3996 |
| 37 | 6434044H1 | 3665 | 4132 |
| 37 | 4710907H1 | 3710 | 3990 |
| 37 | 7234428H1 | 3753 | 4245 |
| 37 | 2858673H1 | 3903 | 4178 |
| 37 | 4029137H1 | 4031 | 4292 |
| 37 | 5015760F6 | 4037 | 4340 |
| 37 | 5015760H1 | 4037 | 4275 |
| 37 | g846225 | 4127 | 4487 |
| 37 | 70615817V1 | 4144 | 4521 |
| 37 | 70619303V1 | 4186 | 4682 |
| 37 | 70616967V1 | 4186 | 4621 |
| 37 | 70620336V1 | 4186 | 4723 |
| 37 | 70623727V1 | 4186 | 4438 |
| 37 | 70879730V1 | 4186 | 4809 |
| 37 | 70616808V1 | 4186 | 4780 |
| 37 | 70621137V1 | 4186 | 4698 |
| 37 | 2583232F6 | 4186 | 4675 |
| 37 | 70621004V1 | 4186 | 4714 |
| 37 | 70627216V1 | 4186 | 4535 |
| 37 | 2583232H1 | 4186 | 4499 |
| 37 | g273352 | 4223 | 4554 |
| 37 | g273351 | 4223 | 4511 |
| 37 | 6772536H1 | 4229 | 4365 |
| 37 | 6792454H1 | 4247 | 4809 |
| 37 | 512449R6 | 4249 | 4619 |
| 37 | 512449H1 | 4249 | 4438 |
| 37 | 70619775V1 | 4264 | 4860 |
| 37 | 7181830H1 | 4337 | 4861 |
| 37 | 70879670V1 | 4427 | 5087 |
| 37 | 6894475H1 | 4442 | 4838 |
| 37 | 5981116H1 | 4464 | 4755 |
| 37 | 70617408V1 | 4473 | 5161 |
| 37 | 70616799V1 | 4495 | 5126 |
| 37 | 70617065V1 | 4500 | 4850 |
| 37 | 3490263H1 | 4506 | 4813 |
| 37 | g1859643 | 4567 | 4932 |
| 37 | g4087359 | 4571 | 4982 |
| 37 | g3000977 | 4574 | 4983 |
| 37 | g4989741 | 4574 | 4940 |
| 37 | g2000399 | 4580 | 4815 |
| 37 | 3256884H1 | 4642 | 4905 |
| 37 | 70613861V1 | 4650 | 5146 |
| 37 | 70615630V1 | 4675 | 5042 |
| 37 | 3409903H1 | 4666 | 4940 |
| 37 | 70620029V1 | 4686 | 5307 |
| 37 | 70616134V1 | 4698 | 5317 |
| 37 | 70615913V1 | 4709 | 5342 |
| 37 | 3472617H1 | 4738 | 4994 |
| 37 | 70616331V1 | 4758 | 5407 |
| 37 | 70879269V1 | 4760 | 5260 |
| 37 | 70616143V1 | 4803 | 5484 |
| 37 | 70619735V1 | 4837 | 5303 |
| 37 | 7359868H1 | 4826 | 5403 |
| 37 | 70618473V1 | 4836 | 5424 |
| 37 | 70617594V1 | 4839 | 5512 |
| 37 | 70615967V1 | 4854 | 5424 |
| 37 | 70616258V1 | 4872 | 5558 |
| 37 | 70618513V1 | 4872 | 5502 |
| 37 | 5636144H1 | 4877 | 5171 |
| 37 | 70616075V1 | 4881 | 5586 |
| 37 | 70619645V1 | 4886 | 5554 |
| 37 | 70621119V1 | 4890 | 5408 |
| 37 | g1484780 | 4893 | 5372 |
| 37 | 2208615F6 | 4892 | 5326 |
| 37 | 2208615H1 | 4892 | 5180 |
| 37 | g1938974 | 4900 | 5441 |
| 37 | 6534152H1 | 4900 | 5213 |
| 37 | 6416152H1 | 4900 | 5191 |
| 37 | 512449T6 | 4910 | 5294 |
| 37 | g1859530 | 4917 | 5335 |
| 37 | 7069813H1 | 4932 | 5511 |
| 37 | g1999705 | 4935 | 5233 |
| 37 | 70619554V1 | 4938 | 5557 |
| 37 | 70880135V1 | 4954 | 5576 |
| 37 | 70616830V1 | 5008 | 5634 |
| 37 | 70620540V1 | 5004 | 5493 |
| 37 | 70625529V1 | 5006 | 5596 |
| 37 | 7240188H1 | 5007 | 5436 |
| 37 | 70881149V1 | 5025 | 5658 |
| 37 | 70620009V1 | 5038 | 5484 |
| 37 | 70615572V1 | 5040 | 5582 |
| 37 | 70818665V1 | 5051 | 5653 |
| 37 | 70615553V1 | 5064 | 5522 |
| 37 | 70617505V1 | 5064 | 5424 |
| 37 | 70618274V1 | 5064 | 5658 |
| 37 | 70616969V1 | 5067 | 5474 |
| 37 | g1269975 | 5068 | 5435 |
| 37 | g5437930 | 7703 | 8018 |
| 37 | g2010328 | 7739 | 8038 |
| 37 | g851258 | 7740 | 8059 |
| 37 | g1999704 | 7753 | 8038 |
| 37 | g1968457 | 7754 | 8032 |
| 37 | 3695683H1 | 7756 | 8032 |
| 37 | 3992490H1 | 7764 | 8042 |
| 37 | g1237712 | 7776 | 8043 |
| 37 | 2568186H1 | 7779 | 8040 |
| 37 | 3090238H1 | 7786 | 8018 |
| 37 | 2755544H1 | 7791 | 8028 |
| 37 | 5304805H1 | 7791 | 7852 |
| 37 | 5186625H1 | 7798 | 8038 |
| 37 | 6399472H1 | 7835 | 8038 |
| 37 | g1484726 | 7836 | 8047 |
| 37 | 4464454H1 | 7937 | 8038 |
| 37 | 1954133H1 | 6925 | 7182 |
| 37 | g4685458 | 6927 | 7426 |
| 37 | g4072884 | 6932 | 7428 |
| 37 | g4072585 | 6932 | 7387 |
| 37 | g4301838 | 6932 | 7349 |
| 37 | g4971726 | 6932 | 7328 |
| 37 | g5706977 | 6932 | 7258 |
| 37 | g4982867 | 6932 | 7325 |
| 37 | g5444767 | 6936 | 7432 |
| 37 | 4435811H1 | 6939 | 7223 |
| 37 | g4531759 | 6943 | 7428 |
| 37 | g6040411 | 6943 | 7372 |
| 37 | g5545712 | 6943 | 7311 |
| 37 | 71287859V1 | 6955 | 7613 |
| 37 | g4991288 | 6954 | 7324 |
| 37 | g5111387 | 6957 | 7452 |
| 37 | 6207366H1 | 6957 | 7366 |
| 37 | g851259 | 6964 | 7272 |
| 37 | g5913695 | 6967 | 7431 |
| 37 | 2428623H1 | 6973 | 7220 |
| 37 | 2768162H1 | 6972 | 7242 |
| 37 | 5609278H1 | 6974 | 7263 |
| 37 | g848048 | 6975 | 7259 |
| 37 | g844877 | 6975 | 7232 |
| 37 | 5608655H1 | 6974 | 7165 |
| 37 | g848452 | 6980 | 7281 |
| 37 | g2912608 | 6988 | 7412 |
| 37 | g2789254 | 6992 | 7462 |
| 37 | g2010327 | 6997 | 7317 |
| 37 | 452519H1 | 6996 | 7196 |
| 37 | g1696891 | 7007 | 7370 |
| 37 | g1927797 | 7013 | 7427 |
| 37 | 2952058H1 | 7018 | 7316 |
| 37 | 1632083H1 | 7026 | 7252 |
| 37 | 1634329F6 | 7026 | 7583 |
| 37 | g4113770 | 7028 | 7329 |
| 37 | 1634329H1 | 7026 | 7260 |
| 37 | 6023847H1 | 7040 | 7258 |
| 37 | g1955189 | 7044 | 7363 |
| 37 | 1880801H1 | 7070 | 7228 |
| 37 | 70961362V1 | 7079 | 7662 |
| 37 | g1114689 | 7080 | 7235 |
| 37 | 2558518H2 | 7092 | 7383 |
| 37 | 3417896H2 | 7107 | 7366 |
| 37 | 4314435H1 | 7117 | 7402 |
| 37 | 2611381H1 | 7141 | 7403 |
| 37 | 2345823H1 | 7156 | 7396 |
| 37 | g848451 | 7179 | 7508 |
| 37 | g848137 | 7179 | 7509 |
| 37 | 70960910V1 | 7180 | 7787 |
| 37 | 5857373H1 | 7187 | 7467 |
| 37 | 2578818H1 | 7245 | 7494 |
| 37 | 6410914H1 | 7255 | 7606 |
| 37 | 877308H1 | 7260 | 7510 |
| 37 | 1677670H1 | 7272 | 7531 |
| 37 | 856524H1 | 7274 | 7491 |
| 37 | 5756606H1 | 7270 | 7640 |
| 37 | 856524R1 | 7275 | 7708 |
| 37 | 4597131H1 | 7283 | 7569 |
| 37 | 4597139H1 | 7284 | 7566 |
| 37 | 3047862H1 | 7298 | 7628 |
| 37 | 2721155H1 | 7319 | 7585 |
| 37 | 2367879H1 | 7326 | 7575 |
| 37 | 2367940H1 | 7326 | 7565 |
| 37 | 2561929H1 | 7348 | 7640 |
| 37 | g789819 | 7372 | 7676 |
| 37 | g1893853 | 7379 | 7868 |
| 37 | 1386867H1 | 7411 | 7694 |
| 37 | 2102141H1 | 7411 | 7664 |
| 37 | 70961741V1 | 7419 | 7998 |
| 37 | 4062984H1 | 7418 | 7717 |
| 37 | 6609732T1 | 7423 | 7993 |
| 37 | 71288471V1 | 7438 | 8037 |
| 37 | 542802H1 | 7435 | 7687 |
| 37 | 3183469H1 | 7439 | 7753 |
| 37 | 2889919H1 | 7448 | 7745 |
| 37 | 71287912V1 | 7454 | 8031 |
| 37 | 542420T6 | 7463 | 8001 |
| 37 | g1696890 | 7457 | 7674 |
| 37 | 1992524H1 | 7468 | 7735 |
| 37 | 7315008H1 | 7477 | 8036 |
| 37 | 6498781H1 | 7485 | 8037 |
| 37 | 2363076H1 | 7491 | 7743 |
| 37 | 70962526V1 | 7505 | 8038 |
| 37 | 2541325T6 | 7508 | 8030 |
| 37 | 5439901H1 | 7508 | 7781 |
| 37 | 70960239V1 | 7521 | 8139 |
| 37 | 70959969V1 | 7516 | 8138 |
| 37 | 70962026V1 | 7521 | 7999 |
| 37 | g1058797 | 7531 | 7842 |
| 37 | g844876 | 7532 | 7854 |
| 37 | 70961850V1 | 7536 | 8042 |
| 37 | g6199311 | 7544 | 7773 |
| 37 | 2262402H1 | 7545 | 7800 |
| 37 | 70960408V1 | 7558 | 8043 |
| 37 | 1677670T6 | 7558 | 7995 |
| 37 | 2870359H1 | 7574 | 7879 |
| 37 | 70962112V1 | 7573 | 8038 |
| 37 | 3476271H1 | 7578 | 7871 |
| 37 | 5287659H1 | 7583 | 7851 |
| 37 | 2622502H1 | 7584 | 7868 |
| 37 | 943201R1 | 7592 | 7941 |
| 37 | 943201H1 | 7592 | 7915 |
| 37 | 943201T1 | 7592 | 7898 |
| 37 | 1376382T6 | 7598 | 7997 |
| 37 | 2317068H1 | 7600 | 7870 |
| 37 | 1242031H1 | 7601 | 7832 |
| 37 | 3992490R6 | 7603 | 8032 |
| 37 | 877515H1 | 7623 | 7717 |
| 37 | 877515T1 | 7623 | 8002 |
| 37 | 2922847H1 | 7627 | 7906 |
| 37 | 467740H1 | 7635 | 7761 |
| 37 | 1299807T6 | 7661 | 8006 |
| 37 | 3116753H1 | 7661 | 7964 |
| 37 | 1634329T6 | 7664 | 7988 |
| 37 | 1299807F6 | 7666 | 8046 |
| 37 | 1302535H1 | 7666 | 7925 |
| 37 | 4381913H1 | 7669 | 7885 |
| 37 | g1955188 | 7671 | 8028 |
| 37 | g1023550 | 7676 | 7877 |
| 37 | 3506258H1 | 7699 | 7987 |
| 37 | 3190265H1 | 7703 | 8019 |
| 37 | 3640143H1 | 5850 | 6067 |
| 37 | g1058796 | 5863 | 6279 |
| 37 | 70879938V1 | 5903 | 6331 |
| 37 | 6619878H1 | 5884 | 6248 |
| 37 | 70879734V1 | 5954 | 6378 |
| 37 | 70618709V1 | 5900 | 6607 |
| 37 | 5896140F8 | 5944 | 6198 |
| 37 | 5902302H1 | 5925 | 6216 |
| 37 | g836224 | 5932 | 6315 |
| 37 | 70618193V1 | 5971 | 6538 |
| 37 | 70620855V1 | 5963 | 6415 |
| 37 | g863194 | 5933 | 6079 |
| 37 | 6191238H1 | 5937 | 6216 |
| 37 | 70618603V1 | 5998 | 6489 |
| 37 | 4344847H1 | 5946 | 6174 |
| 37 | 5710016H1 | 5946 | 6086 |
| 37 | 5958086H1 | 5957 | 6352 |
| 37 | 1456815H1 | 5983 | 6240 |
| 37 | 70615575V1 | 6005 | 6067 |
| 37 | 5075968H1 | 6005 | 6316 |
| 37 | 3049230H1 | 6046 | 6358 |
| 37 | 5904031H1 | 6023 | 6268 |
| 37 | 5896849H1 | 6021 | 6258 |
| 37 | 5271038H1 | 6026 | 6217 |
| 37 | 7236094H1 | 6128 | 6380 |
| 37 | 3605141H1 | 6109 | 6376 |
| 37 | 70880732V1 | 6119 | 6798 |
| 37 | 70621039V1 | 6120 | 6715 |
| 37 | 70617737V1 | 6127 | 6361 |
| 37 | 70618993V1 | 6128 | 6687 |
| 37 | 4414769F7 | 6128 | 6645 |
| 37 | 70881349V1 | 6128 | 6536 |
| 37 | 7353816H1 | 6128 | 6524 |
| 37 | 70616076V1 | 6128 | 6508 |
| 37 | 70621068V1 | 6128 | 6454 |
| 37 | 1893190H1 | 6128 | 6313 |
| 37 | 4414737H1 | 6128 | 6311 |
| 37 | 70880409V1 | 6128 | 6245 |
| 37 | 5552794H1 | 6135 | 6392 |
| 37 | 6721737H1 | 6159 | 6670 |
| 37 | 1620586F6 | 6172 | 6775 |
| 37 | 1620586H1 | 6172 | 6418 |
| 37 | 6554848H1 | 6178 | 6323 |
| 37 | 2670967H1 | 6197 | 6488 |
| 37 | g1812162 | 6262 | 6573 |
| 37 | 6754444J1 | 6298 | 6912 |
| 37 | g1165739 | 6307 | 6641 |
| 37 | 6323675H1 | 6344 | 6619 |
| 37 | 70615665V1 | 6361 | 6466 |
| 37 | 70619085V1 | 6398 | 6854 |
| 37 | g6402120 | 6414 | 6886 |
| 37 | 70882125V1 | 6416 | 6912 |
| 37 | g1938861 | 6416 | 6874 |
| 37 | g3918125 | 6419 | 6903 |
| 37 | g2963681 | 6423 | 6874 |
| 37 | g5858735 | 6427 | 6921 |
| 37 | 6377385H1 | 6428 | 6703 |
| 37 | g3742521 | 6436 | 6921 |
| 37 | g3744266 | 6445 | 6921 |
| 37 | g3740229 | 6449 | 6921 |
| 37 | g5425723 | 6450 | 6921 |
| 37 | g5451756 | 6452 | 6878 |
| 37 | g2840870 | 6463 | 6874 |
| 37 | g5658296 | 6466 | 6912 |
| 37 | g6140517 | 6481 | 6920 |
| 37 | 6303334H1 | 6482 | 6791 |
| 37 | g5658227 | 6488 | 6921 |
| 37 | 70505089V1 | 6504 | 6667 |
| 37 | 542420R6 | 6508 | 6863 |
| 37 | 542420H1 | 6508 | 6762 |
| 37 | g3872787 | 6520 | 6875 |
| 37 | 1837912H1 | 6545 | 6824 |
| 37 | g3872172 | 6548 | 6903 |
| 37 | g1813182 | 6551 | 6874 |
| 37 | 7334638H1 | 6569 | 6912 |
| 37 | g5849171 | 6575 | 6874 |
| 37 | g3179214 | 6588 | 6875 |
| 37 | g5658864 | 6592 | 6921 |
| 37 | 6428653H1 | 6610 | 6912 |
| 37 | g1226529 | 6615 | 6892 |
| 37 | g5857904 | 6636 | 6891 |
| 37 | g836225 | 6686 | 6913 |
| 37 | g1231446 | 6700 | 6920 |
| 37 | 70613460V1 | 6761 | 6913 |
| 37 | 70624483V1 | 6809 | 6913 |
| 37 | 4582057H1 | 6814 | 7103 |
| 37 | 2541325F6 | 6837 | 7357 |
| 37 | 2541325H1 | 6837 | 7098 |
| 37 | g1273237 | 6847 | 7420 |
| 37 | 70962108V1 | 6907 | 7533 |
| 37 | 70961943V1 | 6917 | 7464 |
| 37 | g4268343 | 6914 | 7451 |
| 37 | g4243879 | 6914 | 7427 |
| 37 | g5435797 | 6914 | 7436 |
| 37 | g3765880 | 6914 | 7419 |
| 37 | g3920399 | 6914 | 7422 |
| 37 | g5837342 | 6914 | 7400 |
| 37 | g4438024 | 6914 | 7349 |
| 37 | g4110613 | 6914 | 7355 |
| 37 | g3149204 | 6914 | 7324 |
| 37 | g5367473 | 6914 | 7311 |
| 37 | g4891812 | 6914 | 7310 |
| 37 | g2902901 | 6914 | 7411 |
| 37 | g4269453 | 6914 | 7429 |
| 37 | g4875491 | 6914 | 7431 |
| 37 | g4267673 | 6914 | 7427 |
| 37 | g6504581 | 6915 | 7330 |
| 37 | g6504381 | 6915 | 7326 |
| 37 | g3173140 | 6914 | 7283 |
| 37 | g3058614 | 6914 | 7255 |
| 37 | g1193529 | 6913 | 7182 |
| 37 | g1893755 | 6914 | 7160 |
| 37 | g3433586 | 6915 | 7468 |
| 37 | g6138320 | 6915 | 7313 |
| 37 | 1362613F6 | 6917 | 7542 |
| 37 | 71288170V1 | 6917 | 7498 |
| 37 | 70962658V1 | 6917 | 7465 |
| 37 | 71286924V1 | 6917 | 7465 |
| 37 | 70961637V1 | 6917 | 7486 |
| 37 | 70956912V1 | 6917 | 7299 |
| 37 | 1362613H1 | 6917 | 7140 |
| 37 | g3658929 | 6918 | 7421 |
| 37 | 7109112H1 | 6923 | 7198 |
| 38 | 2788120H1 | 217 | 469 |
| 38 | 6469701H1 | 244 | 630 |
| 38 | 70319065D1 | 309 | 706 |
| 38 | 70320239D1 | 331 | 908 |
| 38 | 70320883D1 | 331 | 761 |
| 38 | 70318969D1 | 332 | 889 |
| 38 | 70318139D1 | 517 | 1043 |
| 38 | 70317613D1 | 517 | 971 |
| 38 | 70318020D1 | 517 | 970 |
| 38 | 70317863D1 | 517 | 906 |
| 38 | 70319554D1 | 517 | 906 |
| 38 | 70317969D1 | 517 | 906 |
| 38 | 70319562D1 | 517 | 857 |
| 38 | 70318409D1 | 517 | 811 |
| 38 | 70317555D1 | 517 | 751 |
| 38 | 70318235D1 | 518 | 1040 |
| 38 | 70320237D1 | 518 | 966 |
| 38 | 70318920D1 | 518 | 927 |
| 38 | 70320035D1 | 530 | 857 |
| 38 | 70318273D1 | 536 | 970 |
| 38 | 4120208H1 | 545 | 788 |
| 38 | 70320719D1 | 565 | 970 |
| 38 | 70317918D1 | 584 | 1042 |
| 38 | 70321152D1 | 584 | 1043 |
| 38 | 70320434D1 | 584 | 989 |
| 38 | 70320713D1 | 584 | 970 |
| 38 | 70320635D1 | 584 | 970 |
| 38 | 70320227D1 | 584 | 969 |
| 38 | 70317509D1 | 584 | 970 |
| 38 | 70320694D1 | 586 | 970 |
| 38 | 70320790D1 | 584 | 953 |
| 38 | 70318744D1 | 584 | 993 |
| 38 | 70318259D1 | 585 | 1042 |
| 38 | 70318517D1 | 599 | 1043 |
| 38 | 70317458D1 | 599 | 989 |
| 38 | 70318194D1 | 599 | 970 |
| 38 | 70320117D1 | 639 | 970 |
| 38 | 70319709D1 | 676 | 1100 |
| 38 | 70319736D1 | 755 | 1245 |
| 38 | 70319869D1 | 833 | 970 |
| 38 | 2797803T6 | 850 | 1390 |
| 38 | 70320906D1 | 859 | 1245 |
| 38 | 70320282D1 | 859 | 970 |
| 38 | 3873636H1 | 897 | 1195 |
| 38 | 6932926H1 | 965 | 1317 |
| 38 | 70319282D1 | 990 | 1423 |
| 38 | 7076550H1 | 998 | 1422 |
| 38 | 70319385D1 | 1017 | 1245 |
| 38 | g2329469 | 1076 | 1366 |
| 38 | 3882715H1 | 117 | 429 |
| 38 | 2797803F6 | 214 | 667 |
| 38 | 2797803H1 | 214 | 458 |
| 38 | 6990517H1 | 1 | 513 |
| 38 | 3873636T6 | 1149 | 1488 |
| 39 | 71282176V1 | 208 | 705 |
| 39 | 70947005V1 | 228 | 721 |
| 39 | 70948844V1 | 229 | 721 |
| 39 | 70942023V1 | 240 | 925 |
| 39 | 70947964V1 | 287 | 743 |
| 39 | 5509756F6 | 208 | 693 |
| 39 | 6881073H1 | 294 | 841 |
| 39 | 70944164V1 | 208 | 694 |
| 39 | 5511496R6 | 301 | 739 |
| 39 | 3786301H1 | 329 | 524 |
| 39 | 71281455V1 | 353 | 832 |
| 39 | 70944870V1 | 208 | 677 |
| 39 | 5513185F6 | 430 | 674 |
| 39 | 5509756H1 | 208 | 469 |
| 39 | 71031868V1 | 208 | 427 |
| 39 | 70949830V1 | 223 | 721 |
| 39 | 5513185H1 | 430 | 650 |
| 39 | 5514146R6 | 461 | 721 |
| 39 | 71031732V1 | 528 | 1128 |
| 39 | 70941524V1 | 571 | 1129 |
| 39 | 71281828V1 | 578 | 1150 |
| 39 | 71281414V1 | 587 | 1150 |
| 39 | 70944681V1 | 626 | 1129 |
| 39 | 70947006V1 | 174 | 721 |
| 39 | 70947327V1 | 182 | 721 |
| 39 | 70950064V1 | 1 | 551 |
| 39 | 5514146F6 | 1 | 450 |
| 39 | 5514146H1 | 1 | 253 |
| 39 | 70941486V1 | 208 | 801 |
| 39 | 70948652V1 | 1 | 500 |
| 39 | 70941948V1 | 208 | 791 |
| 39 | 70941325V1 | 208 | 779 |
| 39 | 70944422V1 | 208 | 733 |
| 39 | 70948684V1 | 64 | 639 |
| 39 | 71281664V1 | 208 | 710 |
| 39 | 70948520V1 | 118 | 721 |
| 39 | 70949432V1 | 143 | 721 |
| 39 | 70950259V1 | 167 | 721 |
| 39 | 71281141V1 | 208 | 711 |
| 39 | 70944537V1 | 688 | 1129 |
| 39 | 70941941V1 | 694 | 1129 |
| 39 | 70941413V1 | 628 | 1129 |
| 39 | 70942815V1 | 642 | 899 |
| 39 | 6770687J1 | 726 | 1309 |
| 39 | 4491504H1 | 941 | 1521 |
| 39 | 70944142V1 | 644 | 1129 |
| 39 | 2286793R6 | 979 | 1416 |
| 39 | 2286793H1 | 979 | 1221 |
| 39 | 70944214V1 | 644 | 1129 |
| 39 | 4319902H1 | 663 | 959 |
| 39 | 3786595H1 | 1024 | 1317 |
| 39 | 4319902T6 | 1063 | 1547 |
| 39 | 4319902F6 | 663 | 1057 |
| 39 | 5509756R6 | 675 | 1128 |
| 39 | 70944571V1 | 678 | 1129 |
| 39 | 71282102V1 | 677 | 1129 |
| 39 | 70400930D1 | 1073 | 1582 |
| 39 | 70400517D1 | 1073 | 1521 |
| 39 | 70403161D1 | 1073 | 1352 |
| 39 | 5513185R6 | 1077 | 1462 |
| 39 | 70403184D1 | 1092 | 1590 |
| 39 | 70943645V1 | 683 | 1129 |
| 39 | 70402851D1 | 1092 | 1582 |
| 39 | 70408976D1 | 1092 | 1586 |
| 39 | 70409982D1 | 1092 | 1586 |
| 39 | 70400662D1 | 1092 | 1582 |
| 39 | 70403896D1 | 1093 | 1593 |
| 39 | 2286793T6 | 1106 | 1546 |
| 39 | 3789471H1 | 1130 | 1318 |
| 39 | 6517607H1 | 1193 | 1586 |
| 39 | 7111257H1 | 1234 | 1565 |
| 39 | 70402275D1 | 1353 | 1582 |
| 39 | 70401863D1 | 1377 | 1761 |
| 39 | 532083R6 | 1378 | 1587 |
| 39 | 532083H1 | 1378 | 1594 |
| 39 | 70410195D1 | 1483 | 1542 |
| 40 | 5640011H1 | 1 | 251 |
| 40 | 5152317H1 | 11 | 258 |
| 40 | 6731218H1 | 30 | 603 |
| 40 | 6948820H1 | 280 | 873 |
| 40 | 2421526H1 | 530 | 761 |
| 40 | 6073677H1 | 552 | 616 |
| 40 | 3738019F6 | 561 | 931 |
| 40 | 3738019H1 | 561 | 848 |
| 40 | 4818484H1 | 684 | 950 |
| 40 | 2844496F6 | 690 | 1186 |
| 40 | g5365267 | 722 | 1017 |
| 40 | 5913982H1 | 781 | 1038 |
| 40 | 626987R6 | 856 | 1174 |
| 40 | 626987T6 | 856 | 1137 |
| 40 | 626987H1 | 856 | 1127 |
| 40 | g1229837 | 888 | 991 |
| 40 | g1757731 | 912 | 1213 |
| 40 | 2844496H1 | 956 | 1186 |
| 40 | 1787217H1 | 976 | 1219 |
| 40 | 3024706H1 | 992 | 1272 |
| 40 | 6833501H1 | 1019 | 1579 |
| 40 | 6495087H1 | 1099 | 1672 |
| 40 | 3759406F6 | 1207 | 1605 |
| 40 | 6129674H1 | 1218 | 1777 |
| 40 | 3759406H1 | 1296 | 1605 |
| 40 | g1329304 | 1415 | 1897 |
| 40 | 3529782H1 | 1548 | 1834 |
| 40 | g5589620 | 1561 | 1983 |
| 40 | g4391274 | 1604 | 1981 |
| 40 | g1327148 | 1607 | 1980 |
| 40 | g1202759 | 1697 | 2000 |
| 40 | 5664383H1 | 1797 | 2028 |
| 40 | 3738019T6 | 1943 | 2138 |
| 41 | g6143336 | 1 | 144 |
| 41 | g2879555 | 1 | 209 |
| 41 | 963305H1 | 1 | 293 |
| 41 | 4663761H1 | 10 | 156 |
| 41 | 3081565H1 | 12 | 110 |
| 41 | 3581329H1 | 32 | 334 |
| 41 | 3581329F6 | 32 | 521 |
| 41 | g1958399 | 51 | 636 |
| 41 | g2537611 | 149 | 655 |
| 41 | g2537515 | 149 | 624 |
| 41 | 5909807H1 | 278 | 437 |
| 41 | 4741915H1 | 587 | 870 |
| 41 | 4741915F6 | 587 | 999 |
| 41 | g711286 | 905 | 1109 |
| 41 | g762724 | 905 | 1183 |
| 41 | g704731 | 905 | 1156 |
| 41 | 7210740H1 | 985 | 1345 |
| 42 | g4186965 | 4402 | 4699 |
| 42 | g749963 | 4403 | 4665 |
| 42 | 3735811H1 | 4400 | 4570 |
| 42 | g817541 | 4407 | 4703 |
| 42 | 5470839H1 | 4408 | 4627 |
| 42 | g5857465 | 4409 | 4690 |
| 42 | g4089465 | 4416 | 4698 |
| 42 | g5673112 | 4434 | 4698 |
| 42 | g832504 | 4432 | 4707 |
| 42 | 239758H1 | 4434 | 4672 |
| 42 | 239683H1 | 4434 | 4675 |
| 42 | g5630172 | 4436 | 4696 |
| 42 | g5847690 | 4442 | 4696 |
| 42 | g3144984 | 4443 | 4665 |
| 42 | 1343411H1 | 4452 | 4676 |
| 42 | g2569470 | 4474 | 4695 |
| 42 | g4989544 | 4479 | 4696 |
| 42 | g560358 | 4490 | 4690 |
| 42 | g894410 | 4511 | 4656 |
| 42 | g2732080 | 4516 | 4696 |
| 42 | 571723H1 | 4519 | 4690 |
| 42 | g858663 | 4524 | 4661 |
| 42 | 3328937H1 | 4533 | 4695 |
| 42 | g3418413 | 4586 | 4690 |
| 42 | g5662220 | 4602 | 4690 |
| 42 | 2741443H1 | 4643 | 4700 |
| 42 | 6620633H1 | 3565 | 3910 |
| 42 | 5017862H1 | 3568 | 3888 |
| 42 | 6436182H1 | 3575 | 4177 |
| 42 | 2991108H1 | 3568 | 3895 |
| 42 | 70593249V1 | 3604 | 4184 |
| 42 | 4949408H1 | 3573 | 3885 |
| 42 | 1582264H1 | 3573 | 3758 |
| 42 | g2011219 | 3574 | 3973 |
| 42 | 1694330F6 | 3575 | 4133 |
| 42 | 1694330H1 | 3575 | 3805 |
| 42 | 70596251V1 | 3605 | 4113 |
| 42 | 70592924V1 | 3600 | 4113 |
| 42 | 6040536H1 | 3651 | 4116 |
| 42 | 4729196H1 | 3661 | 3924 |
| 42 | 008776H1 | 3679 | 3953 |
| 42 | 3712443H1 | 3682 | 3965 |
| 42 | 70595569V1 | 3693 | 4227 |
| 42 | 3893442H1 | 3703 | 3987 |
| 42 | 70594859V1 | 3705 | 4113 |
| 42 | 2674702H1 | 3707 | 3953 |
| 42 | g843423 | 3723 | 3961 |
| 42 | 4166512H1 | 3729 | 4032 |
| 42 | 4490386H1 | 3768 | 4346 |
| 42 | 439941R6 | 3800 | 4179 |
| 42 | 447820H1 | 3801 | 4033 |
| 42 | 70592549V1 | 3809 | 4113 |
| 42 | 6045881H1 | 3811 | 4361 |
| 42 | 2331457H1 | 3811 | 4063 |
| 42 | g1745095 | 3819 | 4246 |
| 42 | g894451 | 3824 | 4209 |
| 42 | g750070 | 3824 | 4064 |
| 42 | g2565679 | 3834 | 4200 |
| 42 | 3359221H1 | 3855 | 4137 |
| 42 | g2565784 | 3861 | 4200 |
| 42 | 5283035H1 | 3883 | 4166 |
| 42 | 6879635H1 | 3902 | 4480 |
| 42 | 6045881J1 | 3906 | 4391 |
| 42 | g1165520 | 3908 | 4221 |
| 42 | g2537466 | 3908 | 4007 |
| 42 | 987342R1 | 3909 | 4502 |
| 42 | 987342H1 | 3909 | 4235 |
| 42 | 3414596H1 | 3918 | 4171 |
| 42 | 1968627H1 | 3944 | 4210 |
| 42 | 2079587H1 | 3963 | 4238 |
| 42 | 1942952R6 | 3975 | 4493 |
| 42 | 1942952H1 | 3975 | 4279 |
| 42 | 4266394H1 | 3977 | 4042 |
| 42 | 4547820H1 | 3985 | 4270 |
| 42 | 5566036H1 | 3987 | 4274 |
| 42 | 1495578H1 | 3993 | 4245 |
| 42 | 2290489H1 | 4000 | 4265 |
| 42 | 70636253V1 | 4001 | 4113 |
| 42 | 4837424H1 | 4018 | 4326 |
| 42 | 1942952T6 | 4024 | 4650 |
| 42 | 2415411H1 | 4047 | 4237 |
| 42 | 4597196H1 | 4052 | 4319 |
| 42 | 038747H1 | 4055 | 4308 |
| 42 | 416329H1 | 4080 | 4326 |
| 42 | 411642H1 | 4080 | 4297 |
| 42 | 413609H1 | 4080 | 4303 |
| 42 | g858610 | 4095 | 4388 |
| 42 | 6168330H1 | 4105 | 4470 |
| 42 | g1577324 | 4113 | 4324 |
| 42 | 4862551H1 | 4128 | 4322 |
| 42 | g844960 | 4134 | 4537 |
| 42 | g848470 | 4135 | 4445 |
| 42 | 1216752H1 | 4142 | 4377 |
| 42 | 2603819H1 | 4157 | 4401 |
| 42 | 2544424H1 | 4158 | 4402 |
| 42 | 2754442H1 | 4171 | 4452 |
| 42 | 917070T1 | 4185 | 4683 |
| 42 | 917070H1 | 4185 | 4357 |
| 42 | 5897064H1 | 4192 | 4506 |
| 42 | 5902845H1 | 4192 | 4491 |
| 42 | 5896842H1 | 4192 | 4449 |
| 42 | g2713617 | 4193 | 4682 |
| 42 | 1630325H1 | 4197 | 4431 |
| 42 | 1630138H1 | 4197 | 4420 |
| 42 | 4229783H1 | 4203 | 4496 |
| 42 | g4136910 | 4219 | 4695 |
| 42 | 2292673H1 | 4219 | 4483 |
| 42 | g4269943 | 4221 | 4690 |
| 42 | g4739630 | 4223 | 4695 |
| 42 | g4736707 | 4226 | 4698 |
| 42 | 2626005H1 | 4229 | 4505 |
| 42 | 2625829H1 | 4229 | 4468 |
| 42 | g1481868 | 4234 | 4694 |
| 42 | 5017059H1 | 4234 | 4484 |
| 42 | g3281238 | 4236 | 4692 |
| 42 | 4505368H1 | 4236 | 4512 |
| 42 | g3144087 | 4242 | 4695 |
| 42 | g5858526 | 4245 | 4695 |
| 42 | g2713666 | 4259 | 4696 |
| 42 | g3421998 | 4261 | 4699 |
| 42 | 6396625H1 | 4265 | 4510 |
| 42 | g4333743 | 4268 | 4690 |
| 42 | g5527375 | 4277 | 4695 |
| 42 | g4329772 | 4283 | 4698 |
| 42 | g1891693 | 4284 | 4696 |
| 42 | g3741927 | 4287 | 4699 |
| 42 | g4187402 | 4288 | 4694 |
| 42 | g1744997 | 4288 | 4689 |
| 42 | g917141 | 4291 | 4673 |
| 42 | g3244774 | 4292 | 4695 |
| 42 | g3648187 | 4292 | 4601 |
| 42 | g3839663 | 4294 | 4698 |
| 42 | g2106394 | 4294 | 4690 |
| 42 | g4270115 | 4296 | 4690 |
| 42 | g4089300 | 4300 | 4696 |
| 42 | g4988925 | 4303 | 4672 |
| 42 | g6027944 | 4304 | 4690 |
| 42 | g3751446 | 4306 | 4695 |
| 42 | g3163418 | 4309 | 4690 |
| 42 | g3869704 | 4315 | 4695 |
| 42 | g3423446 | 4316 | 4690 |
| 42 | g1486975 | 4319 | 4697 |
| 42 | g892196 | 4324 | 4703 |
| 42 | g3279131 | 4329 | 4701 |
| 42 | g2358649 | 4331 | 4696 |
| 42 | g843378 | 4349 | 4695 |
| 42 | 3622954H1 | 4349 | 4654 |
| 42 | 6839954H1 | 4349 | 4707 |
| 42 | g2140547 | 4355 | 4698 |
| 42 | g678534 | 4357 | 4690 |
| 42 | 2319411H1 | 4376 | 4640 |
| 42 | 6772602H1 | 1 | 519 |
| 42 | 6772602J1 | 8 | 525 |
| 42 | 6880022H2 | 89 | 573 |
| 42 | g1957521 | 270 | 750 |
| 42 | 6930168H1 | 405 | 575 |
| 42 | 3531464H1 | 629 | 926 |
| 42 | 1723245F6 | 735 | 1178 |
| 42 | 1723245H1 | 735 | 947 |
| 42 | 7172590H1 | 768 | 1332 |
| 42 | 6942842H1 | 786 | 1130 |
| 42 | 6828957J1 | 844 | 1493 |
| 42 | 6880022J1 | 878 | 1539 |
| 42 | 1866114H1 | 917 | 1169 |
| 42 | 7098620H1 | 966 | 1536 |
| 42 | g3758210 | 1033 | 1436 |
| 42 | 4895760H1 | 1062 | 1163 |
| 42 | 1655312F6 | 1125 | 1572 |
| 42 | 1655312H1 | 1125 | 1359 |
| 42 | 6308462H1 | 1153 | 1635 |
| 42 | 6313582H1 | 1154 | 1238 |
| 42 | 4192005H1 | 1159 | 1335 |
| 42 | 1928561H1 | 1195 | 1462 |
| 42 | 6828957H1 | 1307 | 1778 |
| 42 | 6595762H2 | 1350 | 1934 |
| 42 | 5425831H1 | 1374 | 1520 |
| 42 | 6595762J1 | 1379 | 1955 |
| 42 | 3454330H1 | 1389 | 1640 |
| 42 | 2210403H1 | 1391 | 1657 |
| 42 | 2210296H1 | 1391 | 1628 |
| 42 | 2210403F6 | 1391 | 1868 |
| 42 | 4031020H1 | 1497 | 1753 |
| 42 | g2112520 | 1547 | 2048 |
| 42 | 1655312T6 | 1596 | 2108 |
| 42 | 3174512H1 | 1612 | 1856 |
| 42 | 1723245T6 | 1636 | 2109 |
| 42 | 2210403T6 | 1636 | 2109 |
| 42 | g5393042 | 1674 | 2150 |
| 42 | 3413243H1 | 1686 | 1938 |
| 42 | g4174736 | 1701 | 2153 |
| 42 | 6338656H1 | 1709 | 2147 |
| 42 | 2406763H1 | 1745 | 1982 |
| 42 | g5639058 | 1815 | 2150 |
| 42 | 3243872F6 | 1850 | 2227 |
| 42 | 3243872H1 | 1850 | 2046 |
| 42 | 7076585H1 | 1955 | 2471 |
| 42 | 3899958H1 | 1959 | 2225 |
| 42 | 5902403H1 | 1960 | 2272 |
| 42 | 5902568H1 | 1960 | 2263 |
| 42 | 7013892H1 | 2058 | 2334 |
| 42 | 3596766H1 | 2065 | 2361 |
| 42 | 3596766F6 | 2065 | 2323 |
| 42 | 961692H1 | 2131 | 2331 |
| 42 | g2884006 | 2191 | 2658 |
| 42 | 6419411H1 | 2203 | 2420 |
| 42 | 6483672H1 | 2218 | 2734 |
| 42 | 6064745H1 | 2218 | 2518 |
| 42 | 5111344H1 | 2218 | 2483 |
| 42 | 5199520H1 | 2218 | 2466 |
| 42 | 6268649H1 | 2232 | 2513 |
| 42 | 4030823H1 | 2231 | 2492 |
| 42 | 2531619H1 | 2243 | 2492 |
| 42 | 2493928H1 | 2244 | 2566 |
| 42 | 1835162T6 | 2247 | 2699 |
| 42 | 1835162R6 | 2247 | 2638 |
| 42 | 1835162H1 | 2247 | 2491 |
| 42 | g1986374 | 2255 | 2573 |
| 42 | 6914575J1 | 2280 | 2839 |
| 42 | g3052903 | 2356 | 2756 |
| 42 | g3099254 | 2357 | 2756 |
| 42 | g5837122 | 2360 | 2749 |
| 42 | 6991132H1 | 2405 | 2945 |
| 42 | 1295850H1 | 2523 | 2759 |
| 42 | g5811286 | 2592 | 2753 |
| 42 | 7178785H1 | 2599 | 2951 |
| 42 | g892202 | 2629 | 3026 |
| 42 | 5405377H1 | 2641 | 2861 |
| 42 | 3798053H1 | 2667 | 2900 |
| 42 | 3243517H1 | 2670 | 2924 |
| 42 | 1871338F6 | 2714 | 3141 |
| 42 | 1871338H1 | 2714 | 2830 |
| 42 | g2030276 | 2756 | 3095 |
| 42 | g4989487 | 2757 | 3221 |
| 42 | g831781 | 2776 | 3012 |
| 42 | 816801H1 | 2796 | 3046 |
| 42 | 100914H1 | 2977 | 3152 |
| 42 | g3174505 | 3012 | 3221 |
| 42 | 6879635J1 | 3027 | 3604 |
| 42 | g880203 | 3043 | 3367 |
| 42 | g706026 | 3042 | 3351 |
| 42 | g3360412 | 3078 | 4696 |
| 42 | g770838 | 3097 | 3247 |
| 42 | 6914575H1 | 3109 | 3678 |
| 42 | g697357 | 3109 | 3483 |
| 42 | g1891750 | 3173 | 3577 |
| 42 | g1481321 | 3183 | 3582 |
| 42 | 3212095H1 | 3195 | 3473 |
| 42 | g2046501 | 3221 | 3602 |
| 42 | 665620H1 | 3231 | 3408 |
| 42 | 3214669H1 | 3234 | 3491 |
| 42 | 6589976H1 | 3241 | 3736 |
| 42 | 7033558H1 | 3248 | 3852 |
| 42 | 5544513H1 | 3249 | 3452 |
| 42 | 2851066H1 | 3257 | 3527 |
| 42 | 5659979H1 | 3285 | 3558 |
| 42 | 5680812H1 | 3305 | 3517 |
| 42 | 70591287V1 | 3310 | 3916 |
| 42 | 1599669F6 | 3328 | 3855 |
| 42 | 1599669H1 | 3328 | 3556 |
| 42 | 3944108H1 | 3328 | 3452 |
| 42 | 473931H1 | 3348 | 3586 |
| 42 | 2260945H1 | 3350 | 3614 |
| 42 | 6438815H1 | 3377 | 3988 |
| 42 | 1337442H1 | 3388 | 3651 |
| 42 | 1468618H1 | 3392 | 3519 |
| 42 | 4340801H1 | 3406 | 3589 |
| 42 | 6952811H1 | 3419 | 4094 |
| 42 | 70591139V1 | 3437 | 4158 |
| 42 | 6737173H1 | 3473 | 4057 |
| 42 | 2408518H1 | 3482 | 3732 |
| 42 | 5101625H1 | 3516 | 3651 |
| 42 | 3555394H1 | 3536 | 3863 |
| 42 | 2401170H1 | 3545 | 3820 |
| 42 | 5101725F9 | 3545 | 3607 |
| 42 | g1486974 | 3552 | 3873 |
| 43 | 4762482F8 | 1 | 578 |
| 43 | 4762482F9 | 1 | 675 |
| 43 | 70300728D1 | 225 | 828 |
| 43 | 70300734D1 | 251 | 716 |
| 43 | 70300231D1 | 257 | 795 |
| 43 | 70300221D1 | 261 | 801 |
| 43 | 70300270D1 | 261 | 716 |
| 43 | 4767039H1 | 292 | 569 |
| 43 | 4219425H1 | 295 | 598 |
| 43 | 70300328D1 | 297 | 876 |
| 43 | 1892972H1 | 310 | 579 |
| 43 | g1201811 | 320 | 676 |
| 43 | 7057207H1 | 329 | 967 |
| 43 | 7058929H1 | 329 | 919 |
| 43 | 3979505H1 | 336 | 624 |
| 43 | 3031026H1 | 339 | 654 |
| 43 | 2926473H1 | 346 | 611 |
| 43 | 1866123H1 | 360 | 607 |
| 43 | 5005344H1 | 389 | 662 |
| 43 | 1851313T6 | 390 | 928 |
| 43 | 3167557H1 | 390 | 693 |
| 43 | 735311H1 | 389 | 615 |
| 43 | g943247 | 408 | 585 |
| 43 | g1062500 | 409 | 716 |
| 43 | 70300862D1 | 410 | 722 |
| 43 | 2184742H1 | 413 | 706 |
| 43 | 728410R6 | 434 | 773 |
| 43 | 728410H1 | 434 | 686 |
| 43 | g1664931 | 443 | 828 |
| 43 | 2368303H2 | 442 | 694 |
| 43 | 699622H1 | 447 | 647 |
| 43 | 6774559H1 | 447 | 966 |
| 43 | 4976546H1 | 448 | 662 |
| 43 | 7353452H1 | 473 | 998 |
| 43 | g4084403 | 481 | 840 |
| 43 | 1715928H1 | 500 | 747 |
| 43 | 1300651H1 | 501 | 739 |
| 43 | 201520H1 | 504 | 832 |
| 43 | 4987575H1 | 503 | 758 |
| 43 | 202340H1 | 505 | 905 |
| 43 | 1308775H1 | 517 | 785 |
| 43 | 4128110H1 | 519 | 708 |
| 43 | 871027H1 | 527 | 769 |
| 43 | g1300912 | 530 | 771 |
| 43 | 1657105H1 | 529 | 711 |
| 43 | 1254981H1 | 530 | 775 |
| 43 | 1255176H1 | 530 | 762 |
| 43 | 1855454H1 | 536 | 795 |
| 43 | 6774559J1 | 539 | 1216 |
| 43 | g6132872 | 538 | 971 |
| 43 | 1341435H1 | 545 | 792 |
| 43 | 957179H1 | 585 | 868 |
| 43 | 2137224H1 | 586 | 867 |
| 43 | 2078670H1 | 600 | 863 |
| 43 | 6051601J1 | 600 | 854 |
| 43 | 6051601H1 | 600 | 854 |
| 43 | 3084378H1 | 601 | 830 |
| 43 | 6102879H1 | 600 | 798 |
| 43 | 123181H1 | 602 | 823 |
| 43 | g2038744 | 609 | 848 |
| 43 | 5183851H1 | 610 | 867 |
| 43 | 6199560H1 | 623 | 886 |
| 43 | 2299212H2 | 638 | 922 |
| 43 | 6755996H1 | 674 | 1299 |
| 43 | g5363964 | 677 | 970 |
| 43 | 109199H1 | 681 | 860 |
| 43 | g990884 | 686 | 970 |
| 43 | g3161907 | 693 | 1069 |
| 43 | g6476617 | 699 | 969 |
| 43 | 1957314H1 | 740 | 829 |
| 43 | g3004317 | 743 | 968 |
| 43 | 1661047H1 | 746 | 993 |
| 43 | 1660543H1 | 746 | 988 |
| 43 | 4972478H1 | 745 | 951 |
| 43 | g4569699 | 770 | 969 |
| 43 | g3861537 | 770 | 863 |
| 43 | g3846933 | 773 | 944 |
| 43 | 4399877H1 | 784 | 904 |
| 43 | g6041377 | 785 | 927 |
| 43 | g4599313 | 793 | 942 |
| 43 | g3848897 | 793 | 896 |
| 43 | g3848961 | 793 | 885 |
| 44 | 4212211H1 | 3429 | 3720 |
| 44 | 610669H1 | 3437 | 3709 |
| 44 | 7102970H1 | 3515 | 4027 |
| 44 | 2885307H1 | 3534 | 3793 |
| 44 | 3714143H1 | 3548 | 3827 |
| 44 | 70742997V1 | 3622 | 4221 |
| 44 | 4072957H1 | 3629 | 3729 |
| 44 | 2693562T6 | 3659 | 4246 |
| 44 | 70382494D1 | 3665 | 3772 |
| 44 | 70387993D1 | 3665 | 3825 |
| 44 | 2361176H1 | 3692 | 3955 |
| 44 | 4531511H1 | 3738 | 3987 |
| 44 | 4582451T9 | 3739 | 4167 |
| 44 | 4321119H1 | 3740 | 4001 |
| 44 | 2159154H1 | 3752 | 4023 |
| 44 | 5155185H1 | 3754 | 3999 |
| 44 | 70381521D1 | 3765 | 3826 |
| 44 | 2854981H1 | 3769 | 4031 |
| 44 | 70378943D1 | 3773 | 3826 |
| 44 | 5806161H1 | 3794 | 4073 |
| 44 | g4243139 | 3812 | 4278 |
| 44 | 1880015H1 | 3830 | 4095 |
| 44 | g4598394 | 3847 | 4270 |
| 44 | g6439575 | 3851 | 4269 |
| 44 | 3021981H1 | 3862 | 4151 |
| 44 | g6476034 | 3867 | 4269 |
| 44 | g3086570 | 3870 | 4268 |
| 44 | g3416423 | 3873 | 4269 |
| 44 | 6733021H1 | 3879 | 4269 |
| 44 | g1695458 | 3881 | 4269 |
| 44 | 5292403H2 | 3899 | 4146 |
| 44 | 5341347H1 | 3905 | 4148 |
| 44 | 1712455H1 | 3948 | 4152 |
| 44 | g5513470 | 3963 | 4374 |
| 44 | g3446060 | 3964 | 4374 |
| 44 | g4568404 | 3968 | 4269 |
| 44 | g4876405 | 4001 | 4385 |
| 44 | g4435068 | 4003 | 4374 |
| 44 | 2270005H1 | 4019 | 4269 |
| 44 | g4301776 | 4027 | 4374 |
| 44 | g2240627 | 4054 | 4374 |
| 44 | 2410679H1 | 4059 | 4276 |
| 44 | g1231923 | 4061 | 4272 |
| 44 | 211813H1 | 4104 | 4184 |
| 44 | g1784545 | 4139 | 4276 |
| 44 | 6180170H1 | 4157 | 4273 |
| 44 | 2587751H1 | 4168 | 4275 |
| 44 | g819902 | 4171 | 4386 |
| 44 | 1530352H1 | 4173 | 4375 |
| 44 | 2555081H1 | 4180 | 4269 |
| 44 | 3077509H1 | 4191 | 4269 |
| 44 | g5635105 | 4214 | 4374 |
| 44 | 5718794H1 | 4217 | 4285 |
| 44 | 4833360H1 | 4231 | 4430 |
| 44 | 995064H1 | 1 | 306 |
| 44 | 7183605H1 | 164 | 706 |
| 44 | 1945549H1 | 288 | 517 |
| 44 | 7383801H1 | 294 | 751 |
| 44 | 6549205H1 | 308 | 754 |
| 44 | 2832846T6 | 341 | 754 |
| 44 | 6819064J1 | 435 | 810 |
| 44 | 3280967H1 | 504 | 754 |
| 44 | 2397313H1 | 607 | 834 |
| 44 | 6830025J1 | 746 | 1064 |
| 44 | 6053477H1 | 762 | 1329 |
| 44 | 4348719H1 | 775 | 1035 |
| 44 | g3163362 | 788 | 1129 |
| 44 | 4248617H1 | 798 | 1030 |
| 44 | 6940669H1 | 802 | 1101 |
| 44 | 6886338J1 | 801 | 1162 |
| 44 | 5623119H1 | 819 | 987 |
| 44 | 3597238H1 | 820 | 1104 |
| 44 | 914670H1 | 825 | 1091 |
| 44 | 915108H1 | 825 | 1131 |
| 44 | 5339305H1 | 846 | 919 |
| 44 | 5701708H1 | 880 | 1160 |
| 44 | g1985651 | 889 | 1175 |
| 44 | 679430H1 | 900 | 1165 |
| 44 | 3767690H1 | 961 | 1253 |
| 44 | 429994H1 | 993 | 1269 |
| 44 | 4425802H1 | 1013 | 1275 |
| 44 | 3768769H1 | 1017 | 1287 |
| 44 | 2358712H1 | 1069 | 1330 |
| 44 | 2359316H1 | 1069 | 1331 |
| 44 | 2835111H1 | 1097 | 1370 |
| 44 | 5875970H1 | 1119 | 1401 |
| 44 | 5505305H1 | 1125 | 1342 |
| 44 | 3000263H1 | 1149 | 1444 |
| 44 | 6782153H1 | 1160 | 1671 |
| 44 | 6053477J1 | 1203 | 1692 |
| 44 | 3157719H1 | 1215 | 1498 |
| 44 | 3765631H1 | 1235 | 1519 |
| 44 | 1908860T6 | 1257 | 1675 |
| 44 | 4551786H1 | 1265 | 1463 |
| 44 | 4521416H1 | 1288 | 1531 |
| 44 | 3031440H1 | 1303 | 1442 |
| 44 | 890596H1 | 1315 | 1574 |
| 44 | 2991495H1 | 1356 | 1617 |
| 44 | 764400H1 | 1367 | 1610 |
| 44 | 4906007H2 | 1395 | 1663 |
| 44 | 7267741H1 | 1469 | 1999 |
| 44 | 6769245H1 | 1503 | 2048 |
| 44 | 997621R6 | 1575 | 1956 |
| 44 | 7328544H1 | 1580 | 2183 |
| 44 | 2832846H1 | 1580 | 1808 |
| 44 | 2860343H1 | 1580 | 1826 |
| 44 | 2830834H1 | 1580 | 1807 |
| 44 | 2860379H1 | 1580 | 1847 |
| 44 | 3374480H1 | 1588 | 1863 |
| 44 | 6623866J1 | 1592 | 2173 |
| 44 | 3077919H1 | 1614 | 1907 |
| 44 | 3739575H1 | 1614 | 1877 |
| 44 | 4959060H1 | 1628 | 1875 |
| 44 | 4739571H1 | 1632 | 1886 |
| 44 | 5076688H1 | 1633 | 1891 |
| 44 | 688318H1 | 1634 | 1905 |
| 44 | 2445716H1 | 1664 | 1914 |
| 44 | 6044677H1 | 1665 | 1972 |
| 44 | 1386547H1 | 1665 | 1891 |
| 44 | 2005094H1 | 1670 | 1929 |
| 44 | 6329762H1 | 1714 | 2197 |
| 44 | 2898215H1 | 1710 | 2021 |
| 44 | 2243695H1 | 1728 | 1968 |
| 44 | 899794H1 | 1731 | 1996 |
| 44 | 5826773H1 | 1740 | 2266 |
| 44 | 518068H1 | 1739 | 1966 |
| 44 | 7060429H1 | 1745 | 2323 |
| 44 | 6955408H1 | 1748 | 2290 |
| 44 | 6332331H1 | 1747 | 2192 |
| 44 | 6420565H1 | 1756 | 2147 |
| 44 | 70741619V1 | 3350 | 3955 |
| 44 | 6773924H1 | 1756 | 2263 |
| 44 | 3490671H1 | 1759 | 1926 |
| 44 | 5633425H1 | 1760 | 2016 |
| 44 | 2171658H1 | 1759 | 1959 |
| 44 | 4840412H1 | 1770 | 2029 |
| 44 | 5639607H1 | 1839 | 1967 |
| 44 | 2512143H1 | 1858 | 2174 |
| 44 | 3243167H1 | 1970 | 2205 |
| 44 | 2371305H1 | 1981 | 2202 |
| 44 | 71303275V1 | 2021 | 2416 |
| 44 | 71130980V1 | 2217 | 2338 |
| 44 | 7213740H1 | 2222 | 2436 |
| 44 | 6332191H1 | 2329 | 2863 |
| 44 | 4730102H1 | 2375 | 2634 |
| 44 | 1519940H1 | 2397 | 2598 |
| 44 | 1932345H1 | 2461 | 2718 |
| 44 | 4631720H1 | 2467 | 2747 |
| 44 | 5516438H1 | 2467 | 2720 |
| 44 | 4631626H1 | 2468 | 2655 |
| 44 | g772275 | 2472 | 2763 |
| 44 | 6073520H1 | 2478 | 2745 |
| 44 | 4973296H1 | 2483 | 2621 |
| 44 | 5086726H1 | 2488 | 2742 |
| 44 | 6701387H1 | 2488 | 2948 |
| 44 | 5687234H1 | 2488 | 2753 |
| 44 | g1967335 | 2488 | 3017 |
| 44 | 6073522H1 | 2490 | 2745 |
| 44 | 4793022H1 | 2502 | 2616 |
| 44 | 2228536H1 | 2505 | 2721 |
| 44 | 4590907H1 | 2507 | 2780 |
| 44 | 4548974H1 | 2527 | 2743 |
| 44 | 3464267H1 | 2598 | 2832 |
| 44 | 3492779H1 | 2605 | 2877 |
| 44 | 1435636H1 | 2620 | 2842 |
| 44 | g2023037 | 2631 | 2947 |
| 44 | 70743767V1 | 3351 | 3875 |
| 44 | 3162341H1 | 2638 | 2911 |
| 44 | 1257054H1 | 2641 | 2874 |
| 44 | 1258294H1 | 2641 | 2863 |
| 44 | 6375444H1 | 2652 | 2922 |
| 44 | 3781848H1 | 2700 | 3010 |
| 44 | 2772194H1 | 2706 | 2957 |
| 44 | 4633723H1 | 2723 | 2985 |
| 44 | 023904H1 | 2728 | 2988 |
| 44 | 2763883H1 | 2738 | 2984 |
| 44 | g3400659 | 2748 | 3194 |
| 44 | 4574816H1 | 2780 | 3041 |
| 44 | 3882424H1 | 2796 | 3066 |
| 44 | 5687093H1 | 2819 | 3075 |
| 44 | g920527 | 2832 | 3168 |
| 44 | 5281394H1 | 2844 | 3007 |
| 44 | 449764H1 | 2846 | 3059 |
| 44 | 4140731H1 | 2864 | 3170 |
| 44 | g897406 | 2874 | 3188 |
| 44 | g2254156 | 2935 | 3169 |
| 44 | g1641497 | 2981 | 3180 |
| 44 | 3223084H1 | 2993 | 3290 |
| 44 | 1801096H1 | 2999 | 3085 |
| 44 | 3766823H1 | 3028 | 3310 |
| 44 | 454112H1 | 3070 | 3289 |
| 44 | 2661320H1 | 3075 | 3277 |
| 44 | 2688727H1 | 3091 | 3328 |
| 44 | 4595028H1 | 3292 | 3552 |
| 44 | 70741735V1 | 3312 | 3923 |
| 45 | 6572570J1 | 1 | 436 |
| 45 | g4435582 | 1 | 380 |
| 45 | 4345534F6 | 21 | 269 |
| 45 | 6073452H1 | 72 | 231 |
| 45 | 4345534H1 | 105 | 269 |
| 45 | 7204038H1 | 148 | 663 |
| 45 | 6981431H1 | 294 | 825 |
| 45 | 2630960F6 | 730 | 1090 |
| 45 | 2630960H1 | 730 | 965 |
| 45 | 609082H1 | 802 | 1065 |
| 45 | g564302 | 805 | 1003 |
| 45 | g1139137 | 837 | 1181 |
| 45 | 2554584H1 | 957 | 1231 |
| 45 | 2555307H1 | 957 | 1233 |
| 45 | 6990332H1 | 984 | 1308 |
| 45 | g781616 | 1020 | 1332 |
| 45 | 5904340H1 | 1041 | 1335 |
| 45 | 5901269H1 | 1041 | 1283 |
| 45 | 5893679H1 | 1042 | 1343 |
| 45 | 5904340F6 | 1057 | 1609 |
| 45 | 6937534H1 | 1070 | 1684 |
| 45 | 6092295H1 | 1129 | 1391 |
| 45 | g1398375 | 1133 | 1551 |
| 45 | 6917411H1 | 1143 | 1733 |
| 45 | 3142603H1 | 1278 | 1440 |
| 45 | 3142603F6 | 1279 | 1701 |
| 45 | g4985474 | 1317 | 1751 |
| 45 | 5099033H1 | 1339 | 1615 |
| 45 | 4147460H1 | 1349 | 1541 |
| 45 | 3142603T6 | 1395 | 1863 |
| 45 | 3689942H1 | 1434 | 1717 |
| 45 | g4394682 | 1460 | 1909 |
| 45 | g3418148 | 1462 | 1908 |
| 45 | g1398292 | 1472 | 1885 |
| 45 | 6197152H1 | 1476 | 1923 |
| 45 | 2630960T6 | 1491 | 1867 |
| 45 | g3424422 | 1526 | 1906 |
| 45 | 5638207H1 | 1551 | 1833 |
| 45 | g827423 | 1566 | 1771 |
| 45 | 1299193F6 | 1583 | 1900 |
| 45 | 6073452T6 | 1583 | 1862 |
| 45 | 1299193H1 | 1583 | 1777 |
| 45 | 1299193T6 | 1583 | 1857 |
| 45 | 2107254H1 | 1590 | 1866 |
| 45 | g560355 | 1684 | 1900 |
| 45 | g1784513 | 1726 | 1848 |
| 45 | 2223448H1 | 1760 | 1896 |
| 45 | g6505941 | 1763 | 2406 |
| 45 | 2660260H1 | 1785 | 2032 |
| 45 | 4857157H1 | 1816 | 2022 |
| 45 | 3242659H1 | 1933 | 2154 |
| 45 | g823764 | 2079 | 2417 |
| 45 | 6572570H1 | 2278 | 2762 |
| 45 | 6924120H1 | 2500 | 3013 |
| 45 | 6168506H1 | 2644 | 2967 |
| 45 | 6987381H1 | 2657 | 3220 |
| 45 | 3508325H1 | 2828 | 3116 |
| 46 | 7061915H1 | 1 | 477 |
| 46 | g2155606 | 172 | 658 |
| 46 | g2381480 | 353 | 4979 |
| 46 | g2842283 | 794 | 1156 |
| 46 | g2034110 | 827 | 1154 |
| 46 | g1109149 | 1845 | 2143 |
| 46 | g1167026 | 1888 | 2061 |
| 46 | 5610451F6 | 2289 | 2861 |
| 46 | 5610451H1 | 2289 | 2532 |
| 46 | g1969027 | 2438 | 2801 |
| 46 | g1142377 | 2447 | 2779 |
| 46 | g1151984 | 2615 | 3003 |
| 46 | g4242953 | 2628 | 2914 |
| 46 | g1109150 | 2745 | 2961 |
| 47 | 6772168H1 | 1565 | 2062 |
| 47 | 2788543H1 | 1463 | 1697 |
| 47 | 5858122H1 | 1534 | 1797 |
| 47 | 2705868F6 | 1563 | 1929 |
| 47 | 5022301H1 | 1563 | 1833 |
| 47 | 2705868H1 | 1564 | 1835 |
| 47 | 4340882T6 | 1795 | 2060 |
| 47 | 1479486T6 | 1569 | 2058 |
| 47 | 2761062T6 | 1568 | 2057 |
| 47 | 2705868T6 | 1581 | 2056 |
| 47 | 3468165H1 | 1577 | 1844 |
| 47 | 1993688T6 | 1645 | 2057 |
| 47 | 1993688F6 | 1652 | 2097 |
| 47 | 1993688H1 | 1652 | 1899 |
| 47 | 666099H1 | 1670 | 1895 |
| 47 | g3423957 | 1725 | 2098 |
| 47 | g3241592 | 1770 | 2104 |
| 47 | 1479372H1 | 1175 | 1422 |
| 47 | 2761062H1 | 1178 | 1443 |
| 47 | 2761062R6 | 1178 | 1435 |
| 47 | 6001120H1 | 1235 | 1723 |
| 47 | 6977932H1 | 1414 | 1612 |
| 47 | 7033269H1 | 1452 | 1982 |
| 47 | 3466972H1 | 1462 | 1719 |
| 47 | 5016835H1 | 1842 | 2094 |
| 47 | 4774836H1 | 1845 | 2099 |
| 47 | 4229676H1 | 1889 | 2098 |
| 47 | 4254348H1 | 1999 | 2098 |
| 47 | 2708491F6 | 715 | 1057 |
| 47 | 6953084H1 | 1 | 555 |
| 47 | 5373405H1 | 1 | 224 |
| 47 | 5373405F8 | 21 | 542 |
| 47 | 3942701H1 | 129 | 421 |
| 47 | 4340882H1 | 331 | 526 |
| 47 | 4340882F6 | 331 | 807 |
| 47 | 3323337H1 | 389 | 656 |
| 47 | 1479486F6 | 393 | 809 |
| 47 | 1479486H1 | 393 | 628 |
| 47 | 1993010H1 | 423 | 703 |
| 47 | 3471007H1 | 621 | 893 |
| 47 | 3915086H1 | 658 | 817 |
| 47 | 5373405T6 | 1566 | 2093 |
| 47 | 3783242H1 | 1041 | 1328 |
| 47 | 2708491H1 | 716 | 1011 |
| 47 | 6307323H1 | 787 | 1123 |
| 47 | 551890H1 | 881 | 1067 |
| 47 | 4492287H1 | 882 | 1261 |
| 48 | 70572210V1 | 884 | 1469 |
| 48 | 70572495V1 | 955 | 1286 |
| 48 | 70571926V1 | 754 | 1202 |
| 48 | 70571502V1 | 745 | 1349 |
| 48 | 70569574V1 | 826 | 1486 |
| 48 | 70570142V1 | 836 | 1398 |
| 48 | 70570141V1 | 844 | 1193 |
| 48 | 70570482V1 | 849 | 1305 |
| 48 | 70569465V1 | 855 | 1274 |
| 48 | 70569269V1 | 556 | 1188 |
| 48 | 70569417V1 | 559 | 1188 |
| 48 | 70570991V1 | 584 | 1118 |
| 48 | 70569428V1 | 571 | 1259 |
| 48 | 70568632V1 | 620 | 1317 |
| 48 | 70572355V1 | 635 | 1302 |
| 48 | 70564434V1 | 639 | 1062 |
| 48 | 70571851V1 | 657 | 1264 |
| 48 | 70570748V1 | 704 | 1295 |
| 48 | 7003760H1 | 729 | 1123 |
| 48 | 70563349V1 | 947 | 1323 |
| 48 | 70570762V1 | 960 | 1527 |
| 48 | 70571845V1 | 969 | 1601 |
| 48 | 6866835H1 | 983 | 1631 |
| 48 | 70562712V1 | 984 | 1298 |
| 48 | 6157305H1 | 993 | 1269 |
| 48 | 5516852H1 | 1028 | 1312 |
| 48 | 2450339F6 | 1 | 224 |
| 48 | 2450339H1 | 1 | 135 |
| 48 | 6854122H1 | 5 | 557 |
| 48 | 7081863H1 | 20 | 596 |
| 48 | 2237351H1 | 363 | 650 |
| 48 | 70569676V1 | 378 | 981 |
| 48 | 70552498V1 | 387 | 1058 |
| 48 | 70563545V1 | 416 | 639 |
| 48 | 3460952H1 | 24 | 248 |
| 48 | 70569565V1 | 210 | 797 |
| 48 | g4703329 | 426 | 774 |
| 48 | 70570223V1 | 438 | 1185 |
| 48 | 70570920V1 | 440 | 898 |
| 48 | 70570781V1 | 440 | 897 |
| 48 | 70567983V1 | 475 | 904 |
| 48 | 70569223V1 | 248 | 856 |
| 48 | 70570943V1 | 250 | 912 |
| 48 | 70571295V1 | 259 | 821 |
| 48 | 70568330V1 | 355 | 1012 |
| 48 | 70568216V1 | 363 | 1033 |
| 48 | 2237351F6 | 363 | 725 |
| 48 | 2905811H1 | 1091 | 1408 |
| 48 | 70568512V1 | 1109 | 1378 |
| 48 | 2522478F6 | 1137 | 1449 |
| 48 | 2522478F7 | 1137 | 1471 |
| 48 | 2522478H1 | 1137 | 1402 |
| 48 | 2522389H1 | 1137 | 1396 |
| 48 | 70570776V1 | 1149 | 1704 |
| 48 | 70570912V1 | 1149 | 1797 |
| 48 | 70569442V1 | 1176 | 1474 |
| 48 | 2450339T6 | 1168 | 1666 |
| 48 | 70568046V1 | 1222 | 1524 |
| 48 | 70572193V1 | 1248 | 1704 |
| 48 | 5494475H1 | 1250 | 1538 |
| 48 | 70569585V1 | 489 | 1228 |
| 48 | 70568685V1 | 490 | 914 |
| 48 | 70568639V1 | 500 | 1181 |
| 48 | 70568617V1 | 505 | 1145 |
| 48 | 70568629V1 | 528 | 1239 |
| 49 | 7260572H1 | 1 | 542 |
| 49 | 2839721H2 | 23 | 287 |
| 49 | 2839721F6 | 77 | 411 |
| 49 | 4705119F8 | 77 | 655 |
| 49 | 4705119H1 | 77 | 277 |
| 49 | 3843861H1 | 193 | 497 |
| 49 | 4542028F8 | 230 | 721 |
| 49 | 4542028H1 | 229 | 502 |
| 49 | 7337215H1 | 396 | 581 |
| 49 | 5727315H1 | 491 | 1020 |
| 49 | 4880713H1 | 564 | 798 |
| 49 | 2843763H1 | 603 | 866 |
| 49 | 3097805H1 | 670 | 819 |
| 49 | 4631337F6 | 678 | 737 |
| 49 | 4631337H1 | 679 | 753 |
| 49 | 1971904H1 | 712 | 971 |
| 49 | 1971904F6 | 712 | 1114 |
| 49 | g3989039 | 765 | 832 |
| 49 | g4004553 | 765 | 832 |
| 49 | 732931H1 | 765 | 972 |
| 49 | 2235515T6 | 807 | 1469 |
| 49 | 1514627T6 | 814 | 1176 |
| 49 | 4080269H1 | 870 | 1146 |
| 49 | 6832335H1 | 897 | 1445 |
| 49 | 4116133H1 | 949 | 1215 |
| 49 | 2258867H1 | 950 | 1206 |
| 49 | 4492763H1 | 962 | 1540 |
| 49 | 1386487H1 | 1074 | 1292 |
| 49 | 4431852H1 | 1082 | 1334 |
| 49 | 3599043H1 | 1121 | 1403 |
| 49 | 1313052H1 | 1130 | 1364 |
| 49 | 4825915H1 | 1179 | 1465 |
| 49 | 1578741H1 | 1199 | 1411 |
| 49 | 6521182H1 | 1240 | 1806 |
| 49 | 5506584H1 | 1243 | 1449 |
| 49 | 3905468H1 | 1244 | 1490 |
| 49 | 5718792H1 | 1246 | 1761 |
| 49 | 2211706H1 | 1251 | 1517 |
| 49 | g4523198 | 1435 | 1853 |
| 49 | 3818989F6 | 1449 | 1915 |
| 49 | 3818989H1 | 1449 | 1668 |
| 49 | 6942236H1 | 1467 | 2063 |
| 49 | g2219561 | 1488 | 1811 |
| 49 | g1712378 | 1488 | 1668 |
| 49 | 1974505F6 | 1535 | 1853 |
| 49 | 1974505H1 | 1535 | 1668 |
| 49 | 6978865H1 | 1547 | 1936 |
| 49 | g1856293 | 1557 | 1943 |
| 49 | 6839538H1 | 1559 | 2161 |
| 49 | 6073945H1 | 1564 | 1765 |
| 49 | g2537789 | 1605 | 1892 |
| 49 | 3818989T6 | 1753 | 2232 |
| 49 | 4631337T6 | 1759 | 2243 |
| 49 | g4684927 | 1775 | 2271 |
| 49 | 2364406H1 | 1776 | 1931 |
| 49 | g4188351 | 1787 | 2267 |
| 49 | g3412954 | 1794 | 2267 |
| 49 | g5590415 | 1794 | 2272 |
| 49 | 1974505T6 | 1795 | 2231 |
| 49 | g3647432 | 1802 | 2269 |
| 49 | g3330983 | 1815 | 2271 |
| 49 | g4261154 | 1829 | 2269 |
| 49 | 1781695H1 | 1833 | 2127 |
| 49 | 1781695R6 | 1833 | 2266 |
| 49 | g3932044 | 1843 | 2267 |
| 49 | 4243158H1 | 1857 | 2235 |
| 49 | 1514627F6 | 1888 | 2269 |
| 49 | g3415551 | 1879 | 2269 |
| 49 | g3108736 | 1910 | 2274 |
| 49 | g1550011 | 1918 | 2269 |
| 49 | g3921822 | 1920 | 2273 |
| 49 | g4330608 | 1923 | 2269 |
| 49 | g5630278 | 1926 | 2272 |
| 49 | g2189478 | 1928 | 2260 |
| 49 | g4073608 | 1929 | 2276 |
| 49 | g4109783 | 1929 | 2271 |
| 49 | g4392501 | 1929 | 2260 |
| 49 | 1971904T6 | 1930 | 2232 |
| 49 | g1856294 | 1940 | 2269 |
| 49 | g3095415 | 1941 | 2271 |
| 49 | g5630271 | 1944 | 2272 |
| 49 | 546355H1 | 1961 | 2220 |
| 49 | g3896543 | 1970 | 2269 |
| 49 | 1781695T6 | 1983 | 2229 |
| 49 | g2219452 | 2007 | 2269 |
| 49 | g6463319 | 2010 | 2273 |
| 49 | g1722646 | 2038 | 2269 |
| 49 | g5326403 | 2077 | 2269 |
| 49 | 652012H1 | 2089 | 2352 |
| 49 | g3756725 | 2122 | 2272 |
| 49 | 1312172H1 | 2140 | 2324 |
| 49 | 1514627H1 | 2190 | 2269 |
| 50 | 2638422T6 | 1 | 140 |
| 50 | 2638422F6 | 1 | 373 |
| 50 | 6981422H1 | 7 | 572 |
| 50 | g4148173 | 53 | 490 |
| 50 | 2638422H1 | 113 | 373 |
| 50 | 6768020J1 | 195 | 727 |
| 50 | 7359784H1 | 207 | 757 |
| 50 | g6199167 | 365 | 749 |
| 50 | 5900048H1 | 409 | 699 |
| 50 | 6989532H1 | 559 | 1155 |
| 50 | 70803892V1 | 654 | 1303 |
| 50 | 70800990V1 | 720 | 1318 |
| 50 | 70803809V1 | 835 | 1513 |
| 50 | 70804202V1 | 838 | 1520 |
| 50 | 70802115V1 | 859 | 1472 |
| 50 | 6307959H1 | 872 | 1397 |
| 50 | 70804306V1 | 1070 | 1567 |
| 50 | 70803140V1 | 1088 | 1463 |
| 50 | 70804898V1 | 1133 | 1715 |
| 50 | 70800978V1 | 1132 | 1618 |
| 50 | 70804775V1 | 1147 | 1612 |
| 50 | 7056347H1 | 1181 | 1581 |
| 50 | 70801141V1 | 1186 | 1827 |
| 50 | 70803370V1 | 1405 | 1947 |
| 50 | 70799868V1 | 1417 | 1961 |
| 50 | 70804130V1 | 1516 | 2035 |
| 50 | g3162700 | 1645 | 1978 |
| 50 | 70805243V1 | 1687 | 2355 |
| 50 | 70804667V1 | 1765 | 2369 |
| 50 | 70800147V1 | 1774 | 2372 |
| 50 | 70802731V1 | 1850 | 2545 |
| 50 | 70801205V1 | 1863 | 2516 |
| 50 | 70802853V1 | 1885 | 2354 |
| 50 | 70804916V1 | 1896 | 2382 |
| 50 | 70799899V1 | 1936 | 2639 |
| 50 | 70803805V1 | 2003 | 2638 |
| 50 | 70801138V1 | 2037 | 2684 |
| 50 | 70801844V1 | 2074 | 2737 |
| 50 | 70803750V1 | 2156 | 2834 |
| 50 | 70802422V1 | 2157 | 2719 |
| 50 | 6854011H1 | 2187 | 2549 |
| 50 | 70802774V1 | 2212 | 2605 |
| 50 | 70803192V1 | 2204 | 2760 |
| 50 | 70799608V1 | 2284 | 2822 |
| 50 | 6311222H1 | 2340 | 2883 |
| 50 | 70799712V1 | 2430 | 3039 |
| 50 | 70803364V1 | 2588 | 2975 |
| 50 | 70804594V1 | 2646 | 3250 |
| 50 | 70801808V1 | 2683 | 3255 |
| 50 | 70800968V1 | 2688 | 3348 |
| 50 | 3462322F6 | 2749 | 3282 |
| 50 | 70803000V1 | 2775 | 3325 |
| 50 | 3462322H1 | 2963 | 3283 |
| 50 | g2882851 | 3179 | 3525 |
| 50 | 7067268H1 | 3180 | 3742 |
| 50 | 5119843F6 | 3186 | 3640 |
| 50 | 6984347H1 | 3214 | 3705 |
| 50 | 5119843H1 | 3375 | 3640 |
| 50 | g1874645 | 3618 | 4111 |
| 51 | 7228427H1 | 1 | 573 |
| 51 | 7091223H1 | 358 | 648 |
| 51 | 5996621H1 | 456 | 977 |
| 51 | 3784279H1 | 556 | 862 |
| 51 | 2858090F6 | 603 | 820 |
| 51 | 2858090H1 | 603 | 682 |
| 51 | 6756153H1 | 771 | 1318 |
| 51 | 5967687H1 | 773 | 1194 |
| 51 | 7101263H1 | 1029 | 1223 |
| 51 | 5972491H1 | 1076 | 1457 |
| 51 | g317931 | 1290 | 1685 |
| 51 | g317603 | 1313 | 1685 |
| 51 | 5877835F6 | 1348 | 1872 |
| 51 | 5877835H1 | 1348 | 1630 |
| 51 | 6756153J1 | 1704 | 2225 |
| 51 | 1806404F6 | 1823 | 2286 |
| 51 | 1806404H1 | 1823 | 2078 |
| 51 | 1806342H1 | 1823 | 2101 |
| 51 | 5877835T6 | 1912 | 2353 |
| 51 | 1806404T6 | 1926 | 2353 |
| 52 | 1284836F6 | 10 | 356 |
| 52 | 1284836H1 | 10 | 255 |
| 52 | 4831767H1 | 8 | 274 |
| 52 | 70249801V1 | 11 | 180 |
| 52 | 70248113V1 | 10 | 351 |
| 52 | 70252775V1 | 18 | 504 |
| 52 | 3613968H1 | 43 | 353 |
| 52 | 70255782V1 | 710 | 1161 |
| 52 | 70693658V1 | 2319 | 2959 |
| 52 | 3934733H1 | 2328 | 2628 |
| 52 | 70696721V1 | 2349 | 2664 |
| 52 | 70697189V1 | 2352 | 2883 |
| 52 | 70693987V1 | 2387 | 2733 |
| 52 | 70693793V1 | 2392 | 2906 |
| 52 | 70691748V1 | 2402 | 2965 |
| 52 | 70695598V1 | 2575 | 3130 |
| 52 | 70701933V1 | 2584 | 2746 |
| 52 | 6571494H1 | 2608 | 3105 |
| 52 | 70696700V1 | 2618 | 2974 |
| 52 | 70694905V1 | 2652 | 3207 |
| 52 | 70698828V1 | 2656 | 2912 |
| 52 | g3898245 | 2680 | 3119 |
| 52 | 3737128F6 | 2701 | 3256 |
| 52 | 3737128H1 | 2701 | 3001 |
| 52 | 70694991V1 | 2719 | 3251 |
| 52 | g2933892 | 2734 | 3208 |
| 52 | 70695524V1 | 2779 | 3272 |
| 52 | 70255499V1 | 1937 | 2215 |
| 52 | 70696984V1 | 2787 | 3244 |
| 52 | 70695420V1 | 2799 | 3015 |
| 52 | 70694656V1 | 2806 | 3295 |
| 52 | 70696762V1 | 2811 | 3295 |
| 52 | 70692751V1 | 2837 | 3295 |
| 52 | 70694835V1 | 2853 | 3313 |
| 52 | g2821799 | 1953 | 2224 |
| 52 | 70257206V1 | 1975 | 2202 |
| 52 | g3154360 | 2000 | 2215 |
| 52 | 70249502V1 | 2056 | 2215 |
| 52 | 70696280V1 | 2123 | 2801 |
| 52 | 3737128T6 | 2868 | 3297 |
| 52 | g3645644 | 2896 | 3264 |
| 52 | 5175059H1 | 2901 | 3163 |
| 52 | 70250389V1 | 2145 | 2215 |
| 52 | 70692131V1 | 2910 | 3294 |
| 52 | 70691832V1 | 2912 | 3295 |
| 52 | 70691710V1 | 2152 | 2757 |
| 52 | 70693686V1 | 2952 | 3295 |
| 52 | g4535021 | 3028 | 3301 |
| 52 | 1711081F6 | 2152 | 2641 |
| 52 | 70697119V1 | 2152 | 2630 |
| 52 | 70693270V1 | 2152 | 2608 |
| 52 | 70696536V1 | 2152 | 2729 |
| 52 | 70692071V1 | 2152 | 2630 |
| 52 | 1711081H1 | 2152 | 2385 |
| 52 | 70693128V1 | 2193 | 2699 |
| 52 | 1854271H1 | 2311 | 2580 |
| 52 | 70255440V1 | 1097 | 1598 |
| 52 | 70254994V1 | 1119 | 1647 |
| 52 | 70254806V1 | 1141 | 1680 |
| 52 | 2057866H1 | 1170 | 1417 |
| 52 | 70254564V1 | 1204 | 1732 |
| 52 | 2348065F6 | 1277 | 1691 |
| 52 | 2348065H1 | 1277 | 1496 |
| 52 | g1694359 | 1300 | 1481 |
| 52 | 6799978J1 | 1302 | 1898 |
| 52 | 70255800V1 | 1339 | 1716 |
| 52 | 70250827V1 | 1343 | 1523 |
| 52 | 70257217V1 | 1351 | 1740 |
| 52 | 3856416H1 | 1357 | 1598 |
| 52 | 70255611V1 | 1378 | 1879 |
| 52 | 70248591V1 | 1394 | 1596 |
| 52 | 1322784H1 | 1427 | 1657 |
| 52 | 70255841V1 | 1459 | 1963 |
| 52 | 6799978H1 | 1476 | 2078 |
| 52 | 70257425V1 | 1481 | 1644 |
| 52 | 7007871H1 | 74 | 670 |
| 52 | 70252822V1 | 87 | 505 |
| 52 | 6496939H1 | 102 | 663 |
| 52 | 70248840V1 | 695 | 1099 |
| 52 | 70252852V1 | 121 | 517 |
| 52 | 70254883V1 | 704 | 1215 |
| 52 | 70257807V1 | 135 | 301 |
| 52 | 70258095V1 | 135 | 301 |
| 52 | 70247658V1 | 165 | 301 |
| 52 | 70252777V1 | 170 | 520 |
| 52 | 70252891V1 | 182 | 663 |
| 52 | 70252829V1 | 186 | 718 |
| 52 | 70252868V1 | 207 | 662 |
| 52 | 6575878H1 | 231 | 787 |
| 52 | 70252913V1 | 231 | 747 |
| 52 | 70252807V1 | 238 | 771 |
| 52 | 70254964V1 | 277 | 800 |
| 52 | 70250751V1 | 384 | 687 |
| 52 | 5097321F6 | 390 | 883 |
| 52 | 5097321H1 | 390 | 657 |
| 52 | 70250298V1 | 404 | 684 |
| 52 | 70252721V1 | 418 | 757 |
| 52 | 70248985V1 | 515 | 677 |
| 52 | 70247676V1 | 516 | 759 |
| 52 | 70249928V1 | 525 | 996 |
| 52 | 70255416V1 | 541 | 1010 |
| 52 | 70249098V1 | 555 | 646 |
| 52 | 70248520V1 | 558 | 889 |
| 52 | 70248316V1 | 561 | 888 |
| 52 | 70258290V1 | 582 | 873 |
| 52 | 70252961V1 | 665 | 1255 |
| 52 | 5097673H1 | 680 | 937 |
| 52 | 70252728V1 | 1 | 443 |
| 52 | 70252939V1 | 1 | 545 |
| 52 | 70252948V1 | 1 | 536 |
| 52 | 70252942V1 | 1 | 545 |
| 52 | 70252875V1 | 1 | 500 |
| 52 | 70252809V1 | 1 | 438 |
| 52 | 70252971V1 | 1 | 441 |
| 52 | 70252862V1 | 1 | 395 |
| 52 | 70252977V1 | 1 | 341 |
| 52 | 2640403H1 | 1 | 250 |
| 52 | 2640465H1 | 1 | 257 |
| 52 | 70252968V1 | 1 | 207 |
| 52 | 70252879V1 | 1 | 218 |
| 52 | 70253339V1 | 1 | 129 |
| 52 | 70252864V1 | 1 | 458 |
| 52 | 70252944V1 | 8 | 517 |
| 52 | 70252854V1 | 7 | 417 |
| 52 | 4830790H1 | 854 | 1092 |
| 52 | 70248055V1 | 879 | 1376 |
| 52 | 4999054H1 | 896 | 1140 |
| 52 | 4999939H2 | 910 | 1196 |
| 52 | 70258363V1 | 912 | 1079 |
| 52 | g1525795 | 981 | 1309 |
| 52 | 70255663V1 | 1009 | 1544 |
| 52 | 3616714H1 | 1011 | 1327 |
| 52 | 70257537V1 | 1046 | 1393 |
| 52 | 70255304V1 | 1064 | 1516 |
| 52 | 70254849V1 | 1078 | 1548 |
| 52 | 70255814V1 | 1077 | 1499 |
| 52 | 4513535H1 | 756 | 1022 |
| 52 | 70248026V1 | 769 | 1215 |
| 52 | 70255094V1 | 780 | 1277 |
| 52 | 70257552V1 | 805 | 1219 |
| 52 | 70252896V1 | 813 | 1400 |
| 52 | 70246987V1 | 815 | 1027 |
| 52 | 70248642V1 | 834 | 1228 |
| 52 | 2640465T6 | 834 | 1360 |
| 52 | g1525794 | 844 | 1078 |
| 52 | 70248207V1 | 851 | 1020 |
| 52 | 70255236V1 | 42 | 594 |
| 52 | 70256133V1 | 42 | 575 |
| 52 | 70246781V1 | 48 | 269 |
| 52 | 70252880V1 | 49 | 438 |
| 52 | 70256162V1 | 53 | 635 |
| 52 | 70252834V1 | 54 | 503 |
| 52 | 70255234V1 | 57 | 594 |
| 52 | 5317447H1 | 1845 | 2072 |
| 52 | 5315839H1 | 1845 | 1988 |
| 52 | 3727579T1 | 1851 | 2179 |
| 52 | g3070073 | 1873 | 2179 |
| 52 | 70255218V1 | 1540 | 2114 |
| 52 | 70257386V1 | 1596 | 1780 |
| 52 | 1284836T6 | 1607 | 2170 |
| 52 | 3727547H1 | 1615 | 1913 |
| 52 | 5095010H1 | 1615 | 1891 |
| 52 | 70255952V1 | 1645 | 2175 |
| 52 | 5097869H1 | 1645 | 1892 |
| 52 | 7344776H1 | 1680 | 2203 |
| 52 | 70256309V1 | 1737 | 2099 |
| 52 | 70254641V1 | 1742 | 2215 |
| 52 | 70257103V1 | 1743 | 1851 |
| 52 | 70250099V1 | 1751 | 1924 |
| 52 | 70249987V1 | 1752 | 1925 |
| 52 | 2348065T6 | 1755 | 2187 |
| 52 | 70254831V1 | 1756 | 2215 |
| 52 | 5002821H1 | 1757 | 2001 |
| 52 | 70256296V1 | 1762 | 2215 |
| 52 | 6783161H1 | 1766 | 2374 |
| 52 | 70249593V1 | 1789 | 1980 |
| 52 | 5095212H1 | 1791 | 2023 |
| 52 | 5867448H1 | 1794 | 2079 |
| 52 | 70257289V1 | 1818 | 2014 |
| 52 | 5317679H1 | 1845 | 2082 |
| 53 | 2836841H1 | 1 | 268 |
| 53 | 6552443H1 | 18 | 593 |
| 53 | 6914654J1 | 187 | 694 |
| 53 | 2928638H1 | 280 | 548 |
| 53 | 3278811F6 | 367 | 979 |
| 53 | 2137575F6 | 383 | 773 |
| 53 | 2137575H1 | 383 | 643 |
| 53 | 6914654H1 | 411 | 921 |
| 53 | 4008767H1 | 413 | 687 |
| 53 | 041252H1 | 446 | 640 |
| 53 | 1537441H1 | 532 | 777 |
| 53 | 2829631T7 | 652 | 1120 |
| 53 | 2502234H1 | 674 | 921 |
| 53 | 3278811T6 | 716 | 965 |
| 53 | 4957707H1 | 760 | 932 |
| 53 | 6768050J1 | 779 | 1398 |
| 53 | 6768050H1 | 860 | 1424 |
| 53 | 4775909H1 | 911 | 1113 |
| 53 | 4147184H1 | 1023 | 1259 |
| 53 | 6757024H1 | 1051 | 1701 |
| 53 | 2441247H1 | 1284 | 1514 |
| 53 | 6757024J1 | 1368 | 1874 |
| 53 | 1400881H1 | 1376 | 1605 |
| 53 | 1401450F6 | 1376 | 1884 |
| 53 | 1401450H1 | 1376 | 1621 |
| 53 | 3556941H1 | 1546 | 1834 |
| 54 | g3405927 | 1 | 245 |
| 54 | 4061048T8 | 1 | 543 |
| 54 | 5961372H1 | 217 | 755 |
| 55 | 6595513H2 | 1 | 455 |
| 55 | g3237891 | 1 | 431 |
| 55 | g1406421 | 1 | 383 |
| 55 | g5863035 | 1 | 313 |
| 55 | g2714241 | 1 | 468 |
| 55 | 7069348H1 | 10 | 209 |
| 55 | 70834691V1 | 13 | 578 |
| 55 | 71219243V1 | 13 | 540 |
| 55 | 4906280F6 | 13 | 523 |
| 55 | 70832787V1 | 13 | 476 |
| 55 | 4906280H2 | 13 | 256 |
| 55 | g680955 | 14 | 384 |
| 55 | 70822785V1 | 13 | 105 |
| 55 | 7339618H1 | 35 | 589 |
| 55 | 5970464H1 | 50 | 599 |
| 55 | 70833853V1 | 72 | 632 |
| 55 | 6772419J1 | 93 | 664 |
| 55 | 71220574V1 | 156 | 731 |
| 55 | g2433743 | 185 | 324 |
| 55 | 072410H1 | 202 | 432 |
| 55 | g1441845 | 234 | 323 |
| 55 | 71220458V1 | 256 | 858 |
| 55 | 71220313V1 | 258 | 856 |
| 55 | 2897226F6 | 308 | 762 |
| 55 | g1406525 | 312 | 723 |
| 55 | 2897226H1 | 310 | 585 |
| 55 | 3903661H1 | 326 | 619 |
| 55 | 2897226T6 | 341 | 869 |
| 55 | 5280549H1 | 418 | 688 |
| 55 | 7164092H1 | 442 | 978 |
| 55 | 71219526V1 | 554 | 1144 |
| 55 | g5527320 | 587 | 981 |
| 55 | 71219762V1 | 614 | 1143 |
| 55 | 71219779V1 | 691 | 849 |
| 55 | 6618366J2 | 702 | 1289 |
| 55 | 1479279F6 | 837 | 1300 |
| 55 | 1479279H1 | 837 | 1005 |
| 55 | 71220581V1 | 879 | 1134 |
| 55 | 1648685H1 | 895 | 1132 |
| 55 | 4631458H1 | 912 | 1183 |
| 55 | 2733185H1 | 990 | 1256 |
| 55 | g2111602 | 998 | 1417 |
| 55 | g685314 | 1017 | 1332 |
| 55 | 1721129F6 | 1104 | 1449 |
| 55 | 1721129H1 | 1104 | 1300 |
| 55 | 5742466H1 | 1118 | 1413 |
| 55 | 3902467H1 | 1118 | 1398 |
| 55 | 3902459H1 | 1118 | 1371 |
| 55 | 6918681H1 | 1176 | 1725 |
| 55 | 762794R1 | 1211 | 1711 |
| 55 | 762794F1 | 1221 | 1711 |
| 55 | 762794H1 | 1222 | 1451 |
| 55 | 7099404H1 | 1272 | 1774 |
| 55 | 6818682H1 | 1320 | 1676 |
| 55 | 70831777V1 | 1325 | 1840 |
| 55 | 2767081H1 | 1674 | 1918 |
| 55 | 5633412R8 | 1703 | 1788 |
| 55 | 6988282H1 | 1716 | 2079 |
| 55 | 5675457H1 | 1736 | 2001 |
| 55 | 1621977H1 | 1746 | 1826 |
| 55 | g6476364 | 1821 | 2213 |
| 55 | g1146532 | 1850 | 2203 |
| 55 | 5966829H1 | 1871 | 2436 |
| 55 | 1721129T6 | 1893 | 2173 |
| 55 | 7236449H1 | 2047 | 2275 |
| 55 | 6825137H1 | 2074 | 2522 |
| 55 | 656980H1 | 2237 | 2477 |
| 56 | 70563822V1 | 1350 | 1929 |
| 56 | 6140441H1 | 1312 | 1552 |
| 56 | 70674104V1 | 1320 | 1499 |
| 56 | 70567008V1 | 1333 | 1494 |
| 56 | g882792 | 2005 | 2419 |
| 56 | g6047387 | 729 | 1139 |
| 56 | g3033883 | 803 | 1144 |
| 56 | g4111129 | 828 | 1047 |
| 56 | g2397417 | 733 | 1106 |
| 56 | g2690499 | 829 | 1097 |
| 56 | g2620172 | 734 | 1066 |
| 56 | g1187613 | 797 | 982 |
| 56 | g5741320 | 737 | 1193 |
| 56 | g3765855 | 737 | 1063 |
| 56 | g6142132 | 833 | 1040 |
| 56 | g3770103 | 842 | 1154 |
| 56 | g6462630 | 796 | 965 |
| 56 | 70666243V1 | 781 | 1320 |
| 56 | 70669273V1 | 737 | 1186 |
| 56 | 4590906H1 | 799 | 983 |
| 56 | 344501H1 | 740 | 1113 |
| 56 | 5464077H1 | 770 | 1030 |
| 56 | 1989939H1 | 775 | 1044 |
| 56 | 2342785T6 | 795 | 1371 |
| 56 | 70564198V1 | 779 | 1223 |
| 56 | 588426T6 | 771 | 1207 |
| 56 | 5096743H1 | 881 | 1110 |
| 56 | 70566592V1 | 819 | 1438 |
| 56 | 70668498V1 | 855 | 1353 |
| 56 | 2653741H1 | 840 | 1122 |
| 56 | g1962977 | 845 | 1314 |
| 56 | 588426R6 | 842 | 1207 |
| 56 | 6803050H1 | 887 | 1254 |
| 56 | 1297330T6 | 931 | 1073 |
| 56 | 70567006V1 | 903 | 1458 |
| 56 | 70562861V1 | 917 | 1563 |
| 56 | 4408168H1 | 1000 | 1255 |
| 56 | 70564258V1 | 942 | 1569 |
| 56 | 4083330H1 | 1008 | 1286 |
| 56 | 70662295V1 | 965 | 1066 |
| 56 | 70662354V1 | 1055 | 1155 |
| 56 | 70562675V1 | 982 | 1511 |
| 56 | 588426H1 | 1048 | 1279 |
| 56 | 5620393R8 | 1017 | 1245 |
| 56 | 70564166V1 | 1034 | 1599 |
| 56 | g573475 | 1052 | 1378 |
| 56 | 70669958V1 | 1053 | 1323 |
| 56 | 70570002V1 | 1041 | 1456 |
| 56 | g882849 | 1056 | 1327 |
| 56 | 70663697V1 | 1058 | 1346 |
| 56 | 6706725H1 | 1062 | 1567 |
| 56 | 70668887V1 | 1062 | 1559 |
| 56 | 883631H1 | 1055 | 1298 |
| 56 | 4376346H1 | 1074 | 1327 |
| 56 | g5674715 | 1063 | 1301 |
| 56 | g882957 | 1080 | 1329 |
| 56 | g5837269 | 1068 | 1291 |
| 56 | 70670665V1 | 1082 | 1462 |
| 56 | 70662627V1 | 1090 | 1644 |
| 56 | 70658327V1 | 1099 | 1239 |
| 56 | 70565737V1 | 1112 | 1707 |
| 56 | 6459664H1 | 1122 | 1708 |
| 56 | 6459764H1 | 1156 | 1331 |
| 56 | 70566802V1 | 1118 | 1858 |
| 56 | 6459772H1 | 1206 | 1358 |
| 56 | g766175 | 1134 | 1200 |
| 56 | 70563249V1 | 1142 | 1653 |
| 56 | 2175353F6 | 1138 | 1346 |
| 56 | 2175353H1 | 1138 | 1388 |
| 56 | 70564319V1 | 1146 | 1662 |
| 56 | 70564161V1 | 1148 | 1705 |
| 56 | 3957616H2 | 1158 | 1372 |
| 56 | 3958977H1 | 1159 | 1445 |
| 56 | 70566156V1 | 1159 | 1550 |
| 56 | 70664157V1 | 1189 | 1428 |
| 56 | 729045H1 | 1194 | 1422 |
| 56 | 195736R6 | 1195 | 1438 |
| 56 | 195736H1 | 1195 | 1400 |
| 56 | 729045R6 | 1195 | 1424 |
| 56 | 1297330F6 | 1202 | 1653 |
| 56 | 6751614H1 | 1206 | 1810 |
| 56 | 5493653H1 | 1206 | 1481 |
| 56 | 6735456H1 | 1230 | 1348 |
| 56 | 5552258H1 | 1248 | 1342 |
| 56 | 653456H1 | 1276 | 1418 |
| 56 | 4703350H1 | 1276 | 1348 |
| 56 | 70566134V1 | 1331 | 1923 |
| 56 | 70563519V1 | 1299 | 1861 |
| 56 | 6631102H1 | 1301 | 1782 |
| 56 | 2006255H1 | 1302 | 1513 |
| 56 | 195736T6 | 1736 | 2373 |
| 56 | 70565915V1 | 1589 | 2352 |
| 56 | 1687411H1 | 1659 | 1891 |
| 56 | 3497631H1 | 1707 | 2009 |
| 56 | 3930656T9 | 1884 | 2275 |
| 56 | 1727562T6 | 1874 | 2376 |
| 56 | 70562875V1 | 1935 | 2413 |
| 56 | 3255274H1 | 1776 | 2026 |
| 56 | 70565475V1 | 1935 | 2345 |
| 56 | 201433H1 | 1807 | 2142 |
| 56 | 4799413H1 | 1966 | 2233 |
| 56 | 200319H1 | 1807 | 2154 |
| 56 | 1262834H1 | 2017 | 2252 |
| 56 | 1989939T6 | 1856 | 2152 |
| 56 | g822901 | 2079 | 2419 |
| 56 | 4046894F8 | 1 | 602 |
| 56 | 5622335H1 | 34 | 306 |
| 56 | 5620393F8 | 37 | 506 |
| 56 | 5620393H1 | 37 | 310 |
| 56 | 6746680H1 | 45 | 629 |
| 56 | 7184188H1 | 87 | 506 |
| 56 | 2839152F6 | 95 | 550 |
| 56 | 1727562F6 | 97 | 507 |
| 56 | 2839152H1 | 95 | 383 |
| 56 | 3386005H1 | 95 | 350 |
| 56 | 70564762V1 | 97 | 598 |
| 56 | 1727562H1 | 97 | 312 |
| 56 | 5044068H1 | 105 | 379 |
| 56 | 5044068F6 | 105 | 555 |
| 56 | 70566005V1 | 148 | 570 |
| 56 | 2452790H1 | 183 | 403 |
| 56 | 7156278H1 | 232 | 814 |
| 56 | 70565482V1 | 441 | 1010 |
| 56 | 6630501U1 | 445 | 1010 |
| 56 | 70565168V1 | 457 | 1122 |
| 56 | 5044068R6 | 519 | 939 |
| 56 | 70565572V1 | 549 | 1013 |
| 56 | 70563167V1 | 589 | 1157 |
| 56 | 4414516H1 | 597 | 862 |
| 56 | 70564872V1 | 599 | 1148 |
| 56 | 2187472H1 | 651 | 928 |
| 56 | 70563453V1 | 669 | 1239 |
| 56 | 70564150V1 | 678 | 1265 |
| 56 | 70564317V1 | 705 | 1376 |
| 56 | 70562729V1 | 718 | 1328 |
| 56 | 70562680V1 | 726 | 1288 |
| 56 | 70564226V1 | 742 | 1421 |
| 56 | g4988332 | 2176 | 2411 |
| 56 | g564908 | 2177 | 2411 |
| 56 | 3590745H1 | 2330 | 2634 |
| 56 | 476150H1 | 2331 | 2602 |
| 56 | 1577439H1 | 2440 | 2654 |
| 56 | g2347702 | 2176 | 2413 |
| 56 | g4124357 | 2097 | 2417 |
| 56 | g882958 | 2124 | 2419 |
| 56 | 3791454H1 | 2139 | 2415 |
| 56 | 4905965H2 | 2176 | 2361 |
| 56 | g6473997 | 2176 | 2415 |
| 56 | 6141362H1 | 1555 | 1901 |
| 56 | 4597106H1 | 1559 | 1853 |
| 56 | 70564268V1 | 1362 | 1678 |
| 56 | 70564413V1 | 1378 | 1930 |
| 56 | 3553956H1 | 1381 | 1537 |
| 56 | 70669318V1 | 1404 | 1644 |
| 56 | 1241441H1 | 1429 | 1701 |
| 56 | 1297330H1 | 1489 | 1643 |
| 56 | 70565761V1 | 1509 | 2000 |
| 56 | 4857906H1 | 1550 | 1860 |
| 57 | g5664193 | 1 | 377 |
| 57 | g2619178 | 5 | 333 |
| 57 | 6441247H1 | 12 | 543 |
| 57 | 453769H1 | 184 | 377 |
| 57 | 3762476H1 | 235 | 538 |
| 57 | 6729757H1 | 348 | 910 |
| 57 | 3116387F6 | 383 | 761 |
| 57 | 3116387H1 | 384 | 664 |
| 57 | 5661301H1 | 391 | 644 |
| 57 | g1977456 | 394 | 782 |
| 57 | 3496148F6 | 397 | 981 |
| 57 | 70139347V1 | 397 | 887 |
| 57 | 3496148H1 | 397 | 675 |
| 57 | 1008165H1 | 455 | 761 |
| 57 | 6518018H1 | 464 | 1031 |
| 57 | 4177624H1 | 490 | 696 |
| 57 | 826356H1 | 517 | 846 |
| 57 | 6441415H1 | 554 | 1059 |
| 57 | 4539787H1 | 619 | 878 |
| 57 | 70142580V1 | 634 | 1044 |
| 57 | 6710170H1 | 649 | 1209 |
| 57 | 6203306H1 | 668 | 1199 |
| 57 | 70131575V1 | 692 | 1096 |
| 57 | 7362891H1 | 712 | 1260 |
| 57 | 2798030F6 | 720 | 1287 |
| 57 | 2798030H1 | 720 | 971 |
| 57 | 2659046H1 | 723 | 968 |
| 57 | g1748057 | 740 | 899 |
| 57 | 1832131H1 | 748 | 960 |
| 57 | 4822509H1 | 763 | 1012 |
| 57 | 6980545H1 | 771 | 1103 |
| 57 | 5219156H1 | 843 | 1062 |
| 57 | 5767782H1 | 859 | 1315 |
| 57 | 5644717H1 | 859 | 1095 |
| 57 | 6917456H1 | 880 | 1413 |
| 57 | 3788651H1 | 923 | 1087 |
| 57 | 4571245H1 | 925 | 1203 |
| 57 | 3436728H1 | 928 | 1087 |
| 57 | 2231388F6 | 953 | 1455 |
| 57 | 2231388H1 | 953 | 1202 |
| 57 | 5040546H1 | 956 | 1087 |
| 57 | 70133707V1 | 976 | 1464 |
| 57 | 2113640H1 | 977 | 1226 |
| 57 | 6583623H1 | 1015 | 1355 |
| 57 | g2004410 | 1019 | 1322 |
| 57 | 4314146H1 | 1020 | 1302 |
| 57 | g2010211 | 1020 | 1353 |
| 57 | g1189835 | 1027 | 1229 |
| 57 | 70131494V1 | 1104 | 1537 |
| 57 | 2737895H1 | 1123 | 1333 |
| 57 | 6443615H1 | 1123 | 1627 |
| 57 | 666344H1 | 1123 | 1300 |
| 57 | 70143472V1 | 1128 | 1500 |
| 57 | 4112076H1 | 1170 | 1444 |
| 57 | 469801H1 | 1232 | 1464 |
| 57 | 469801R6 | 1232 | 1464 |
| 57 | 70138703V1 | 1237 | 1803 |
| 57 | 817498H1 | 1266 | 1558 |
| 57 | 817306R1 | 1266 | 1836 |
| 57 | 817306H1 | 1266 | 1484 |
| 57 | 6440332H1 | 1269 | 1829 |
| 57 | 4226121H1 | 1286 | 1563 |
| 57 | 2888006H1 | 1291 | 1565 |
| 57 | 5326773H1 | 1333 | 1512 |
| 57 | 4827817H1 | 1343 | 1622 |
| 57 | 2837990H1 | 1343 | 1585 |
| 57 | 181567H1 | 1351 | 1537 |
| 57 | 064652H1 | 1351 | 1526 |
| 57 | 1566979H1 | 1353 | 1562 |
| 57 | 5489077H1 | 1372 | 1494 |
| 57 | 70132097V1 | 1377 | 1687 |
| 57 | g853275 | 1388 | 1725 |
| 57 | 6132302H1 | 1388 | 1663 |
| 57 | 2744049H1 | 1388 | 1648 |
| 57 | 826818H1 | 1400 | 1733 |
| 57 | 3467063H1 | 1437 | 1705 |
| 57 | 70133770V1 | 1439 | 1833 |
| 57 | 3449015H1 | 1463 | 1629 |
| 57 | 3121922H1 | 1463 | 1773 |
| 57 | 2538144H1 | 1485 | 1813 |
| 57 | 70136427V1 | 1488 | 1931 |
| 57 | 2008648H1 | 1506 | 1685 |
| 57 | 3145660T6 | 1511 | 2119 |
| 57 | 70136864V1 | 1526 | 1925 |
| 57 | 469801T6 | 1526 | 2094 |
| 57 | 3116387T6 | 1579 | 2142 |
| 57 | 4760891H1 | 1610 | 1891 |
| 57 | 1572810T6 | 1637 | 2138 |
| 57 | 3917658H1 | 1652 | 1962 |
| 57 | g5764969 | 1660 | 2134 |
| 57 | 6752149H1 | 1663 | 2144 |
| 57 | g5232494 | 1668 | 2138 |
| 57 | g3835399 | 1696 | 2137 |
| 57 | g3804418 | 1702 | 2137 |
| 57 | 1814612T6 | 1704 | 2141 |
| 57 | 3879011H1 | 1725 | 2014 |
| 57 | g3145312 | 1767 | 2184 |
| 57 | g5658515 | 1767 | 2183 |
| 57 | g4896513 | 1777 | 2189 |
| 57 | g3041462 | 1783 | 2183 |
| 57 | 1268321F6 | 1789 | 2170 |
| 57 | 1268321H1 | 1789 | 2040 |
| 57 | 2798030T6 | 1790 | 2144 |
| 57 | 4855273H1 | 1844 | 2031 |
| 57 | 3947216H1 | 1844 | 2020 |
| 57 | 2232569H1 | 1859 | 2088 |
| 57 | g6507253 | 1866 | 2134 |
| 57 | 2244523H1 | 1863 | 2114 |
| 57 | 2044580H1 | 1880 | 2159 |
| 57 | g4088814 | 1891 | 2181 |
| 57 | 1268321T6 | 1903 | 2130 |
| 57 | 4850944H1 | 1927 | 2158 |
| 57 | 1492447H1 | 1928 | 2134 |
| 57 | g5658077 | 1940 | 2134 |
| 57 | g1211221 | 1951 | 2196 |
| 57 | 621406H1 | 1954 | 2171 |
| 57 | g852913 | 1962 | 2111 |
| 57 | 2231388T6 | 1974 | 2142 |
| 57 | g1155579 | 2012 | 2197 |
| 57 | 2245140H1 | 2088 | 2172 |
| 57 | 3496148T7 | 1537 | 2094 |
| 57 | 6175211H1 | 1541 | 1824 |
| 57 | 1814612F6 | 1548 | 2013 |
| 57 | 1814612H1 | 1548 | 1801 |
| 57 | 2370362H1 | 1551 | 1807 |
| 57 | 2372634H1 | 1551 | 1793 |
| 57 | 70131661V1 | 1563 | 1852 |
| 58 | 2174866H1 | 1 | 146 |
| 58 | 2698567H1 | 1 | 197 |
| 58 | 5541622H1 | 1 | 194 |
| 58 | 2930567F6 | 33 | 479 |
| 58 | 2930567H1 | 33 | 338 |
| 58 | 4982447H1 | 269 | 540 |
| 58 | 6255850H1 | 303 | 576 |
| 58 | 6258186H1 | 303 | 577 |
| 58 | 5721674H1 | 321 | 908 |
| 58 | 4152487H1 | 322 | 596 |
| 58 | 4028078H1 | 339 | 611 |
| 58 | 7279087H1 | 343 | 928 |
| 58 | 3097754H1 | 343 | 649 |
| 58 | 5668252H1 | 343 | 574 |
| 58 | 5538912H2 | 342 | 416 |
| 58 | 2816646H1 | 349 | 612 |
| 58 | 2816646F6 | 349 | 871 |
| 58 | 2610076H1 | 349 | 585 |
| 58 | 2777845H1 | 349 | 580 |
| 58 | 3293887H1 | 350 | 593 |
| 58 | g1753994 | 350 | 559 |
| 58 | 3355981H1 | 351 | 608 |
| 58 | g29167 | 376 | 691 |
| 58 | 2476803H1 | 387 | 650 |
| 58 | 2476803F6 | 387 | 595 |
| 58 | g1646650 | 406 | 767 |
| 58 | 3796918H1 | 778 | 1068 |
| 58 | 7217683H1 | 812 | 1267 |
| 58 | 5608991H1 | 821 | 1011 |
| 58 | 1811369F6 | 853 | 1303 |
| 58 | 1811369H1 | 853 | 1163 |
| 58 | 71041470V1 | 943 | 1602 |
| 58 | 4514405H1 | 957 | 1207 |
| 58 | 3860484H1 | 982 | 1279 |
| 58 | 6708640H1 | 1017 | 1588 |
| 58 | 207612H1 | 1015 | 1241 |
| 58 | 3461827H1 | 1088 | 1182 |
| 58 | 6753368J1 | 1105 | 1745 |
| 58 | g1629548 | 1195 | 1610 |
| 58 | 7156421J1 | 1209 | 1390 |
| 58 | 7156421H1 | 1209 | 1374 |
| 58 | 7202481H1 | 1235 | 1816 |
| 58 | 653612R6 | 1252 | 1492 |
| 58 | 6325601H1 | 1252 | 1547 |
| 58 | 653612H1 | 1252 | 1494 |
| 58 | 653612T6 | 1253 | 1462 |
| 58 | 2667659H1 | 1309 | 1395 |
| 58 | 2206550H1 | 1293 | 1492 |
| 58 | 6598025H1 | 1340 | 1850 |
| 58 | 3928253H1 | 1315 | 1633 |
| 58 | 3928196H1 | 1316 | 1628 |
| 58 | 5039319T9 | 1355 | 1949 |
| 58 | 3414970H1 | 1347 | 1620 |
| 58 | 1743113H1 | 1381 | 1664 |
| 58 | g2115578 | 1382 | 1750 |
| 58 | 71042069V1 | 1422 | 2060 |
| 58 | 5691992H1 | 1409 | 1710 |
| 58 | 2816646T6 | 1435 | 2011 |
| 58 | 71041726V1 | 1509 | 2118 |
| 58 | 4689116H1 | 1522 | 1787 |
| 58 | 2936253H1 | 1524 | 1803 |
| 58 | 3528601T6 | 1552 | 2014 |
| 58 | 5203894H1 | 1559 | 1823 |
| 58 | 3415070T6 | 1578 | 2015 |
| 58 | 5423080H1 | 1583 | 1854 |
| 58 | 5422280H1 | 1583 | 1828 |
| 58 | g434100 | 1753 | 2008 |
| 58 | 1811369T6 | 1780 | 2215 |
| 58 | 6489437H1 | 1800 | 2249 |
| 58 | g1751419 | 1841 | 2048 |
| 58 | g2541732 | 1845 | 2055 |
| 58 | g5530419 | 1870 | 2049 |
| 58 | 3212650T6 | 1875 | 2026 |
| 58 | 3118422H1 | 1882 | 2049 |
| 58 | 3002323F6 | 1882 | 2047 |
| 58 | 3118368T6 | 1885 | 2009 |
| 58 | 830827R1 | 1897 | 2249 |
| 58 | 830827H1 | 1897 | 2160 |
| 58 | 997447H1 | 1899 | 2151 |
| 58 | 6506368H1 | 1903 | 2249 |
| 58 | g1977012 | 1938 | 2287 |
| 58 | 1811076T6 | 1938 | 2221 |
| 58 | 2589380H1 | 1968 | 2238 |
| 58 | 2552865H1 | 1968 | 2215 |
| 58 | g2631458 | 1972 | 2243 |
| 58 | 5573308H1 | 1982 | 2232 |
| 58 | g6439334 | 2088 | 2250 |
| 58 | 3732638H1 | 2100 | 2243 |
| 58 | 3736212H1 | 2116 | 2249 |
| 58 | g1629446 | 2143 | 2250 |
| 58 | g3891127 | 2146 | 2250 |
| 58 | 6131388H1 | 2153 | 2243 |
| 59 | 3625376H1 | 2752 | 2845 |
| 59 | g5675476 | 2517 | 2963 |
| 59 | g3146172 | 2594 | 2958 |
| 59 | 3274786H1 | 2616 | 2858 |
| 59 | 1953692H1 | 111 | 344 |
| 59 | 6539166H1 | 198 | 716 |
| 59 | 2133241F6 | 229 | 647 |
| 59 | 2133241H1 | 229 | 494 |
| 59 | 6365336H1 | 335 | 613 |
| 59 | 1676569H1 | 375 | 591 |
| 59 | g2881893 | 461 | 513 |
| 59 | 6812474H1 | 525 | 1081 |
| 59 | 4934590H1 | 525 | 609 |
| 59 | 045505H1 | 526 | 755 |
| 59 | 2670704F6 | 598 | 980 |
| 59 | 2670704H1 | 598 | 846 |
| 59 | 003688H1 | 628 | 941 |
| 59 | 6812474J1 | 792 | 1344 |
| 59 | 2185753H1 | 923 | 1155 |
| 59 | 1665259H1 | 1333 | 1552 |
| 59 | 5193614H1 | 1718 | 1967 |
| 59 | 4378244H1 | 1994 | 2271 |
| 59 | 70742391V1 | 2036 | 2610 |
| 59 | 419872H1 | 2167 | 2379 |
| 59 | 171741R1 | 2186 | 2661 |
| 59 | 171741H1 | 2186 | 2362 |
| 59 | g2009473 | 2197 | 2490 |
| 59 | g2189171 | 2227 | 2329 |
| 59 | g6330101 | 1 | 2954 |
| 59 | 2669204T6 | 5 | 463 |
| 59 | 5951559H1 | 34 | 348 |
| 59 | 3942253H1 | 2231 | 2521 |
| 59 | 042842H1 | 2237 | 2480 |
| 59 | 2656261H1 | 2243 | 2497 |
| 59 | 1971006F6 | 2319 | 2789 |
| 59 | 1971006H1 | 2319 | 2583 |
| 59 | 2667143T6 | 2341 | 2911 |
| 59 | 001227H1 | 2373 | 2728 |
| 59 | 3716845H1 | 2374 | 2648 |
| 59 | 6514287H1 | 2382 | 2935 |
| 59 | 4120983H1 | 2404 | 2640 |
| 59 | g4189206 | 2487 | 2958 |
| 59 | g2278337 | 2503 | 2957 |
| 60 | 71229143V1 | 621 | 1253 |
| 60 | 6983112H1 | 624 | 904 |
| 60 | g570318 | 633 | 919 |
| 60 | 7046749H1 | 452 | 1052 |
| 60 | 70868787V1 | 465 | 1132 |
| 60 | 753174H1 | 355 | 544 |
| 60 | 4318873H1 | 153 | 369 |
| 60 | 71230534V1 | 162 | 657 |
| 60 | 7322168H1 | 165 | 793 |
| 60 | 6992614H1 | 233 | 746 |
| 60 | g778569 | 676 | 1011 |
| 60 | 744829H1 | 675 | 915 |
| 60 | 7158869H1 | 1 | 478 |
| 60 | 3335250F6 | 28 | 397 |
| 60 | 70870096V1 | 657 | 1340 |
| 60 | 70869315V1 | 674 | 1384 |
| 60 | 744829R1 | 675 | 1254 |
| 60 | 3335250H1 | 28 | 270 |
| 60 | 7077668H1 | 130 | 662 |
| 60 | g518739 | 2296 | 2520 |
| 60 | 60202000D1 | 2303 | 2520 |
| 60 | g3230679 | 2327 | 2520 |
| 60 | g717890 | 2461 | 2535 |
| 60 | 60201999D1 | 2508 | 2561 |
| 60 | 750787H1 | 2258 | 2510 |
| 60 | 667235H1 | 2263 | 2515 |
| 60 | g561290 | 2289 | 2520 |
| 60 | g714831 | 2246 | 2557 |
| 60 | 70818421V1 | 673 | 1279 |
| 60 | g869715 | 675 | 1023 |
| 60 | 4745248H1 | 1 | 238 |
| 60 | 748982H1 | 675 | 914 |
| 60 | 70869543V1 | 746 | 1291 |
| 60 | 70838362V1 | 773 | 900 |
| 60 | 70837422V1 | 778 | 1013 |
| 60 | 71229105V1 | 787 | 1469 |
| 60 | 70870484V1 | 807 | 1465 |
| 60 | 70869894V1 | 829 | 1508 |
| 60 | 70839431V1 | 828 | 1138 |
| 60 | 70837620V1 | 868 | 1305 |
| 60 | g565684 | 918 | 1102 |
| 60 | 71221539V1 | 985 | 1416 |
| 60 | 70869173V1 | 992 | 1418 |
| 60 | 71229615V1 | 1005 | 1492 |
| 60 | 70867752V1 | 1053 | 1804 |
| 60 | 70870784V1 | 1013 | 1466 |
| 60 | 71229687V1 | 1038 | 1742 |
| 60 | 70870712V1 | 1032 | 1587 |
| 60 | 70870207V1 | 1036 | 1656 |
| 60 | g1025621 | 1047 | 1385 |
| 60 | g1059514 | 1047 | 1284 |
| 60 | 71229437V1 | 1156 | 1803 |
| 60 | g714830 | 1131 | 1442 |
| 60 | 70870012V1 | 1147 | 1778 |
| 60 | 4311224H1 | 1231 | 1528 |
| 60 | 70869086V1 | 1284 | 1849 |
| 60 | 2292421R6 | 1498 | 1606 |
| 60 | 71229131V1 | 1473 | 2098 |
| 60 | 2292254R6 | 1506 | 1988 |
| 60 | 2291932H1 | 1506 | 1759 |
| 60 | 530715H1 | 1531 | 1754 |
| 60 | 7090888H1 | 1628 | 1769 |
| 60 | g3086021 | 1626 | 2047 |
| 60 | 71228809V1 | 1628 | 2200 |
| 60 | 60202364B1 | 1657 | 2134 |
| 60 | 60202363B1 | 1657 | 2096 |
| 60 | 2291932T6 | 1669 | 2269 |
| 60 | 60202367B1 | 1665 | 2041 |
| 60 | 3335250T6 | 1672 | 2184 |
| 60 | 70870682V1 | 1770 | 2476 |
| 60 | 70869072V1 | 1785 | 2476 |
| 60 | 70867264V1 | 1828 | 2445 |
| 60 | 70868309V1 | 1828 | 2491 |
| 60 | 6841962H1 | 1858 | 2421 |
| 60 | 70870157V1 | 1978 | 2569 |
| 60 | 6855669H1 | 2008 | 2520 |
| 60 | 6885209J1 | 2001 | 2441 |
| 60 | 746910R6 | 2044 | 2520 |
| 60 | 746910T6 | 2045 | 2516 |
| 60 | 746910H1 | 2044 | 2281 |
| 60 | 6844175H1 | 2075 | 2520 |
| 60 | 2568562H1 | 2123 | 2362 |
| 60 | g4393425 | 2130 | 2563 |
| 60 | g4109519 | 2140 | 2520 |
| 60 | g2694947 | 2170 | 2520 |
| 60 | g2703845 | 2174 | 2520 |
| 60 | g3884077 | 2176 | 2520 |
| 60 | g3278030 | 2179 | 2569 |
| 60 | 4705947H1 | 2239 | 2398 |
| 60 | 5266308H1 | 635 | 795 |
| 61 | 6803748H1 | 1 | 281 |
| 61 | 6803748J1 | 185 | 681 |
| 61 | 7179441H1 | 281 | 837 |
| 61 | 6993211H1 | 431 | 958 |
| 61 | 990041H1 | 585 | 938 |
| 61 | 2647312H1 | 2235 | 2464 |
| 61 | 5303648H2 | 2242 | 2538 |
| 61 | 7236332H1 | 2251 | 2534 |
| 61 | 3606921H1 | 2256 | 2543 |
| 61 | 4423382H1 | 2269 | 2538 |
| 61 | 1976495H1 | 2269 | 2526 |
| 61 | g6400732 | 2279 | 2543 |
| 61 | 6538345H1 | 2292 | 2509 |
| 61 | 1942829H1 | 2284 | 2540 |
| 61 | 1929606H1 | 2292 | 2537 |
| 61 | 1794859H1 | 2312 | 2543 |
| 61 | 6912812H1 | 961 | 1221 |
| 61 | 1602952F6 | 691 | 1143 |
| 61 | 3436025H1 | 691 | 925 |
| 61 | 3025993H1 | 921 | 1218 |
| 61 | 954323H1 | 2326 | 2529 |
| 61 | 954323R1 | 2326 | 2529 |
| 61 | 954323T1 | 2326 | 2483 |
| 61 | g395758 | 2345 | 2528 |
| 61 | 5393045H1 | 2354 | 2470 |
| 61 | 1602953H1 | 691 | 793 |
| 61 | 1602952H1 | 691 | 779 |
| 61 | 1602953F6 | 691 | 909 |
| 61 | 1568974H1 | 1974 | 2177 |
| 61 | 6950168H1 | 1976 | 2470 |
| 61 | 6951183H1 | 1988 | 2470 |
| 61 | 1510868H1 | 1997 | 2126 |
| 61 | 5511854H1 | 2007 | 2266 |
| 61 | g2017578 | 2021 | 2249 |
| 61 | 2225061H1 | 2036 | 2295 |
| 61 | 5685115H1 | 2039 | 2313 |
| 61 | 5503829H1 | 2046 | 2302 |
| 61 | 6736816H1 | 2051 | 2433 |
| 61 | 6914591J1 | 1587 | 2103 |
| 61 | 7267538H1 | 1622 | 1800 |
| 61 | 6559545H1 | 1677 | 2237 |
| 61 | 7287559H1 | 1703 | 2171 |
| 61 | 6912812J1 | 1719 | 2352 |
| 61 | 6714578H1 | 1791 | 2351 |
| 61 | 6384321H1 | 1791 | 2017 |
| 61 | 7124412H1 | 1803 | 2268 |
| 61 | g650235 | 1811 | 2087 |
| 61 | 3934148H1 | 1829 | 1983 |
| 61 | 6914591H1 | 1830 | 2173 |
| 61 | 3934148F6 | 1838 | 2178 |
| 61 | 5950691H1 | 1874 | 2208 |
| 61 | 5950591H1 | 1874 | 1946 |
| 61 | 7336402H1 | 1901 | 2467 |
| 61 | 4000559H1 | 1959 | 2238 |
| 61 | 1570912F6 | 1974 | 2307 |
| 61 | 1570912H1 | 1974 | 2184 |
| 61 | 7152207H1 | 1328 | 1863 |
| 61 | 7152523H1 | 1375 | 1863 |
| 61 | 684121H1 | 1431 | 1674 |
| 61 | 1998986H1 | 1449 | 1545 |
| 61 | 598762H1 | 1472 | 1718 |
| 61 | 2227505H1 | 1546 | 1793 |
| 61 | 3393621H1 | 1557 | 1854 |
| 61 | 3441995H1 | 1570 | 1822 |
| 61 | g5528672 | 2232 | 2527 |
| 61 | 6992150H1 | 1037 | 1416 |
| 61 | 4514493H1 | 1075 | 1162 |
| 61 | 1622836H1 | 1163 | 1292 |
| 61 | 2864609H1 | 1208 | 1358 |
| 61 | 7259857H1 | 1220 | 1855 |
| 61 | 3551930H1 | 1220 | 1512 |
| 61 | 1379503H1 | 1223 | 1458 |
| 61 | 7154005H1 | 1283 | 1863 |
| 61 | 5297140H1 | 2150 | 2448 |
| 61 | 3984036H1 | 2152 | 2347 |
| 61 | g3048902 | 2160 | 2538 |
| 61 | 5297634H1 | 2177 | 2469 |
| 61 | 5297140F8 | 2180 | 2460 |
| 61 | g5367334 | 2217 | 2545 |
| 61 | 2569168H1 | 2218 | 2485 |
| 61 | g3842534 | 2219 | 2536 |
| 61 | 2449539H1 | 2228 | 2458 |
| 61 | 4947830H1 | 2067 | 2189 |
| 61 | 5345386H1 | 2069 | 2260 |
| 61 | g4244699 | 2071 | 2541 |
| 61 | 3934148T6 | 2072 | 2496 |
| 61 | 3480660H1 | 2078 | 2301 |
| 61 | 576039H1 | 2079 | 2319 |
| 61 | g650236 | 2084 | 2482 |
| 61 | 2915418H1 | 2086 | 2383 |
| 61 | g4664830 | 2100 | 2538 |
| 61 | g1017329 | 2107 | 2465 |
| 61 | g704704 | 2107 | 2457 |
| 62 | 1730307H1 | 320 | 543 |
| 62 | 1852850F6 | 344 | 904 |
| 62 | 1852850H1 | 344 | 623 |
| 62 | 1786201H1 | 1224 | 1347 |
| 62 | 5500430F6 | 4 | 384 |
| 62 | 3751387H1 | 26 | 298 |
| 62 | 6862090H1 | 34 | 606 |
| 62 | 6939856H1 | 42 | 508 |
| 62 | 1264746H1 | 209 | 445 |
| 62 | 1730307F6 | 320 | 696 |
| 62 | g2782908 | 1611 | 2044 |
| 62 | 4573617F6 | 1197 | 1548 |
| 62 | 4573617H1 | 1197 | 1460 |
| 62 | g953428 | 863 | 1055 |
| 62 | 70576776V1 | 1251 | 1474 |
| 62 | 4516586H1 | 1279 | 1522 |
| 62 | 1964843H1 | 856 | 1126 |
| 62 | 3508361F6 | 1 | 54 |
| 62 | 3277390H1 | 1484 | 1734 |
| 62 | 819859H1 | 1480 | 1708 |
| 62 | 1852850T6 | 1436 | 2015 |
| 62 | g1521345 | 1445 | 1556 |
| 62 | 1966204T6 | 1488 | 2046 |
| 62 | 7183685H1 | 1516 | 2053 |
| 62 | g5394325 | 1583 | 1905 |
| 62 | 1540056H1 | 1599 | 1816 |
| 62 | 71013442V1 | 1295 | 1412 |
| 62 | g1275357 | 1296 | 1768 |
| 62 | 71008110V1 | 1299 | 1866 |
| 62 | 3080148H1 | 1299 | 1445 |
| 62 | g772703 | 1305 | 1422 |
| 62 | g890466 | 1340 | 1492 |
| 62 | 3121030F6 | 1342 | 1806 |
| 62 | 4562285H1 | 1351 | 1630 |
| 62 | 826668H1 | 1403 | 1691 |
| 62 | 6370579H1 | 1404 | 1660 |
| 62 | 71008260V1 | 864 | 1019 |
| 62 | 2542469H1 | 909 | 1139 |
| 62 | g766126 | 956 | 1040 |
| 62 | 1966137H1 | 863 | 1097 |
| 62 | 1966204R6 | 863 | 1107 |
| 62 | 1966137R6 | 863 | 1325 |
| 62 | 5500430R6 | 4 | 432 |
| 62 | 3121030H1 | 1342 | 1653 |
| 62 | 2128205H1 | 1702 | 1971 |
| 62 | 1966137T6 | 1663 | 2049 |
| 62 | 6843171H1 | 1670 | 2093 |
| 62 | 4573617T6 | 1679 | 2086 |
| 62 | 71236524V1 | 1681 | 1927 |
| 62 | g1521346 | 1691 | 2093 |
| 62 | g2030368 | 1694 | 2085 |
| 62 | 4976812H1 | 1656 | 1926 |
| 62 | g5630558 | 1769 | 2096 |
| 62 | 4421852H1 | 1773 | 2020 |
| 62 | g2946092 | 1782 | 2096 |
| 62 | 3324470H1 | 1798 | 2071 |
| 62 | 71236287V1 | 1829 | 2002 |
| 62 | g820432 | 1839 | 2114 |
| 62 | g2899828 | 1848 | 2096 |
| 62 | g4224026 | 1850 | 2093 |
| 62 | g890467 | 1850 | 2077 |
| 62 | g3116690 | 1859 | 2093 |
| 62 | g6476925 | 1890 | 2093 |
| 62 | 3508361T6 | 1912 | 2054 |
| 62 | g518427 | 1917 | 2093 |
| 62 | 71013952V1 | 1980 | 2089 |
| 62 | g5362837 | 1996 | 2101 |
| 62 | g3434471 | 1757 | 2093 |
| 62 | g1219640 | 1768 | 2096 |
| 62 | g822861 | 1727 | 2124 |
| 62 | g5634808 | 1758 | 2101 |
| 62 | 3121030T6 | 1613 | 2054 |
| 62 | 1730307T6 | 1632 | 2053 |
| 62 | 820388H1 | 1644 | 1878 |
| 62 | 70590209V1 | 1648 | 2093 |
| 62 | 5266218T6 | 1655 | 2071 |
| 62 | 5609530H1 | 1237 | 1422 |
| 62 | 6222010U1 | 1248 | 1565 |
| 62 | 3508361H1 | 1 | 297 |
| 62 | 71014845V1 | 611 | 896 |
| 62 | 6618123J1 | 696 | 1329 |
| 62 | 71008490V1 | 732 | 1116 |
| 62 | 71009446V1 | 377 | 1047 |
| 62 | 6221969U2 | 590 | 945 |
| 62 | 4305106H1 | 1008 | 1298 |
| 62 | 1823430H1 | 1026 | 1243 |
| 62 | g1958618 | 1021 | 1382 |
| 62 | 3760149H1 | 1062 | 1376 |
| 62 | 6221994U1 | 1073 | 1569 |
| 62 | g1976176 | 1153 | 1550 |
| 63 | 70684185V1 | 1354 | 1880 |
| 63 | 70682617V1 | 1395 | 1806 |
| 63 | 70707488V1 | 1460 | 1832 |
| 63 | 70683177V1 | 1461 | 1983 |
| 63 | 70707392V1 | 1462 | 1832 |
| 63 | 70680197V1 | 1465 | 1847 |
| 63 | 70679832V1 | 1602 | 2217 |
| 63 | 70681040V1 | 1625 | 2221 |
| 63 | 7282455H1 | 1636 | 2232 |
| 63 | 70684191V1 | 1662 | 1801 |
| 63 | 70687607V1 | 1721 | 2137 |
| 63 | 70680047V1 | 1742 | 2387 |
| 63 | 70679818V1 | 1769 | 2456 |
| 63 | 6543969H1 | 1780 | 2352 |
| 63 | 70683980V1 | 270 | 750 |
| 63 | 3925359H1 | 270 | 546 |
| 63 | 3925359F6 | 270 | 448 |
| 63 | 70682278V1 | 270 | 429 |
| 63 | 3531535H1 | 322 | 622 |
| 63 | 70680431V1 | 396 | 861 |
| 63 | 1560619F6 | 422 | 805 |
| 63 | 1560619H1 | 422 | 632 |
| 63 | 3699779H1 | 467 | 637 |
| 63 | g6399045 | 695 | 1131 |
| 63 | 70681329V1 | 709 | 873 |
| 63 | 70708067V1 | 711 | 811 |
| 63 | 2992541F6 | 780 | 1329 |
| 63 | 2992541H1 | 781 | 1083 |
| 63 | 70680523V1 | 889 | 1539 |
| 63 | 1560619T6 | 958 | 1101 |
| 63 | 70683064V1 | 971 | 1406 |
| 63 | 70684594V1 | 985 | 1592 |
| 63 | 70680581V1 | 1006 | 1548 |
| 63 | 70683735V1 | 1037 | 1587 |
| 63 | 70682555V1 | 1322 | 1950 |
| 63 | 70704188V1 | 1327 | 1535 |
| 63 | 999076R1 | 1323 | 1695 |
| 63 | 999076R6 | 1323 | 1695 |
| 63 | 999076H1 | 1323 | 1568 |
| 63 | 6623832J1 | 1349 | 1520 |
| 63 | 4378643F7 | 1825 | 2433 |
| 63 | 4379312F7 | 1825 | 2363 |
| 63 | 4379312H1 | 1825 | 2112 |
| 63 | 4378643H1 | 1826 | 2123 |
| 63 | 70682506V1 | 1843 | 2439 |
| 63 | 6623832H1 | 1843 | 2366 |
| 63 | 70708640V1 | 1901 | 2124 |
| 63 | 3925359T6 | 1915 | 2464 |
| 63 | 5100839T9 | 1935 | 2253 |
| 63 | 4378643T7 | 1966 | 2392 |
| 63 | 70682105V1 | 1968 | 2496 |
| 63 | 4071965T6 | 1989 | 2503 |
| 63 | 5328012T6 | 2014 | 2458 |
| 63 | 5328012F6 | 2021 | 2417 |
| 63 | 4071965F6 | 1 | 359 |
| 63 | 4071965H1 | 1 | 295 |
| 63 | 7125750H1 | 1 | 395 |
| 63 | 70682403V1 | 270 | 773 |
| 63 | 5328012H1 | 2021 | 2275 |
| 63 | g3871281 | 2037 | 2485 |
| 63 | g2934374 | 2056 | 2496 |
| 63 | 1431377H1 | 2204 | 2445 |
| 63 | g3047856 | 2222 | 2499 |
| 63 | g4371425 | 2237 | 2496 |
| 63 | 70686041V1 | 2264 | 2420 |
| 63 | g4327870 | 2279 | 2496 |
| 63 | g3134381 | 2279 | 2496 |
| 63 | 70684032V1 | 1100 | 1538 |
| 63 | 70684362V1 | 1110 | 1587 |
| 63 | 70682629V1 | 1151 | 1740 |
| 63 | 70683547V1 | 1185 | 1635 |
| 63 | 3832836H1 | 1163 | 1384 |
| 63 | 70684463V1 | 1170 | 1734 |
| 63 | 70682920V1 | 1196 | 1755 |
| 63 | 70703741V1 | 1215 | 1394 |
| 63 | 70682638V1 | 1223 | 1736 |
| 63 | 70682682V1 | 1223 | 1749 |
| 63 | 70682459V1 | 1249 | 1908 |
| 63 | 70681204V1 | 1257 | 1766 |
| 63 | 70685069V1 | 1300 | 1653 |
| 63 | 6931315H1 | 1291 | 1695 |
| 64 | 3254414H1 | 69 | 317 |
| 64 | 698585H1 | 70 | 286 |
| 64 | 595891H1 | 74 | 292 |
| 64 | 6175776H1 | 79 | 356 |
| 64 | g2159426 | 78 | 188 |
| 64 | 657830H1 | 80 | 273 |
| 64 | 3457909H1 | 81 | 348 |
| 64 | 6301420H1 | 81 | 349 |
| 64 | 3575481H1 | 85 | 394 |
| 64 | g3430798 | 912 | 1371 |
| 64 | g5110040 | 919 | 1372 |
| 64 | g5590010 | 921 | 1377 |
| 64 | 1620383H1 | 543 | 765 |
| 64 | g848117 | 553 | 822 |
| 64 | 2448547H1 | 572 | 801 |
| 64 | 838081H1 | 598 | 828 |
| 64 | 1866438H1 | 678 | 930 |
| 64 | 1283358H1 | 826 | 1062 |
| 64 | g6504962 | 862 | 1372 |
| 64 | g4764556 | 888 | 1358 |
| 64 | g5675975 | 891 | 1372 |
| 64 | g5848125 | 902 | 1372 |
| 64 | g5764743 | 904 | 1374 |
| 64 | g5545082 | 905 | 1373 |
| 64 | g5886738 | 906 | 1372 |
| 64 | g4691015 | 907 | 1373 |
| 64 | g4486385 | 908 | 1374 |
| 64 | g4287684 | 909 | 1371 |
| 64 | 1520534H1 | 911 | 1135 |
| 64 | 1647964H1 | 452 | 606 |
| 64 | 1648365H1 | 452 | 624 |
| 64 | 2054919H1 | 478 | 749 |
| 64 | 2770417H1 | 478 | 698 |
| 64 | g1933783 | 493 | 719 |
| 64 | 943317H1 | 501 | 752 |
| 64 | 2796760H1 | 503 | 780 |
| 64 | 2209885H1 | 503 | 669 |
| 64 | 1348306H1 | 523 | 753 |
| 64 | 1345963H1 | 523 | 741 |
| 64 | 2840447H1 | 536 | 670 |
| 64 | 3483204H1 | 1 | 253 |
| 64 | 6178578H1 | 1 | 218 |
| 64 | 3574891H1 | 12 | 173 |
| 64 | 666288H1 | 1189 | 1371 |
| 64 | g6141365 | 1191 | 1372 |
| 64 | g6044743 | 1197 | 1372 |
| 64 | 2011575H1 | 1211 | 1303 |
| 64 | g4264614 | 1185 | 1372 |
| 64 | g5233294 | 1187 | 1372 |
| 64 | 3617375H1 | 222 | 428 |
| 64 | 7141668H1 | 231 | 698 |
| 64 | 5272669H1 | 248 | 468 |
| 64 | 2824332H1 | 248 | 536 |
| 64 | 1521787H1 | 267 | 468 |
| 64 | g6472427 | 923 | 1374 |
| 64 | g3679316 | 924 | 1372 |
| 64 | g5768136 | 931 | 1372 |
| 64 | g5813683 | 939 | 1374 |
| 64 | 1622824H1 | 938 | 1078 |
| 64 | g2910940 | 939 | 1372 |
| 64 | g4390160 | 945 | 1371 |
| 64 | g6451090 | 953 | 1376 |
| 64 | g5864498 | 957 | 1372 |
| 64 | g6073735 | 960 | 1377 |
| 64 | g6035456 | 963 | 1372 |
| 64 | g4300047 | 971 | 1372 |
| 64 | g2782529 | 1004 | 1372 |
| 64 | g3870189 | 1005 | 1373 |
| 64 | g2555234 | 1032 | 1371 |
| 64 | g6506601 | 1039 | 1372 |
| 64 | g4073949 | 1047 | 1373 |
| 64 | g4435426 | 1050 | 1377 |
| 64 | g4298104 | 1051 | 1373 |
| 64 | g5630151 | 1055 | 1376 |
| 64 | g3750504 | 1074 | 1374 |
| 64 | g4509918 | 1077 | 1372 |
| 64 | g3884253 | 1079 | 1373 |
| 64 | g5590634 | 1087 | 1373 |
| 64 | g2874129 | 1097 | 1316 |
| 64 | g2222026 | 1109 | 1373 |
| 64 | g1933727 | 1109 | 1374 |
| 64 | g4331539 | 1123 | 1372 |
| 64 | g4987875 | 1124 | 1374 |
| 64 | 3343619H1 | 1127 | 1372 |
| 64 | g2913373 | 1132 | 1372 |
| 64 | 4204339H1 | 1134 | 1372 |
| 64 | g2115784 | 1144 | 1376 |
| 64 | 3999467H1 | 1144 | 1366 |
| 64 | g5325930 | 1150 | 1371 |
| 64 | 1497968T6 | 1155 | 1322 |
| 64 | 1491584F6 | 1162 | 1372 |
| 64 | 1491584H1 | 1162 | 1360 |
| 64 | g4123527 | 1174 | 1372 |
| 64 | g5233350 | 1181 | 1373 |
| 64 | g774782 | 31 | 202 |
| 64 | g766776 | 38 | 379 |
| 64 | 1417835H1 | 44 | 296 |
| 64 | 3390866H1 | 15 | 247 |
| 64 | 3295609H1 | 67 | 345 |
| 64 | 1630345H1 | 69 | 301 |
| 64 | 1630524H1 | 69 | 298 |
| 64 | 3359727H1 | 129 | 229 |
| 64 | 6149745H1 | 142 | 693 |
| 64 | 6817217J1 | 162 | 754 |
| 64 | g4606728 | 165 | 384 |
| 64 | g2063565 | 171 | 590 |
| 64 | 1987618H1 | 188 | 407 |
| 64 | 6735147H1 | 85 | 195 |
| 64 | 552603H1 | 89 | 286 |
| 64 | 1988618R6 | 91 | 498 |
| 64 | 1988618H1 | 91 | 316 |
| 64 | 6577808H1 | 91 | 254 |
| 64 | 6432246H1 | 101 | 473 |
| 64 | 3231513H1 | 101 | 331 |
| 64 | 2642693H1 | 100 | 322 |
| 64 | 6265064H1 | 115 | 300 |
| 64 | 3447578H2 | 124 | 390 |
| 64 | 1746508H1 | 343 | 620 |
| 64 | 6817217H1 | 344 | 811 |
| 64 | 7386534H1 | 350 | 837 |
| 64 | 5204331H1 | 367 | 623 |
| 64 | 7240357H1 | 306 | 493 |
| 64 | 2526407H1 | 306 | 556 |
| 64 | 3187627H1 | 315 | 615 |
| 64 | 2768194H1 | 319 | 572 |
| 64 | 2814432H1 | 317 | 602 |
| 64 | 3729901H1 | 1212 | 1372 |
| 64 | 4194682H1 | 1236 | 1343 |
| 64 | 1288019H1 | 1266 | 1372 |
| 64 | 5225561H1 | 1266 | 1371 |
| 64 | 5077632H1 | 1293 | 1376 |
| 64 | g2464354 | 1295 | 1371 |
| 64 | g2986696 | 1218 | 1372 |
| 64 | 6795195H1 | 67 | 633 |
| 64 | 2372795H1 | 379 | 601 |
| 64 | 6945477H1 | 436 | 775 |
| 64 | 6826837H1 | 64 | 366 |
| 64 | 6826837J1 | 64 | 366 |
| 64 | 1418532H1 | 44 | 241 |
| 64 | g4223892 | 52 | 551 |
| 64 | 2855769F6 | 54 | 195 |
| 64 | 7162693H1 | 55 | 632 |
| 64 | 2855769H1 | 54 | 323 |
| 64 | 3276579H1 | 54 | 314 |
| 64 | 6497645H1 | 57 | 708 |
| 64 | 3232792H1 | 58 | 353 |
| 64 | 478616H1 | 65 | 358 |
| 65 | 2132236T6 | 1004 | 1401 |
| 65 | 2132236H1 | 1004 | 1263 |
| 65 | g1887025 | 1093 | 1444 |
| 65 | 2086267H1 | 1132 | 1266 |
| 65 | 6175915H1 | 1148 | 1402 |
| 65 | g4876495 | 1204 | 1445 |
| 65 | g5365413 | 1204 | 1445 |
| 65 | 2132236R6 | 1004 | 1434 |
| 65 | 1667496H1 | 609 | 860 |
| 65 | 1300507F6 | 678 | 1121 |
| 65 | 1300507H1 | 678 | 918 |
| 65 | 1670894T6 | 892 | 1399 |
| 65 | 1300507T6 | 930 | 1415 |
| 65 | 1670894F6 | 609 | 1072 |
| 65 | 1670914H1 | 609 | 863 |
| 65 | 2682534H1 | 444 | 676 |
| 65 | g1887075 | 233 | 454 |
| 65 | 4888904H1 | 1 | 277 |
| 65 | 4888904F8 | 25 | 578 |
| 65 | 4888904F9 | 27 | 472 |
| 66 | 7266271H1 | 192 | 555 |
| 66 | 7266415H1 | 192 | 764 |
| 66 | 4828047H1 | 658 | 939 |
| 66 | 5506726H1 | 736 | 923 |
| 66 | 6491143H1 | 855 | 1416 |
| 66 | 5614913H1 | 984 | 1235 |
| 66 | g1921859 | 1104 | 1499 |
| 66 | 5903582T6 | 1122 | 1468 |
| 66 | g2437372 | 1135 | 1552 |
| 66 | 6037758H1 | 1141 | 1523 |
| 66 | g2112991 | 1169 | 1578 |
| 66 | 2075750F6 | 1372 | 1546 |
| 66 | 5903582F6 | 1 | 473 |
| 66 | 5903582H1 | 1 | 273 |
| 67 | 70985136V1 | 1070 | 1350 |
| 67 | 70985260V1 | 1073 | 1455 |
| 67 | 70983054V1 | 1073 | 1558 |
| 67 | 71295531V1 | 1073 | 1453 |
| 67 | 3717638T6 | 1080 | 1571 |
| 67 | 70984050V1 | 1221 | 1738 |
| 67 | 70986990V1 | 1372 | 1585 |
| 67 | g1980540 | 1447 | 1629 |
| 67 | g760823 | 1462 | 1594 |
| 67 | 71295238V1 | 1066 | 1226 |
| 67 | 71295235V1 | 1067 | 1708 |
| 67 | 3717638F6 | 762 | 1251 |
| 67 | 3717638H1 | 762 | 822 |
| 67 | 71269157V1 | 1057 | 1278 |
| 67 | 70984218V1 | 1064 | 1297 |
| 67 | 3365081H1 | 1 | 160 |
| 67 | 6535437H1 | 18 | 468 |
| 67 | 71295036V1 | 289 | 822 |
| 67 | 71295290V1 | 321 | 570 |
| 67 | 70985880V1 | 321 | 580 |
| 67 | 70986588V1 | 321 | 517 |
| 67 | 71295432V1 | 604 | 822 |
| 67 | 70985853V1 | 677 | 1325 |
| 67 | 2261815H1 | 654 | 822 |
| 67 | 71294916V1 | 670 | 1292 |
| 67 | 71296536V1 | 568 | 1197 |
| 67 | 70986118V1 | 545 | 822 |
| 67 | 70986361V1 | 546 | 1189 |
| 67 | 70985351V1 | 321 | 549 |
| 67 | 3384358H1 | 328 | 499 |
| 67 | 71123582V1 | 328 | 431 |
| 67 | 70986562V1 | 379 | 822 |
| 67 | 70985543V1 | 397 | 824 |
| 67 | 71295516V1 | 397 | 820 |
| 67 | 70984072V1 | 397 | 734 |
| 67 | 71295044V1 | 397 | 763 |
| 67 | 70986344V1 | 397 | 650 |
| 67 | 70983024V1 | 510 | 1139 |
| 67 | 71294736V1 | 510 | 822 |
| 67 | 70983429V1 | 646 | 1207 |
| 68 | g2884969 | 1 | 149 |
| 68 | 6702215H1 | 1 | 640 |
| 69 | 3524645H1 | 1160 | 1405 |
| 69 | 70365425D1 | 247 | 658 |
| 69 | 3030907F6 | 263 | 672 |
| 69 | 3030907H1 | 263 | 558 |
| 69 | g574359 | 341 | 660 |
| 69 | 70378343D1 | 342 | 793 |
| 69 | 70365801D1 | 372 | 867 |
| 69 | 2656315T6 | 1358 | 1946 |
| 69 | 70376806D1 | 1386 | 1894 |
| 69 | g770844 | 438 | 691 |
| 69 | 6178165H1 | 499 | 799 |
| 69 | 6552117H1 | 584 | 1167 |
| 69 | 6552017H1 | 584 | 1047 |
| 69 | 70377552D1 | 1193 | 1657 |
| 69 | g766638 | 710 | 999 |
| 69 | g564283 | 710 | 957 |
| 69 | 5710302H2 | 721 | 969 |
| 69 | 4093587H1 | 1283 | 1553 |
| 69 | 70378136D1 | 1397 | 1884 |
| 69 | 7010739H1 | 1234 | 1657 |
| 69 | 70365505D1 | 1359 | 1894 |
| 69 | 70365928D1 | 1 | 611 |
| 69 | 6729581H1 | 1 | 416 |
| 69 | 2998772F6 | 1 | 174 |
| 69 | 7172430H1 | 1 | 425 |
| 69 | 2998772H1 | 1 | 75 |
| 69 | 70362802D1 | 17 | 511 |
| 69 | 2656315F6 | 17 | 568 |
| 69 | 2656315H1 | 17 | 245 |
| 69 | 6179484H1 | 90 | 350 |
| 69 | 6201177H1 | 105 | 612 |
| 69 | 70364249D1 | 1394 | 1894 |
| 69 | 70451849V1 | 1414 | 1599 |
| 69 | 70451495V1 | 1418 | 1657 |
| 69 | 70446527V1 | 1443 | 1879 |
| 69 | 70378406D1 | 1447 | 1881 |
| 69 | 70364892D1 | 1466 | 1869 |
| 69 | g723744 | 1686 | 1942 |
| 69 | g749908 | 1756 | 1879 |
| 69 | 70377452D1 | 1767 | 1880 |
| 69 | 70366252D1 | 1787 | 1884 |
| 69 | 70365521D1 | 1787 | 1893 |
| 69 | 661155T6 | 1787 | 1950 |
| 69 | 1292366F6 | 1203 | 1689 |
| 69 | 1292366H1 | 1203 | 1452 |
| 69 | 70377916D1 | 1315 | 1657 |
| 69 | 70364727D1 | 1315 | 1657 |
| 69 | 70366014D1 | 1315 | 1880 |
| 69 | 70450434V1 | 1318 | 1850 |
| 69 | 5675995H1 | 1317 | 1571 |
| 69 | 70364614D1 | 1339 | 1894 |
| 69 | 70364566D1 | 1338 | 1894 |
| 69 | 70364017D1 | 651 | 1226 |
| 69 | 2295159R6 | 660 | 1121 |
| 69 | 70362705D1 | 645 | 882 |
| 69 | 70366027D1 | 651 | 1243 |
| 69 | 70451316V1 | 1240 | 1648 |
| 69 | 1292366F1 | 1203 | 1595 |
| 69 | 70378088D1 | 408 | 888 |
| 69 | 7071779H1 | 410 | 731 |
| 69 | 70365775D1 | 1419 | 1922 |
| 69 | 70378121D1 | 1386 | 1894 |
| 69 | 70446570V1 | 916 | 1260 |
| 69 | 70447538V1 | 1056 | 1507 |
| 69 | 2295159H1 | 660 | 911 |
| 69 | 5081872H1 | 690 | 891 |
| 69 | g872874 | 709 | 1076 |
| 70 | 765092H1 | 184 | 414 |
| 70 | 6351315H2 | 235 | 422 |
| 70 | 765092R6 | 1 | 412 |
| 70 | 765092T6 | 37 | 412 |
| 70 | 6351215H2 | 237 | 597 |
| 71 | g4523614 | 641 | 887 |
| 71 | g3203614 | 1 | 374 |
| 71 | 1485642H1 | 86 | 348 |
| 71 | 6963127H1 | 98 | 348 |
| 71 | 3599520H1 | 200 | 492 |
| 71 | 1485642T6 | 318 | 963 |
| 71 | 6954177H1 | 512 | 1103 |
| 71 | g820653 | 538 | 876 |
| 71 | 3214706H1 | 629 | 894 |
| 71 | g5369990 | 637 | 887 |
| 71 | 1485642F6 | 86 | 661 |
| 71 | 1483336H1 | 86 | 391 |
| 72 | 183176R6 | 27 | 491 |
| 72 | 183176H1 | 27 | 251 |
| 72 | 2733388H1 | 110 | 339 |
| 72 | 5616358H1 | 120 | 396 |
| 72 | 71238219V1 | 165 | 770 |
| 72 | g4762579 | 398 | 832 |
| 72 | 71020348V1 | 429 | 1029 |
| 72 | 71019924V1 | 467 | 918 |
| 72 | 7104793H1 | 478 | 998 |
| 72 | 71019186V1 | 519 | 967 |
| 72 | 4004284H1 | 525 | 792 |
| 72 | 71020080V1 | 771 | 1365 |
| 72 | 71238694V1 | 772 | 1352 |
| 72 | 71237183V1 | 791 | 1317 |
| 72 | 71019222V1 | 822 | 1224 |
| 72 | 71240267V1 | 868 | 1118 |
| 72 | 71240088V1 | 868 | 1118 |
| 72 | 71237170V1 | 1030 | 1386 |
| 72 | g2358498 | 1 | 382 |
| 72 | 183176R1 | 27 | 640 |
| 73 | 277677H1 | 1431 | 1536 |
| 73 | 3873445H1 | 946 | 1227 |
| 73 | 6448059H1 | 968 | 1478 |
| 73 | 000843H1 | 1046 | 1539 |
| 73 | 867412H1 | 1039 | 1238 |
| 73 | 160137T6 | 1041 | 1517 |
| 73 | 1928767T6 | 1083 | 1496 |
| 73 | 1928767R6 | 1099 | 1542 |
| 73 | 1928767H1 | 1099 | 1376 |
| 73 | 2937207H1 | 1099 | 1339 |
| 73 | 5001393H1 | 1100 | 1297 |
| 73 | g5631970 | 1101 | 1539 |
| 73 | 5989817H1 | 1102 | 1292 |
| 73 | g5747028 | 1103 | 1536 |
| 73 | 6074562H1 | 1113 | 1412 |
| 73 | 6074662H1 | 1113 | 1338 |
| 73 | g2719198 | 1477 | 1539 |
| 73 | 5902113H1 | 1501 | 1728 |
| 73 | 4931449H1 | 1501 | 1770 |
| 73 | g673993 | 182 | 474 |
| 73 | g1267101 | 196 | 545 |
| 73 | 4826378H1 | 262 | 524 |
| 73 | 556057H1 | 270 | 478 |
| 73 | 556041H1 | 270 | 479 |
| 73 | 3449244H1 | 1427 | 1651 |
| 73 | 3521301H1 | 1274 | 1416 |
| 73 | 6511601H1 | 1280 | 1531 |
| 73 | 7060576H1 | 1283 | 1718 |
| 73 | g1424041 | 1315 | 1536 |
| 73 | g4523497 | 1231 | 1652 |
| 73 | g1193170 | 1237 | 1536 |
| 73 | 3051747H1 | 1272 | 1562 |
| 73 | 3996830H1 | 1513 | 1643 |
| 73 | 453660H1 | 1653 | 1728 |
| 73 | 355514H1 | 1212 | 1424 |
| 73 | g2899582 | 1156 | 1536 |
| 73 | 3802944H1 | 1179 | 1451 |
| 73 | g5862645 | 1187 | 1536 |
| 73 | 4001379H1 | 1198 | 1339 |
| 73 | 4819034H1 | 45 | 131 |
| 73 | 3725796H1 | 76 | 160 |
| 73 | g1273189 | 87 | 309 |
| 73 | g2141610 | 1148 | 1539 |
| 73 | g4224292 | 1149 | 1536 |
| 73 | g1861043 | 1153 | 1541 |
| 73 | 6074694H1 | 1113 | 1357 |
| 73 | g2969771 | 1116 | 1536 |
| 73 | 2877117H1 | 1 | 139 |
| 73 | g1934264 | 1321 | 1728 |
| 73 | 816059R1 | 1351 | 1728 |
| 73 | 816059R6 | 1351 | 1549 |
| 73 | 816059H1 | 1351 | 1589 |
| 73 | 6716201H1 | 1400 | 1664 |
| 73 | 4439494H1 | 1412 | 1548 |
| 73 | 2417379F6 | 342 | 682 |
| 73 | 2417379H1 | 342 | 509 |
| 73 | 433802H1 | 342 | 509 |
| 73 | g1640167 | 342 | 492 |
| 73 | 3723919H1 | 360 | 471 |
| 73 | 4443121H1 | 401 | 705 |
| 73 | 6309581H1 | 458 | 948 |
| 73 | 2779428H1 | 473 | 715 |
| 73 | 4829932H2 | 595 | 840 |
| 73 | g1891400 | 596 | 1068 |
| 73 | 6368954H1 | 647 | 926 |
| 73 | 160137H1 | 660 | 901 |
| 73 | 160137R6 | 664 | 1020 |
| 73 | 1968940H1 | 726 | 989 |
| 73 | 3899549H1 | 785 | 1048 |
| 73 | 2667776H1 | 815 | 1045 |
| 73 | 4871959H1 | 830 | 1026 |
| 73 | 2608521H1 | 904 | 1097 |
| 73 | 5433362H1 | 913 | 1091 |
| 73 | g2963571 | 1473 | 1662 |
| 73 | g2910204 | 1121 | 1536 |
| 73 | g5592759 | 1126 | 1537 |
| 73 | 1227022H1 | 1139 | 1269 |
| 73 | 795156H1 | 1140 | 1383 |
| 73 | g748850 | 11 | 227 |
| 73 | g678626 | 24 | 445 |
| 73 | 556041R6 | 270 | 622 |
| 73 | 6717668H1 | 277 | 609 |
| 74 | 5947068H1 | 65 | 379 |
| 74 | g673182 | 66 | 311 |
| 74 | 70585819V1 | 66 | 223 |
| 74 | g784700 | 81 | 171 |
| 74 | 4071087H1 | 84 | 393 |
| 74 | g3401588 | 316 | 738 |
| 74 | g3412723 | 338 | 732 |
| 74 | g6038772 | 338 | 732 |
| 74 | g784495 | 365 | 732 |
| 74 | 3346654H1 | 49 | 339 |
| 74 | 70585937V1 | 67 | 573 |
| 74 | 2198034H1 | 54 | 310 |
| 74 | 6559467H1 | 53 | 662 |
| 74 | 2554485H1 | 55 | 292 |
| 74 | 3213289H1 | 55 | 220 |
| 74 | 7154394H1 | 56 | 646 |
| 74 | 999535H1 | 55 | 245 |
| 74 | 2446468H1 | 58 | 303 |
| 74 | 138735H1 | 61 | 441 |
| 74 | 70576514V1 | 117 | 242 |
| 74 | 5529836H1 | 83 | 260 |
| 74 | 2642157H1 | 83 | 233 |
| 74 | 2854411H1 | 84 | 281 |
| 74 | 3394115H1 | 84 | 265 |
| 74 | 2457270H1 | 84 | 217 |
| 74 | 7246868H2 | 85 | 171 |
| 74 | 5467726H1 | 91 | 265 |
| 74 | 5048151H1 | 112 | 277 |
| 74 | 4055118T9 | 1 | 653 |
| 74 | 2571441H1 | 11 | 258 |
| 74 | 6297876H1 | 49 | 332 |
| 74 | g1389377 | 68 | 470 |
| 74 | 4621236H1 | 70 | 366 |
| 74 | 2718503H1 | 69 | 184 |
| 74 | g813048 | 71 | 501 |
| 74 | g570365 | 71 | 376 |
| 74 | 5478847H1 | 74 | 317 |
| 74 | g613447 | 81 | 330 |
| 74 | 70589118V1 | 67 | 741 |
| 74 | 7344794H1 | 68 | 664 |
| 74 | 1649902F6 | 67 | 377 |
| 74 | 1384494H1 | 67 | 331 |
| 74 | 1649839H1 | 67 | 311 |
| 74 | 1649902H1 | 67 | 310 |
| 74 | 6389250H1 | 68 | 379 |
| 74 | 2729287T6 | 122 | 700 |
| 74 | 4055118T7 | 124 | 624 |
| 74 | 906915H1 | 132 | 286 |
| 74 | 5621907H1 | 135 | 475 |
| 74 | g1281834 | 137 | 550 |
| 74 | 3773136H1 | 160 | 490 |
| 74 | 70588034V1 | 182 | 477 |
| 74 | 70588682V1 | 186 | 734 |
| 74 | 1649902T6 | 192 | 689 |
| 74 | 70590189V1 | 243 | 734 |
| 74 | g2908534 | 249 | 701 |
| 74 | 70573192V1 | 282 | 565 |
| 74 | g4070388 | 291 | 730 |
| 74 | 70578389V1 | 295 | 439 |
| 74 | 2584982H1 | 299 | 556 |
| 74 | g3924424 | 317 | 650 |
| 74 | g4896209 | 440 | 747 |
| 74 | 70590586V1 | 409 | 565 |
| 74 | 4171532H1 | 427 | 728 |
| 75 | g864641 | 472 | 730 |
| 75 | g854955 | 498 | 853 |
| 75 | 7018723H1 | 526 | 1079 |
| 75 | 1634405F6 | 537 | 1019 |
| 75 | 1634405H1 | 537 | 753 |
| 75 | 2135762H1 | 598 | 889 |
| 75 | 6754472H1 | 666 | 1291 |
| 75 | 1573637F6 | 689 | 1107 |
| 75 | 1573637H1 | 689 | 906 |
| 75 | 000132H1 | 691 | 1122 |
| 75 | g1164431 | 695 | 972 |
| 75 | 2893510H1 | 729 | 1003 |
| 75 | 5687323H1 | 796 | 1064 |
| 75 | 4369688H1 | 854 | 1120 |
| 75 | 6130274H1 | 858 | 971 |
| 75 | g778259 | 940 | 1189 |
| 75 | 5076483H1 | 1055 | 1318 |
| 75 | 3926443F6 | 1083 | 1488 |
| 75 | 3926443H1 | 1084 | 1284 |
| 75 | 439597T6 | 1142 | 1776 |
| 75 | 1634405T6 | 1166 | 1762 |
| 75 | 2530791F6 | 1225 | 1590 |
| 75 | 2530791H1 | 1225 | 1466 |
| 75 | 3926443T6 | 1285 | 1789 |
| 75 | 567711T6 | 1298 | 1783 |
| 75 | 567711R6 | 1298 | 1759 |
| 75 | 567703H1 | 1298 | 1551 |
| 75 | g2785236 | 1315 | 1793 |
| 75 | g3649430 | 1318 | 1747 |
| 75 | g2779890 | 1324 | 1813 |
| 75 | 1573637T6 | 1340 | 1773 |
| 75 | g4176290 | 1343 | 1817 |
| 75 | g3765500 | 1344 | 1814 |
| 75 | 5032276H1 | 1380 | 1620 |
| 75 | g2569759 | 1383 | 1814 |
| 75 | g3433438 | 1386 | 1804 |
| 75 | g2903602 | 1395 | 1814 |
| 75 | 2530791T6 | 1411 | 1773 |
| 75 | 2994433H1 | 1433 | 1644 |
| 75 | g1148705 | 1461 | 1817 |
| 75 | g2903846 | 1465 | 1793 |
| 75 | g1880344 | 1474 | 1588 |
| 75 | g777248 | 1484 | 1755 |
| 75 | g6450447 | 1495 | 1793 |
| 75 | g3753249 | 1527 | 1813 |
| 75 | g863905 | 1529 | 1804 |
| 75 | g778854 | 1529 | 1795 |
| 75 | g854914 | 1530 | 1773 |
| 75 | g5365595 | 1535 | 1816 |
| 75 | g778166 | 1662 | 1796 |
| 75 | g2269957 | 1680 | 1813 |
| 75 | g3056293 | 1703 | 1814 |
| 75 | 7341877H1 | 194 | 778 |
| 75 | 6092377H1 | 332 | 608 |
| 75 | 3074534H1 | 1 | 263 |
| 75 | 3293881H1 | 38 | 284 |
| 75 | 439597R6 | 177 | 719 |
| 75 | 439597H1 | 177 | 404 |
| 75 | 6558204H1 | 182 | 612 |
| 75 | 062726H1 | 374 | 548 |
| 75 | 062712H1 | 374 | 541 |
| 75 | 5565196H1 | 413 | 626 |
| 76 | 1375031F6 | 785 | 1262 |
| 76 | 1375031F1 | 784 | 1041 |
| 76 | 2133064H1 | 763 | 1041 |
| 76 | 6827035J1 | 1 | 659 |
| 76 | 3507907H1 | 885 | 1175 |
| 76 | 114238H1 | 915 | 988 |
| 76 | 128209H1 | 915 | 1030 |
| 76 | 6530747H1 | 1096 | 1708 |
| 76 | 5291702H1 | 1127 | 1366 |
| 76 | 1375031H1 | 784 | 1034 |
| 76 | 684570H1 | 829 | 1046 |
| 76 | g2786676 | 2256 | 2677 |
| 76 | g959893 | 2259 | 2671 |
| 76 | g6033820 | 2259 | 2666 |
| 76 | 2180264T6 | 2265 | 2625 |
| 76 | g6074973 | 2269 | 2673 |
| 76 | g1507095 | 2273 | 2669 |
| 76 | g5741986 | 2275 | 2672 |
| 76 | g4004471 | 2278 | 2672 |
| 76 | g1201567 | 2280 | 2668 |
| 76 | g2786837 | 2282 | 2677 |
| 76 | g6086742 | 2293 | 2671 |
| 76 | 70091053V1 | 2301 | 2666 |
| 76 | 70092520V1 | 2230 | 2635 |
| 76 | 600490H1 | 2233 | 2502 |
| 76 | 70090081V1 | 2238 | 2676 |
| 76 | g4985261 | 2241 | 2670 |
| 76 | g3047609 | 2241 | 2666 |
| 76 | g898301 | 2240 | 2666 |
| 76 | g1194822 | 2242 | 2669 |
| 76 | g697708 | 2249 | 2675 |
| 76 | g3245902 | 2242 | 2672 |
| 76 | g3778060 | 2242 | 2672 |
| 76 | g3675008 | 2247 | 2672 |
| 76 | g3700953 | 2253 | 2680 |
| 76 | 70090551V1 | 2252 | 2654 |
| 76 | 70088450V1 | 2252 | 2654 |
| 76 | g4987033 | 2306 | 2666 |
| 76 | 7346532H1 | 2315 | 2669 |
| 76 | g1195921 | 2333 | 2667 |
| 76 | g3307378 | 2351 | 2673 |
| 76 | g1424265 | 2356 | 2666 |
| 76 | g1994824 | 2368 | 2666 |
| 76 | 70092273V1 | 2369 | 2666 |
| 76 | 70088685V1 | 2434 | 2683 |
| 76 | g1489884 | 2497 | 2667 |
| 76 | g4298447 | 2503 | 2666 |
| 76 | g1227527 | 2522 | 2669 |
| 76 | g2836709 | 2227 | 2666 |
| 76 | 404342H1 | 2229 | 2497 |
| 76 | 401524H1 | 2229 | 2445 |
| 76 | 402039H1 | 2229 | 2426 |
| 76 | 401524F1 | 2229 | 2666 |
| 76 | 70093923V1 | 2230 | 2654 |
| 76 | 6912204J1 | 189 | 491 |
| 76 | 6912204H1 | 191 | 490 |
| 76 | g1146598 | 386 | 763 |
| 76 | 7338712H1 | 426 | 1013 |
| 76 | 4951401H2 | 596 | 877 |
| 76 | 3225692H1 | 728 | 993 |
| 76 | 2133064F6 | 763 | 1210 |
| 76 | g2657310 | 41 | 563 |
| 76 | 2081047F6 | 1132 | 1650 |
| 76 | 2081047H1 | 1132 | 1375 |
| 76 | g2969649 | 1175 | 1525 |
| 76 | 873831H1 | 1198 | 1406 |
| 76 | 5187690H1 | 1219 | 1495 |
| 76 | 4089635H1 | 1236 | 1369 |
| 76 | 6596535H1 | 1272 | 1417 |
| 76 | 2581130F6 | 1301 | 1702 |
| 76 | 2581130H1 | 1301 | 1552 |
| 76 | g1994825 | 1382 | 1595 |
| 76 | 7040047H1 | 1449 | 1702 |
| 76 | 6630452U1 | 1468 | 1702 |
| 76 | 6630374U1 | 1578 | 2150 |
| 76 | 4143678H1 | 1593 | 1692 |
| 76 | g959892 | 1613 | 1702 |
| 76 | 6903811H1 | 1646 | 2243 |
| 76 | 4332838H1 | 1648 | 1702 |
| 76 | g697795 | 1774 | 2119 |
| 76 | g704969 | 1774 | 2043 |
| 76 | 2888581H1 | 1951 | 2236 |
| 76 | 70089240V1 | 1973 | 2539 |
| 76 | 70090145V1 | 1973 | 2674 |
| 76 | 70089170V1 | 1973 | 2538 |
| 76 | 70092550V1 | 1973 | 2489 |
| 76 | 70092490V1 | 1973 | 2499 |
| 76 | 70089927V1 | 1973 | 2437 |
| 76 | 70092932V1 | 1973 | 2447 |
| 76 | 70091424V1 | 1973 | 2516 |
| 76 | 70090074V1 | 1973 | 2480 |
| 76 | 70090511V1 | 1973 | 2426 |
| 76 | 70092562V1 | 1973 | 2452 |
| 76 | 70090451V1 | 1973 | 2487 |
| 76 | 70093155V1 | 1973 | 2333 |
| 76 | 70093381V1 | 1973 | 2335 |
| 76 | 2180264F6 | 1973 | 2319 |
| 76 | 70091927V1 | 1973 | 2286 |
| 76 | 2180264H1 | 1973 | 2143 |
| 76 | 70091191V1 | 1974 | 2497 |
| 76 | 2956706H1 | 1982 | 2065 |
| 76 | g1239260 | 1989 | 2120 |
| 76 | g3658694 | 1993 | 2448 |
| 76 | g2369409 | 1995 | 2221 |
| 76 | g1489980 | 1995 | 2193 |
| 76 | 2915268H1 | 1995 | 2189 |
| 76 | 5103731H1 | 1995 | 2143 |
| 76 | 5083588H1 | 1995 | 2127 |
| 76 | g2036913 | 2011 | 2270 |
| 76 | 1375031T1 | 2047 | 2289 |
| 76 | 70090620V1 | 2054 | 2455 |
| 76 | 1006083H1 | 2056 | 2329 |
| 76 | g1315008 | 2071 | 2546 |
| 76 | 70090041V1 | 2079 | 2597 |
| 76 | 2275715H1 | 2082 | 2355 |
| 76 | 70089944V1 | 2092 | 2664 |
| 76 | g1507094 | 2121 | 2366 |
| 76 | 2503051T6 | 2138 | 2254 |
| 76 | 2871614H1 | 2143 | 2443 |
| 76 | 2081047T6 | 2151 | 2289 |
| 76 | 1543664H1 | 2164 | 2375 |
| 76 | g3734766 | 2174 | 2672 |
| 76 | 2133064T6 | 2177 | 2289 |
| 76 | g4175843 | 2179 | 2669 |
| 76 | g2705950 | 2190 | 2669 |
| 76 | 70092616V1 | 2190 | 2680 |
| 76 | 6017144H1 | 2186 | 2462 |
| 76 | g1424317 | 2192 | 2665 |
| 76 | 70092373V1 | 2191 | 2664 |
| 76 | 70091541V1 | 2202 | 2654 |
| 76 | 920317H1 | 2208 | 2457 |
| 76 | 920309H1 | 2208 | 2455 |
| 76 | 70091726V1 | 2216 | 2666 |
| 76 | g1317327 | 2225 | 2673 |
| 76 | 70089181V1 | 2228 | 2655 |
| 76 | 4592652H1 | 2227 | 2372 |
| 77 | 5386182H1 | 2434 | 2533 |
| 77 | 5929032F6 | 2513 | 3092 |
| 77 | 5929032H1 | 2513 | 2807 |
| 77 | 5928821H1 | 2513 | 2602 |
| 77 | 2404117R6 | 2533 | 2936 |
| 77 | 2247162H1 | 2533 | 2802 |
| 77 | 5845721H1 | 2532 | 2686 |
| 77 | 2404117H1 | 2533 | 2717 |
| 77 | 6274929H2 | 2557 | 3085 |
| 77 | 4839303H1 | 2572 | 2867 |
| 77 | 70936812V1 | 2588 | 3163 |
| 77 | 6559464H1 | 2597 | 3142 |
| 77 | 2404117T6 | 2613 | 3183 |
| 77 | 2424729H1 | 2630 | 2886 |
| 77 | 6557735H1 | 2666 | 3250 |
| 77 | 70936842V1 | 2716 | 3372 |
| 77 | 1414767T6 | 2737 | 3276 |
| 77 | g5768528 | 2761 | 3225 |
| 77 | g1212489 | 2763 | 3057 |
| 77 | 659884H1 | 2764 | 3006 |
| 77 | 3331210T6 | 2779 | 3276 |
| 77 | 4942039H1 | 2778 | 3048 |
| 77 | 3331210F6 | 2781 | 3196 |
| 77 | 3331210H1 | 2781 | 2955 |
| 77 | 2043050H1 | 2878 | 3139 |
| 77 | g678229 | 2896 | 3222 |
| 77 | g4186860 | 2906 | 3322 |
| 77 | g561452 | 2927 | 3226 |
| 77 | g817283 | 2954 | 3237 |
| 77 | g1242796 | 3055 | 3317 |
| 77 | g678324 | 3120 | 3276 |
| 77 | g671188 | 3142 | 3276 |
| 77 | 5821129H1 | 3194 | 3328 |
| 77 | 1414767F6 | 1261 | 1693 |
| 77 | 70407644D1 | 901 | 1138 |
| 77 | 71042670V1 | 1006 | 1659 |
| 77 | 71039461V1 | 1032 | 1644 |
| 77 | 7290781H1 | 1077 | 1648 |
| 77 | 71039094V1 | 1110 | 1784 |
| 77 | 71039976V1 | 1157 | 1628 |
| 77 | 71040172V1 | 1160 | 1738 |
| 77 | 71042825V1 | 1179 | 1788 |
| 77 | 71042505V1 | 1191 | 1806 |
| 77 | 71041242V1 | 1224 | 1881 |
| 77 | 70938142V1 | 1261 | 1803 |
| 77 | g574829 | 1615 | 1881 |
| 77 | g767565 | 1616 | 1876 |
| 77 | 70937073V1 | 1646 | 2215 |
| 77 | 71041370V1 | 1653 | 2311 |
| 77 | g947001 | 1665 | 2012 |
| 77 | 3334355H1 | 1666 | 1945 |
| 77 | g705805 | 1704 | 1982 |
| 77 | 6763269H1 | 1740 | 2365 |
| 77 | 71041012V1 | 1745 | 2398 |
| 77 | 1254953H1 | 1759 | 1993 |
| 77 | 6507931H1 | 1798 | 2269 |
| 77 | 1833479R6 | 1771 | 2216 |
| 77 | 1833479H1 | 1771 | 2075 |
| 77 | 6271456H2 | 1777 | 2341 |
| 77 | 3332037H1 | 1779 | 2041 |
| 77 | 59241921H1 | 1784 | 2080 |
| 77 | 7292851H1 | 1788 | 2312 |
| 77 | 1833479T6 | 1796 | 2354 |
| 77 | 70935438V1 | 1825 | 2345 |
| 77 | 5205918H1 | 1835 | 2094 |
| 77 | 4710070H1 | 1851 | 2150 |
| 77 | 71040258V1 | 1855 | 2399 |
| 77 | 70936878V1 | 1868 | 2349 |
| 77 | g876649 | 1880 | 2293 |
| 77 | g570326 | 1880 | 2195 |
| 77 | g792017 | 1880 | 1970 |
| 77 | 7252042H1 | 1893 | 2490 |
| 77 | 5918594H1 | 1898 | 2195 |
| 77 | 4308614H1 | 1922 | 2266 |
| 77 | 5775902H1 | 1951 | 2547 |
| 77 | 900678T6 | 1952 | 2320 |
| 77 | 1267888F1 | 1965 | 2533 |
| 77 | 1267888H1 | 1965 | 2211 |
| 77 | g1991900 | 1988 | 2282 |
| 77 | g946386 | 2032 | 2371 |
| 77 | g891688 | 2079 | 2407 |
| 77 | 70947602V1 | 2088 | 2333 |
| 77 | 70947760V1 | 2095 | 2333 |
| 77 | 2524117H1 | 2129 | 2395 |
| 77 | g943716 | 2133 | 2371 |
| 77 | 70935414V1 | 2153 | 2809 |
| 77 | 70938201V1 | 2175 | 2740 |
| 77 | g953501 | 2230 | 2370 |
| 77 | 70937638V1 | 2264 | 2847 |
| 77 | 5207284H1 | 2273 | 2516 |
| 77 | g823406 | 2281 | 2415 |
| 77 | 6747059H1 | 2291 | 2882 |
| 77 | 5108466H1 | 2305 | 2395 |
| 77 | 750487H1 | 556 | 782 |
| 77 | 6983706H1 | 37 | 535 |
| 77 | g2000717 | 382 | 570 |
| 77 | 6855956H1 | 517 | 998 |
| 77 | g953502 | 1 | 212 |
| 77 | g2331234 | 1 | 2384 |
| 77 | 7359814H1 | 27 | 485 |
| 77 | 5351059H1 | 2335 | 2472 |
| 77 | 70942966V1 | 2341 | 2513 |
| 77 | 2152647H1 | 2383 | 2636 |
| 77 | 70941613V1 | 2429 | 2567 |
| 77 | 1414767H1 | 1261 | 1503 |
| 77 | g769023 | 1271 | 1589 |
| 77 | 71039996V1 | 1278 | 1834 |
| 77 | 71041640V1 | 1276 | 1859 |
| 77 | 661243H1 | 1311 | 1572 |
| 77 | 70845562V1 | 1318 | 1677 |
| 77 | g891480 | 1320 | 1516 |
| 77 | 71042682V1 | 1370 | 2012 |
| 77 | 6437672H1 | 1366 | 1900 |
| 77 | g705704 | 1384 | 1755 |
| 77 | 71040965V1 | 1418 | 1876 |
| 77 | 4695010H1 | 1441 | 1721 |
| 77 | 70938843V1 | 1447 | 1894 |
| 77 | 71041816V1 | 1463 | 2053 |
| 77 | 71040541V1 | 1463 | 2021 |
| 77 | 4178826H1 | 1494 | 1762 |
| 77 | 2525545H1 | 1496 | 1726 |
| 77 | 70936642V1 | 1550 | 2202 |
| 77 | 71243429V1 | 1557 | 1786 |
| 77 | 6124654H1 | 1590 | 2086 |
| 77 | 6560433H1 | 1614 | 2187 |
| 77 | g389618 | 1615 | 1914 |
| 77 | 960213H1 | 814 | 1101 |
| 77 | g774455 | 786 | 1139 |
| 77 | 962582R6 | 795 | 1260 |
| 77 | 962582H1 | 795 | 849 |
| 77 | g879553 | 796 | 1117 |
| 77 | 4028250H1 | 574 | 833 |
| 77 | g572875 | 750 | 1009 |
| 78 | 1504601F6 | 383 | 747 |
| 78 | 1258447H1 | 1616 | 1755 |
| 78 | 71225337V1 | 1618 | 2089 |
| 78 | 982079R6 | 2378 | 2741 |
| 78 | 982079H1 | 2378 | 2691 |
| 78 | 982079T6 | 2378 | 2744 |
| 78 | 4859367H1 | 1503 | 1670 |
| 78 | g1056444 | 1527 | 1812 |
| 78 | 2881063H1 | 1478 | 1786 |
| 78 | 4546751H1 | 1485 | 1761 |
| 78 | 6219786H1 | 2241 | 2558 |
| 78 | 5044584H1 | 1072 | 1352 |
| 78 | 5731888H1 | 1086 | 1332 |
| 78 | 2668983H1 | 1013 | 1254 |
| 78 | 5869109H1 | 1423 | 1674 |
| 78 | 4175935F6 | 1476 | 2104 |
| 78 | 71287876V1 | 1476 | 2061 |
| 78 | 71286863V1 | 1476 | 2009 |
| 78 | 70995743V1 | 1476 | 1941 |
| 78 | 4175935H1 | 1476 | 1769 |
| 78 | 70175363V1 | 1478 | 2039 |
| 78 | 2881063F6 | 1478 | 1993 |
| 78 | 1405756H1 | 1101 | 1358 |
| 78 | 6212807H1 | 1168 | 1473 |
| 78 | 6789736H1 | 1286 | 1749 |
| 78 | g866161 | 2297 | 2612 |
| 78 | 4933985H1 | 2263 | 2430 |
| 78 | g865342 | 2297 | 2641 |
| 78 | 70961841V1 | 2250 | 2783 |
| 78 | 6843570H1 | 124 | 268 |
| 78 | 5447930H2 | 212 | 443 |
| 78 | 6819333H1 | 375 | 837 |
| 78 | 063360H1 | 575 | 793 |
| 78 | 2717804H1 | 736 | 995 |
| 78 | 1336510H1 | 542 | 798 |
| 78 | 1336565H1 | 542 | 794 |
| 78 | 2260777H1 | 522 | 781 |
| 78 | 6819333J1 | 525 | 1141 |
| 78 | 1504601H1 | 383 | 641 |
| 78 | 6935560H1 | 497 | 997 |
| 78 | 2260777R6 | 522 | 929 |
| 78 | 2417475H1 | 1999 | 2100 |
| 78 | 70858537V1 | 2011 | 2620 |
| 78 | 70962120V1 | 2020 | 2649 |
| 78 | g5595178 | 2416 | 2791 |
| 78 | g2569464 | 2421 | 2792 |
| 78 | g2848983 | 2422 | 2790 |
| 78 | g1507026 | 2424 | 2792 |
| 78 | g1645469 | 2491 | 2786 |
| 78 | 70962041V1 | 2226 | 2750 |
| 78 | 2805557T6 | 2240 | 2753 |
| 78 | 6307990H1 | 2241 | 2695 |
| 78 | g3701921 | 2399 | 2789 |
| 78 | 70856260V1 | 1659 | 2200 |
| 78 | 71287735V1 | 1680 | 2356 |
| 78 | 71226071V1 | 1619 | 2077 |
| 78 | 70960213V1 | 1653 | 2205 |
| 78 | 1258447F6 | 1616 | 2083 |
| 78 | 5886340H1 | 1960 | 2108 |
| 78 | 5880788H1 | 1959 | 2089 |
| 78 | 5883358H1 | 1960 | 2230 |
| 78 | 5882287H1 | 1960 | 2207 |
| 78 | 70996202V1 | 1968 | 2079 |
| 78 | 5884261H1 | 1959 | 2100 |
| 78 | 5886308H1 | 1959 | 2238 |
| 78 | 70172850V1 | 1547 | 2052 |
| 78 | 70172946V1 | 1554 | 1896 |
| 78 | 5109345H1 | 1773 | 1894 |
| 78 | 70858458V1 | 1829 | 2390 |
| 78 | 70861427V1 | 1872 | 2034 |
| 78 | g1645468 | 2153 | 2569 |
| 78 | 70857537V1 | 2132 | 2748 |
| 78 | 70961205V1 | 2134 | 2784 |
| 78 | 6465155H1 | 817 | 1387 |
| 78 | 3737993H1 | 865 | 1101 |
| 78 | 2805557F6 | 888 | 1348 |
| 78 | 2805557H1 | 888 | 1199 |
| 78 | 7166418H1 | 1 | 544 |
| 78 | 6845517H1 | 18 | 576 |
| 78 | 7216754H1 | 109 | 637 |
| 78 | g3895948 | 2405 | 2789 |
| 78 | g1506843 | 2407 | 2792 |
| 78 | g3934604 | 2413 | 2783 |
| 78 | 71362378V1 | 1586 | 1776 |
| 78 | 71288334V1 | 1613 | 2200 |
| 78 | 71288102V1 | 2182 | 2787 |
| 78 | 70858569V1 | 2361 | 2790 |
| 78 | 981508H1 | 2378 | 2661 |
| 78 | g3280794 | 2348 | 2786 |
| 78 | g4900509 | 2359 | 2783 |
| 78 | 858094H1 | 2047 | 2315 |
| 78 | 71288239V1 | 2117 | 2721 |
| 78 | 2056939R6 | 2154 | 2463 |
| 78 | 2056939H1 | 2154 | 2430 |
| 78 | 1641318H1 | 2168 | 2384 |
| 78 | 1298674H1 | 2174 | 2448 |
| 78 | 1298674F1 | 2174 | 2342 |
| 78 | 70172181V1 | 1769 | 2279 |
| 78 | 56995561H1 | 1678 | 1913 |
| 78 | 7027634H1 | 1695 | 1971 |
| 78 | 71225035V1 | 1707 | 2313 |
| 78 | 71225177V1 | 1746 | 2398 |
| 78 | 70961047V1 | 1745 | 2324 |
| 78 | 1692282F6 | 1749 | 2316 |
| 78 | 1692282H1 | 1749 | 1985 |
| 78 | 1305007H1 | 1754 | 2002 |
| 78 | 5887009H1 | 1958 | 2240 |
| 78 | 5889943H1 | 1958 | 2243 |
| 78 | 5888977H1 | 1958 | 2176 |
| 78 | 5884968H1 | 1959 | 2101 |
| 78 | g2347469 | 1895 | 2202 |
| 78 | 2727193H1 | 1942 | 2100 |
| 78 | 2083456H1 | 1955 | 2102 |
| 78 | 70856801V1 | 1881 | 2488 |
| 78 | 2408695H1 | 2219 | 2334 |
| 78 | 1504601T6 | 2211 | 2755 |
| 78 | 1692282T6 | 2211 | 2740 |
| 78 | 71287317V1 | 2192 | 2816 |
| 78 | 6376946H1 | 2205 | 2491 |
| 78 | 3408549H1 | 2215 | 2526 |
| 78 | 2461537H1 | 982 | 1199 |
| 78 | 2056939T6 | 2523 | 2754 |
| 78 | 3819114H1 | 2568 | 2764 |
| 78 | 767808H1 | 2576 | 2783 |
| 78 | g3871569 | 2579 | 2783 |
| 78 | g5638296 | 2585 | 2789 |
| 78 | g2969829 | 2618 | 2783 |
| 78 | 6368851H1 | 2678 | 2783 |
| 79 | 4404143T6 | 1 | 604 |
| 79 | 3566814H1 | 199 | 382 |
| 79 | 5639354R6 | 465 | 918 |

TABLE 3

| SEQ | |
|---|---|
| ID NO: | Tissue Distribution |
| 1 | Unclassified/Mixed - 71%, Endocrine System - 16% |
| 2 | Embryonic Structures - 82%, Nervous System - 18% |
| 3 | Germ Cells - 62%, Connective Tissue - 17%, Unclassified/Mixed - 16% |
| 4 | Endocrine System - 71%, Nervous System - 29% |
| 5 | Nervous System - 76%, Endocrine System - 18% |
| 6 | Endocrine System - 63%, Liver - 23% |
| 7 | Endocrine System - 29%, Hemic and Immune System - 24%, Exocrine |
| | Glands - 19% |
| 8 | Endocrine System - 42%, Germ Cells - 19% |
| 9 | Endocrine System - 66%, Sense Organs - 21%, Nervous System - 11% |
| 10 | Germ Cells - 30%, Sense Organs - 24%, Nervous System - 17% |
| 11 | Nervous System - 100% |
| 12 | Embryonic Structures - 18%, Pancreas - 17%, Hemic and Immune |
| | System - 17% |
| 13 | Exocrine Glands - 42%, Cardiovascular System - 21%, Respiratory |
| | System - 16% |
| 14 | Nervous System - 86% |
| 15 | Unclassified/Mixed - 17%, Sense Organs - 12% |
| 16 | Unclassified/Mixed - 28%, Embryonic Structures - 20%, Liver - 16% |
| 17 | Endocrine System - 100% |
| 18 | Digestive System - 57%, Hemic and Immune System - 29%, Nervous |
| | System - 14% |
| 19 | Unclassified/Mixed - 90% |
| 20 | Exocrine Glands - 50%, Respiratory System - 38%, Nervous System - 13% |
| 21 | Unclassified/Mixed - 20%, Connective Tissue - 10% |
| 22 | Sense Organs - 17% |
| 23 | Female Genitalia - 32%, Hemic and Immune System - 24%, Endocrine |
| | System - 20% |
| 24 | Sense Organs - 15%, Embryonic Structures - 13% |
| 25 | Sense Organs - 43%, Unclassified/Mixed - 11% |
| 26 | Unclassified/Mixed - 16%, Embryonic Structures - 13%, Germ Cells - 13% |
| 27 | Respiratory System - 43%, Nervous System - 21%, Female Genitalia - 21% |
| 28 | Liver - 33%, Germ Cells - 12% |
| 29 | Endocrine System - 31%, Germ Cells - 25%, Liver - 12% |
| 30 | Exocrine Glands - 29%, Germ Cells - 28% |
| 31 | Exocrine Glands - 18%, Cardiovascular System - 10% |
| 32 | Sense Organs - 23%, Embryonic Structures - 15%, Endocrine System - 15% |
| 33 | Sense Organs - 17%, Endocrine System 11%, Skin - 10% |
| 34 | Musculoskeletal System - 86% |
| 35 | Connective Tissue - 16%, Embryonic Structures - 14%, Digestive |
| | System - 11% |
| 36 | Pancreas - 40%, Female Genitalia - 20%, Cardiovascular System - 16% |
| 37 | Respiratory System - 12%, Urinary Tract - 12% |
| 38 | Embryonic Structures - 53%, Digestive System - 17%, Nervous System - 12% |
| 39 | Digestive System - 40%, Nervous System - 32%, Nervous System - 28% |
| 40 | Exocrine Glands - 47%, Sense Organs - 21% |
| 41 | Connective Tissue - 60%, Unclassified/Mixed - 22% |
| 42 | Hemic and Immune System - 15% |
| 43 | Sense Organs - 21%, Female Genitalia - 13% |
| 44 | Hemic and Immune System - 18%, Female Genitalia - 17% |
| 45 | Embryonic Structures - 32%, Female Genitalia - 17% |
| 46 | Male Genitalia - 49%, Skin - 32% |
| 47 | Nervous System - 42%, Nervous System - 40%, Endocrine System - 14% |
| 48 | Digestive System - 41%, Nervous System - 15%, Liver - 14% |
| 49 | Exocrine Glands - 34%, Unclassified/Mixed - 22% |
| 50 | Nervous System - 57%, Unclassified/Mixed - 11% |
| 51 | Nervous System - 52%, Digestive System - 25%, Connective Tissue - 10% |
| 52 | Digestive System - 24%, Germ Cells - 22%, Exocrine Glands - 11% |
| 53 | Endocrine System - 32%, Digestive System - 19% |
| 54 | Embryonic Structures - 58%, Nervous System - 37% |
| 55 | Female Genitalia - 18%, Nervous System - 18%, Embryonic Structures - 18% |
| 56 | Connective Tissue - 29%, Germ Cells - 22%, Liver - 13% |
| 57 | Musculoskeletal System - 15%, Embryonic Structures - 11% |
| 58 | Stomatognathic System - 27%, Urinary Tract - 11% |
| 59 | Sense Organs - 25%, Endocrine System - 16% |
| 60 | Exocrine Glands - 33%, Urinary Tract - 27%, Nervous System - 14% |
| 61 | Endocrine System - 16%, Musculoskeletal System - 13% |
| 62 | Exocrine Glands - 27%, Skin - 15%, Female Genitalia - 15% |
| 63 | Female Genitalia - 36%, Urinary Tract - 22%, Digestive System - 22% |
| 64 | Germ Cells - 16%, Endocrine System - 11%, Liver - 11% |
| 65 | Pancreas - 24%, Unclassified/Mixed - 21%, Male Genitalia - 17% |
| 66 | Endocrine System - 43%, Embryonic Structures - 14%, |
| | Unclassified/Mixed - 12% |
| 67 | Embryonic Structures - 42%, Male Genitalia - 19%, Female Genitalia - 19% |
| 68 | Male Genitalia - 67%, Nervous System - 33% |
| 69 | Urinary Tract - 50%, Endocrine System - 18% |
| 70 | Respiratory System - 100% |
| 71 | Skin - 43%, Nervous System - 19%, Nervous System - 19% |
| 72 | Cardiovascular System - 35%, Nervous System - 27%, Male Genitalia - 19% |
| 73 | Female Genitalia - 12%, Germ Cells - 11% |
| 74 | Male Genitalia - 28% |
| 75 | Embryonic Structures - 30%, Urinary Tract - 22%, Respiratory |
| | System - 16% |
| 76 | Musculoskeletal System - 16%, Respiratory System - 12% |
| 77 | Nervous System - 35%, Male Genitalia - 31%, Germ Cells - 11% |
| 78 | Stomatognathic System - 19%, Liver - 12%, Exocrine Glands - 11% |
| 79 | Respiratory System - 38%, Female Genitalia - 38%, Male Genitalia - 25% |

TABLE 4

| Program | Description | Reference | Parameter Threshold |
|---|---|---|---|
| ABI | A program that removes vector sequences and masks | Applied Biosystems, Foster City, CA. | |
| FACTURA | ambiguous bases in nucleic acid sequences. | | |
| ABI/ | A Fast Data Finder useful in comparing and annotating | Applied Biosystems, Foster City, CA; | Mismatch <50% |
| PARACEL | amino acid or nucleic acid sequences. | Paracel Inc., Pasadena, CA. | |
| FDF | | | |
| ABI | A program that assembles nucleic acid sequences. | Applied Biosystems, Foster City, CA. | |
| Auto- | | | |
| Assembler | | | |
| BLAST | A Basic Local Alignment Search Tool useful in sequence | Altschul, S. F. et al. (1990) J. Mol. Biol. | ESTs: Probability |
| | similarity search for amino acid and nucleic acid | 215: 403-410; Altschul, S. F. et al. (1997) | value = 1.0E−8 |
| | sequences. BLAST includes five functions: blastp, blastn, | Nucleic Acids Res. 25: 3389-3402. | or less Full Length |
| | blastx, tblastn, and tblastx. | | sequences: Probability |
| | | | value = 1.0E−10 |
| | | | or less |
| FASTA | A Pearson and Lipman algorithm that searches for | Pearson, W. R. and D. J. Lipman (1988) Proc. | ESTs: fasta E |
| | similarity between a query sequence and a group of | Natl Acad Sci. USA 85: 2444-2448; Pearson, | value = 1.06E−6 |
| | sequences of the same type. FASTA comprises as least | W. R. (1990) Methods Enzymol. 183: 63-98; | Assembled ESTs: |
| | five functions: fasta, tfasta, fastx, tfastx, and ssearch. | and Smith, T. F. and M. S. Waterman (1981) | fasta Identity = 95% |
| | | Adv. Appl. Math. 2: 482-489. | or greater and Match |
| | | | length = 200 |
| | | | bases or greater; |
| | | | fastx E value = |
| | | | 1.0E−8 or less |
| | | | Full Length sequences: |
| | | | fastx score = 100 |
| | | | or greater |
| BLIMPS | A BLocks IMProved Searcher that matches a sequence | Henikoff, S. and J. G. Henikoff (1991) Nucleic | Probability value = |
| | against those in BLOCKS, PRINTS, DOMO, PRODOM, | Acids Res. 19: 6565-6572; Henikoff, J. G. and | 1.0E−3 or less |
| | and PFAM databases to search for gene families, | S. Henikoff (1996) Methods Enzymol. | |
| | sequence homology, and structural | 266: 88-105; and Attwood, T. K. et al. (1997) .J. | |
| | fingerprint regions. | Chem. Inf. Comput. Sci. 37: 417-424. | |
| HMMER | An algorithm for searching a query sequence against | Krogh, A. et al. (1994) J. Mol. Biol., | PFAM hits: |
| | hidden Markov model (HMM)-based databases of protein | 235: 1501-1531; Sonnhammer, E. L. L. et al. | Probability value = |
| | family consensus sequences, such as PFAM. | (1988) Nucleic Acids Res. 26: 320-322; | 1.0E−3 or less Signal |
| | | Durbin, R et al. (1998) Our World View, in a | peptide hits: Score = |
| | | Nutshell, Cambridge Univ. Press, pp. 1-350. | 0 or greater |
| ProfileScan | An algorithm that searches for structural and sequence | Gribskov, M. et al. (1988) CABIOS 4: 61-66; | Normalized quality |
| | motifs in protein sequences that match sequence patterns | Gribskov, M. et al. (1989) Methods Enzymol. | score ≧ GCG- |
| | defined in Prosite. | 183: 146-159; Bairoch, A. et al. (1997) Nucleic | specified “HIGH” |
| | | Acids Res. 25: 217-221. | value for that |
| | | | particular Prosite |
| | | | motif. Generally, |
| | | | score = 1.4−2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. Appl. | Score = 120 |
| | CrossMatch, programs based on efficient implementation | Math. 2: 482-489; Smith, T. F. and M. S. | or greater; Match |
| | of the Smith-Waterman algorithm, useful in searching | Waterman (1981) J. Mol. Biol. 147: 195-197; and | length = 56 |
| | sequence homology and assembling DNA sequences. | Green, P., University of Washington, | or greater |
| | | Seattle, WA. | |
| Consed | A graphical tool for viewing and editing | Gordon, D. et al. (1998) Genome Res. 8: 195-202. | |
| | Phrap assemblies. | | |
| SPScan | A weight matrix analysis program that scans protein | Nielson, H. et al. (1997) Protein Engineering 10: 1- | Score = 3.5 |
| | sequences for the presence of secretory signal peptides. | 6; Claverie, J. M. and S. Audic (1997) CABIOS | or greater |
| | | 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. (1998) Proc. Sixth Intl. | |
| | delineate transmembrane segments on protein sequences | Conf. on Intelligent Systems for Mol. Biol., | |
| | and determine orientation. | Glasgow et al., eds., The Am. Assoc. for Artificial | |
| | | Intelligence Press, Menlo Park, CA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program Manual, | |
| | | version 9, page M51-59, Genetics Computer | |
| | | Group, Madison, WI. | |

What is claimed is:

1. An isolated polynucleotide comprising a polynucleotide sequence selected from the group consisting of:
a) a polynucleotide sequence selected from the group consisting of SEQ ID NO: 1-79,
b) a naturally occurring polynucleotide sequence having at least 90% sequence identity to a polynucleotide sequence selected from the group consisting of SEQ ID NO: 1-79,
c) a polynucleotide sequence complementary to a),
d) a polynucleotide sequence complementary to b), and
e) an RNA equivalent of a) through d).

2. An isolated polynucleotide of claim 1, comprising a polynucleotide sequence selected from the group consisting of SEQ ID NO: 1-79.

3. An isolated polynucleotide comprising at least 60 contiguous nucleotides of a polynucleotide of claim 1.

4. A composition for the detection of expression of secretory polynucleotides comprising at least one of the polynucleotides of claim 1 and a detectable label.

5. A method for detecting a target polynucleotide in a sample, said target polynucleotide having a sequence of a polynucleotide of claim 1, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

6. A method for detecting a target polynucleotide in a sample, said target polynucleotide comprising a sequence of a polynucleotide of claim 1, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

7. A method of claim 5, wherein the probe comprises at least 30 contiguous nucleotides.

8. A method of claim 5, wherein the probe comprises at least 60 contiguous nucleotides.

9. A recombinant polynucleotide comprising a promoter sequence operably linked to a polynucleotide of claim 1.

10. A cell transformed with a recombinant polynucleotide of claim 9.

11. A transgenic organism comprising a recombinant polynucleotide of claim 9.

12. A method for producing a secretory polypeptide, the method comprising:
a) culturing a cell under conditions suitable for expression of the secretory polypeptide, wherein said cell is transformed with a recombinant polynucleotide of claim 9, and
b) recovering the secretory polypeptide so expressed.

13. A purified secretory polypeptide (SPTM) encoded by at least one of the polynucleotides of claim 2.

14. An isolated antibody which specifically binds to a secretory polypeptide of claim 13.

15. A method of identifying a test compound which specifically binds to the secretory polypeptide of claim 13, the method comprising the steps of:
a) providing a test compound;
b) combining the secretory polypeptide with the test compound for a sufficient time and under suitable conditions for binding; and
c) detecting binding of the secretory polypeptide to the test compound, thereby identifying the test compound which specifically binds the secretory polypeptide.

16. A microarray wherein at least one element of the microarray is a polynucleotide of claim 3.

17. A method for generating a transcript image of a sample which contains polynucleotides, the method comprising the steps of:
a) labeling the polynucleotides of the sample,
b) contacting the elements of the microarray of claim 16 with the labeled polynucleotides of the sample under conditions suitable for the formation of a hybridization complex, and
c) quantifying the expression of the polynucleotides in the sample.

18. A method for screening a compound for effectiveness in altering expression of a target polynucleotide, wherein said target polynucleotide comprises a polynucleotide sequence of claim 1, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of is the compound and in the absence of the compound.

19. A method for assessing toxicity of a test compound, said method comprising:
a) treating a biological sample containing nucleic acids with the test compound;
b) hybridizing the nucleic acids of the treated biological sample with a probe comprising at least 20 contiguous nucleotides of a polynucleotide of claim 1 under conditions whereby a specific hybridization complex is formed between said probe and a target polynucleotide in the biological sample, said target polynucleotide comprising a polynucleotide sequence of a polynucleotide of claim 1 or fragment thereof;
c) quantifying the amount of hybridization complex; and
d) comparing the amount of hybridization complex in the treated biological sample with the amount of hybridization complex in an untreated biological sample, wherein a difference in the amount of hybridization complex in the treated biological sample is indicative of toxicity of the test compound.

20. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, said target polynucleotide having a sequence of claim 1.

21. An array of claim 20, wherein said first oligonucleotide or polynucleotide sequence is completely complementary to at least 30 contiguous nucleotides of said target polynucleotide.

22. An array of claim 20, wherein said first oligonucleotide or polynucleotide sequence is completely complementary to at least 60 contiguous nucleotides of said target polynucleotide

23. An array of claim 20, which is a microarray.

24. An array of claim 20, further comprising said target polynucleotide hybridized to said first oligonucleotide or polynucleotide.

25. An array of claim 20, wherein a linker joins at least one of said nucleotide molecules to said solid substrate.

26. An array of claim 20, wherein each distinct physical location on the substrate contains multiple nucleotide molecules having the same sequence, and each distinct physical location on the substrate contains nucleotide molecules having a sequence which differs from the sequence of nucleotide molecules at another physical location on the substrate.
